(19) United States
(12) Patent Application Publication
(10) Pub. No.: US 2026/0062385 A1
(43) Pub. Date: Mar. 5, 2026

(54) ORGANIC COMPOUND

(71) Applicant: SEMICONDUCTOR ENERGY LABORATORY CO., LTD., Atsugi-shi (JP)

(72) Inventors: Takuya HARUYAMA, Isehara (JP); Yui YOSHIYASU, Atsugi (JP); Shinya FUKUZAKI, Atsugi (JP); Nobuharu OHSAWA, Zama (JP)

(21) Appl. No.: 19/295,812

(22) Filed: Aug. 11, 2025

(30) Foreign Application Priority Data
Aug. 30, 2024 (JP) 2024-148422

(51) Int. Cl. C07D 213/74; C07B 59/00; C07D 213/69; C07D 213/70; C07D 213/85; C07D 401/14; C07D 405/14; C07D 519/00; C07F 7/08; H10K 50/11; H10K 85/40; H10K 85/60

(52) U.S. Cl. CPC C07D 213/74; C07B 59/002; C07D 213/69; C07D 213/70; C07D 213/85; C07D 401/14; C07D 405/14; C07D 519/00; C07F 7/0812; C07F 7/0814; H10K 85/40; H10K 85/633; H10K 85/636; H10K 50/11; H10K 85/615; H10K 85/654; H10K 85/6572

(57) ABSTRACT

To provide a novel organic compound and a light-emitting device including the organic compound. In General Formula (G1-1), Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1); and at least one of R¹ to R³ and at least one of R⁴ to R⁶ each represent a group represented by General Formula (g2).

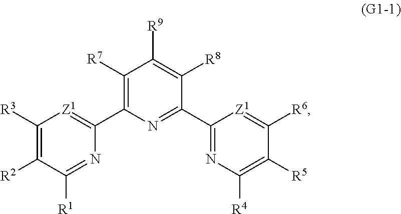

### BACKGROUND OF THE INVENTION

### 1. Field of the Invention

One embodiment of the present invention relates to an organic compound, an organic semiconductor element, a light-emitting device, a photodiode sensor, a display module, a lighting module, a display device, an electronic appliance, a lighting device, and an electronic device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Thus, specific examples of the technical field of one embodiment of the present invention disclosed in this specification include a semiconductor device, a display device, a liquid crystal display device, a lighting device, a power storage device, a memory device, an image capturing device, a driving method thereof, and a manufacturing method thereof.

### 2. Description of the Related Art

A light-emitting device (also referred to as an organic EL element) including an organic compound that is a light-emitting substance between a pair of electrodes has characteristics such as being thin and light in weight, high-speed response, and low voltage driving. Thus, displays including such light-emitting devices have been developed.

Since light-emitting layers of such light-emitting devices can be formed two-dimensionally as continuous planar layers, planar light emission can be achieved. This feature is difficult to achieve with point light sources typified by incandescent lamps and LEDs or linear light sources typified by fluorescent lamps; thus, the light-emitting devices also have great potential as planar light sources which can be used for lighting devices and the like.

For example, a functional panel in which a pixel provided in a display region includes a light-emitting element (light-emitting device) and a photoelectric conversion element (light-receiving device) is known (Patent Document 1).

Although displays or lighting devices including light-emitting devices can be suitably used for a variety of electronic appliances as described above, their performance and cost competitiveness have plenty of room to improve. Therefore, a material that is more excellent in characteristics and easier to handle and an easy method for synthesizing the material are required.

Although the characteristics of light-emitting devices have been improved considerably, advanced requirements for various characteristics including efficiency and durability are not yet satisfied. In particular, to solve a problem such as burn-in, which is an issue peculiar to EL, it is preferable to inhibit a reduction in efficiency due to deterioration as much as possible.

Deterioration largely depends on an emission center substance and its surrounding materials; therefore, organic compound materials having favorable characteristics have been actively developed.

### REFERENCE

[Patent Document 1] PCT International Publication No. WO2020/152556

### SUMMARY OF THE INVENTION

An object of one embodiment of the present invention is to provide a novel organic compound. Another object of one embodiment of the present invention is to provide a method for synthesizing a novel organic compound. Another object of one embodiment of the present invention is to provide an organic compound that can be used for a light-emitting device. Another object of one embodiment of the present invention is to provide a novel organic compound that can be used for an intermediate layer of a tandem light-emitting device. Another object of one embodiment of the present invention is to provide a light-emitting device having high emission efficiency. Another object of one embodiment of the present invention is to provide a high-color-purity light-emitting device. Another object of one embodiment of the present invention is to provide a highly reliable light-emitting device. Another object of one embodiment of the present invention is to provide any of a low-power-consumption display device, a low-power-consumption electronic appliance, and a low-power-consumption lighting device. Another object of one embodiment of the present invention is to provide any of a highly reliable display device, a highly reliable electronic appliance, and a highly reliable lighting device. Another object of one embodiment of the present invention is to provide any of a high-color-purity display device, a high-color-purity electronic appliance, and a high-color-purity lighting device.

The present invention achieves at least one of the above-described objects.

One embodiment of the present invention is an organic compound represented by General Formula (G1-1).

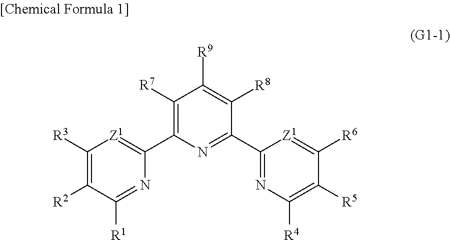

In General Formula (G1-1), Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g2) below.

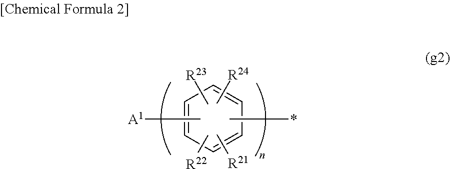

In General Formula (g2) above, A¹ represents the aliphatic amine group represented by General Formula (g1) below; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G1-1).

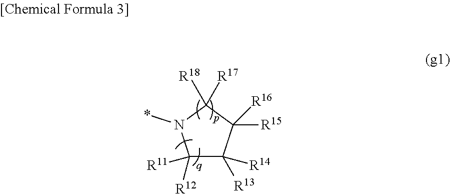

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G1-1).

One embodiment of the present invention is an organic compound represented by General Formula (G1-2).

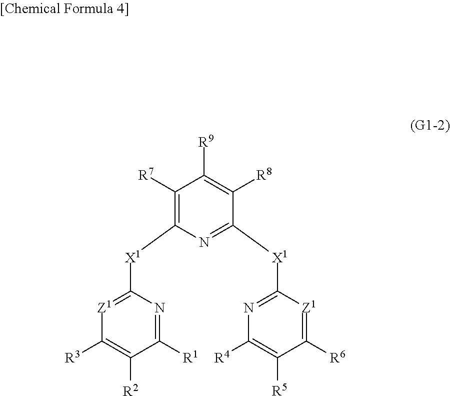

In General Formula (G1-2), X¹ represents oxygen, sulfur, or nitrogen; when X¹ represents nitrogen, the nitrogen has, as a substituent, a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g2) below.

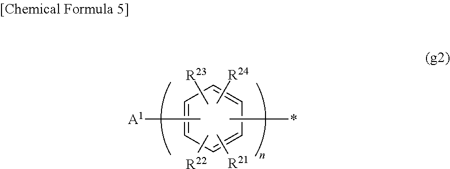

In General Formula (g2) above, A¹ represents the aliphatic amine group represented by General Formula (g1) below; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G1-2).

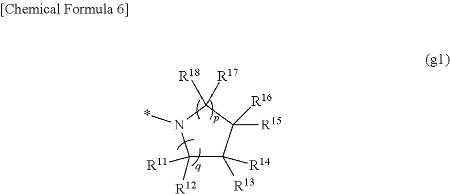

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G1-2).

One embodiment of the present invention is an organic compound represented by General Formula (G1-3).

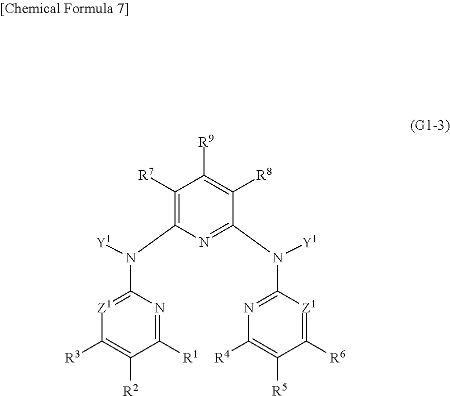

In General Formula (G1-3), Y¹ represents a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g2) below.

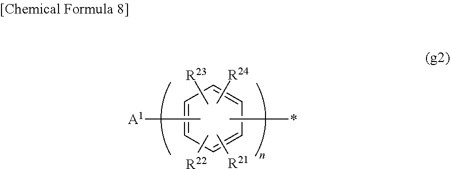

In General Formula (g2) above, A¹ represents the aliphatic amine group represented by General Formula (g1) below; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G1-3).

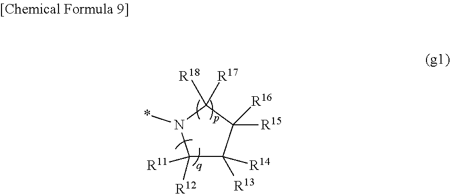

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G1-3).

One embodiment of the present invention is an organic compound represented by General Formula (G2-1).

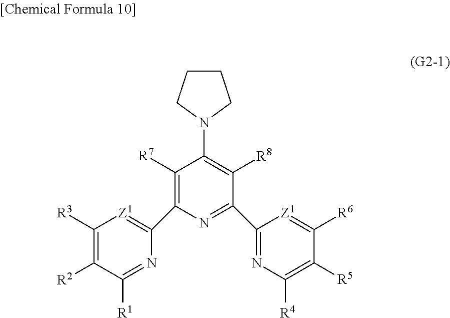

In General Formula (G2-1), Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g3) below.

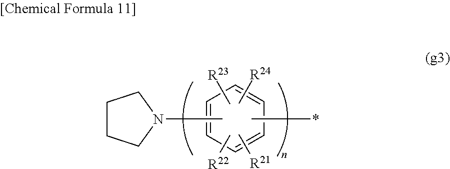

In General Formula (g3) above, each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G2-1).

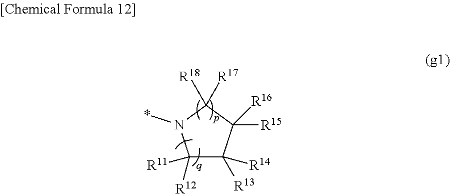

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G2-1).

One embodiment of the present invention is an organic compound represented by General Formula (G2-2).

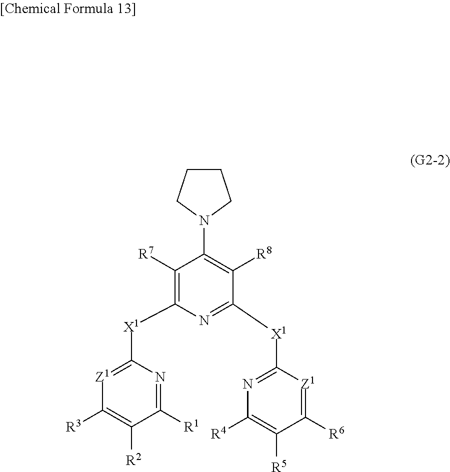

In General Formula (G2-2), X¹ represents oxygen, sulfur, or nitrogen; when X¹ represents nitrogen, the nitrogen has, as a substituent, a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g3) below.

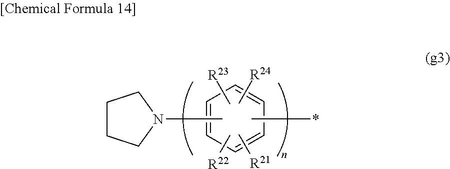

In General Formula (g3) above, each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G2-2).

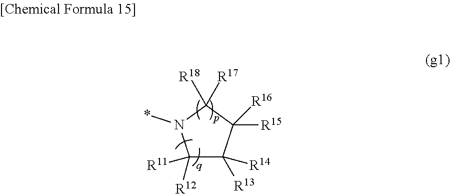

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G2-2).

One embodiment of the present invention is an organic compound represented by General Formula (G2-3).

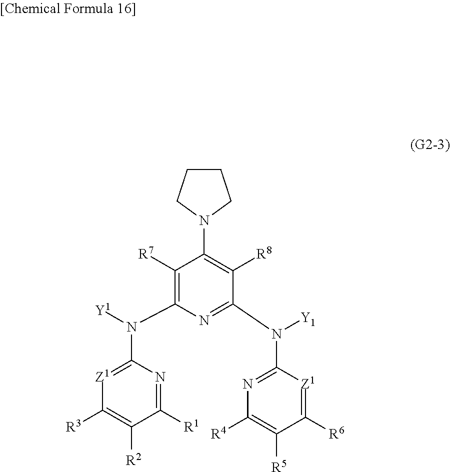

In General Formula (G2-3), Y¹ represents a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g3) below.

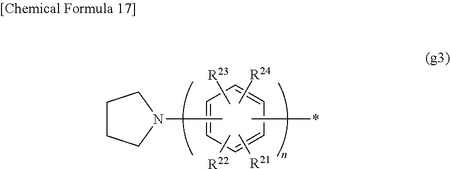

In General Formula (g3) above, each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; n represents an integer of 0 to 2; and an asterisk (*) represents a bond in the organic compound represented by General Formula (G2-3).

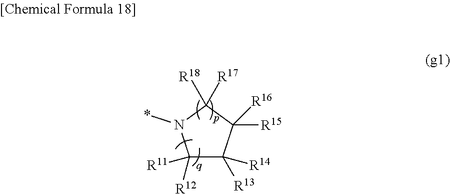

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G2-3).

One embodiment of the present invention is an organic compound represented by General Formula (G3).

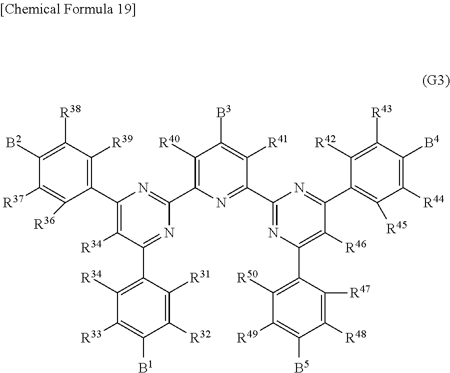

In General Formula (G3), each of B¹ to B⁵ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; at least one of B¹ to B⁵ represents the aliphatic amine group represented by General Formula (g1) below; and each of R³¹ to R⁵⁰ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below.

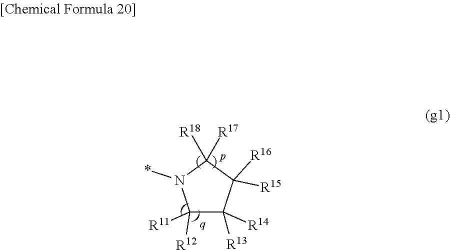

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G3).

One embodiment of the present invention is an organic compound represented by General Formula (G4).

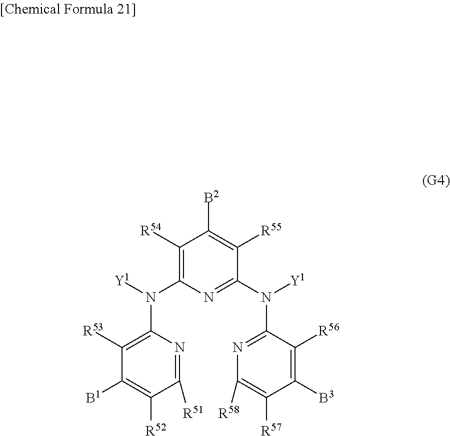

In General Formula (G4), Y¹ represents a substituted or unsubstituted aryl group having 6 to 30 carbon atoms or a substituted or unsubstituted heteroaryl group having 1 to 30 carbon atoms; each of B¹ to B³ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; at least one of B¹ to B³ represents the aliphatic amine group represented by General Formula (g1) below; and each of R⁵¹ to R⁵⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below.

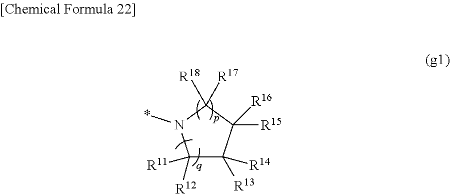

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G4).

One embodiment of the present invention is an organic compound represented by General Formula (G5).

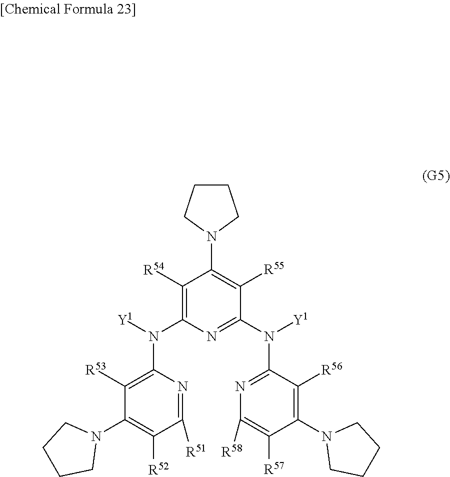

In General Formula (G5), Y¹ represents a substituted or unsubstituted aryl group having 6 to 30 carbon atoms or a substituted or unsubstituted heteroaryl group having 1 to 30 carbon atoms; and each of R⁵¹ to R⁵⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below.

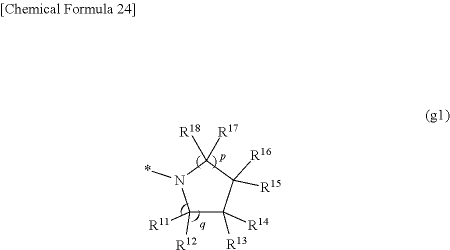

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; any two of R¹¹ to R¹⁸ bonded to different carbon atoms may be bonded to each other to form a ring; and an asterisk (*) represents a bond in the organic compound represented by General Formula (G5).

One embodiment of the present invention is an organic compound represented by any one of Structural Formulae (100) to (102).

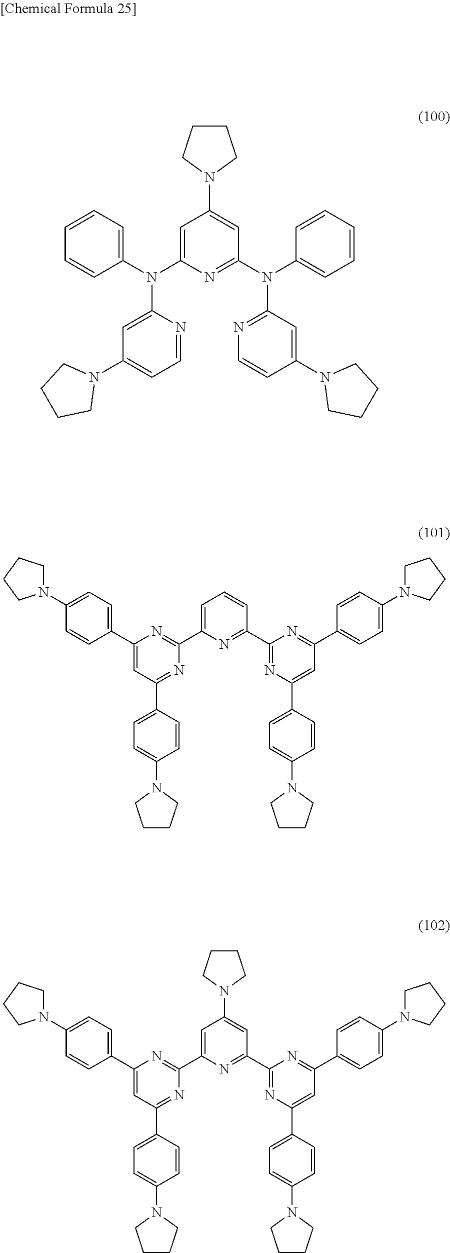 [SMILES: c1ccc(N(c2cc(N3CCCC3)ccn2)c2cc(N3CCCC3)cc(N(c3ccccc3)c3cc(N4CCCC4)ccn3)n2)cc1 ; c1cc(-c2nc(-c3ccc(N4CCCC4)cc3)cc(-c3ccc(N4CCCC4)cc3)n2)nc(-c2nc(-c3ccc(N4CCCC4)cc3)cc(-c3ccc(N4CCCC4)cc3)n2)c1 ; c1cc(N2CCCC2)ccc1-c1cc(-c2ccc(N3CCCC3)cc2)nc(-c2cc(N3CCCC3)cc(-c3nc(-c4ccc(N5CCCC5)cc4)cc(-c4ccc(N5CCCC5)cc4)n3)n2)n1]

Another embodiment of the present invention is a display device provided with a light-emitting device including any of the above-described organic compounds.

Another embodiment of the present invention is an electronic appliance provided with a sensor, an operation button, a speaker, or a microphone, and a light-emitting device including any of the above-described organic compounds.

Another embodiment of the present invention is a lighting device provided with a housing and a light-emitting device including any of the above-described organic compounds.

One embodiment of the present invention can provide a novel organic compound. Another embodiment of the present invention can provide a method for synthesizing a novel organic compound. Another embodiment of the present invention can provide an organic compound that can be used for a light-emitting device. Another embodiment of the present invention can provide a novel organic compound that can be used for an intermediate layer of a tandem light-emitting device. Another embodiment of the present invention can provide a light-emitting device having high emission efficiency. Another embodiment of the present invention can provide a high-color-purity light-emitting device. Another embodiment of the present invention can provide a highly reliable light-emitting device. Another embodiment of the present invention can provide any of a low-power-consumption display device, a low-power-consumption electronic appliance, and a low-power-consumption lighting device. Another embodiment of the present invention can provide any of a highly reliable display device, a highly reliable electronic appliance, and a highly reliable lighting device. Another embodiment of the present invention can provide any of a high-color-purity display device, a high-color-purity electronic appliance, and a high-color-purity lighting device.

Note that the description of these effects does not preclude the existence of other effects. One embodiment of the present invention does not necessarily have all of these effects. Those skilled in the art can find and extract other effects from the description of the specification, the drawings, the claims, and the like.

### BRIEF DESCRIPTION OF THE DRAWINGS

FIGS. 1A and 1B are schematic diagrams of a light-emitting device.

FIGS. 2A to 2E each illustrate a structure of a light-emitting device.

FIGS. 3A and 3B are a top view and a cross-sectional view, respectively, of a light-emitting apparatus.

FIGS. 4A to 4E are cross-sectional views illustrating an example of a method for manufacturing a display device.

FIGS. 5A and 5B are cross-sectional views illustrating an example of a method for manufacturing a display device.

FIGS. 6A to 6D are cross-sectional views illustrating an example of a method for manufacturing a display device.

FIGS. 7A to 7C are cross-sectional views illustrating an example of a method for manufacturing a display device.

FIGS. 8A to 8C are cross-sectional views illustrating an example of a method for manufacturing a display device.

FIGS. 9A to 9C are cross-sectional views illustrating an example of a method for manufacturing a display device.

FIGS. 10A to 10G are top views each illustrating a structure example of a pixel.

FIGS. 11A to 11I are top views each illustrating a structure example of a pixel.

FIGS. 12A and 12B are perspective views illustrating a structure example of a display module.

FIGS. 13A and 13B are cross-sectional views each illustrating a structure example of a display device.

FIG. 14 is a perspective view illustrating a structure example of a display device.

FIG. 15 is a cross-sectional view illustrating a structure example of a display device.

FIG. 16 is a cross-sectional view illustrating a structure example of a display device.

FIGS. 17A to 17C illustrate a structure example of a display device.

FIG. 18 is a cross-sectional view illustrating a structure example of a display device.

FIGS. 19A to 19C illustrate a structure example of a display device.

FIGS. 20A to 20D illustrate examples of electronic appliances.

FIGS. 21A to 21F illustrate examples of electronic appliances.

FIGS. 22A to 22G illustrate examples of electronic appliances.

FIG. 23 shows a ¹H NMR spectrum of an organic compound formed in an example.

FIG. 24 shows a ¹H NMR spectrum of an organic compound formed in an example.

FIG. 25 illustrates a structure of a light-emitting device.

FIG. 26 shows luminance-current density characteristics of a light-emitting device.

FIG. 27 shows luminance-voltage characteristics of a light-emitting device.

FIG. 28 shows current efficiency-current density characteristics of a light-emitting device.

FIG. 29 shows current density-voltage characteristics of a light-emitting device.

FIG. 30 shows an electroluminescence spectrum of a light-emitting device.

FIG. 31 shows a driving time-dependent change in luminance of a light-emitting device.

FIG. 32 shows luminance-current density characteristics of a light-emitting device.

FIG. 33 shows luminance-voltage characteristics of a light-emitting device.

FIG. 34 shows current efficiency-current density characteristics of a light-emitting device.

FIG. 35 shows current density-voltage characteristics of a light-emitting device.

FIG. 36 shows an electroluminescence spectrum of a light-emitting device.

FIG. 37 shows a driving time-dependent change in luminance of a light-emitting device.

FIG. 38 shows a ¹H NMR spectrum of an organic compound formed in an example.

FIG. 39 shows a ¹H NMR spectrum of an organic compound formed in an example.

FIG. 40 shows a ¹H NMR spectrum of an organic compound formed in an example.

FIG. 41 shows a ¹H NMR spectrum of an organic compound formed in an example.

FIG. 42 illustrates a structure of a light-emitting device.

FIG. 43 shows luminance-current density characteristics of a light-emitting device.

FIG. 44 shows luminance-voltage characteristics of a light-emitting device.

FIG. 45 shows current efficiency-current density characteristics of a light-emitting device.

FIG. 46 shows current density-voltage characteristics of a light-emitting device.

FIG. 47 shows an electroluminescence spectrum of a light-emitting device.

### DETAILED DESCRIPTION OF THE INVENTION

Embodiments of the present invention will be described in detail below with reference to the drawings. Note that the present invention is not limited to the following description, and it will be readily appreciated by those skilled in the art that modes and details of the present invention can be modified in various ways without departing from the spirit and scope of the present invention. Thus, the present invention should not be construed as being limited to the description in the following embodiments.

In this specification and the like, a device manufactured using a metal mask or a fine metal mask (FMM) is sometimes referred to as a device having a metal mask (MM) structure. In this specification and the like, a device formed without using a metal mask or a fine metal mask is sometimes referred to as a device having a metal maskless (MML) structure.

### Embodiment 1

In this embodiment, organic compounds of embodiments of the present invention will be described.

The organic compound of one embodiment of the present invention can be used for a functional layer of a light-emitting device. For example, the organic compound of one embodiment of the present invention can be suitably used for an electron-transport layer or an n-type layer of an intermediate layer. Specifically, when the organic compound of one embodiment of the present invention is mixed with a metal or a metal compound, the metal or the metal compound can be coordinated to a nitrogen atom contained in the organic compound of one embodiment of the present invention (a chelate complex can be formed). Thus, when mixed with a metal or a metal compound, the organic compound of one embodiment of the present invention can stabilize the metal or the metal compound functioning as an electron donor. That is, using the organic compound of one embodiment of the present invention for an intermediate layer or an electron-transport layer of a tandem light-emitting device allows the tandem light-emitting device to be driven at a low voltage.

### Organic Compound Example

The organic compounds of embodiments of the present invention can be represented by General Formulae (G1-1) to (G1-3), (G2-1) to (G2-3), (G3), (G4), and (G5) below.

### Organic Compound Example 1-1

One embodiment of the present invention is an organic compound represented by General Formula (G1-1).

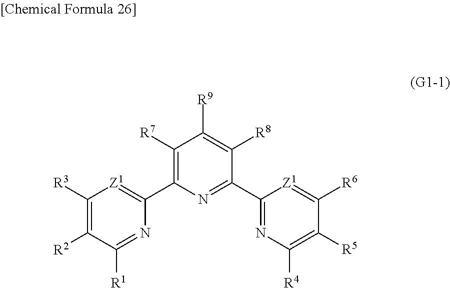

In General Formula (G1-1), Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g2) below.

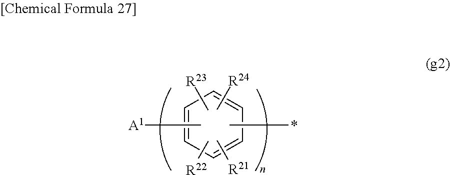

In General Formula (g2) above, A¹ represents the aliphatic amine group represented by General Formula (g1) below; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; and n represents an integer of 0 to 2.

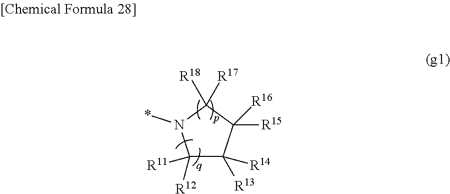

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; and any two of R¹¹ to R¹⁸ bonded to different carbon atoms may be bonded to each other to form a ring.

In General Formulae (g1) and (g2), an asterisk (*) represents a bond in the organic compound represented by General Formula (G1-1) above or any of General Formulae (G1-2), (G1-3), (G2-1) to (G2-3), and (G3) to (G5) described later.

### Organic Compound Example 1-2

One embodiment of the present invention is an organic compound represented by General Formula (G1-2).

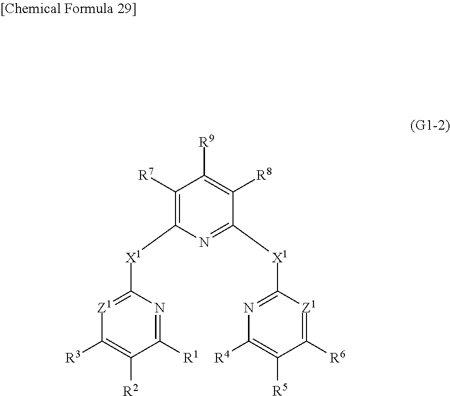

In General Formula (G1-2), X¹ represents oxygen, sulfur, or nitrogen; when X¹ represents nitrogen, the nitrogen has, as a substituent, a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g2) above.

In General Formula (G1-2), X¹ preferably represents oxygen or sulfur, in which case nitrogen atoms each have a wide space therearound to facilitate interaction with the metal or the metal compound.

In General Formula (G1-2), when X¹ represents nitrogen having the aryl group or the heteroaryl group, the nitrogen atom of the ring having Z¹ and the nitrogen atom of the central pyridine ring have a conformation that facilitates coordination of the nitrogen atoms to the metal or the metal compound (formation of a chelate complex). Thus, the organic compound represented by General Formula (G1-2) and the metal or the metal compound easily interact with each other, which is preferable.

### Organic Compound Example 1-3

One embodiment of the present invention is an organic compound represented by General Formula (G1-3).

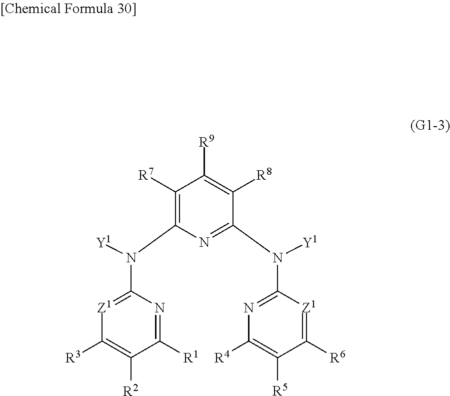

In General Formula (G1-3), Y¹ represents a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g2) above.

### Organic Compound Example 2-1

One embodiment of the present invention is an organic compound represented by General Formula (G2-1).

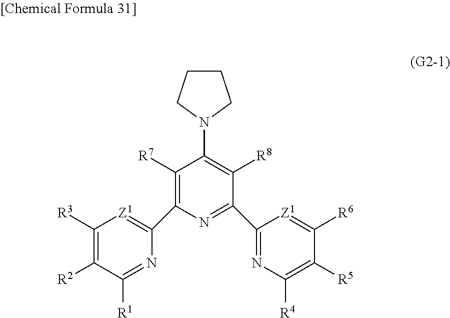

In General Formula (G2-1), Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g3) below.

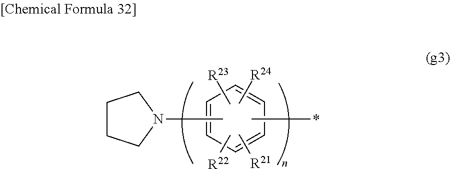

In General Formula (g3) above, each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) above; and n represents an integer of 0 to 2.

In General Formula (g3), an asterisk (*) represents a bond in the organic compound represented by General Formula (G2-1) above or General Formula (G2-2) or (G2-3) described later.

Since the organic compound represented by General Formula (G2-1) has the pyrrolidinyl group, which is an electron-donating group, at the 4-position of the central pyridine ring, the electron density of the nitrogen of the central pyridine ring can be increased. Thus, the organic compound represented by General Formula (G2-1) and the metal or the metal compound easily interact with each other, which is preferable.

### Organic Compound Example 2-2

One embodiment of the present invention is an organic compound represented by General Formula (G2-2).

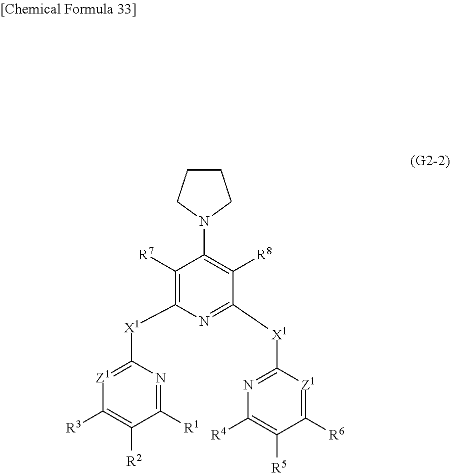

In General Formula (G2-2), X¹ represents oxygen, sulfur, or nitrogen; when X¹ represents nitrogen, the nitrogen has, as a substituent, a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g3) above.

In General Formula (G2-2), as in General Formula (G1-2), X¹ preferably represents oxygen or sulfur, in which case nitrogen atoms each have a wide space therearound to facilitate interaction with the metal or the metal compound.

In General Formula (G2-2), when X¹ represents nitrogen having the aryl group or the heteroaryl group, the nitrogen atom of the ring having Z¹ and the nitrogen atom of the central pyridine ring have a conformation that facilitates coordination of the nitrogen atoms to the metal or the metal compound (formation of a chelate complex). Thus, the organic compound represented by General Formula (G2-2) and the metal or the metal compound easily interact with each other, which is preferable.

### Organic Compound Example 2-3

One embodiment of the present invention is an organic compound represented by General Formula (G2-3).

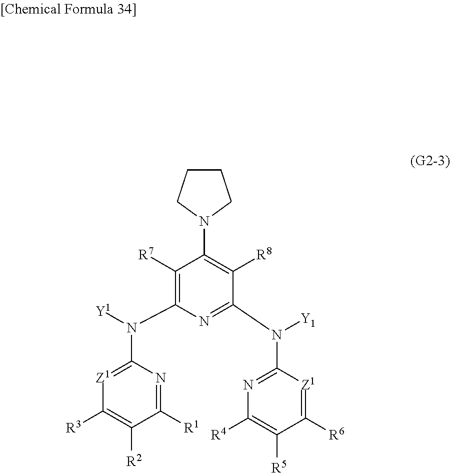

In General Formula (G2-3), Y¹ represents a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g3) above.

In General Formulae (G1-1) to (G1-3) and (G2-1) to (G2-3) above, Z¹ representing nitrogen would allow the nitrogen atoms of the pyrimidine rings having Z¹ to maintain a conformation in which the nitrogen atoms of the two pyrimidine rings face each other if the pyrimidine rings are rotated. Thus, the organic compound of one embodiment of the present invention in which Z¹ represents nitrogen is easily coordinated to the metal or the metal compound (a chelate complex is easily formed) and is thus preferable.

In the case where Z¹ represents carbon in General Formulae (G1-1) to (G1-3) and (G2-1) to (G2-3) above, it is preferable that R³ and R⁶ each represent the group represented by General Formula (g2) or (g3) and n be 1 or 2. This structure improves heat resistance and is thus preferable.

### Organic Compound Example 3

Another embodiment of the present invention is an organic compound represented by General Formula (G3).

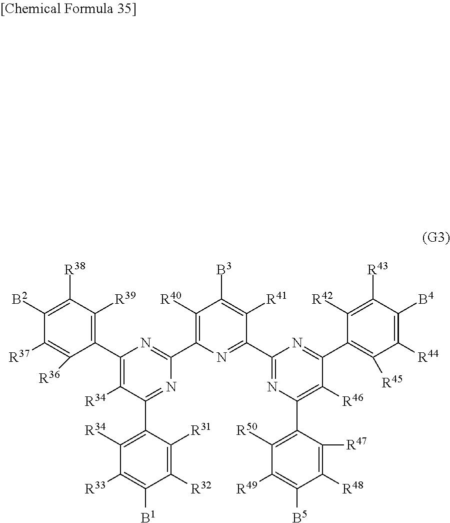

In General Formula (G3), each of B¹ to B⁵ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; at least one of B¹ to B⁵ represents the aliphatic amine group represented by General Formula (g1) above; and each of R³¹ to R⁵⁰ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) above.

In the organic compound represented by General Formula (G3), the nitrogen atoms of one pyrimidine ring and the nitrogen atoms of the other pyrimidine ring adjacent to the one pyrimidine ring with the pyridine ring therebetween can maintain a conformation in which the nitrogen atoms of the two pyrimidine rings face each other even when the pyrimidine ring(s) or the pyridine ring is rotated, and thus, the nitrogen atoms of the one pyrimidine ring and the nitrogen atoms of the other pyrimidine ring adjacent to the one pyrimidine ring with the pyridine ring therebetween are coordinated to the metal or the metal compound (a chelate complex is formed), which is preferable. Moreover, since the organic compound has the aliphatic amine group represented by General Formula (g1), which is an electron-donating group, as a substituent, the electron density of the nitrogen of each of the pyridine ring and the pyrimidine ring can be increased and accordingly the interaction with the metal or the metal compound is facilitated, which is preferable.

### Organic Compound Example 4

Another embodiment of the present invention is an organic compound represented by General Formula (G4).

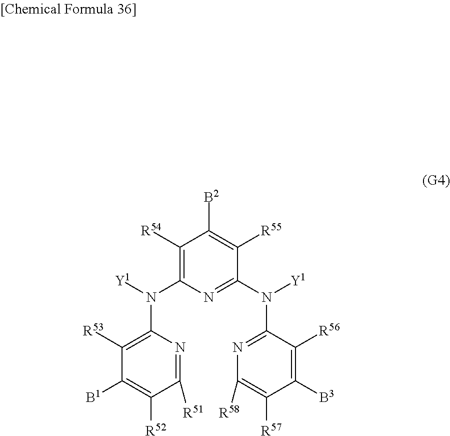

In General Formula (G4), Y¹ represents a substituted or unsubstituted aryl group having 6 to 30 carbon atoms or a substituted or unsubstituted heteroaryl group having 1 to 30 carbon atoms; each of B¹ to B³ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; at least one of B¹ to B³ represents the aliphatic amine group represented by General Formula (g1) above; and each of R⁵¹ to R⁵⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) above.

In the organic compound represented by General Formula (G4), when B¹ to B³ each represent a substituent, a conformation can be maintained in which the nitrogen atoms of the three pyridine rings face each other owing to the steric effect of the substituents as B¹ to B³. Thus, the organic compound represented by General Formula (G4) is coordinated to the metal or the metal compound (a chelate complex is formed), which is preferable.

### Organic Compound Example 5

Another embodiment of the present invention is an organic compound represented by General Formula (G5).

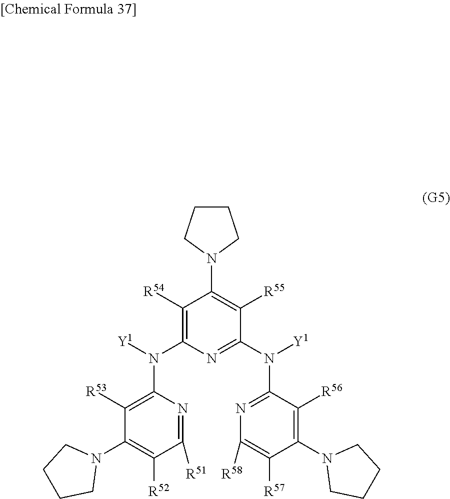

In General Formula (G5), Y¹ represents a substituted or unsubstituted aryl group having 6 to 30 carbon atoms or a substituted or unsubstituted heteroaryl group having 1 to 30 carbon atoms; and each of R⁵¹ to R⁵⁸ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above.

Since the organic compound represented by General Formula (G5) has the pyrrolidinyl groups, a conformation can be maintained in which the nitrogen atoms of the three pyridine rings face each other owing to the steric effect of the pyrrolidinyl groups and thus, the nitrogen atoms of the three pyridine rings are coordinated to the metal or the metal compound (a chelate complex is formed), which is preferable.

Specific examples of the substituent represented by Y¹ or R^{m} (m is a given natural number) in General Formulae (G1-1) to (G1-3), (G2-1) to (G2-3), (G3) to (G5), and (g1) to (g3) are described below.

Examples of the substituted or unsubstituted aryl group having 6 to 30 carbon atoms include a phenyl group, a biphenyl group, a terphenyl group, a naphthyl group, an anthryl group, a fluorenyl group, a dibenzofluorenyl group, a diphenylfluorenyl group, a spirobifluorenyl group, a pyrenyl group, a phenanthryl group, a triphenylenyl group, a perylenyl group, a tetracenyl group, and a chrysenyl group.

Examples of the substituted or unsubstituted heteroaryl group having 1 to 30 carbon atoms include a group having a pyridine ring, a pyrazine ring, a pyrimidine ring, a pyridazine ring, a triazine ring, a quinoline ring, a quinazoline ring, an isoquinoline ring, a pyrrole ring, a naphthyridine ring, a phenanthridine ring, a phenanthroline ring, a quinoxaline ring, an imidazole ring, a benzimidazole ring, an oxazole ring, an isoxazole ring, a thiazole ring, an isothiazole ring, or a benzofuran ring.

Examples of the alkyl group having 1 to 10 carbon atoms include a methyl group, an ethyl group, a propyl group, an isopropyl group, a butyl group, a sec-butyl group, an isobutyl group, a tert-butyl group, a pentyl group, an isopentyl group, a sec-pentyl group, a tert-pentyl group, a neopentyl group, a hexyl group, an isohexyl group, a sec-hexyl group, a tert-hexyl group, a neohexyl group, a 3-methylpentyl group, a 2-methylpentyl group, a 2-ethylbutyl group, a 1,2-dimethylbutyl group, a 2,3-dimethylbutyl group, an octyl group, an isooctyl group, a sec-octyl group, a tert-octyl group, a nonyl group, an isononyl group, a sec-nonyl group, a tert-nonyl group, a decanyl group, an isodecanyl group, a sec-decanyl group, a tert-decanyl group, an undecanyl group, and an isoundecanyl group.

Examples of the alkoxy group having 1 to 10 carbon atoms include a methoxy group, an ethoxy group, a propoxy group, a t-butoxy group, a pentyloxy group, an octyloxy group, an allyloxy group, a cyclohexyloxy group, a phenoxy group, and a benzyloxy group.

Examples of the silyl group having 1 to 20 carbon atoms include a trimethylsilyl group, a triethylsilyl group, a dimethylpropylsilyl group, a tert-butyldimethylsilyl group, a triisopropylsilyl group, a dimethyloctadecylsilyl group, a tert-butyldiphenylsilyl group, a benzyldimethylsilyl group, and a triphenylsilyl group.

In the case where the aryl group or the heteroaryl group has a substituent, an alkyl group having 1 to 6 carbon atoms, a cycloalkyl group having 3 to 6 carbon atoms, a cyano group, or a hydroxy group can be selected as the substituent.

In the case where the substituent bonded to the aryl group and the heteroaryl group is the alkyl group having 1 to 6 carbon atoms, specific examples of the alkyl group having 1 to 6 carbon atoms include a methyl group, an ethyl group, a propyl group, an isopropyl group, a butyl group, an isobutyl group, a tert-butyl group, and an n-hexyl group. Examples of the cycloalkyl group having 3 to 6 carbon atoms include a cyclopropyl group, a cyclobutyl group, a cyclopentyl group, and a cyclohexyl group.

Note that in General Formulae (G1-1) to (G1-3), (G2-1) to (G2-3), (G3) to (G5), and (g1) to (g3), hydrogen may be replaced with deuterium as appropriate.

### Specific Example

The following are specific examples of the organic compound of one embodiment of the present invention having the structure represented by any of General Formulae (G1-1) to (G1-3), (G2-1) to (G2-3), and (G3) to (G5).

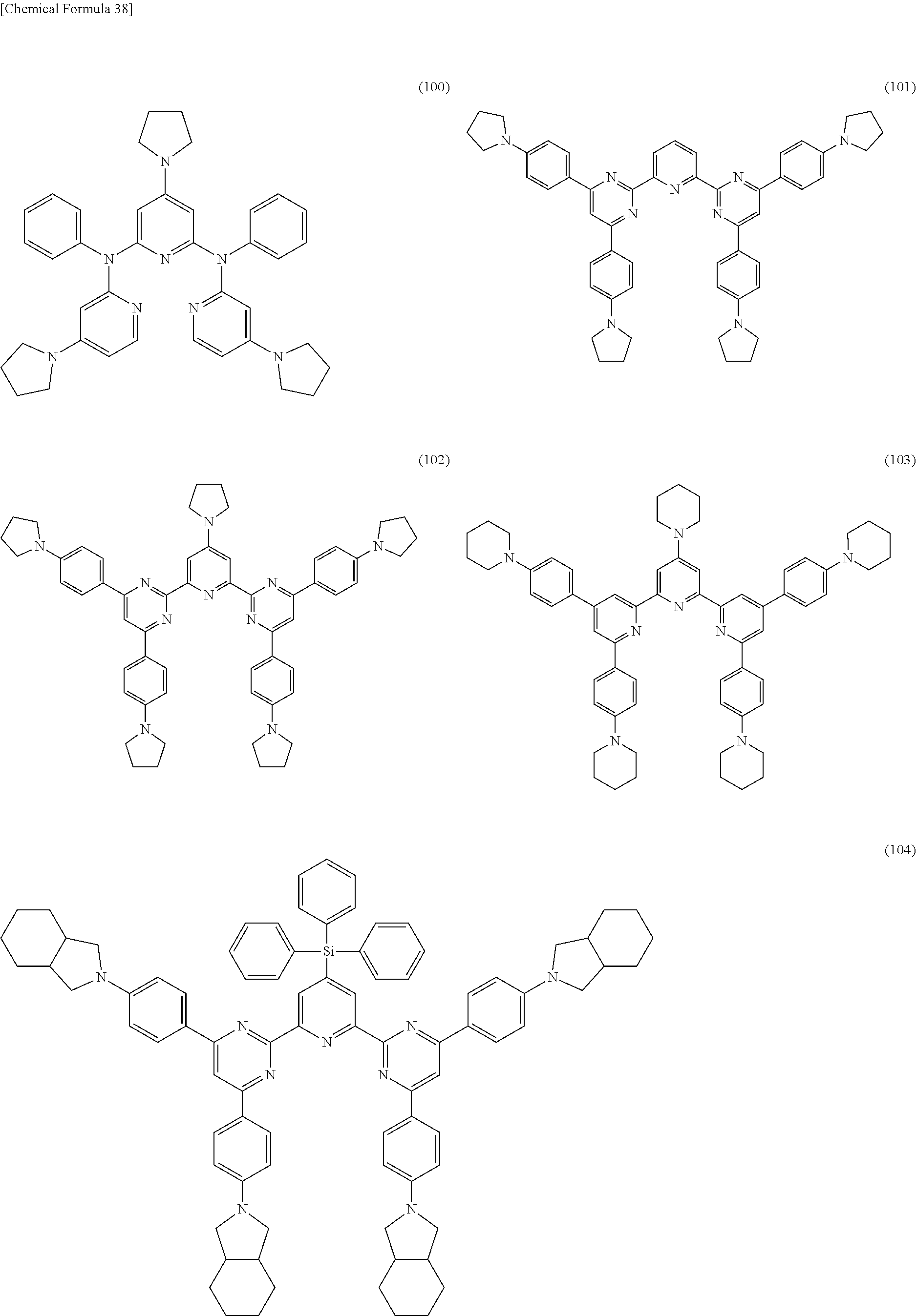

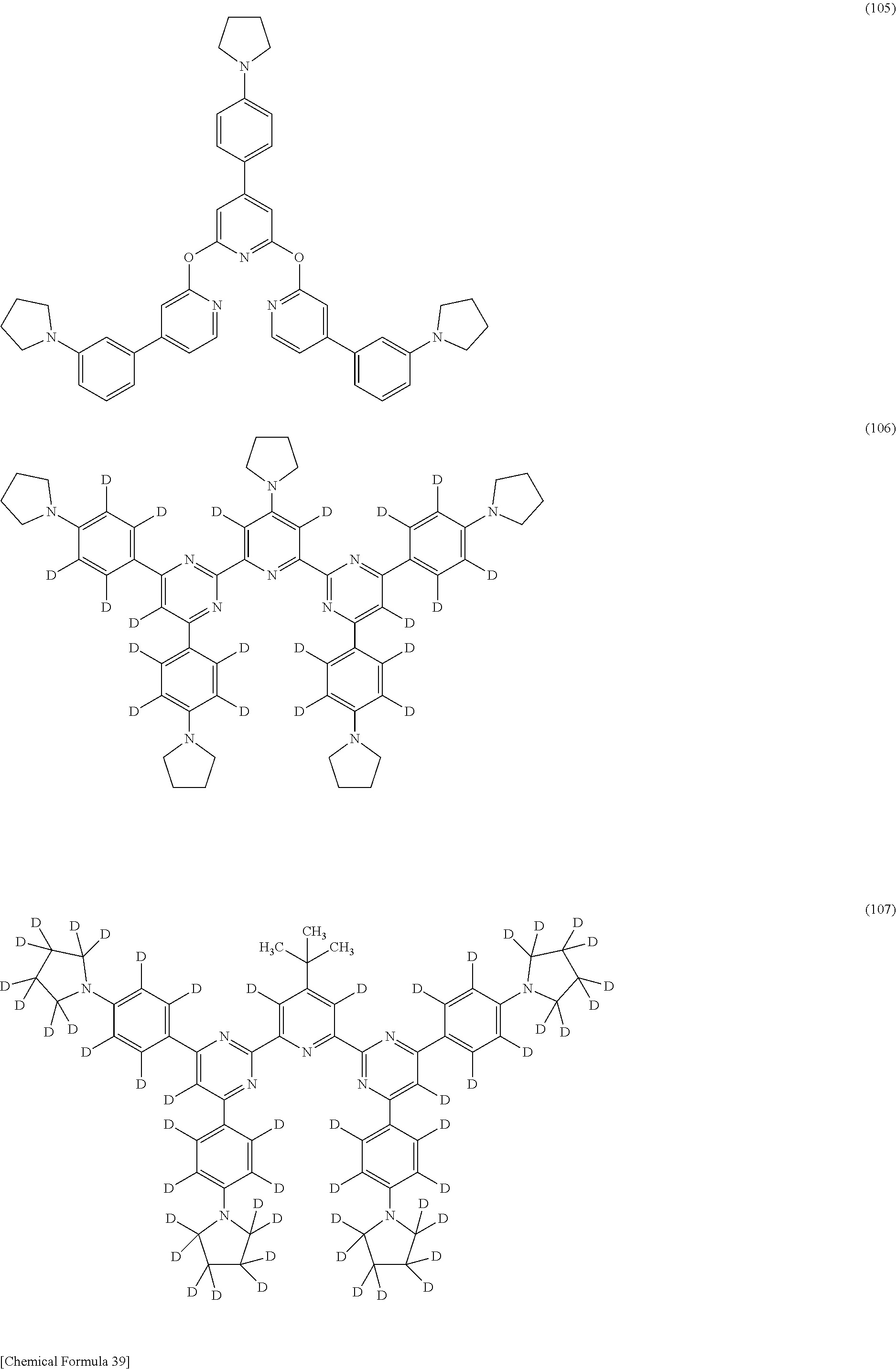

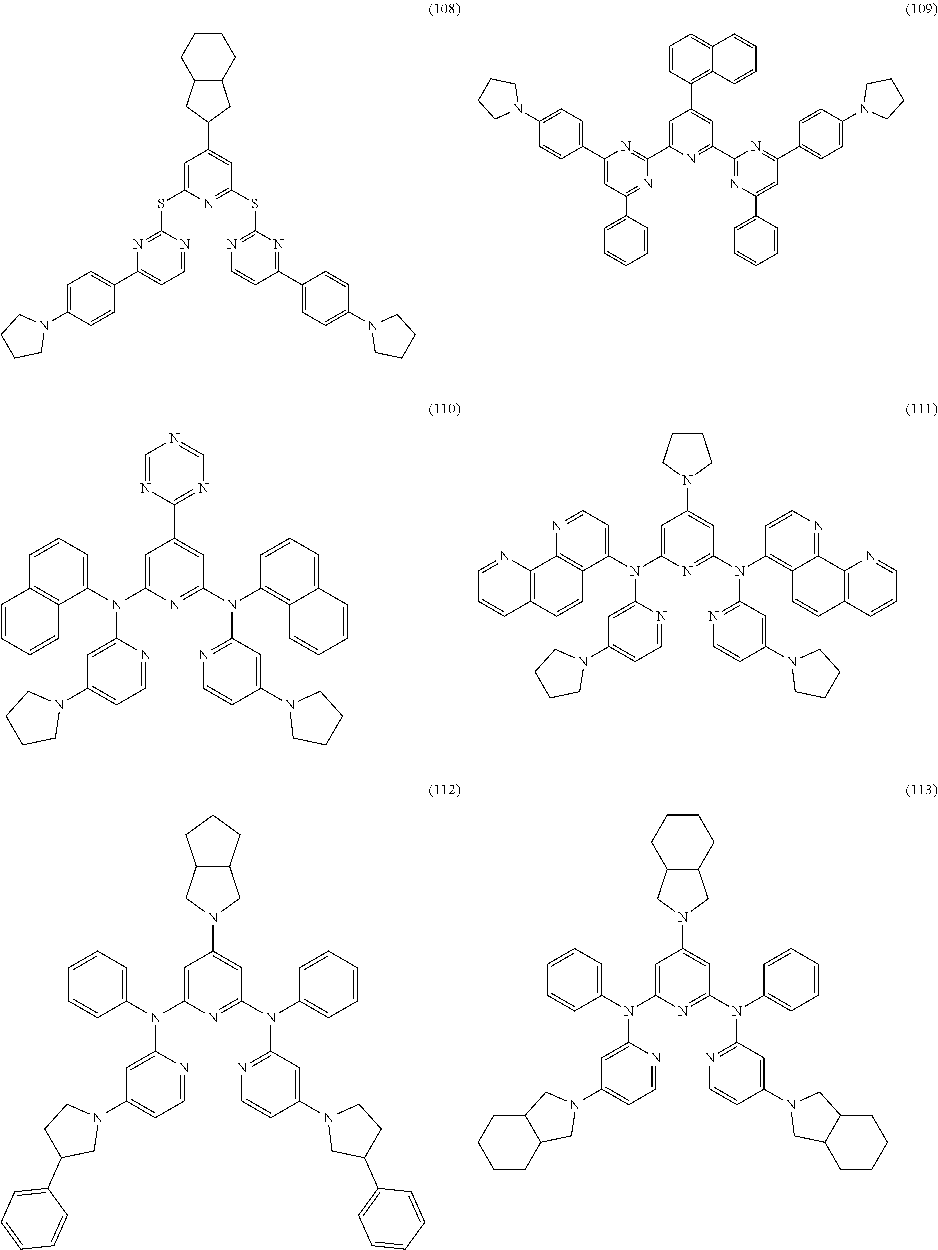

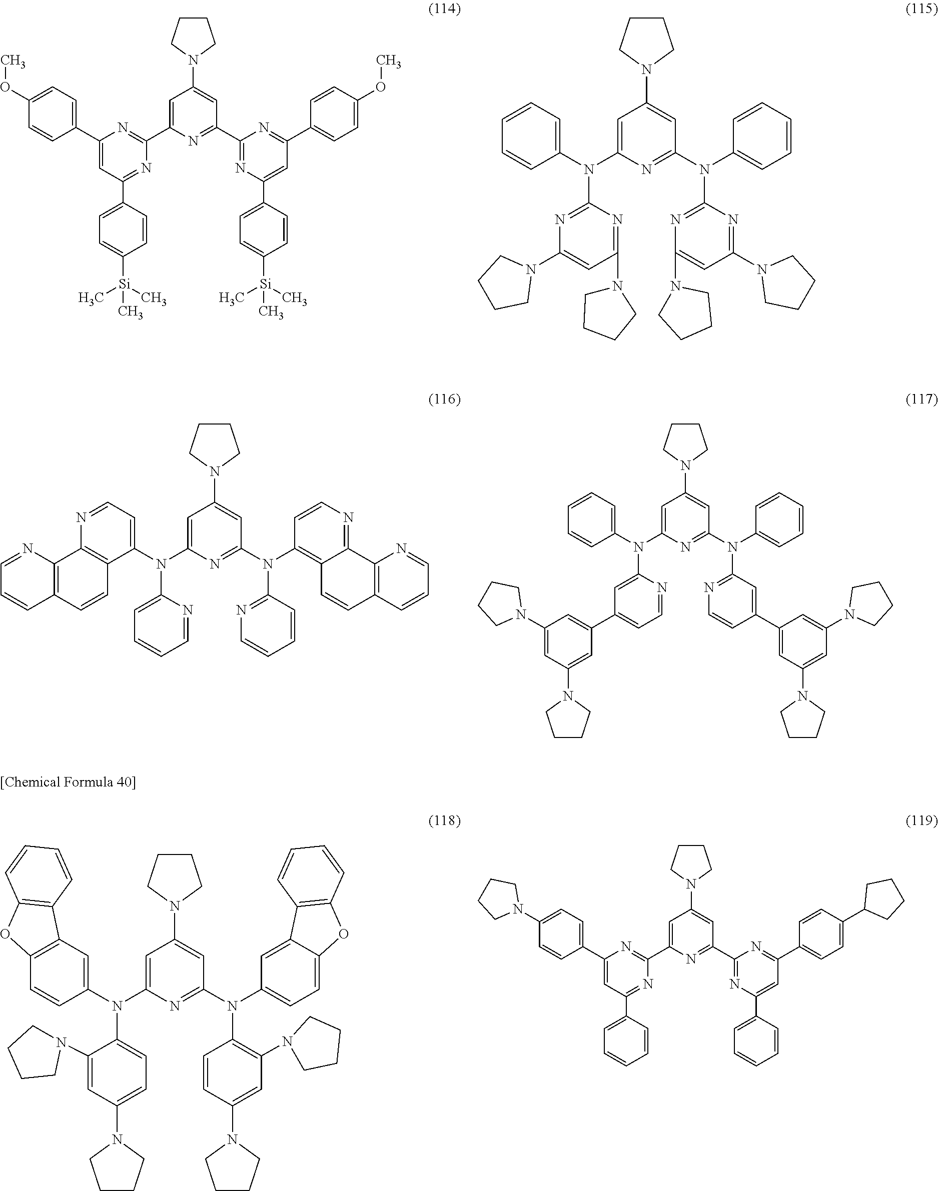

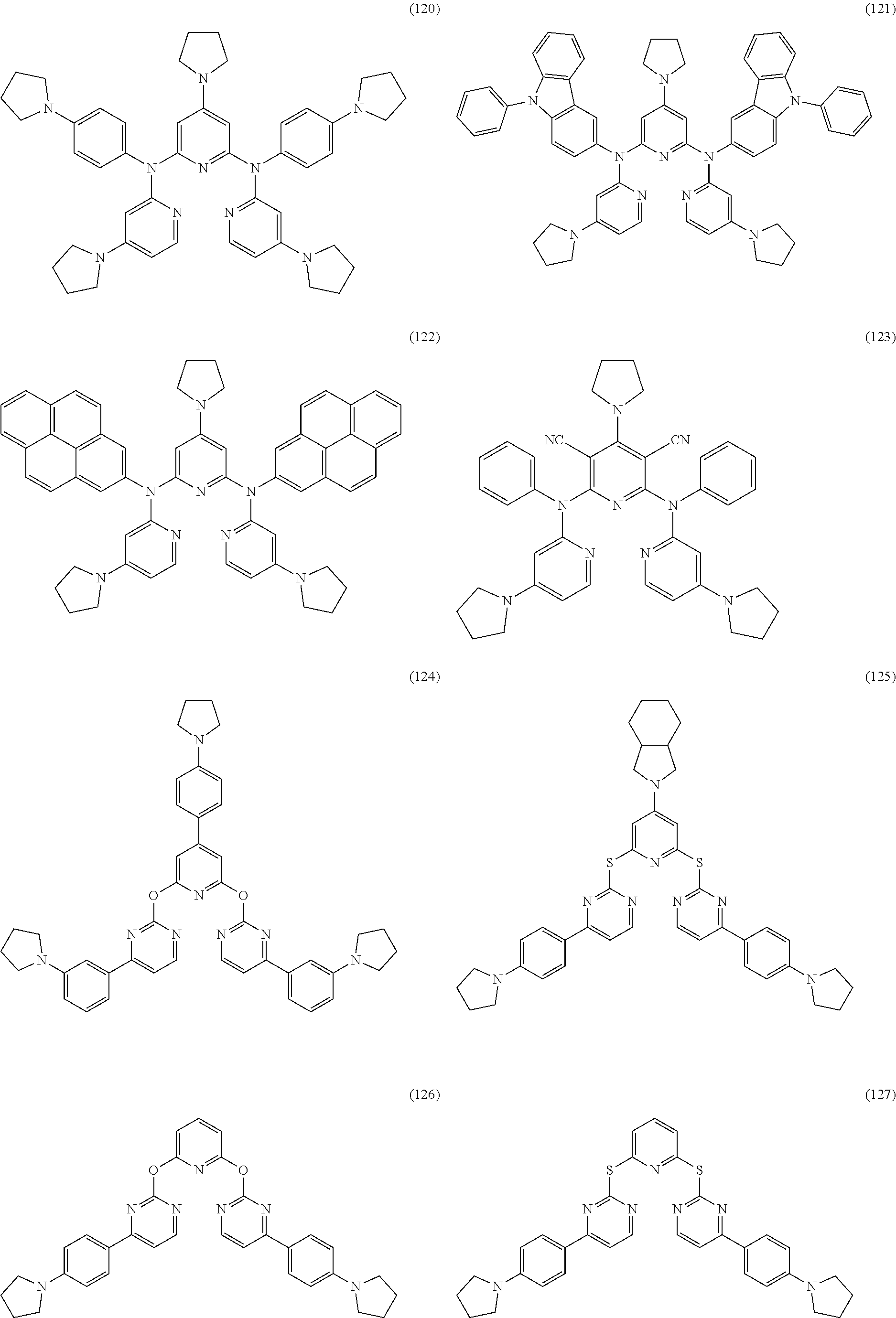

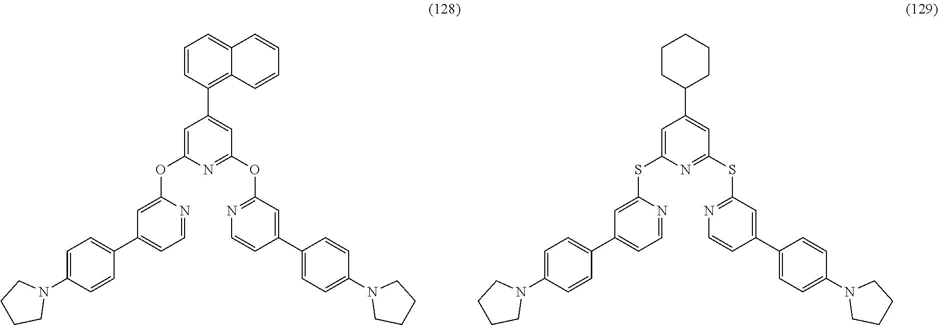

The organic compounds represented by Structural Formulae (100) to (129) above are examples of the organic compound represented by any of General Formulae (G1-1) to (G1-3), (G2-1) to (G2-3), and (G3) to (G5); however, the organic compound of one embodiment of the present invention is not limited to these examples.

### Synthesis Method of Organic Compound

Next, as an example of a synthesis method of the organic compound of one embodiment of the present invention, a synthesis method of the organic compound represented by General Formula (G1-1) below is described. Note that the method for synthesizing the organic compound represented by General Formula (G1-1) can employ a variety of reactions and is not limited to the following synthesis method.

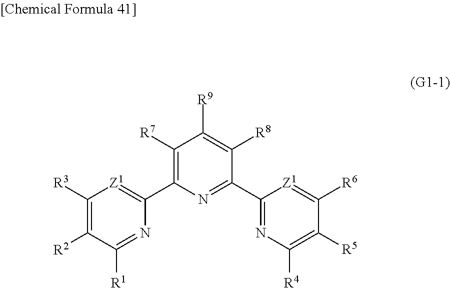

In General Formula (G1-1), Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) below; and at least one of R¹ to R³ and at least one of R⁴ to R⁶ each represent a group represented by General Formula (g2) below.

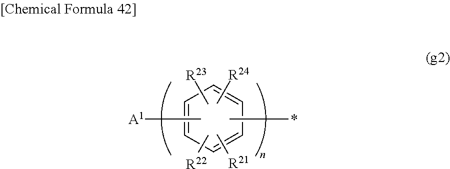

In General Formula (g2) above, A¹ represents the aliphatic amine group represented by General Formula (g1) below; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1) below; and n represents an integer of 0 to 2.

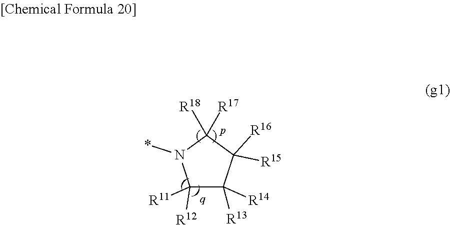

In General Formula (g1) above, each of R¹¹ to R¹⁸ independently represents any one of hydrogen (including deuterium), an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group; each of p and q independently represents an integer of 0 to 3; and any two of R¹¹ to R¹⁸ bonded to different carbon atoms may be bonded to each other to form a ring.

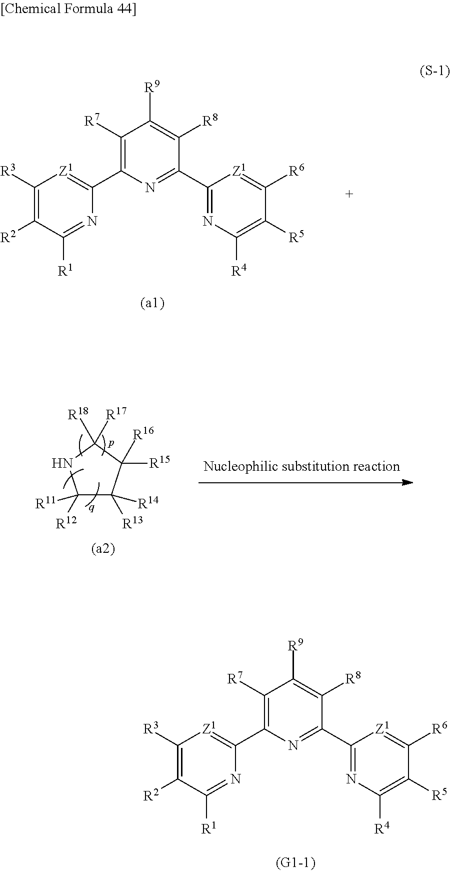

In a compound (a1) above, at least one of R¹ to R³ and at least one of R⁴ to R⁶ each represent a group represented by General Formula (g4) below, and Z¹ and R¹ to R⁹ are similar to those in General Formula (G1-1).

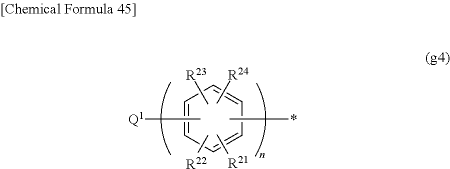

In General Formula (g4) above, Q¹ represents a halogen or a trifluoromethanesulfonyl group; each of R²¹ to R²⁴ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1); and n represents an integer of 0 to 2.

In a compound (a2) above, R¹¹ to R¹⁸, p, and q are similar to those in General Formula (g1).

In Synthesis Scheme (S-1), the compound (a1) that is a derivative and the compound (a2) that is an aliphatic cyclic amine derivative are subjected to a nucleophilic substitution reaction using an appropriate solvent and an appropriate base, whereby the organic compound represented by General Formula (G1-1) can be obtained.

Examples of the base that can be used in the nucleophilic substitution reaction represented by Synthesis Scheme (S-1) above include organic bases such as diazabicycloundecene (DBU), triethylamine, and potassium tert-butoxide, and inorganic bases such as potassium carbonate, cesium carbonate, sodium carbonate, sodium hydrogen carbonate, potassium acetate, sodium acetate, tripotassium phosphate, and trisodium phosphate.

Examples of the solvent that can be used in the nucleophilic substitution reaction represented by Synthesis Scheme (S-1) above include N-methyl-2-pyrrolidone, N,N-dimethylformamide, toluene, tetrahydrofuran, dioxane, and ethanol. However, the solvent that can be used is not limited to these solvents. In the case where an organic base is employed, the organic base may be used as a base and a solvent.

The reaction employed in Synthesis Scheme (S-1) above is not limited to the nucleophilic substitution reaction; a Buchwald-Hartwig reaction, a coupling reaction using copper or a copper compound, or the like can be used.

Next, as an example of a synthesis method of the organic compound of one embodiment of the present invention, a synthesis method of the organic compound represented by General Formula (G1-2) below is described. Note that the method for synthesizing the organic compound represented by General Formula (G1-2) can employ a variety of reactions and is not limited to the following synthesis method.

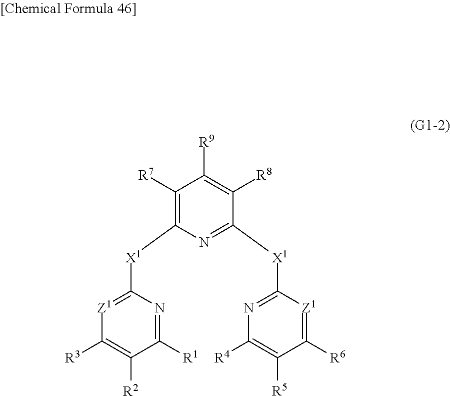

In General Formula (G1-2), X¹ represents oxygen, sulfur, or nitrogen; when X¹ represents nitrogen, the nitrogen has, as a substituent, a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g2) above.

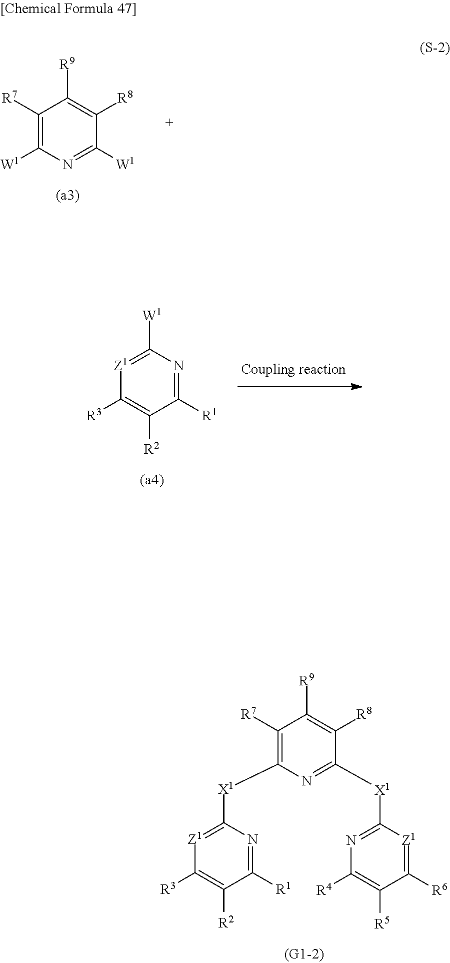

In a compound (a3) above, W¹ represents a hydroxy group, a thiol group, a halogen, a trifluoromethanesulfonyl group, or an imino group; when W¹ represents an imino group, the imino group has, as a substituent, a substituted or unsubstituted aryl group having 6 to 30 carbon atoms or a substituted or unsubstituted heteroaryl group having 1 to 30 carbon atoms. For R⁷ to R⁹, the description of General Formula (G1-2) can be referred to.

In a compound (a4) above, W¹ represents a hydroxy group, a thiol group, a halogen, a trifluoromethanesulfonyl group, or an imino group; when W¹ represents an imino group, the imino group has, as a substituent, a substituted or unsubstituted aryl group having 6 to 30 carbon atoms or a substituted or unsubstituted heteroaryl group having 1 to 30 carbon atoms. For R¹ to R³, the description of General Formula (G1-2) can be referred to.

In Synthesis Scheme (S-2), the compound (a3) that is a derivative and the compound (a4) that is a derivative are coupled by a Buchwald-Hartwig reaction, whereby the organic compound represented by General Formula (G1-2) can be obtained.

Examples of a palladium catalyst that can be used in the coupling reaction represented by Synthesis Scheme (S-2) above include palladium(II) acetate, tetrakis(triphenylphosphine)palladium(0), bis(triphenylphosphine)palladium(II) dichloride, and tris(dibenzylideneacetone)dipalladium(0).

Examples of a ligand in the above palladium catalyst include (±)-2,2′-bis(diphenylphosphino)-1,1′-binaphthyl, tri(ortho-tolyl)phosphine, triphenylphosphine, tricyclohexylphosphine, and 4,5-bis(diphenylphosphino)-9,9-dimethylxanthene.

Examples of a base that can be used in the coupling reaction represented by Synthesis Scheme (S-2) above include organic bases such as sodium tert-butoxide and potassium tert-butoxide and inorganic bases such as potassium carbonate and sodium carbonate.

Examples of a solvent that can be used in the coupling reaction represented by Synthesis Scheme (S-2) above include toluene, xylene, mesitylene, benzene, tetrahydrofuran, and dioxane. However, the solvent that can be used is not limited to these solvents.

The reaction employed in Synthesis Scheme (S-2) above is not limited to the Buchwald-Hartwig reaction. A Migita-Kosugi-Stille coupling reaction using an organotin compound, a coupling reaction using a Grignard reagent, an Ullmann reaction using copper or a copper compound, a nucleophilic substitution reaction, or the like can be used.

Next, as an example of a synthesis method of the organic compound of one embodiment of the present invention, a synthesis method of the organic compound represented by General Formula (G1-3) below is described. Note that the method for synthesizing the organic compound represented by General Formula (G1-3) can employ a variety of reactions and is not limited to the following synthesis method.

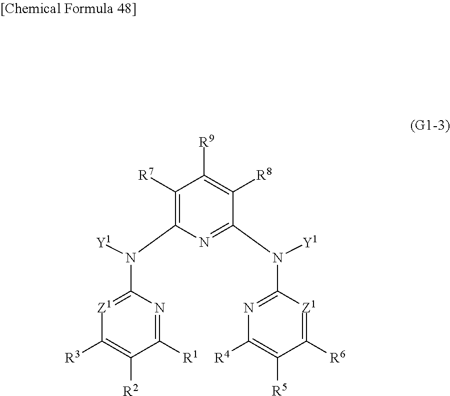

In General Formula (G1-3), Y¹ represents a substituted or unsubstituted aryl group having 6 to 30 carbon atoms or a substituted or unsubstituted heteroaryl group having 1 to 30 carbon atoms; Z¹ represents carbon or nitrogen; when Z¹ represents carbon, the carbon has hydrogen (including deuterium); each of R¹ to R⁹ independently represents any one of hydrogen (including deuterium), an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1) above; and at least one of R¹ to R³ and at least one of R⁴ to R⁶ each represent a group represented by General Formula (g2) above.

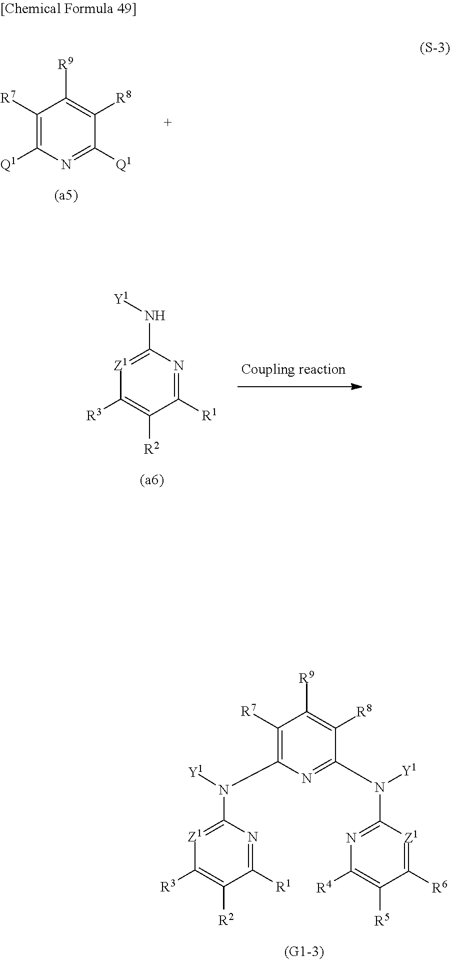

In a compound (a5) above, Q¹ represents a halogen or a trifluoromethanesulfonyl group, and for R⁷ to R⁹, the description of General Formula (G1-3) can be referred to.

For Y¹ and R¹ to R³ in a compound (a6) above, the description of General Formula (G1-3) can be referred to.

In Synthesis Scheme (S-3), the compound (a5) that is a derivative and the compound (a6) that is a derivative are coupled by a Buchwald-Hartwig reaction, whereby the organic compound represented by General Formula (G1-3) can be obtained.

Examples of a palladium catalyst that can be used in the coupling reaction represented by Synthesis Scheme (S-3) above include palladium(II) acetate, tetrakis(triphenylphosphine)palladium(0), bis(triphenylphosphine)palladium(II) dichloride, and tris(dibenzylideneacetone)dipalladium(0).

Examples of a ligand in the above palladium catalyst include (±)-2,2′-bis(diphenylphosphino)-1,1′-binaphthyl, tri(ortho-tolyl)phosphine, triphenylphosphine, tricyclohexylphosphine, and 4,5-bis(diphenylphosphino)-9,9-dimethylxanthene.

Examples of a base that can be used in the coupling reaction represented by Synthesis Scheme (S-3) above include an organic base such as potassium tert-butoxide and an inorganic base such as potassium carbonate or sodium carbonate.

Examples of a solvent that can be used in the coupling reaction represented by Synthesis Scheme (S-3) above include toluene, xylene, mesitylene, benzene, tetrahydrofuran, and dioxane. However, the solvent that can be used is not limited to these solvents.

The reaction employed in Synthesis Scheme (S-3) above is not limited to the Buchwald-Hartwig reaction. A Migita-Kosugi-Stille coupling reaction using an organotin compound, a coupling reaction using a Grignard reagent, an Ullmann reaction using copper or a copper compound, a nucleophilic substitution reaction, or the like can be used.

The organic compound of one embodiment of the present invention can be synthesized in the above manner, but the present invention is not limited thereto, and other synthesis methods may be employed.

This embodiment can be freely combined with any of the other embodiments and the examples.

### Embodiment 2

In this embodiment, structures of a light-emitting device including the organic compound described in Embodiment 1 will be described.

It is a long time since displays (organic EL displays) that include organic EL elements (hereinafter also referred to as light-emitting devices) as display elements were put into practical use. These displays are usually provided with pixels emitting light with at least three colors of red, green, and blue to achieve full-color display.

The pixels are provided with light-emitting devices for the respective emission colors. In a display fabricated by a side-by-side method, or what is called a separate coloring method, light-emitting devices include light-emitting substances corresponding to the respective emission colors of the pixels.

When the organic compound described in Embodiment 1 is used for a charge-generation layer (intermediate layer) or a layer in contact with the anode side of the charge-generation layer (intermediate layer) used in a tandem light-emitting device or the like, the degree of crosstalk can be reduced, which facilitates provision of a display device with high display quality. The charge-generation layer will be described in detail in a later description of light-emitting devices.

Thus, one embodiment of the present invention provides a tandem light-emitting device in which the organic compound described in Embodiment 1 is used as a material for an intermediate layer.

The organic compound described in Embodiment 1, which has a favorable carrier-transport property, particularly an excellent electron-transport property, is suitable for a host material or a carrier-transport layer, particularly an electron-transport layer or an electron-injection layer, in a light-emitting device.

One embodiment of the present invention provides a light-emitting device in which the organic compound described in Embodiment 1 is used for a charge-generation layer (intermediate layer). Alternatively, one embodiment of the present invention provides a light-emitting device in which the organic compound described in Embodiment 1 is used as an electron-transport material.

### Structure Example of Light-Emitting Device

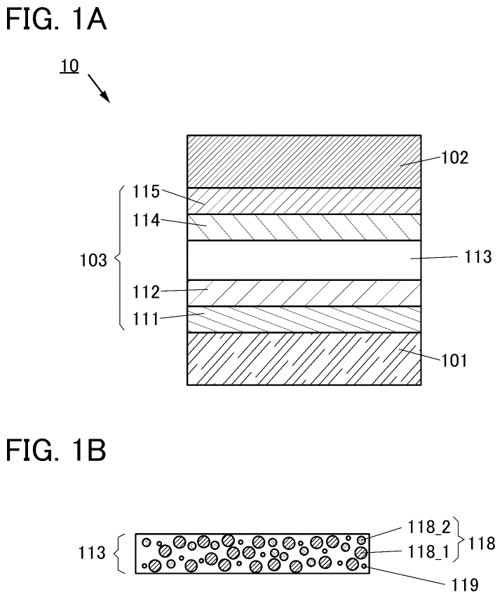

FIG. 1A is a schematic cross-sectional view of a light-emitting device 10 of one embodiment of the present invention. The light-emitting device 10 includes a pair of electrodes (a first electrode 101 and a second electrode 102) and an organic compound layer 103 between the pair of electrodes. The organic compound layer 103 includes at least a light-emitting layer 113.

The organic compound layer 103 illustrated in FIG. 1A includes functional layers such as a hole-injection layer 111, a hole-transport layer 112, an electron-transport layer 114, and an electron-injection layer 115, in addition to the light-emitting layer 113.

Although description is given in this embodiment assuming that the first electrode 101 and the second electrode 102 of the pair of electrodes serve as an anode and a cathode, respectively, the structure of the light-emitting device 10 is not limited thereto. That is, the first electrode 101 may be a cathode, the second electrode 102 may be an anode, and the stacking order of the layers between the electrodes may be reversed. In other words, the hole-injection layer 111, the hole-transport layer 112, the light-emitting layer 113, the electron-transport layer 114, and the electron-injection layer 115 can be stacked in this order from the anode side.

The structure of the organic compound layer 103 is not limited to the structure illustrated in FIG. 1A, and a structure including at least one layer selected from the hole-injection layer 111, the hole-transport layer 112, the electron-transport layer 114, and the electron-injection layer 115 can be employed. Alternatively, the organic compound layer 103 may include a functional layer which has a function of lowering a hole- or electron-injection barrier, improving a hole- or electron-transport property, inhibiting a hole- or electron-transport property, or reducing quenching by an electrode, for example. Note that the functional layer may be either a single layer or stacked layers.

FIG. 1B is a schematic cross-sectional view illustrating an example of the light-emitting layer 113 in FIG. 1A. The light-emitting layer 113 illustrated in FIG. 1B includes host materials 118 (an organic compound 118_1 and an organic compound 118_2) and a guest material 119 (a light-emitting substance).

The guest material 119 may be a light-emitting organic compound, and the light-emitting organic compound is preferably a substance capable of emitting phosphorescent light (hereinafter also referred to as a phosphorescent compound).

In the light-emitting layer 113, the host materials 118 are present in the largest proportion by weight, and the guest material 119 is dispersed in the host materials 118. The lowest triplet excitation energy levels (T₁ levels) of the host materials 118 (the organic compounds 118_1 and 118_2) in the light-emitting layer 113 are preferably higher than the T₁ level of the guest material 119 in the light-emitting layer 113.

The host materials 118 (the organic compounds 118_1 and 118_2) in the light-emitting layer 113 preferably form an exciplex. Note that an exciplex is an excited state formed by two or more kinds of substances. In photoexcitation, the exciplex is formed by interaction between one substance in an excited state and another substance in a ground state.

### Basic Structure of Light-Emitting Device

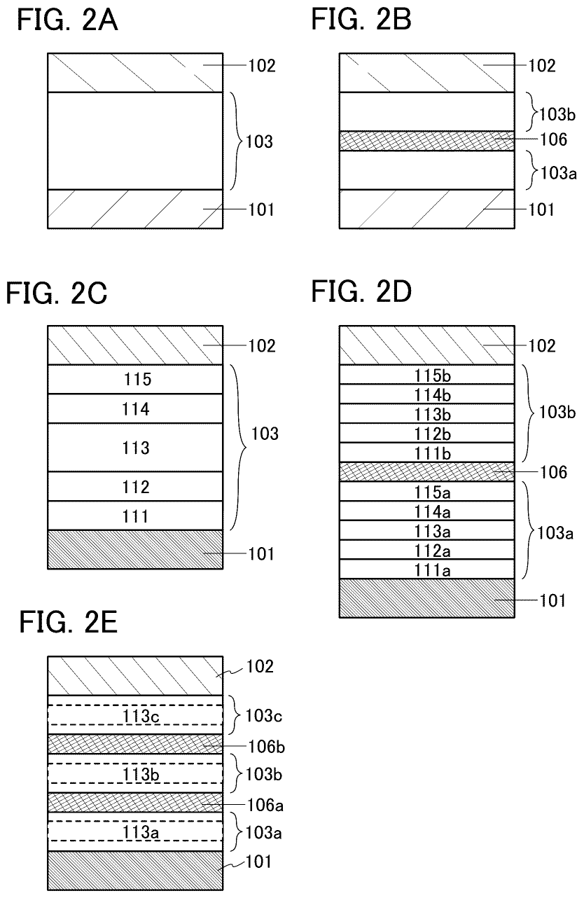

Basic structures of the light-emitting device will be specifically described below with reference to FIGS. 2A to 2E. FIG. 2A illustrates a light-emitting device having a structure (single structure) in which an organic compound layer (also referred to as an EL layer) including a light-emitting layer is provided between a pair of electrodes. Specifically, the organic compound layer 103 is sandwiched between the first electrode 101 and the second electrode 102.

FIG. 2B illustrates a light-emitting device that has a stacked-layer structure (tandem structure) in which a plurality of organic compound layers (two organic compound layers 103a and 103b in FIG. 2B) are provided between a pair of electrodes and a charge-generation layer 106 is provided between the organic compound layers. A light-emitting device having the tandem structure enables fabrication of a light-emitting apparatus that has high efficiency without changing the amount of current.

The charge-generation layer 106 has a function of injecting electrons into one of the organic compound layers 103a and 103b and injecting holes into the other of the organic compound layers 103a and 103b when a potential difference is caused between the first electrode 101 and the second electrode 102. Thus, when voltage is applied in FIG. 2B such that the potential of the first electrode 101 is higher than that of the second electrode 102, the charge-generation layer 106 injects electrons into the organic compound layer 103a and injects holes into the organic compound layer 103b.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

FIG. 2C illustrates a stacked-layer structure of the organic compound layer 103 in the light-emitting device of one embodiment of the present invention. In this case, the first electrode 101 is regarded as functioning as an anode, and the second electrode 102 is regarded as functioning as a cathode. The organic compound layer 103 has a structure in which the hole-injection layer 111, the hole-transport layer 112, the light-emitting layer 113, the electron-transport layer 114, and the electron-injection layer 115 are stacked in this order over the first electrode 101. Note that the light-emitting layer 113 may have a stacked-layer structure of a plurality of light-emitting layers that emit light of different colors. For example, a light-emitting layer including a light-emitting substance that emits red light, a light-emitting layer including a light-emitting substance that emits green light, and a light-emitting layer including a light-emitting substance that emits blue light may be stacked with or without a layer including a carrier-transport material therebetween. Alternatively, a light-emitting layer including a light-emitting substance that emits yellow light and a light-emitting layer including a light-emitting substance that emits blue light may be used in combination. Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer including a light-emitting substance that emits blue light and a second light-emitting layer including a light-emitting substance that emits blue light may be stacked with or without a layer including a carrier-transport material therebetween. The structure in which a plurality of light-emitting layers that emit light of the same color are stacked can sometimes achieve higher reliability than a single-layer structure. In the case where a plurality of light-emitting layers are provided as in the tandem structure illustrated in FIG. 2B, the layers in the organic compound layer 103 are sequentially stacked from the anode side as described above. When the first electrode 101 is the cathode and the second electrode 102 is the anode, the stacking order of the layers in the organic compound layer 103 is reversed. Specifically, the layer 111 over the first electrode 101 serving as the cathode is an electron-injection layer; the layer 112 is an electron-transport layer; the layer 113 is a light-emitting layer; the layer 114 is a hole-transport layer; and the layer 115 is a hole-injection layer.

The light-emitting layer 113 included in the organic compound layers (103, 103a, and 103b) includes an appropriate combination of a light-emitting substance and a plurality of substances, so that fluorescent light of a desired color or phosphorescent light of a desired color can be obtained. The light-emitting layer 113 may have a stacked-layer structure having different emission colors. In that case, the light-emitting substance and other substances are different between the stacked light-emitting layers. Alternatively, the plurality of organic compound layers (103a and 103b) in FIG. 2B may exhibit their respective emission colors. Also in that case, the light-emitting substance and other substances can be different between the light-emitting layers.

The light-emitting device of one embodiment of the present invention can have a micro optical resonator (microcavity) structure when, for example, the first electrode 101 is a reflective electrode and the second electrode 102 is a transflective electrode in FIG. 2C. Thus, light from the light-emitting layer 113 in the organic compound layer 103 can be resonated between the electrodes and light emitted through the second electrode 102 can be intensified. Thus, high resolution can be easily achieved. In addition, emission intensity at a predetermined wavelength in the front direction can be increased, whereby power consumption can be reduced.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer greater than or equal to 1) or close to mλ/2.

To amplify desired light (wavelength: λ) obtained from the light-emitting layer 113, each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) is preferably adjusted to be (2m′+1)λ/4 (m′ is an integer greater than or equal to 1) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

The light-emitting device illustrated in FIG. 2D is a light-emitting device having the tandem structure. The tandem structure enables a light-emitting device to emit light with high luminance. Furthermore, the amount of current needed for obtaining a predetermined luminance can be smaller in the tandem structure than in the single structure; thus, the tandem structure enables higher reliability. In addition, power consumption can be reduced.

The light-emitting device illustrated in FIG. 2E is an example of the light-emitting device having the tandem structure illustrated in FIG. 2B, and includes three organic compound layers (103a, 103b, and 103c) stacked with charge-generation layers (106a and 106b) positioned therebetween, as illustrated in FIG. 2E. The three organic compound layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light; alternatively, the light-emitting layer 113a can emit red light, the light-emitting layer 113b can emit blue light, green light, or yellow light, and the light-emitting layer 113c can emit red light.

In the above light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity lower than or equal to 1×10⁻² Ω·cm.

When one of the first electrode 101 and the second electrode 102 is a reflective electrode in the above light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity lower than or equal to 1×10⁻² Ω·cm.

### Specific Structure of Light-Emitting Device

Next, a specific structure of the light-emitting device of one embodiment of the present invention will be described. Here, the description is made using FIG. 2D illustrating the tandem structure. Note that the structure of the organic compound layer applies also to the structure of the light-emitting devices having the single structure in FIGS. 2A and 2C. When the light-emitting device in FIG. 2D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the organic compound layer 103b, with the use of a material selected as appropriate.

### Materials of Light-Emitting Device

### Light-Emitting Layer

The light-emitting layers (113, 113a, and 113b) include a light-emitting substance. Note that as a light-emitting substance that can be used in the light-emitting layers (113, 113a, and 113b), a substance whose emission color is blue, violet, bluish violet, green, yellowish green, yellow, orange, red, or the like can be used as appropriate. When a plurality of light-emitting layers are provided, the use of different light-emitting substances for the light-emitting layers enables exhibiting different emission colors (e.g., white light emission obtained by a combination of complementary emission colors). Furthermore, a stacked-layer structure in which one light-emitting layer contains two or more kinds of light-emitting substances may be employed.

The light-emitting layers (113, 113a, and 113b) may each include one or more kinds of organic compounds (e.g., a host material) in addition to a light-emitting substance (a guest material).

Specifically, the light-emitting layer 113 can have the structure that is described with reference to FIG. 1B. In the light-emitting layer 113, the host materials 118 are present in the largest proportion by weight, and the guest material 119 (phosphorescent compound) is dispersed in the host materials 118. The T₁ levels of the host materials 118 (the organic compounds 118_1 and 118_2) in the light-emitting layer 113 are preferably higher than the T₁ level of the guest material 119 in the light-emitting layer 113.

The lowest triplet excitation energy level (T₁ level) can be calculated, using a thin film of a sample, from an emission edge obtained by measurement of an emission spectrum (phosphorescence spectrum) at a low temperature (e.g., 10 K). Note that the emission spectrum of an emission center substance may be measured using a sample in the form of a thin film or a solution; however, a sample in the form of a solution is preferably used for examination of the state of an isolated molecule. As a solvent of the solution, a solvent with relatively low polarity, such as toluene or chloroform, is preferably used. In the case where the emission center substance is a phosphorescent compound, the temperature at which the lowest triplet excitation energy level (T₁ level) is measured may be either low temperature (e.g., 10 K) or room temperature (e.g., 298 K), and the lowest triplet excitation energy level can be calculated from an emission edge obtained by measurement of an emission spectrum (phosphorescence spectrum). Note that the emission edge can be determined as the intersection of a tangent and the horizontal axis (representing wavelength) or the baseline. The tangent is drawn at a point at which the slope on a shorter wavelength side of the shortest-wavelength peak (or the shortest-wavelength shoulder peak) of the emission spectrum (phosphorescence spectrum) has the maximum absolute value.

Examples of the light-emitting substance that can be used as the guest material include a substance emitting red light. In addition, the substance emitting red light is preferably a substance emitting phosphorescent light, particularly preferably an organometallic complex. Examples of the light-emitting substances include organometallic iridium complexes with a pyrimidine skeleton, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and bis[4,6-di(naphthalen-1-yl)pyrimidinato](dipivaloylmethanato)iridium(III) (abbreviation: [Ir(d1npm)₂(dpm)]); organometallic iridium complexes with a pyrazine skeleton, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), and (acetylacetonato)bis[2,3-bis(4-fluorophenyl)quinoxalinato]iridium(III) (abbreviation: [Ir(Fdpq)₂(acac)]); organometallic iridium complexes with a pyridine skeleton, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), (3,7-diethyl-4,6-nonanedionato-κO⁴,κO⁶)bis[2,4-dimethyl-6-[7-(1-methylethyl)-1-isoquinolinyl-κN]phenyl-κC]iridium(III), and (3,7-diethyl-4,6-nonanedionato-κO⁴,κO⁶)bis[2,4-dimethyl-6-[5-(1-methylethyl)-2-quinolinyl-κN]phenyl-κC]iridium(III); a platinum complex such as 2,3,7,8,12,13,17,18-octaethyl-21H,23H-porphyrinplatinum(II) (abbreviation: PtOEP); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato](monophenanthroline)europium(III) (abbreviation: [Eu(TTA)₃(Phen)]). These compounds have an emission peak in the wavelength range from 600 nm to 700 nm. Furthermore, the organometallic iridium complexes with a pyrazine skeleton can provide red light emission with favorable chromaticity. Note that other known red phosphorescent substances can also be used. A compound obtained by substituting deuterium for part of hydrogen in any of these compounds can also be used.

In the case where a light-emitting apparatus does not use a red-light-emitting substance as the light-emitting substance or includes light-emitting devices with different structures, the light-emitting substance may be a fluorescent substance, a phosphorescent substance, a substance exhibiting thermally activated delayed fluorescence (TADF), or any other light-emitting substance.

Examples of the material that can be used as a light-emitting substance that emits fluorescent light (a fluorescent substance) in the light-emitting layer 113 are as follows. Any other fluorescent substance can also be used.

The examples include 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N′-diphenyl-N,N′-bis[4-(9-phenyl-9H-fluoren-9-yl)phenyl]pyrene-1,6-diamine (abbreviation: 1,6FLPAPrn), N,N′-bis(3-methylphenyl)-N,N′-bis[3-(9-phenyl-9H-fluoren-9-yl)phenyl]pyrene-1,6-diamine (abbreviation: 1,6mMemFLPAPrn), N,N′-bis[4-(9H-carbazol-9-yl)phenyl]-N,N′-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), perylene, 2,5,8,11-tetra-tert-butylperylene (abbreviation: TBP), 4-(10-phenyl-9-anthryl)-4′-(9-phenyl-9H-carbazol-3-yl)triphenylamine (abbreviation: PCBAPA), N,N″-(2-tert-butylanthracene-9,10-diyldi-4,1-phenylene)bis(N,N′,N′-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N′,N′-triphenyl-1,4-phenylenediamine (abbreviation: 2DPAPPA), N,N,N′,N′,N″,N″,N′″,N′″-octaphenyldibenzo[g,p]chrysene-2,7,10,15-tetraamine (abbreviation: DBC1), coumarin 30, N-(9,10-diphenyl-2-anthryl)-N,9-diphenyl-9H-carbazol-3-amine (abbreviation: 2PCAPA), N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N′,N′-triphenyl-1,4-phenylenediamine (abbreviation: 2DPAPA), N-[9,10-bis(biphenyl-2-yl)-2-anthryl]-N,N′,N′-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N′-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N′,N′-tetrakis(4-methylphenyl)tetracene-5,11-diamine (abbreviation: p-mPhTD), 7,14-diphenyl-N,N,N′,N′-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJTM), N,N′-diphenyl-N,N′-(1,6-pyrene-diyl)bis[(6-phenylbenzo[b]naphtho[1,2-d]furan)-8-amine](abbreviation: 1,6BnfAPrn-03), N,N′-diphenyl-N,N′-bis(9-phenyl-9H-carbazol-2-yl)naphtho[2,3-b;6,7-b′]bisbenzofuran-3,10-diamine (abbreviation: 3,10PCA2Nbf(IV)-02), and 3,10-bis[N-(dibenzofuran-3-yl)-N-phenylamino]naphtho[2,3-b;6,7-b′]bisbenzofuran (abbreviation: 3,10FrA2Nbf(IV)-02). Fused aromatic diamine compounds typified by pyrenediamine compounds such as 1,6FLPAPrn, 1,6mMemFLPAPrn, and 1,6BnfAPrn-03 are particularly preferable because they have high hole-trapping properties and have high emission efficiency or high reliability.

A fused heteroaromatic compound containing nitrogen and boron, especially a compound having a diaza-boranaphtho-anthracene skeleton, exhibits a narrow emission spectrum, emits blue light with high color purity, and can thus be used suitably. Examples of the compound include 5,9-diphenyl-5H,9H-[1,4]benzazaborino[2,3,4-kl]phenazaborin (abbreviation: DABNA-1), 9-(biphenyl-3-yl)-N,N,5,11-tetraphenyl-5H,9H-[1,4]benzazaborino[2,3,4-kl]phenazaborin-3-amine (abbreviation: DABNA-2), 2,12-di(tert-butyl)-5,9-di(4-tert-butylphenyl)-N,N′-diphenyl-5H,9H-[1,4]benzazaborino[2,3,4-ki]phenazaborin-7-amine (abbreviation: DPhA-tBu4DABNA), 2,12-di(tert-butyl)-N,N,5,9-tetra(4-tert-butylphenyl)-5H,9H-[1,4]benzazaborino[2,3,4-kl]phenazaborin-7-amine (abbreviation: tBuDPhA-tBu4DABNA), 2,12-di(tert-butyl)-5,9-di(4-tert-butylphenyl)-7-methyl-5H,9H-[1,4]benzazaborino[2,3,4-ki]phenazaborine (abbreviation: Me-tBu4DABNA), N⁷,N⁷,N¹³,N¹³,5,9,11,15-octaphenyl-5H,9H,11H,15H-[1,4]benzazaborino[2,3,4-ki][1,4]benzazaborino[4′,3′,2′:4,5][1,4]benzazaborino[3,2-b]phenazaborine-7,13-diamine (abbreviation: v-DABNA), and 2-(4-tert-butylphenyl)benz[5,6]indolo[3,2,1-jk]benzo[b]carbazole (abbreviation: tBuPBibc).

Besides the above compounds, a compound with an indole skeleton such as 9,10,11-tris[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-k]phenazaborine (abbreviation: BBCz-G) or 9,11-bis[3,6-bis(1,1-dimethylethyl)-9H-carbazolyl-9-yl]-2,5,15,18-tetrakis(1,1-dimethylethyl)indolo[3,2,1-de]indolo[3′,2′,1′:8,1][1,4]benzazaborino[2,3,4-kl]phenazaborine (abbreviation: BBCz-Y) can be suitably used.

Examples of the material that can be used when a phosphorescent substance is used as the light-emitting substance in the light-emitting layer 113 are as follows.

The examples include organometallic iridium complexes with a 4H-triazole skeleton, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]) and tris(5-methyl-3,4-diphenyl-4H-1,2,4-triazolato)iridium(III) (abbreviation: [Ir(Mptz)₃]); organometallic iridium complexes with a 1H-triazole skeleton, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic iridium complexes with an imidazole skeleton, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-{1-[2,6-bis(1-methylethyl)phenyl]-1H-imidazol-2-yl-κN³}-4-cyanophenyl-κC)iridium(III) (abbreviation: CNImIr); an organometallic iridium complex with a benzimidazolidene skeleton, such as tris[(6-tert-butyl-3-phenyl-2H-imidazo[4,5-b]pyrazin-1-yl-κC²)phenyl-κC]iridium(III) (abbreviation: [Ir(cb)₃]); organometallic iridium complexes in which a phenylpyridine derivative with an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)); and a platinum complex such as (2-{3-[3-(3,5-di-tert-butylphenyl)benzimidazol-1-yl-2-ylidene-κC²]phenoxy-κC²}-9-(4-tert-butyl-2-pyridinyl-κN)carbazole-2,1-diyl-κC¹)platinum(II) (abbreviation: PtON-TBBI). These compounds exhibit blue phosphorescence and have an emission peak in the wavelength range from 440 nm to 520 nm. A compound obtained by substituting deuterium for part of hydrogen in any of these compounds can also be used.

Other examples include organometallic iridium complexes with a pyrimidine skeleton, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), and (acetylacetonato)bis(4,6-diphenylpyrimidinato)iridium(III) (abbreviation: [Ir(dppm)₂(acac)]); organometallic iridium complexes with a pyrazine skeleton, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes with a pyridine skeleton, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-(methyl-d₃)-8-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), [2-(4-d₃-methyl-5-phenyl-2-pyridinyl-κN²)phenyl-κC]bis[2-(5-d₃-methyl-2-pyridinyl-κN²)phenyl-κC]iridium(III) (abbreviation: [Ir(5mppy-d₃)₂(mdppy-d₃)]), [2-methyl-8-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), and tris{2-[5-(methyl-d₃)-4-phenyl-2-pyridinyl-N]phenyl-κC}iridium(III) (abbreviation: Ir(5m4dppy-d₃)₃); a rare earth metal complex such as tris(acetylacetonato)(monophenanthroline)terbium(III) (abbreviation: [Tb(acac)₃(Phen)]); organometallic platinum complexes such as (2-{1-(5-tert-butylbiphenyl-2-yl)-4-[3-tert-butyl-5-(4-phenyl-2-pyridinyl-κN)phenyl-κC⁶]-2-benzimidazolyl-κN³}-4,6-di-tert-butylphenolato-κO)platinum(II) (abbreviation: Pt(tBudppymmtBubiz-tBubp)) and [2-(4-(3,5-di-tert-butylphenyl)-6-{3-[4-(5′-tert-butyl[1,1′:3′,1″-terphenyl]-2′-yl)-2-pyridinyl-κN]phenyl-C²}-2-pyridinyl-κN)phenolato-κO]platinum(II) (abbreviation: Pt(4tButpppypyp-mmtBup)). These are mainly compounds that exhibit green phosphorescence and have an emission peak in the wavelength range from 500 nm to 600 nm. Note that organometallic iridium complexes with a pyrimidine skeleton have distinctively high reliability or emission efficiency and thus are particularly preferable. A compound obtained by substituting deuterium for part of hydrogen in any of these compounds can also be used.

Note that any of the aforementioned red phosphorescent materials can also be used. Besides the above phosphorescent compounds, known phosphorescent compounds may be selected and used.

Examples of a TADF material include a fullerene, a derivative thereof, an acridine, a derivative thereof, and an eosin derivative. Other examples include a metal-containing porphyrin, such as a porphyrin containing magnesium (Mg), zinc (Zn), cadmium (Cd), tin (Sn), platinum (Pt), indium (In), or palladium (Pd). Examples of the metal-containing porphyrin include a protoporphyrin-tin fluoride complex (SnF₂(Proto IX)), a mesoporphyrin-tin fluoride complex (SnF₂(Meso IX)), a hematoporphyrin-tin fluoride complex (SnF₂(Hemato IX)), a coproporphyrin tetramethyl ester-tin fluoride complex (SnF₂(Copro III-4Me)), an octaethylporphyrin-tin fluoride complex (SnF₂(OEP)), an etioporphyrin-tin fluoride complex (SnF₂(Etio I)), and an octaethylporphyrin-platinum chloride complex (PtCl₂OEP), which are represented by the following structural formulae.

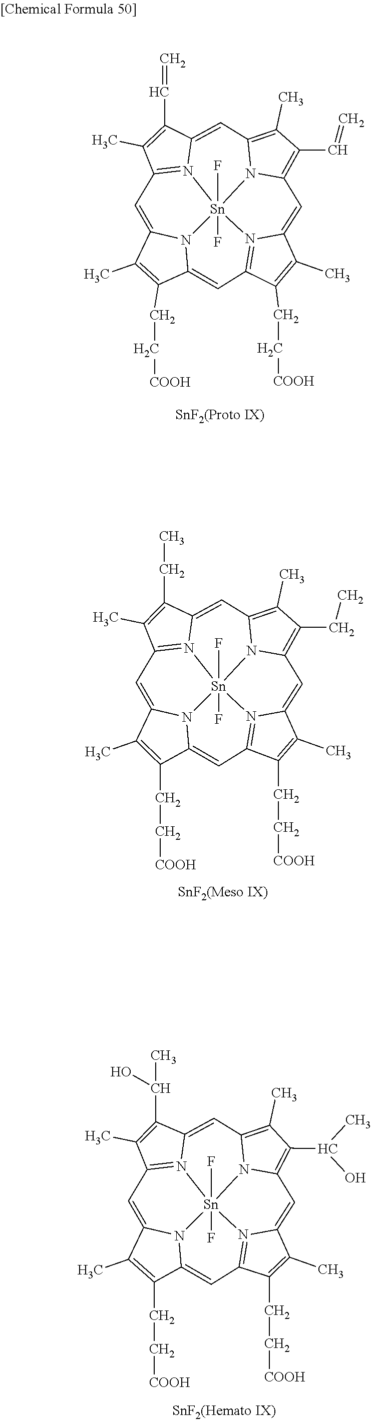

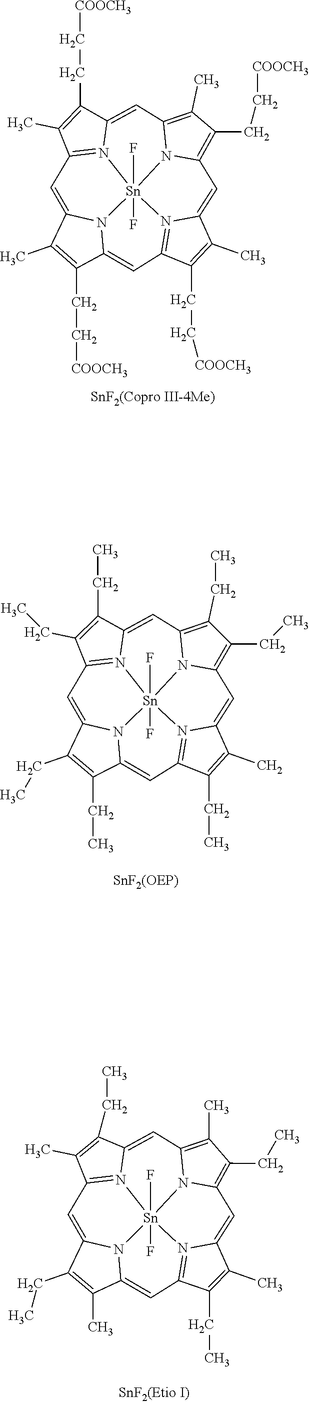

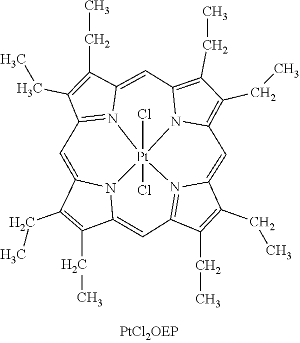

It is also possible to use a heterocyclic compound with one or both of a π-electron rich heteroaromatic ring and a π-electron deficient heteroaromatic ring that is represented by any of the following structural formulae, such as 2-(biphenyl-4-yl)-4,6-bis(12-phenylindolo[2,3-a]carbazol-11-yl)-1,3,5-triazine (abbreviation: PIC-TRZ), 9-(4,6-diphenyl-1,3,5-triazin-2-yl)-9′-phenyl-9H,9′H-3,3′-bicarbazole (abbreviation: PCCzTzn), 9-[4-(4,6-diphenyl-1,3,5-triazin-2-yl)phenyl]-9′-phenyl-3,3′-bi-9H-carbazole (abbreviation: PCCzPTzn), 2-[4-(10H-phenoxazin-10-yl)phenyl]-4,6-diphenyl-1,3,5-triazine (abbreviation: PXZ-TRZ), 3-[4-(5-phenyl-5,10-dihydrophenazin-10-yl)phenyl]-4,5-diphenyl-1,2,4-triazole (abbreviation: PPZ-3TPT), 3-(9,9-dimethyl-9H-acridin-10-yl)-9H-xanthen-9-one (abbreviation: ACRXTN), bis[4-(9,9-dimethyl-9,10-dihydroacridine)phenyl]sulfone (abbreviation: DMAC-DPS), or 10-phenyl-10H,10′H-spiro[acridin-9,9′-anthracen]-10′-one (abbreviation: ACRSA). Such a heterocyclic compound is preferable because of its high electron-transport and hole-transport properties due to the π-electron rich heteroaromatic ring and the π-electron deficient heteroaromatic ring. Among skeletons having the π-electron deficient heteroaromatic ring, a pyridine skeleton, a diazine skeleton (a pyrimidine skeleton, a pyrazine skeleton, or a pyridazine skeleton), and a triazine skeleton are particularly preferable because of their high stability and reliability. In particular, a benzofuropyrimidine skeleton, a benzothienopyrimidine skeleton, a benzofuropyrazine skeleton, and a benzothienopyrazine skeleton are preferable because of their high acceptor properties and high reliability. Among skeletons having the π-electron rich heteroaromatic ring, an acridine skeleton, a phenoxazine skeleton, a phenothiazine skeleton, a furan skeleton, a thiophene skeleton, and a pyrrole skeleton have high stability and reliability; thus, at least one of these skeletons is preferably included. A dibenzofuran skeleton is preferable as a furan skeleton, and a dibenzothiophene skeleton is preferable as a thiophene skeleton. As a pyrrole skeleton, an indole skeleton, a carbazole skeleton, an indolocarbazole skeleton, a bicarbazole skeleton, and a 3-(9-phenyl-9H-carbazol-3-yl)-9H-carbazole skeleton are particularly preferable. Note that a substance in which the π-electron rich heteroaromatic ring is directly bonded to the π-electron deficient heteroaromatic ring is particularly preferred because the electron-donating property of the π-electron rich heteroaromatic ring and the electron-accepting property of the π-electron deficient heteroaromatic ring are both improved, the energy difference between the S₁ level and the T₁ level becomes small, and thus thermally activated delayed fluorescence can be obtained with high efficiency. Note that an aromatic ring to which an electron-withdrawing group such as a cyano group is bonded may be used instead of the π-electron deficient heteroaromatic ring. As a π-electron rich skeleton, an aromatic amine skeleton, a phenazine skeleton, or the like can be used. As a π-electron deficient skeleton, a xanthene skeleton, a thioxanthene dioxide skeleton, an oxadiazole skeleton, a triazole skeleton, an imidazole skeleton, an anthraquinone skeleton, a skeleton containing boron such as phenylborane or boranthrene, an aromatic ring or a heteroaromatic ring with a nitrile group or a cyano group such as benzonitrile or cyanobenzene, a carbonyl skeleton such as benzophenone, a phosphine oxide skeleton, a sulfone skeleton, or the like can be used. As described above, a it-electron deficient skeleton and a it-electron rich skeleton can be used instead of at least one of the it-electron deficient heteroaromatic ring and the it-electron rich heteroaromatic ring.

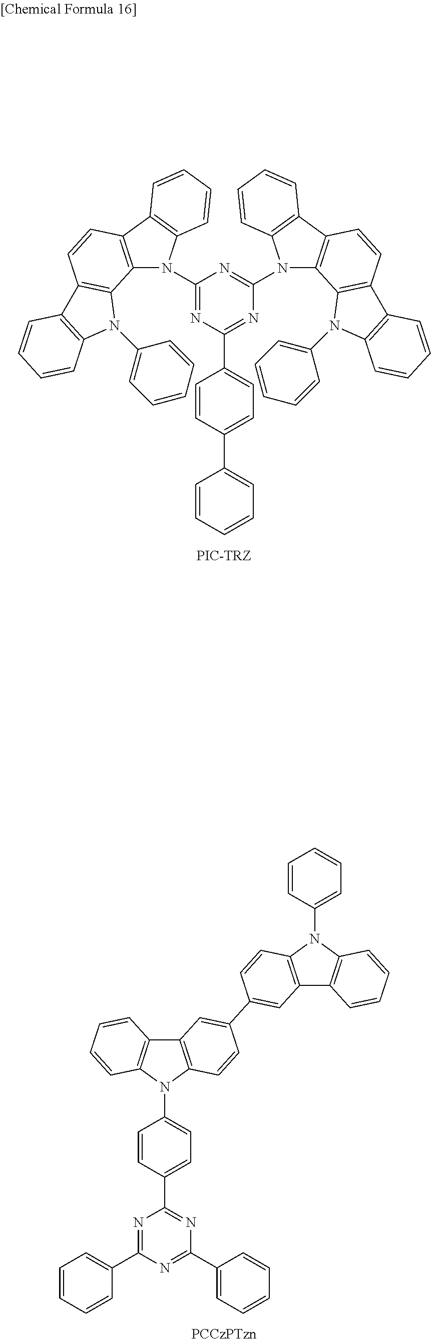 [SMILES: c1ccc(-c2ccc(-c3nc(-n4c5ccccc5c5ccc6c7ccccc7n(-c7ccccc7)c6c54)nc(-n4c5ccccc5c5ccc6c7ccccc7n(-c7ccccc7)c6c54)n3)cc2)cc1 ; c1ccc(-c2nc(-c3ccccc3)nc(-c3ccc(-n4c5ccccc5c5cc(-c6ccc7c(c6)c6ccccc6n7-c6ccccc6)ccc54)cc3)n2)cc1]

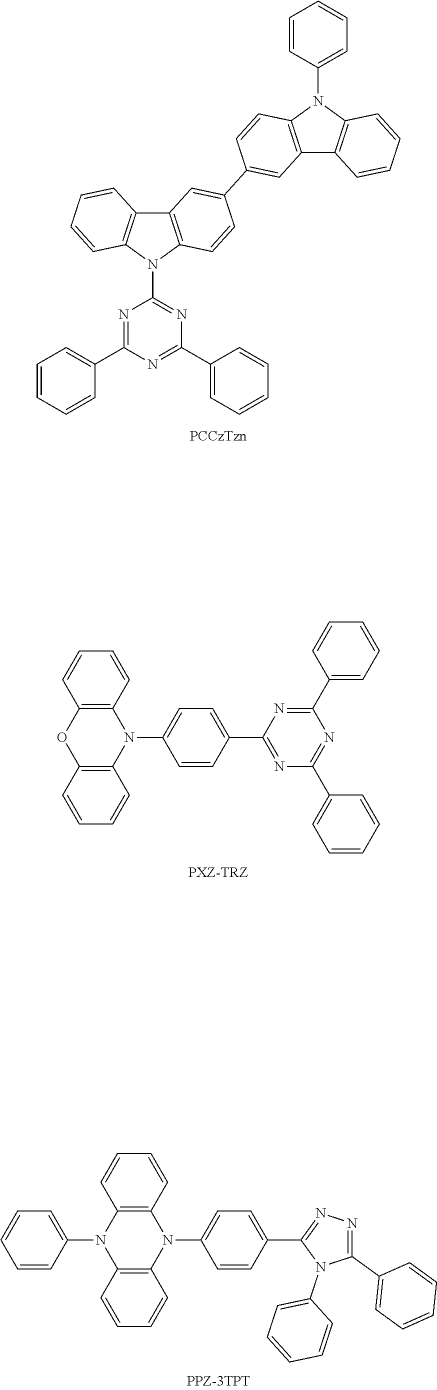 [SMILES: c1ccc(-c2nc(-c3ccccc3)nc(-n3c4ccccc4c4cc(-c5ccc6c(c5)c5ccccc5n6-c5ccccc5)ccc43)n2)cc1 ; c1ccc(-c2nc(-c3ccccc3)nc(-c3ccc(N4c5ccccc5Oc5ccccc54)cc3)n2)cc1 ; c1ccc(-c2nnc(-c3ccc(N4c5ccccc5N(c5ccccc5)c5ccccc54)cc3)n2-c2ccccc2)cc1]

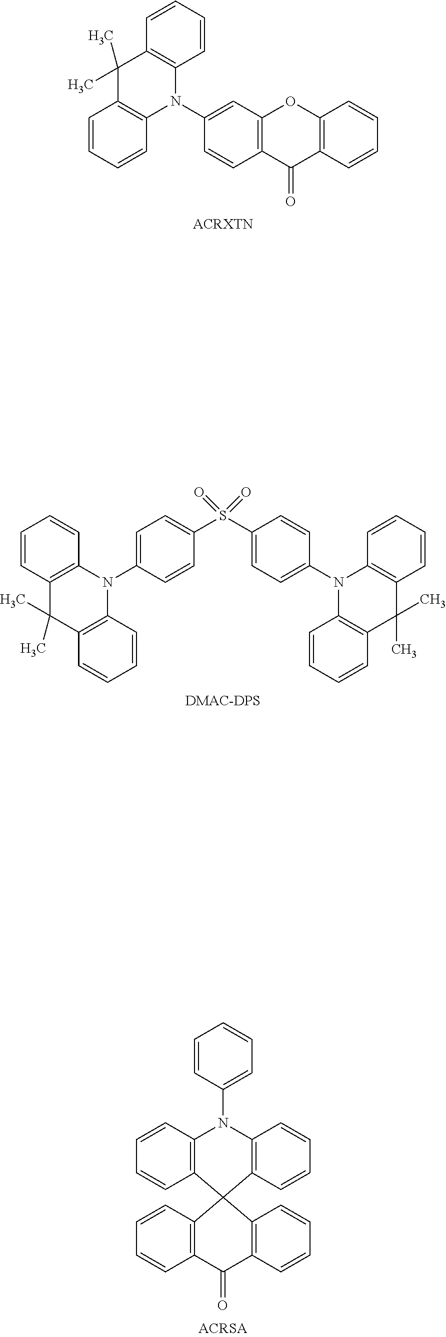 [SMILES: CC1(C)c2ccccc2N(c2ccc3c(=O)c4ccccc4oc3c2)c2ccccc21 ; CC1(C)c2ccccc2N(c2ccc(S(=O)(=O)c3ccc(N4c5ccccc5C(C)(C)c5ccccc54)cc3)cc2)c2ccccc21 ; O=C1c2ccccc2C2(c3ccccc31)c1ccccc1N(c1ccccc1)c1ccccc12]

It is also possible to use a TADF material that enables reversible intersystem crossing at extremely high speed and emits light in accordance with a thermal equilibrium model between a singlet excited state and a triplet excited state. Since such a TADF material has an extremely short emission lifetime (excitation lifetime), an efficiency decrease of a light-emitting element in a high-luminance region can be inhibited. Specifically, a material having the following molecular structure can be used.

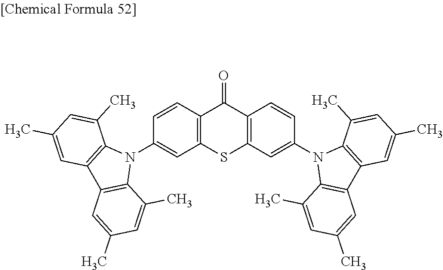

Note that a TADF material is a material having a small difference between the S₁ level and the T₁ level and a function of converting triplet excitation energy into singlet excitation energy by reverse intersystem crossing. Thus, a TADF material can upconvert triplet excitation energy into singlet excitation energy (i.e., reverse intersystem crossing) using a small amount of thermal energy and efficiently generate a singlet excited state. In addition, the triplet excitation energy can be converted into light emission.

An exciplex whose excited state is formed by two kinds of substances has an extremely small difference between the S₁ level and the T₁ level and functions as a TADF material capable of converting triplet excitation energy into singlet excitation energy.

A phosphorescence spectrum observed at a low temperature (e.g., 77 K to 10 K) is used for an index of the T₁ level. When the level of energy of light with a wavelength of the line obtained by extrapolating a tangent to the fluorescence spectrum at a tail on the short wavelength side is the S₁ level and the level of energy of light with a wavelength of the line obtained by extrapolating a tangent to the phosphorescence spectrum at a tail on the short wavelength side is the T₁ level, the difference between the S₁ level and the T₁ level of the TADF material is preferably less than or equal to 0.3 eV, further preferably less than or equal to 0.2 eV.

When a TADF material is used as the light-emitting substance, the S₁ level of the host material is preferably higher than that of the TADF material. In addition, the T₁ level of the host material is preferably higher than that of the TADF material.

As an electron-transport material used as the host material, for example, any of metal complexes such as bis(10-hydroxybenzo[h]quinolinato)beryllium(II) (abbreviation: BeBq₂), bis(2-methyl-8-quinolinolato)(4-phenylphenolato)aluminum(III) (abbreviation: BAlq), bis(8-quinolinolato)zinc(II) (abbreviation: Znq), bis[2-(2-benzoxazolyl)phenolato]zinc(II) (abbreviation: ZnPBO), and bis[2-(2-benzothiazolyl)phenolato]zinc(II) (abbreviation: ZnBTZ); or an organic compound having a π-electron deficient heteroaromatic ring can be used. Examples of the organic compound having a π-electron deficient heteroaromatic ring include an organic compound having a heteroaromatic ring with an azole skeleton, such as 2-(4-biphenyl)-5-(4-tert-butyl-phenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(4-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), or 2-[3-(dibenzothiophen-4-yl)phenyl]-1-phenyl-1H-benzimidazole (abbreviation: mDBTBIm-II); an organic compound having a heteroaromatic ring with a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 2,4-bis[4-(1-naphthyl)phenyl]-6-[4-(3-pyridyl)phenyl]pyrimidine (abbreviation: 2,4NP-6PyPPm), 6-(biphenyl-3-yl)-4-[3,5-bis(9H-carbazol-9-yl)phenyl]-2-phenylpyrimidine (abbreviation: 6mBP-4Cz2PPm), 4-[3,5-bis(9H-carbazol-9-yl)phenyl]-2-phenyl-6-(biphenyl-4-yl)pyrimidine (abbreviation: 6BP-4Cz2PPm), or 7-[4-(9-phenyl-9H-carbazol-2-yl)quinazolin-2-yl]-7H-dibenzo[c,g]carbazole (abbreviation: PC-cgDBCzQz); an organic compound having a heteroaromatic ring with a pyridine skeleton, such as 3,5-bis[3-(9H-carbazol-9-yl)phenyl]pyridine (abbreviation: 35DCzPPy) or 1,3,5-tri[(3-pyridyl)-phenyl-3-yl]benzene (abbreviation: TmPyPB); and an organic compound having a heteroaromatic ring with a triazine skeleton, such as 2-[3′-(9,9-dimethyl-9H-fluoren-2-yl)biphenyl-3-yl]-4,6-diphenyl-1,3,5-triazine (abbreviation: mFBPTzn), 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), or 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn). Among the above materials, the organic compound having a heteroaromatic ring with a diazine skeleton, the organic compound having a heteroaromatic ring with a pyridine skeleton, and the organic compound having a heteroaromatic ring with a triazine skeleton have high reliability and thus are preferable. In particular, the organic compound having a heteroaromatic ring with a diazine (pyrimidine or pyrazine) skeleton and the organic compound having a heteroaromatic ring with a triazine skeleton have a high electron-transport property to contribute to a reduction in driving voltage.

As a hole-transport material used as the host material, an organic compound having an amine skeleton or a π-electron rich heteroaromatic ring can be used. Examples of the organic compound having an amine skeleton or a π-electron rich heteroaromatic ring include a compound having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), or 9,9′-diphenyl-9H,9′H-3,3′-bicarbazole (abbreviation: PCCP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because these compounds are highly reliable and have high hole-transport properties to contribute to a reduction in driving voltage. In addition, the organic compounds given as examples of the material with a hole-transport property that can be used for the hole-transport layer 112 can also be used as the hole-transport material that is the host material.

By mixing the electron-transport material with the hole-transport material, the-transport property of the light-emitting layer 113 can be easily adjusted and a recombination region can be easily controlled. A TADF material can be used as the electron-transport material or the hole-transport material.

As the TADF material that can be used as the host material, the above materials mentioned as the TADF material can also be used. When the TADF material is used as the host material, triplet excitation energy generated in the TADF material is converted into singlet excitation energy by reverse intersystem crossing and transferred to the light-emitting substance, whereby the emission efficiency of the light-emitting device can be increased. Here, the TADF material functions as an energy donor, and the light-emitting substance functions as an energy acceptor.

This is very effective in the case where the light-emitting substance is a fluorescent substance. In that case, the S₁ level of the TADF material is preferably higher than that of the fluorescent substance in order that high emission efficiency can be achieved. Furthermore, the T₁ level of the TADF material is preferably higher than the S₁ level of the fluorescent substance. Therefore, the T₁ level of the TADF material is preferably higher than that of the fluorescent substance.

It is also preferable to use a TADF material that emits light whose wavelength overlaps with the wavelength of the lowest-energy absorption band of the fluorescent substance. This enables smooth transfer of excitation energy from the TADF material to the fluorescent substance and accordingly enables efficient light emission, which is preferable.

In order that singlet excitation energy can be efficiently generated from the triplet excitation energy by reverse intersystem crossing, carrier recombination preferably occurs in the TADF material. It is also preferable that the triplet excitation energy generated in the TADF material not be transferred to the triplet excitation energy of the fluorescent substance. For that reason, the fluorescent substance preferably includes a protective group around a luminophore (a skeleton which causes light emission) of the fluorescent substance. As the protective group, a substituent having no π bond and a saturated hydrocarbon are preferably used. Specific examples include an alkyl group having 3 to 10 carbon atoms, a substituted or unsubstituted cycloalkyl group having 3 to 10 carbon atoms, and a trialkylsilyl group having 3 to 10 carbon atoms. It is further preferable that the fluorescent substance have a plurality of protective groups. The substituents having no π bond are poor in carrier transport performance, whereby the TADF material and the luminophore of the fluorescent substance can be made away from each other with little influence on carrier transport or carrier recombination. Here, the luminophore refers to an atomic group (skeleton) that causes light emission in a fluorescent substance. The luminophore is preferably a skeleton having a π bond, further preferably includes an aromatic ring, and still further preferably includes a fused aromatic ring or a fused heteroaromatic ring. Examples of the luminophore include a phenanthrene skeleton, a stilbene skeleton, an acridone skeleton, a phenoxazine skeleton, a phenothiazine skeleton, a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton. In particular, a fluorescent substance having any of a naphthalene skeleton, an anthracene skeleton, a fluorene skeleton, a chrysene skeleton, a triphenylene skeleton, a tetracene skeleton, a pyrene skeleton, a perylene skeleton, a coumarin skeleton, a quinacridone skeleton, and a naphthobisbenzofuran skeleton is preferred because of its high fluorescence quantum yield.

In the case where a fluorescent substance is used as the light-emitting substance, a material with an anthracene skeleton is suitably used as the host material. The use of a substance with an anthracene skeleton as the host material for the fluorescent substance makes it possible to obtain a light-emitting layer with high emission efficiency and high durability. As the substance with an anthracene skeleton that is used as the host material, a substance having a diphenylanthracene skeleton, or specifically, a 9,10-diphenylanthracene skeleton, is preferable because of being chemically stable. The host material preferably has a carbazole skeleton to have higher hole-injection and hole-transport properties; further preferably, the host material has a benzocarbazole skeleton in which a benzene ring is further fused to a carbazole skeleton, because the HOMO level of the host material having a benzocarbazole skeleton is higher than that of the host material having a carbazole skeleton by approximately 0.1 eV and the host material having a benzocarbazole skeleton is thus easier for holes to enter than the host material having a carbazole skeleton. In particular, the host material preferably has a dibenzocarbazole skeleton, because the HOMO level of the host material having a dibenzocarbazole skeleton is higher than that of the host material having a carbazole skeleton by approximately 0.1 eV, the host material having a dibenzocarbazole skeleton is thus easier for holes to enter than the host material having a carbazole skeleton, and the host material having a dibenzocarbazole skeleton has a higher hole-transport property and higher heat resistance than the host material having a carbazole skeleton. Accordingly, a substance that has both a 9,10-diphenylanthracene skeleton and a carbazole skeleton (or a benzocarbazole or dibenzocarbazole skeleton) is further preferable as the host material. Note that in terms of the hole-injection and hole-transport properties described above, instead of a carbazole skeleton, a benzofluorene skeleton or a dibenzofluorene skeleton may be used. Examples of such a substance include 9-phenyl-3-[4-(10-phenyl-9-anthryl)phenyl]-9H-carbazole (abbreviation: PCzPA), 3-[4-(1-naphthyl)phenyl]-9-phenyl-9H-carbazole (abbreviation: PCPN), 9-[4-(10-phenyl-9-anthryl)phenyl]-9H-carbazole (abbreviation: CzPA), 7-[4-(10-phenyl-9-anthryl)phenyl]-7H-dibenzo[c,g]carbazole (abbreviation: cgDBCzPA), 6-[3-(9,10-diphenyl-2-anthryl)phenyl]benzo[b]naphtho[1,2-d]furan (abbreviation: 2mBnfPPA), 9-phenyl-10-[4′-(9-phenyl-9H-fluoren-9-yl)biphenyl-4-yl]anthracene (abbreviation: FLPPA), 9-(1-naphthyl)-10-(2-naphthyl)anthracene (abbreviation: α,β-ADN), 2-(10-phenylanthracen-9-yl)dibenzofuran, 2-(10-phenyl-9-anthryl)benzo[b]naphtho[2,3-d]furan (abbreviation: Bnf(II)PhA), 9-(2-naphthyl)-10-[3-(2-naphthyl)phenyl]anthracene (abbreviation: βN-mβNPAnth), and 1-{4-[10-(biphenyl-4-yl)-9-anthryl]phenyl}-2-ethyl-1H-benzimidazole (abbreviation: EtBImPBPhA). In particular, CzPA, cgDBCzPA, 2mBnfPPA, and PCzPA have excellent characteristics and thus are preferably selected.

Note that a phosphorescent substance can be used as part of the mixed material. When a fluorescent substance is used as the light-emitting substance, a phosphorescent substance can be used as an energy donor for supplying excitation energy to the fluorescent substance.

An exciplex may be formed of the mixed materials. These mixed materials are preferably selected so as to form an exciplex that exhibits light emission whose wavelength overlaps with the wavelength of the lowest-energy absorption band of the light-emitting substance, in which case energy can be transferred smoothly and light emission can be obtained efficiently. Such a structure is preferably used to reduce the driving voltage.

Note that at least one of the materials forming an exciplex may be a phosphorescent substance. In that case, triplet excitation energy can be efficiently converted into singlet excitation energy by reverse intersystem crossing.

In order to form an exciplex efficiently, a material having an electron-transport property is preferably combined with a material having a hole-transport property and a HOMO level higher than or equal to that of the material having an electron-transport property. In addition, the LUMO level of the material having a hole-transport property is preferably higher than or equal to that of the material having an electron-transport property. Note that the LUMO levels and the HOMO levels of the materials can be derived from the electrochemical characteristics (the reduction potentials and the oxidation potentials) of the materials that are measured by cyclic voltammetry (CV).

The formation of an exciplex can be confirmed by, for example, comparing the emission spectra of the material having a hole-transport property, the material having an electron-transport property, and the mixed film of these materials, and observing the phenomenon in which the emission spectrum of the mixed film is shifted to the longer wavelength side than the emission spectrum of each of the materials (or has another peak on the longer wavelength side). Alternatively, the formation of an exciplex can be confirmed by comparing the transient photoluminescence (PL) of the material having a hole-transport property, the transient PL of the material having an electron-transport property, and the transient PL of the mixed film of the materials, and observing a difference in transient response, such as a phenomenon in which the transient PL lifetime of the mixed film has more long lifetime components or has a larger proportion of delayed components than that of each of the materials. The transient PL can be rephrased as transient electroluminescence (EL). That is, the formation of an exciplex can also be confirmed by a difference in transient response observed by comparison of the transient EL of the material having a hole-transport property, the material having an electron-transport property, and the mixed film of these materials.

Note that the light-emitting layer 113 can be formed by an evaporation method (including a vacuum evaporation method), an ink-jet method, a coating method, gravure printing, or the like. Besides the above-mentioned materials, an inorganic compound such as a quantum dot or a high molecular compound (e.g., an oligomer, a dendrimer, or a polymer) may be used.

### Hole-Injection Layer

The hole-injection layers (111, 111a, and 111b) inject holes from the first electrode 101 serving as the anode and the charge-generation layers (106, 106a, and 106b) into the organic compound layers (103, 103a, and 103b) and include an organic acceptor material and a material having a high hole-injection property.

For the hole-injection layers (111, 111a, and 111b), it is possible to use a compound having an electron-withdrawing group (a halogen group or a cyano group); for example, 7,7,8,8-tetracyano-2,3,5,6-tetrafluoroquinodimethane (abbreviation: F₄-TCNQ), chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F6-TCNNQ), or 2-(7-dicyanomethylen-1,3,4,5,6,8,9,10-octafluoro-7H-pyren-2-ylidene)malononitrile can be used. A compound in which electron-withdrawing groups are bonded to a fused aromatic ring having a plurality of heteroatoms, such as HAT-CN, is particularly preferable because it is thermally stable. A [3]radialene derivative having an electron-withdrawing group (in particular, a cyano group or a halogen group such as a fluoro group) has a very high electron-accepting property and is thus preferable. Specific examples include α,α′,α″-1,2,3-cyclopropanetriylidenetris(4-cyano-2,3,5,6-tetrafluorobenzeneacetonitrile) (abbreviation: Rad), α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile]. As the substance having an acceptor property, molybdenum oxide, vanadium oxide, ruthenium oxide, tungsten oxide, manganese oxide, or the like can be used, other than the above-described organic compounds. Alternatively, the hole-injection layers (111, 111a, and 111b) can be formed using a phthalocyanine-based compound such as phthalocyanine (abbreviation: H₂Pc), a phthalocyanine-based complex compound such as copper phthalocyanine (abbreviation: CuPc), an aromatic amine compound such as 4,4′-bis[N-(4-diphenylaminophenyl)-N-phenylamino]biphenyl (abbreviation: DPAB) or N,N′-bis[4-bis(3-methylphenyl)aminophenyl]-N,N′-diphenyl-4,4′-diaminobiphenyl (abbreviation: DNTPD), or a high molecular compound such as poly(3,4-ethylenedioxythiophene)/polystyrenesulfonic acid (abbreviation: PEDOT/PSS). The substance having an acceptor property can extract electrons from an adjacent hole-transport layer (or hole-transport material) by application of an electric field.

Among substances having an acceptor property, an organic compound having an acceptor property is easy to use because it is easily deposited by evaporation.

Alternatively, a composite material in which a material with a hole-transport property contains any of the aforementioned substances with an acceptor property can be used for the hole-injection layers (111, 111a, and 111b). In the case of using a composite material in which a material with a hole-transport property contains an acceptor substance, a material used to form an electrode can be selected regardless of its work function. In other words, besides a material having a high work function, a material having a low work function can be used for the anode (the first electrode 101).

As the material with a hole-transport property used for the composite material, any of a variety of organic compounds such as aromatic amine compounds, carbazole derivatives, aromatic hydrocarbons, and high molecular compounds (e.g., oligomers, dendrimers, or polymers) can be used. Note that the material with a hole-transport property used for the composite material preferably has a hole mobility higher than or equal to 1×10⁻⁶ cm²/Vs. Organic compounds that can be used as the material with a hole-transport property in the composite material are specifically given below.

Examples of the aromatic amine compound that can be used for the composite material include N,N′-di(p-tolyl)-N,N′-diphenyl-p-phenylenediamine (abbreviation: DTDPPA), 4,4′-bis[N-(4-diphenylaminophenyl)-N-phenylamino]biphenyl (abbreviation: DPAB), N,N′-bis[4-bis(3-methylphenyl)aminophenyl]-N,N′-diphenyl-4,4′-diaminobiphenyl (abbreviation: DNTPD), and 1,3,5-tris[N-(4-diphenylaminophenyl)-N-phenylamino]benzene (abbreviation: DPA3B). Specific examples of the carbazole derivative include 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), 3-[N-(1-naphthyl)-N-(9-phenylcarbazol-3-yl)amino]-9-phenylcarbazole (abbreviation: PCzPCN1), 4,4′-di(N-carbazolyl)biphenyl (abbreviation: CBP), 1,3,5-tris[4-(N-carbazolyl)phenyl]benzene (abbreviation: TCPB), 9-[4-(10-phenyl-9-anthryl)phenyl]-9H-carbazole (abbreviation: CzPA), and 1,4-bis[4-(N-carbazolyl)phenyl]-2,3,5,6-tetraphenylbenzene. Examples of the aromatic hydrocarbon include 2-tert-butyl-9,10-di(2-naphthyl)anthracene (abbreviation: t-BuDNA), 2-tert-butyl-9,10-di(1-naphthyl)anthracene, 9,10-bis(3,5-diphenylphenyl)anthracene (abbreviation: DPPA), 2-tert-butyl-9,10-bis(4-phenylphenyl)anthracene (abbreviation: t-BuDBA), 9,10-di(2-naphthyl)anthracene (abbreviation: DNA), 9,10-diphenylanthracene (abbreviation: DPAnth), 2-tert-butylanthracene (abbreviation: t-BuAnth), 9,10-bis(4-methyl-1-naphthyl)anthracene (abbreviation: DMNA), 2-tert-butyl-9,10-bis[2-(1-naphthyl)phenyl]anthracene, 9,10-bis[2-(1-naphthyl)phenyl]anthracene, 2,3,6,7-tetramethyl-9,10-di(1-naphthyl)anthracene, 2,3,6,7-tetramethyl-9,10-di(2-naphthyl)anthracene, 9,9′-bianthryl, 10,10′-diphenyl-9,9′-bianthryl, 10,10′-bis(2-phenylphenyl)-9,9′-bianthryl, 10,10′-bis[(2,3,4,5,6-pentaphenyl)phenyl]-9,9′-bianthryl, anthracene, tetracene, rubrene, perylene, and 2,5,8,11-tetra(tert-butyl)perylene. Other examples include pentacene and coronene. The aromatic hydrocarbon may have a vinyl skeleton. Examples of the aromatic hydrocarbon having a vinyl group include 4,4′-bis(2,2-diphenylvinyl)biphenyl (abbreviation: DPVBi) and 9,10-bis[4-(2,2-diphenylvinyl)phenyl]anthracene (abbreviation: DPVPA).

Other examples include high molecular compounds such as poly(N-vinylcarbazole) (abbreviation: PVK), poly(4-vinyltriphenylamine) (abbreviation: PVTPA), poly[N-(4-{N-[4-(4-diphenylamino)phenyl]phenyl-N-phenylamino}phenyl)methacrylamide](abbreviation: PTPDMA), and poly[N,N′-bis(4-butylphenyl)-N,N′-bis(phenyl)benzidine](abbreviation: Poly-TPD).

The material with a hole-transport property used for the composite material further preferably has at least any one of a carbazole skeleton, a dibenzofuran skeleton, a dibenzothiophene skeleton, and an anthracene skeleton. In particular, an aromatic amine having a substituent with a dibenzofuran ring or a dibenzothiophene ring, an aromatic monoamine having a naphthalene ring, or an aromatic monoamine in which a 9-fluorenyl group is bonded to the nitrogen of the amine through an arylene group may be used. Note that the material with a hole-transport property preferably has an N,N-bis(4-biphenyl)amino group to enable fabricating a light-emitting device having a long lifetime. Specific examples of the material with a hole-transport property include N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-([2,1′-binaphthyl]-6-yl)triphenylamine (abbreviation: BBAαNβNB), 4,4′-diphenyl-4″-([2,1′-binaphthyl]-7-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-([2,2′-binaphthyl]-6-yl)triphenylamine (abbreviation: BBA(βN2)B), 4,4′-diphenyl-4″-([2,2′-binaphthyl]-7-yl)triphenylamine (abbreviation: BBA(βN2)B-03), 4,4′-diphenyl-4″-([1,2′-binaphthyl]-4-yl)triphenylamine (abbreviation: BBAβNαNB), 4,4′-diphenyl-4″-([1,2′-binaphthyl]-5-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-[4-(9-phenylfluoren-9-yl)phenyl]triphenylamine (abbreviation: BPAFLBi), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), N-phenyl-N-[4-(9-phenyl-9H-carbazol-3-yl)phenyl]-9,9′-spirobi[9H-fluoren]-2-amine (abbreviation: PCBASF), N-(biphenyl-4-yl)-N-[4-(9-phenyl-9H-carbazol-3-yl)phenyl]-9,9-dimethyl-9H-fluoren-2-amine (abbreviation: PCBBiF), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

It is further preferable that the material with a hole-transport property used in the composite material have a relatively low HOMO level higher than or equal to −5.7 eV and lower than or equal to −5.4 eV. Using the material with a hole-transport property having a relatively low HOMO level in the composite material makes it easy to inject holes into the hole-transport layer 112 and to obtain a light-emitting device having a long lifetime. In addition, when the material with a hole-transport property that is used in the composite material has a relatively low HOMO level, induction of holes can be inhibited properly, so that the light-emitting device can have a longer lifetime.

Note that mixing the above composite material with a fluoride of an alkali metal or an alkaline earth metal (the proportion of fluorine atoms in a layer including the mixed material is preferably higher than or equal to 20%) can lower the refractive index of the layer. This also enables a layer with a low refractive index to be formed in the organic compound layer 103, leading to higher external quantum efficiency of the light-emitting device.

The formation of the hole-injection layers (111, 111a, and 111b) can improve the hole-injection property, which allows the light-emitting device to be driven at a low voltage.

### Hole-Transport Layer

The hole-transport layers (112, 112a, and 112b) include a hole-transport material and can be formed using any of the hole-transport materials given as examples of the material of the hole-injection layers (111, 111a, and 111b). In order that the hole-transport layers (112, 112a, and 112b) can have a function of transporting holes injected into the hole-injection layers (111, 111a, and 111b) to the light-emitting layers (113, 113a, and 113b), the HOMO level of the hole-transport layers (112, 112a, and 112b) is preferably equal or close to the HOMO level of the hole-injection layers (111, 111a, and 111b).

As the hole-transport material, a substance having a hole mobility higher than or equal to 1×10⁻⁶ cm²/Vs is preferably used. Note that the hole mobility of the substance may be outside this range as long as the substance has a hole-transport property higher than an electron-transport property. The layer including a substance with a high hole-transport property is not limited to a single layer and may be a stack of two or more layers each including any of the above substances.

Examples of the materials that can be used for the hole-transport layers (112, 112a, and 112b) include a compound having an aromatic amine skeleton, such as 4,4′-bis[N-(1-naphthyl)-N-phenylamino]biphenyl (abbreviation: NPB), N,N′-diphenyl-N,N′-bis(3-methylphenyl)-4,4′-diaminobiphenyl (abbreviation: TPD), N,N′-bis(9,9′-spirobi[9H-fluoren]-2-yl)-N,N′-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), 4-phenyl-4′-(9-phenyl-9H-carbazol-3-yl)triphenylamine (abbreviation: PCBA1BP), 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 9,9-dimethyl-N-phenyl-N-[4-(9-phenyl-9H-carbazol-3-yl)phenyl]fluoren-2-amine (abbreviation: PCBAF), or N-phenyl-N-[4-(9-phenyl-9H-carbazol-3-yl)phenyl]-9,9′-spirobi[9H-fluoren]-2-amine (abbreviation: PCBASF); a compound having a carbazole skeleton, such as 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 9,9′-diphenyl-9H,9′H-3,3′-bicarbazole (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), or 9-(biphenyl-3-yl)-9′-(biphenyl-4-yl)-9H,9′H-3,3′-bicarbazole (abbreviation: mBPCCBP); a compound having a thiophene skeleton, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), or 4-[4-(9-phenyl-9H-fluoren-9-yl)phenyl]-6-phenyldibenzothiophene (abbreviation: DBTFLP-IV); and a compound having a furan skeleton, such as 4,4′,4″-(benzene-1,3,5-triyl)tri(dibenzofuran) (abbreviation: DBF3P-II) or 4-{3-[3-(9-phenyl-9H-fluoren-9-yl)phenyl]phenyl}dibenzofuran (abbreviation: mmDBFFLBi-II). Among the above materials, the compound having an aromatic amine skeleton or the compound having a carbazole skeleton is preferable because these compounds are highly reliable and have high hole-transport properties to contribute to a reduction in driving voltage. Note that any of the substances given as examples of the material with a hole-transport property that is used for the composite material for the hole-injection layer 111 can also be suitably used as the material included in the hole-transport layer 112.

### Electron-Transport Layer

The electron-transport layers (114, 114a, and 114b) have a function of transporting, to the light-emitting layer 113, electrons injected from the other of the pair of electrodes (the first electrode 101 or the second electrode 102) through the electron-injection layers (115, 115a, and 115b). Note that the organic compound described in Embodiment 1 can also be used for the electron-transport layers.

As the electron-transport material, it is preferable to use an organic compound with an electron-transport property and an electron mobility higher than or equal to 1×10⁻⁶ cm²/Vs when the square root of the electric field strength [V/cm] is 600. Note that any other substance can also be used as long as the substance has an electron-transport property higher than a hole-transport property. The above organic compound is preferably an organic compound having a π-electron deficient heteroaromatic ring. The organic compound having a π-electron deficient heteroaromatic ring is preferably one or more of an organic compound having a heteroaromatic ring with an azole skeleton, an organic compound having a heteroaromatic ring with a pyridine skeleton, an organic compound having a heteroaromatic ring with a diazine skeleton, and an organic compound having a heteroaromatic ring with a triazine skeleton, for example.

Specific examples of the organic compound having a π-electron deficient heteroaromatic ring and being usable for the above electron-transport layer include an organic compound having an azole skeleton, such as 2-(4-biphenyl)-5-(4-tert-butyl-phenyl)-1,3,4-oxadiazole (abbreviation: PBD), 3-(4-biphenylyl)-4-phenyl-5-(4-tert-butylphenyl)-1,2,4-triazole (abbreviation: TAZ), 1,3-bis[5-(4-tert-butylphenyl)-1,3,4-oxadiazol-2-yl]benzene (abbreviation: OXD-7), 9-[4-(5-phenyl-1,3,4-oxadiazol-2-yl)phenyl]-9H-carbazole (abbreviation: CO11), 2,2′,2″-(1,3,5-benzenetriyl)tris(1-phenyl-1H-benzimidazole) (abbreviation: TPBI), 2-[3-(dibenzothiophen-4-yl)phenyl]-1-phenyl-1H-benzimidazole (abbreviation: mDBTBIm-II), or 4,4′-bis(5-methylbenzoxazol-2-yl)stilbene (abbreviation: BzOs); an organic compound having a heteroaromatic ring with a pyridine skeleton, such as 3,5-bis[3-(9H-carbazol-9-yl)phenyl]pyridine (abbreviation: 35DCzPPy), 1,3,5-tri[(3-pyridyl)-phenyl-3-yl]benzene (abbreviation: TmPyPB), bathophenanthroline (abbreviation: BPhen), bathocuproine (abbreviation: BCP), or 2,9-di(naphthalen-2-yl)-4,7-diphenyl-1,10-phenanthroline (abbreviation: NBPhen); an organic compound having a diazine skeleton, such as 2-[3-(dibenzothiophen-4-yl)phenyl]dibenzo[f,h]quinoxaline (abbreviation: 2mDBTPDBq-II), 2-[3′-(dibenzothiophen-4-yl)biphenyl-3-yl]dibenzo[f,h]quinoxaline (abbreviation: 2mDBTBPDBq-II), 2-[3′-(9H-carbazol-9-yl)biphenyl-3-yl]dibenzo[f,h]quinoxaline (abbreviation: 2mCzBPDBq), 2-[4′-(9-phenyl-9H-carbazol-3-yl)-3,1′-biphenyl-1-yl]dibenzo[f,h]quinoxaline (abbreviation: 2mpPCBPDBq), 2-[4-(3,6-diphenyl-9H-carbazol-9-yl)phenyl]dibenzo[f,h]quinoxaline (abbreviation: 2CzPDBq-III), 7-[3-(dibenzothiophen-4-yl)phenyl]dibenzo[f,h]quinoxaline (abbreviation: 7mDBTPDBq-II), 6-[3-(dibenzothiophen-4-yl)phenyl]dibenzo[f,h]quinoxaline (abbreviation: 6mDBTPDBq-II), 9-[3′-(dibenzothiophen-4-yl)biphenyl-3-yl]naphtho[1′,2′:4,5]furo[2,3-b]pyrazine (abbreviation: 9mDBtBPNfpr), 9-[3′-(dibenzothiophen-4-yl)biphenyl-4-yl]naphtho[1′,2′:4,5]furo[2,3-b]pyrazine (abbreviation: 9pmDBtBPNfpr), 4,6-bis[3-(phenanthren-9-yl)phenyl]pyrimidine (abbreviation: 4,6mPnP2Pm), 4,6-bis[3-(dibenzothiophen-4-yl)phenyl]pyrimidine (abbreviation: 4,6mDBTP2Pm-II), 4,6-bis[3-(9H-carbazol-9-yl)phenyl]pyrimidine (abbreviation: 4,6mCzP2Pm), 9,9′-[pyrimidine-4,6-diylbis(biphenyl-3,3′-diyl)]bis(9H-carbazole) (abbreviation: 4,6mCzBP2Pm), 8-(biphenyl-4-yl)-4-[3-(dibenzothiophen-4-yl)phenyl]-[1]benzofuro[3,2-d]pyrimidine (abbreviation: 8BP-4mDBtPBfpm), 3,8-bis[3-(dibenzothiophen-4-yl)phenyl]benzofuro[2,3-b]pyrazine (abbreviation: 3,8mDBtP2Bfpr), 8-[3′-(dibenzothiophen-4-yl)biphenyl-3-yl]naphtho[1′,2′:4,5]furo[3,2-d]pyrimidine (abbreviation: 8mDBtBPNfpm), 8-([2,2′-binaphthalen]-6-yl)-4-[3-(dibenzothiophen-4-yl)phenyl]-[1]benzofuro[3,2-d]pyrimidine (abbreviation: 8(βN2)-4mDBtPBfpm), 2,2′-(pyridine-2,6-diyl)bis(4-phenylbenzo[h]quinazoline) (abbreviation: 2,6(P-Bqn)2Py), 2,2′-(pyridine-2,6-diyl)bis{4-[4-(2-naphthyl)phenyl]-6-phenylpyrimidine}(abbreviation: 2,6(NP-PPm)2Py), 6-(biphenyl-3-yl)-4-[3,5-bis(9H-carbazol-9-yl)phenyl]-2-phenylpyrimidine (abbreviation: 6mBP-4Cz2PPm), 2,4-bis[4-(1-naphthyl)phenyl]-6-[4-(3-pyridyl)phenyl]pyrimidine (abbreviation: 2,4NP-6PyPPm), 4-[3,5-bis(9H-carbazol-9-yl)phenyl]-2-phenyl-6-(biphenyl-4-yl)pyrimidine (abbreviation: 6BP-4Cz2PPm), or 7-[4-(9-phenyl-9H-carbazol-2-yl)quinazolin-2-yl]-7H-dibenzo[c,g]carbazole (abbreviation: PC-cgDBCzQz); and an organic compound having a triazine skeleton, such as 2-[3′-(9,9-dimethyl-9H-fluoren-2-yl)biphenyl-3-yl]-4,6-diphenyl-1,3,5-triazine (abbreviation: mFBPTzn), 2-(biphenyl-4-yl)-4-phenyl-6-(9,9′-spirobi[9H-fluoren]-2-yl)-1,3,5-triazine (abbreviation: BP-SFTzn), 2-{3-[3-(benzo[b]naphtho[1,2-d]furan-8-yl)phenyl]phenyl}-4,6-diphenyl-1,3,5-triazine (abbreviation: mBnfBPTzn), 2-{3-[3-(benzo[b]naphtho[1,2-d]furan-6-yl)phenyl]phenyl}-4,6-diphenyl-1,3,5-triazine (abbreviation: mBnfBPTzn-02), 9-[4-(4,6-diphenyl-1,3,5-triazin-2-yl)phenyl]-9′-phenyl-3,3′-bi-9H-carbazole (abbreviation: PCCzPTzn), 9-[3-(4,6-diphenyl-1,3,5-triazin-2-yl)phenyl]-9′-phenyl-2,3′-bi-9H-carbazole (abbreviation: mPCCzPTzn-02), 5-[3-(4,6-diphenyl-1,3,5-triazin-2-yl)phenyl]-7,7-dimethyl-5H,7H-indeno[2,1-b]carbazole (abbreviation: mINc(II)PTzn), 2-{3-[3-(dibenzothiophen-4-yl)phenyl]phenyl}-4,6-diphenyl-1,3,5-triazine (abbreviation: mDBtBPTzn), 2,4,6-tris[3′-(pyridin-3-yl)biphenyl-3-yl]-1,3,5-triazine (abbreviation: TmPPPyTz), 2-[3-(2,6-dimethyl-3-pyridinyl)-5-(9-phenanthryl)phenyl]-4,6-diphenyl-1,3,5-triazine (abbreviation: mPn-mDMePyPTzn), 11-[4-(biphenyl-4-yl)-6-phenyl-1,3,5-triazin-2-yl]-11,12-dihydro-12-phenylindolo[2,3-a]carbazole (abbreviation: BP-Icz(II)Tzn), 2-[3′-(triphenylen-2-yl)biphenyl-3-yl]-4,6-diphenyl-1,3,5-triazine (abbreviation: mTpBPTzn), 3-[9-(4,6-diphenyl-1,3,5-triazin-2-yl)-2-dibenzofuranyl]-9-phenyl-9H-carbazole (abbreviation: PCDBfTzn), or 2-(biphenyl-3-yl)-4-phenyl-6-[8-([1,1′:4′,1″-terphenyl]-4-yl)-1-dibenzofuranyl]-1,3,5-triazine (abbreviation: mBP-TPDBfTzn). Among the above materials, the organic compound having a heteroaromatic ring with a diazine skeleton, the organic compound having a heteroaromatic ring with a pyridine skeleton, and the organic compound having a heteroaromatic ring with a triazine skeleton have high reliability and thus are preferable. In particular, the organic compound having a heteroaromatic ring with a diazine (pyrimidine or pyrazine) skeleton and the organic compound having a heteroaromatic ring with a triazine skeleton have a high electron-transport property to contribute to a reduction in driving voltage.

Each of the electron-transport layers (114, 114a, and 114b) is not limited to a single layer and may be a stack of two or more layers each including any of the above substances.

Between the electron-transport layer (114, 114a, or 114b) and the light-emitting layer (113, 113a, or 113b), a layer that controls transfer of electron carriers may be provided. This is a layer formed by addition of a small amount of a substance having a high electron-trapping property to a material having a high electron-transport property as described above, and the layer is capable of adjusting carrier balance by suppressing transport of electron carriers. Such a structure is very effective in inhibiting a problem (such as a reduction in element lifetime) caused when electrons pass through the light-emitting layer.

### Electron-Injection Layer

The electron-injection layers (115, 115a, and 115b) have a function of reducing a barrier to electron injection from the second electrode 102 to promote electron injection. The organic compound described in Embodiment 1 can also be used for the electron-injection layers.

For the electron-injection layers, a Group 1 metal, a Group 2 metal, an oxide of these metals, a halide of these metals, a carbonate of these metals, or the like can be used. Alternatively, a composite material including any of the electron-transport materials described above and a material having a property of donating electrons to the electron-transport material can also be used. As examples of the material having an electron-donating property, a Group 1 metal, a Group 2 metal, an oxide of any of these metals, and the like can be given. Specifically, an alkali metal, an alkaline earth metal, or a compound thereof, such as lithium fluoride (LiF), sodium fluoride (NaF), cesium fluoride (CsF), calcium fluoride (CaF₂), or lithium oxide (LiOₓ), can be used. Alternatively, a rare earth metal compound like erbium fluoride (ErF₃) can be used. Electride may also be used for the electron-injection layer 115. Examples of the electride include a substance in which electrons are added at high concentration to calcium oxide-aluminum oxide. The electron-injection layers (115, 115a, and 115b) may be formed using the substance that can be used for the electron-transport layers (114, 114a, and 114b).

A composite material in which an organic compound and an electron donor (donor) are mixed may also be used for the electron-injection layers (115, 115a, and 115b). Such a composite material is excellent in an electron-injection property and an electron-transport property because electrons are generated in the organic compound by the electron donor. In this case, the organic compound is preferably a material that is excellent in transporting the generated electrons. Specifically, any of the above-described substances for forming the electron-transport layer 114 (e.g., a metal complex or a heteroaromatic compound) can be used, for example. As the electron donor, a substance having an electron-donating property with respect to the organic compound can be used. Specifically, it is preferable to use an alkali metal, an alkaline earth metal, or a rare earth metal, such as lithium, sodium, cesium, magnesium, calcium, erbium, or ytterbium. It is also preferable to use an alkali metal oxide or an alkaline earth metal oxide, such as lithium oxide, calcium oxide, or barium oxide. Alternatively, a Lewis base such as magnesium oxide can be used. Further alternatively, an organic compound such as tetrathiafulvalene (abbreviation: TTF) can be used.

Note that the light-emitting layer, the hole-injection layer, the hole-transport layer, the electron-transport layer, and the electron-injection layer described above can each be formed by an evaporation method (including a vacuum evaporation method), an ink-jet method, a coating method, a gravure printing method, or the like. Besides the above-mentioned materials, an inorganic compound such as a quantum dot or a high molecular compound (e.g., an oligomer, a dendrimer, or a polymer) may be used for the light-emitting layer, the hole-injection layer, the hole-transport layer, the electron-transport layer, and the electron-injection layer.

The quantum dot may be a colloidal quantum dot, an alloyed quantum dot, a core-shell quantum dot, or a core quantum dot, for example. The quantum dot including elements belonging to Groups 2 and 16, elements belonging to Groups 13 and 15, elements belonging to Groups 13 and 17, elements belonging to Groups 11 and 17, or elements belonging to Groups 14 and 15 may be used. Alternatively, the quantum dot including an element such as cadmium (Cd), selenium (Se), zinc (Zn), sulfur (S), phosphorus (P), indium (In), tellurium (Te), lead (Pb), gallium (Ga), arsenic (As), or aluminum (Al) may be used.

### Pair of Electrodes

The first electrode 101 and the second electrode 102 function as the anode and the cathode of the light-emitting device. The first electrode 101 and the second electrode 102 can be formed using a metal, an alloy, or a conductive compound, a mixture or a stack thereof, or the like.

One of the first electrode 101 and the second electrode 102 is preferably formed using a conductive material having a function of reflecting light. Examples of the conductive material include aluminum (Al), an alloy including Al, and the like. Examples of the alloy including Al include an alloy including Al and L (L represents one or more of titanium (Ti), neodymium (Nd), nickel (Ni), and lanthanum (La)), such as an alloy including Al and Ti or an alloy including Al, Ni, and La. Aluminum has low resistance and high light reflectivity. Aluminum is included in earth's crust in large amount and is inexpensive; thus, it is possible to reduce costs for manufacturing a light-emitting device with aluminum. Alternatively, silver (Ag), an alloy of Ag and N (N represents one or more of yttrium (Y), Nd, magnesium (Mg), ytterbium (Yb), Al, Ti, gallium (Ga), zinc (Zn), indium (In), tungsten (W), manganese (Mn), tin (Sn), iron (Fe), Ni, copper (Cu), palladium (Pd), iridium (Ir), and gold (Au)), or the like may be used. Examples of the alloy including silver include an alloy including silver, palladium, and copper, an alloy including silver and copper, an alloy including silver and magnesium, an alloy including silver and nickel, an alloy including silver and gold, an alloy including silver and ytterbium, and the like. Besides, a transition metal such as tungsten, chromium (Cr), molybdenum (Mo), copper, or titanium can be used.

Light emitted from the light-emitting layer is extracted through the first electrode 101 and/or the second electrode 102. Thus, at least one of the first electrode 101 and the second electrode 102 is preferably formed using a conductive material having a function of transmitting light. As the conductive material, a conductive material having a visible light transmittance higher than or equal to 40% and lower than or equal to 100%, preferably higher than or equal to 60% and lower than or equal to 100%, and a resistivity lower than or equal to 1×10⁻² Ω·cm can be used.

The first electrode 101 and the second electrode 102 may each be formed using a conductive material having functions of transmitting light and reflecting light. As the conductive material, a conductive material having a visible light reflectivity higher than or equal to 20% and lower than or equal to 80%, preferably higher than or equal to 40% and lower than or equal to 70%, and a resistivity lower than or equal to 1×10⁻² Ω·cm can be used. For example, one or more kinds of conductive metals and alloys, conductive compounds, and the like can be used. Specifically, a metal oxide such as indium tin oxide (hereinafter referred to as ITO), indium tin oxide including silicon or silicon oxide (ITSO), indium oxide-zinc oxide (indium zinc oxide), indium oxide-tin oxide including titanium, indium titanium oxide, or indium oxide including tungsten oxide and zinc oxide can be used. A metal thin film having a thickness that allows transmission of light (preferably, a thickness greater than or equal to 1 nm and less than or equal to 30 nm) can also be used. As the metal, Ag can be used, for example. As the alloy, an alloy of Ag and Al, an alloy of Ag and Mg, an alloy of Ag and Au, an alloy of Ag and Yb, or the like can be used.

In this specification and the like, as the material having a function of transmitting light, a material having a function of transmitting visible light and having conductivity is used. Examples of the material include, in addition to the above-described oxide conductor typified by ITO, an oxide semiconductor and an organic conductor including an organic substance. Examples of the organic conductor including an organic substance include a composite material in which an organic compound and an electron donor (donor) are mixed and a composite material in which an organic compound and an electron acceptor (acceptor) are mixed. Alternatively, an inorganic carbon-based material such as graphene may be used. The resistivity of the material is preferably lower than or equal to 1×10⁵ Ω·cm, further preferably lower than or equal to 1×10⁴ Ω·cm.

The first electrode 101 and/or the second electrode 102 may be formed by stacking two or more of the materials described above.

In order to improve the light extraction efficiency, a material whose refractive index is higher than that of an electrode having a function of transmitting light may be formed in contact with the electrode. The material may be electrically conductive or non-conductive as long as it has a function of transmitting visible light. In addition to the oxide conductors described above, an oxide semiconductor and an organic substance are given as the examples of the material. Examples of the organic substance include the example materials for the light-emitting layer, the hole-injection layer, the hole-transport layer, the electron-transport layer, and the electron-injection layer. Alternatively, an inorganic carbon-based material or a metal film thin enough to transmit light can be used. Further alternatively, a plurality of layers each having a thickness of several nanometers to several tens of nanometers may be stacked.

In the case where the first electrode 101 or the second electrode 102 functions as the cathode, the electrode preferably includes a material having a low work function (lower than or equal to 3.8 eV). For example, it is possible to use an element belonging to Group 1 or 2 of the periodic table (e.g., an alkali metal such as lithium, sodium, or cesium, an alkaline earth metal such as calcium or strontium, or magnesium), an alloy including any of these elements (e.g., Ag—Mg or Al—Li), a rare earth metal such as europium (Eu) or Yb, an alloy including any of these rare earth metals, an alloy including aluminum or silver, or the like.

When the first electrode 101 or the second electrode 102 is used as the anode, a material with a high work function (higher than or equal to 4.0 eV) is preferably used.

The first electrode 101 and the second electrode 102 may be a stack of a conductive material having a function of reflecting light and a conductive material having a function of transmitting light. This structure is preferably employed, in which case the first electrode 101 and the second electrode 102 can have a function of adjusting the optical path length so that light with a desired wavelength emitted from each light-emitting layer resonates and is intensified.

As the method for forming the first electrode 101 and the second electrode 102, a sputtering method, an evaporation method, a printing method, a coating method, a molecular beam epitaxy (MBE) method, a CVD method, a pulsed laser deposition method, an atomic layer deposition (ALD) method, or the like can be used as appropriate.

### Charge-Generation Layer (Intermediate Layer)

The charge-generation layer 106 has a function of injecting electrons into the organic compound layer 103a and injecting holes into the organic compound layer 103b when a voltage is applied between the first electrode (anode) 101 and the second electrode (cathode) 102. The charge-generation layer 106 may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer.

In the case where the charge-generation layer 106 is a p-type layer in which an electron acceptor is added to a hole-transport material, which is an organic compound, any of the hole-transport materials described in this embodiment can be used as the hole-transport material. Examples of the electron acceptor include 7,7,8,8-tetracyano-2,3,5,6-tetrafluoroquinodimethane (abbreviation: F₄-TCNQ) and chloranil. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films including the respective materials may be used.

In the case where the charge-generation layer 106 is an electron-injection buffer layer in which an electron donor is added to an electron-transport material, the organic compound described in Embodiment 1 is preferably used as the electron-transport material. Any of the electron-transport materials described in this embodiment may be used.

Note that when the electron-injection buffer layer is formed using the organic compound of one embodiment of the present invention, it is possible to inhibit an increase in driving voltage of the case where the organic compound layers each including the light-emitting layer are stacked. For example, in the case where a layer in which a metal or a metal compound is mixed with the organic compound of one embodiment of the present invention is used as the electron-injection buffer layer, the metal or the metal compound can be coordinated to a nitrogen atom contained in the organic compound of one embodiment of the present invention (a chelate complex can be formed). Thus, when mixed with a metal or a metal compound, the organic compound of one embodiment of the present invention can stabilize the metal or the metal compound functioning as an electron donor. That is, using the organic compound of one embodiment of the present invention for an intermediate layer or an electron-transport layer of a tandem light-emitting device allows the tandem light-emitting device to be driven at a low voltage.

As the electron donor, it is possible to use an alkali metal, an alkaline earth metal, a rare earth metal, a metal belonging to Group 2 or Group 13 of the periodic table, or an oxide or a carbonate thereof. Specifically, lithium (Li), cesium (Cs), magnesium (Mg), calcium (Ca), ytterbium (Yb), indium (In), lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer 106, the electron-relay layer includes at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer 106. Specifically, the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

Although FIG. 2D illustrates the structure in which two of the organic compound layers 103 are stacked, organic compound layers including three or more light-emitting layers may be stacked with charge-generation layers each provided between different light-emitting layers.

### Cap Layer

Although not illustrated in FIGS. 2A to 2E, a cap layer may be provided over the second electrode 102 of the light-emitting device. For example, a material with a high refractive index can be used for the cap layer. When the cap layer is provided over the second electrode 102, extraction efficiency of light emitted through the second electrode 102 can be improved.

Specific examples of a material that can be used for the cap layer include 5,5′-diphenyl-2,2′-di-5H-[1]benzothieno[3,2-c]carbazole (abbreviation: BisBTc) and 4,4′,4″-(benzene-1,3,5-triyl)tri(dibenzothiophene) (abbreviation: DBT3P-II).

### Substrate

A light-emitting device of one embodiment of the present invention can be formed over a substrate of glass, plastic, or the like. As the way of stacking layers over the substrate, layers may be sequentially stacked from the first electrode 101 side or sequentially stacked from the second electrode 102 side.

For the substrate over which the light-emitting device of one embodiment of the present invention can be formed, glass, quartz, plastic, or the like can be used, for example. A flexible substrate may be used. The flexible substrate means a substrate that can be bent, such as a plastic substrate made of polycarbonate or polyarylate, for example. Alternatively, a film, an inorganic vapor deposition film, or the like can be used. Another material may be used as long as the substrate functions as a support in a manufacturing process of the light-emitting device or an optical element. Another material having a function of protecting the light-emitting device or the optical element may be used.

In this specification and the like, a light-emitting device can be formed using any of a variety of substrates, for example. There is no particular limitation on the type of the substrate. Examples of the substrate include a semiconductor substrate (e.g., a single crystal substrate such as a silicon substrate); an SOI substrate; a glass substrate; a quartz substrate; a plastic substrate; a metal substrate; a stainless steel substrate; a substrate including stainless steel foil; a tungsten substrate; a substrate including tungsten foil; a flexible substrate; an attachment film; and cellulose nanofiber (CNF), paper, and a base material film that include a fibrous material. Examples of a glass substrate include a barium borosilicate glass substrate, an aluminoborosilicate glass substrate, and a soda lime glass substrate. Examples of the flexible substrate, the attachment film, the base material film, and the like are substrates of plastics typified by polyethylene terephthalate (PET), polyethylene naphthalate (PEN), polyether sulfone (PES), and polytetrafluoroethylene (PTFE). Another example is an acrylic resin. Furthermore, polypropylene, polyester, polyvinyl fluoride, and polyvinyl chloride can be given as examples. Other examples include a resin such as a polyamide resin, a polyimide resin, an aramid resin, or an epoxy resin, an inorganic vapor deposition film, and paper.

Alternatively, a flexible substrate may be used as the substrate, and a light-emitting device may be provided directly on the flexible substrate. Further alternatively, a separation layer may be provided between the substrate and the light-emitting device. The separation layer can be used to separate part or the whole of the light-emitting device, which is formed over the separation layer, from the substrate and transfer the separated component onto another substrate. In that case, the light-emitting device can be transferred to a substrate having low heat resistance or a flexible substrate as well. For the above separation layer, a stack including inorganic films, which are a tungsten film and a silicon oxide film, or a structure in which a resin film of polyimide or the like is formed over a substrate can be used, for example.

In other words, after the light-emitting device is formed using a substrate, the light-emitting device may be transferred to another substrate. Examples of the substrate to which the light-emitting device is transferred are, in addition to the above substrates, a cellophane substrate, a stone substrate, a wood substrate, a cloth substrate (including a natural fiber (e.g., silk, cotton, or hemp), a synthetic fiber (e.g., nylon, polyurethane, or polyester), a regenerated fiber (e.g., acetate, cupro, rayon, or regenerated polyester), and the like), a leather substrate, a rubber substrate, and the like. When such a substrate is used, a light-emitting device with high durability, high heat resistance, reduced weight, or reduced thickness can be formed.

The light-emitting device may be formed over an electrode electrically connected to a field-effect transistor (FET), for example, that is formed over any of the above-described substrates. In that case, an active matrix display device in which the FET controls the driving of the light-emitting device can be manufactured.

In this embodiment, one embodiment of the present invention has been described. Other embodiments of the present invention are described in other embodiments. Note that one embodiment of the present invention is not limited thereto. In other words, various embodiments of the invention are described in this embodiment and the other embodiments, and one embodiment of the present invention is not limited to a particular embodiment. For example, although the example in which one embodiment of the present invention is applied to a light-emitting device is described, one embodiment of the present invention is not limited thereto. For example, depending on circumstances or conditions, one embodiment of the present invention is not necessarily used in a light-emitting device. One embodiment of the present invention describes, but is not limited to, an example of including a first organic compound, a second organic compound, and a guest material capable of converting triplet excitation energy into light emission, in which the LUMO level of the first organic compound is lower than that of the second organic compound and the HOMO level of the first organic compound is lower than that of the second organic compound. Depending on circumstances or conditions, in one embodiment of the present invention, for example, the LUMO level of the first organic compound is not necessarily lower than that of the second organic compound. Alternatively, the HOMO level of the first organic compound is not necessarily lower than that of the second organic compound. One embodiment of the present invention describes, but is not limited to, an example in which the first organic compound and the second organic compound form an exciplex. Depending on circumstances or conditions, in one embodiment of the present invention, for example, the first organic compound and the second organic compound do not necessarily form an exciplex. One embodiment of the present invention describes, but is not limited to, an example in which the LUMO level of the guest material is higher than that of the first organic compound and the HOMO level of the guest material is lower than that of the second organic compound. Depending on circumstances or conditions, in one embodiment of the present invention, for example, the LUMO level of the guest material is not necessarily higher than that of the first organic compound. Alternatively, the HOMO level of the guest material is not necessarily lower than that of the second organic compound.

The structure described above in this embodiment can be used in combination with any of the structures described in the other embodiments as appropriate.

### Embodiment 3

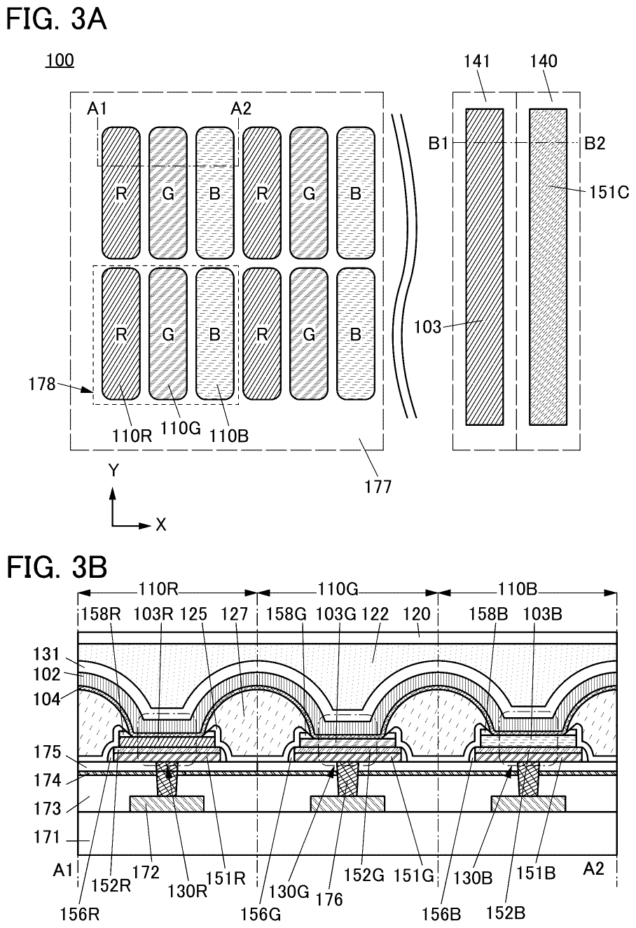

As illustrated in FIGS. 3A and 3B, a plurality of light-emitting devices 130 are formed over an insulating layer 175 to constitute a display device. In this embodiment, a display device of one embodiment of the present invention will be described in detail.

A display device 100 includes a pixel portion 177 in which a plurality of pixels 178 are arranged in matrix. The pixel 178 includes a subpixel 110R, a subpixel 110G, and a subpixel 110B.

In this specification and the like, for example, description common to the subpixels 110R, 110G, and 110B is sometimes made using the collective term “subpixel 110”. As for other components that are distinguished from each other using letters of the alphabet, matters common to the components are sometimes described using reference numerals excluding the letters of the alphabet.

The subpixel 110R emits red light, the subpixel 110G emits green light, and the subpixel 110B emits blue light. Thus, an image can be displayed on the pixel portion 177. Note that in this embodiment, three colors of red (R), green (G), and blue (B) are given as examples of colors of light emitted by the subpixels; however, subpixels of a different combination of colors may be employed. The number of subpixels is not limited to three, and may be four or more. Examples of four subpixels include subpixels emitting light of four colors of R, G, B, and white (W), subpixels emitting light of four colors of R, G, B, and yellow (Y), and four subpixels emitting light of R, G, and B and infrared (IR) light.

In this specification and the like, the row direction and the column direction are sometimes referred to as the X direction and the Y direction, respectively. The X direction and the Y direction intersect with each other and are perpendicular to each other, for example.

FIG. 3A illustrates an example where subpixels of different colors are arranged in the X direction and subpixels of the same color are arranged in the Y direction. Note that subpixels of different colors may be arranged in the Y direction, and subpixels of the same color may be arranged in the X direction.

Outside the pixel portion 177, a connection portion 140 is provided and a region 141 may also be provided. The region 141 is provided between the pixel portion 177 and the connection portion 140. The organic compound layer 103 is provided in the region 141. A conductive layer 151C is provided in the connection portion 140.

Although FIG. 3A illustrates an example where the region 141 and the connection portion 140 are located on the right side of the pixel portion 177, there is no particular limitation on the positions of the region 141 and the connection portion 140. The number of regions 141 and the number of connection portions 140 can each be one or more.

FIG. 3B is an example of a cross-sectional view along the dashed-dotted line A1-A2 in FIG. 3A. As illustrated in FIG. 3B, the display device 100 includes an insulating layer 171, a conductive layer 172 over the insulating layer 171, an insulating layer 173 over the insulating layer 171 and the conductive layer 172, an insulating layer 174 over the insulating layer 173, and the insulating layer 175 over the insulating layer 174. The insulating layer 171 is provided over a substrate (not illustrated). An opening reaching the conductive layer 172 is provided in the insulating layers 175, 174, and 173, and a plug 176 is provided to fill the opening.

In the pixel portion 177, the light-emitting device 130 is provided over the insulating layer 175 and the plug 176. A protective layer 131 is provided to cover the light-emitting device 130. A substrate 120 is attached to the protective layer 131 with a resin layer 122. An inorganic insulating layer 125 and an insulating layer 127 over the inorganic insulating layer 125 are preferably provided between the adjacent light-emitting devices 130.

Although each of the inorganic insulating layer 125 and the insulating layer 127 looks like a plurality of layers in the cross-sectional view in FIG. 3B, each of the inorganic insulating layer 125 and the insulating layer 127 is preferably one continuous layer when the display device 100 is seen from above. In other words, the inorganic insulating layer 125 and the insulating layer 127 preferably include openings over first electrodes.

In FIG. 3B, a light-emitting device 130R, a light-emitting device 130G, and a light-emitting device 130B are each illustrated as the light-emitting device 130. The light-emitting devices 130R, 130G, and 130B emit light of different colors. For example, the light-emitting device 130R can emit red light, the light-emitting device 130G can emit green light, and the light-emitting device 130B can emit blue light. Alternatively, the light-emitting device 130R, 130G, or 130B may emit visible light of another color or infrared light.

The display device of one embodiment of the present invention can be, for example, a top-emission display device where light is emitted in the direction opposite to a substrate over which light-emitting devices are formed. Note that the display device of one embodiment of the present invention may be of a bottom-emission type.

Examples of a light-emitting substance included in the light-emitting device 130 include organic compounds or organometallic complexes such as a substance emitting fluorescent light (a fluorescent material), a substance emitting phosphorescent light (a phosphorescent material), and a substance exhibiting thermally activated delayed fluorescence (a thermally activated delayed fluorescent (TADF) material). Other examples include inorganic compounds (e.g., a quantum dot material).

The light-emitting device 130R has a structure as illustrated in FIG. 1A. The light-emitting device 130R includes the first electrode (pixel electrode) including a conductive layer 151R and a conductive layer 152R, an organic compound layer 103R over the first electrode, a common layer 104 over the organic compound layer 103R, and the second electrode (common electrode) 102 over the common layer 104. Although the common layer 104 is not necessarily provided, it is preferable to provide the common layer 104 to reduce damage to the organic compound layer 103R during processing. In the case where the common layer 104 is provided, the common layer 104 is preferably an electron-injection layer. In the case where the common layer 104 is provided, a stack of the organic compound layer 103R and the common layer 104 corresponds to the organic compound layer 103 described in Embodiment 2.

The light-emitting device 130G has a structure as illustrated in FIG. 1A. The light-emitting device 130G includes the first electrode (pixel electrode) including a conductive layer 151G and a conductive layer 152G, an organic compound layer 103G over the first electrode, the common layer 104 over the organic compound layer 103G, and the second electrode (common electrode) 102 over the common layer 104. Although the common layer 104 is not necessarily provided, it is preferable to provide the common layer 104 to reduce damage to the organic compound layer 103G during processing. In the case where the common layer 104 is provided, the common layer 104 is preferably an electron-injection layer. In the case where the common layer 104 is provided, a stack of the organic compound layer 103G and the common layer 104 corresponds to the organic compound layer 103 described in Embodiment 2.

The light-emitting device 130B has a structure as illustrated in FIG. 1A. The light-emitting device 130B includes the first electrode (pixel electrode) including a conductive layer 151B and a conductive layer 152B, an organic compound layer 103B over the first electrode, the common layer 104 over the organic compound layer 103B, and the second electrode (common electrode) 102 over the common layer 104. Although the common layer 104 is not necessarily provided, it is preferable to provide the common layer 104 to reduce damage to the organic compound layer 103B during processing. In the case where the common layer 104 is provided, the common layer 104 is preferably an electron-injection layer. In the case where the common layer 104 is provided, a stack of the organic compound layer 103B and the common layer 104 corresponds to the organic compound layer 103 described in Embodiment 2.

In the light-emitting device, one of the pixel electrode and the common electrode functions as an anode and the other functions as a cathode. Hereinafter, description is made on the assumption that the pixel electrode functions as the anode and the common electrode functions as the cathode unless otherwise specified.

The organic compound layers 103R, 103G, and 103B are island-shaped layers that are independent of each other on a light-emitting device basis or on an emission color basis. Providing the island-shaped organic compound layer 103 in each of the light-emitting devices 130 can inhibit leakage current between the adjacent light-emitting devices 130 even in a high-resolution display device. This can prevent crosstalk, so that a display device with extremely high contrast can be obtained. Specifically, a display device having high current efficiency at low luminance can be obtained.

The island-shaped organic compound layer 103 is formed by forming an EL film and processing the EL film by a lithography method.

In the display device of one embodiment of the present invention, the first electrode (pixel electrode) of the light-emitting device preferably has a stacked-layer structure. For example, in the example illustrated in FIG. 3B, the first electrode of the light-emitting device 130 is a stack of the conductive layer 151 and the conductive layer 152. In the case where the display device 100 is of a top-emission type and the pixel electrode of the light-emitting device 130 functions as the anode, for example, the conductive layer 151 preferably has high visible light reflectance, and the conductive layer 152 preferably has a visible-light-transmitting property and a high work function. In the case where the display device 100 is of a top-emission type, the higher the visible light reflectance of the pixel electrode is, the higher the efficiency of extraction of the light emitted by the organic compound layer 103 is. In the case where the pixel electrode functions as the anode, the higher the work function of the pixel electrode is, the easier hole injection into the organic compound layer 103 is. Accordingly, when the pixel electrode of the light-emitting device 130 is a stack of the conductive layer 151 with high visible light reflectance and the conductive layer 152 with a high work function, the light-emitting device 130 can have high light extraction efficiency and a low driving voltage.

In the case where the conductive layer 151 has high visible light reflectance, the visible light reflectance of the conductive layer 151 is preferably higher than or equal to 40% and lower than or equal to 100%, further preferably higher than or equal to 70% and lower than or equal to 100%, for example. When used as an electrode having a visible-light-transmitting property, the conductive layer 152 preferably has a visible light transmittance higher than or equal to 40%, for example.

Here, such a pixel electrode being a stack composed of a plurality of layers might change in quality as a result of, for example, a reaction between the plurality of layers. For example, when a film formed after the formation of the pixel electrode is removed by a wet etching method, contact of a chemical solution with the pixel electrode might cause galvanic corrosion.

In view of the above, an insulating layer 156 is formed on the side surfaces of the conductive layers 151 and 152 in the display device 100 of this embodiment. This can inhibit a chemical solution from coming into contact with the conductive layer 151 when a film that is formed after formation of the pixel electrode including the conductive layer 151 and the conductive layer 152 is removed by a wet etching method, for example. Accordingly, occurrence of galvanic corrosion in the pixel electrode can be inhibited, for example. This allows the display device 100 to be manufactured by a high-yield method and to be accordingly inexpensive. In addition, generation of a defect in the display device 100 can be inhibited, which makes the display device 100 highly reliable.

A metal material can be used for the conductive layer 151, for example. Specifically, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals, for example.

For the conductive layer 152, an oxide containing one or more selected from indium, tin, zinc, gallium, titanium, aluminum, and silicon can be used. For example, it is preferable to use a conductive oxide containing one or more of indium oxide, indium tin oxide, indium zinc oxide, zinc oxide, zinc oxide containing gallium, titanium oxide, indium zinc oxide containing gallium, indium zinc oxide containing aluminum, indium tin oxide containing silicon, indium zinc oxide containing silicon, and the like. In particular, indium tin oxide containing silicon can be suitably used for the conductive layer 152 because of having a high work function, for example, a work function higher than or equal to 4.0 eV.

The conductive layer 151 and the conductive layer 152 may each be a stack of a plurality of layers that include different materials. In that case, the conductive layer 151 may include a layer formed using a material that can be used for the conductive layer 152, such as a conductive oxide. Furthermore, the conductive layer 152 may include a layer formed using a material that can be used for the conductive layer 151, such as a metal material. In the case where the conductive layer 151 is a stack of two or more layers, for example, a layer in contact with the conductive layer 152 can be formed using a material that can be used for the conductive layer 152.

Note that an end portion of the insulating layer 156 may have a tapered shape. Specifically, when the end portion of the insulating layer 156 has a tapered shape with a taper angle less than 90°, coverage with a component provided along the side surface of the insulating layer 156 can be improved.

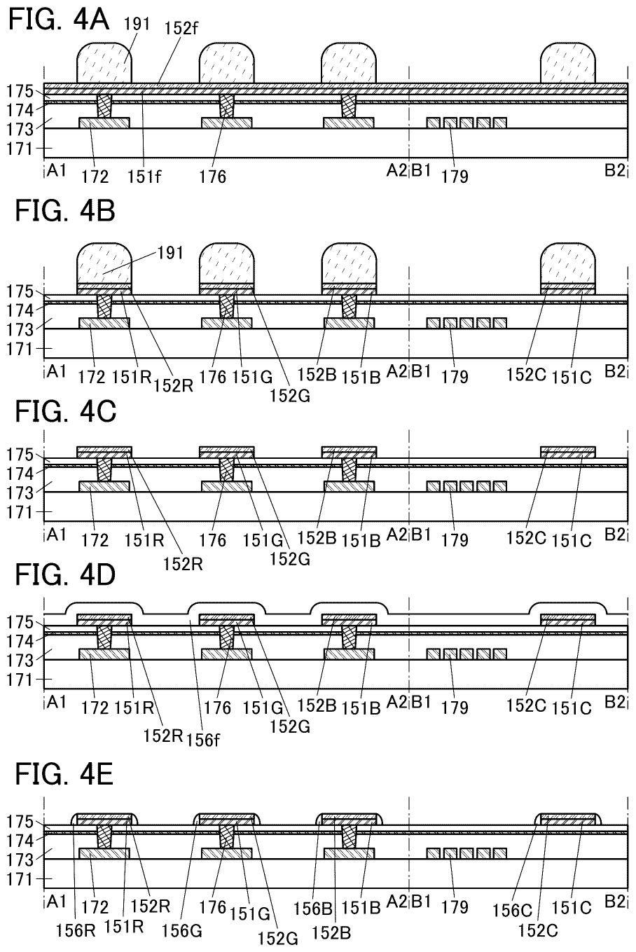

Next, an example of a method for manufacturing the display device 100 having the structure illustrated in FIG. 3A is described with reference to FIGS. 4A to 4E, FIGS. 5A and 5B, FIGS. 6A to 6D, FIGS. 7A to 7C, FIGS. 8A to 8C, and FIGS. 9A to 9C. An organic layer of the light-emitting device included in the display device 100 is formed by manufacturing steps including treatment using water. The use of the organic compound of one embodiment of the present invention for the organic layer of the light-emitting device included in the display device of one embodiment of the present invention prevents problems such as dissolution of the layer containing the organic compound and permeation of a chemical solution into the layer containing the organic compound even in the manufacturing process including treatment using water; consequently, the light-emitting device can have favorable characteristics.

### Manufacturing Method Example

Thin films included in the display device (e.g., insulating films, semiconductor films, and conductive films) can be formed by a sputtering method, a chemical vapor deposition (CVD) method, a vacuum evaporation method, a pulsed laser deposition (PLD) method, an ALD method, or the like. Examples of a CVD method include a plasma-enhanced CVD (PECVD) method and a thermal CVD method. An example of a thermal CVD method is a metal organic CVD (MOCVD) method.

Thin films included in the display device (e.g., insulating films, semiconductor films, and conductive films) can also be formed by a wet film-formation method such as spin coating, dipping, spray coating, ink-jetting, dispensing, screen printing, offset printing, doctor blade coating, slit coating, roll coating, curtain coating, or knife coating.

Specifically, for fabrication of the light-emitting device, a vacuum process such as an evaporation method and a solution process such as a spin coating method or an ink-jet method can be used. Examples of an evaporation method include physical vapor deposition methods (PVD methods) such as a sputtering method, an ion plating method, an ion beam evaporation method, a molecular beam evaporation method, and a vacuum evaporation method, and a chemical vapor deposition method (CVD method). Specifically, the functional layers (e.g., the hole-injection layer, the hole-transport layer, the hole-blocking layer, the light-emitting layer, the electron-blocking layer, the electron-transport layer, and the electron-injection layer) included in the organic compound layer can be formed by an evaporation method (e.g., a vacuum evaporation method), a coating method (e.g., a dip coating method, a die coating method, a bar coating method, a spin coating method, or a spray coating method), a printing method (e.g., ink-jetting, screen printing (stencil), offset printing (planography), flexography (relief printing), gravure printing, or micro-contact printing), or the like.

Thin films included in the display device can be processed by a lithography method, for example. Alternatively, a nanoimprinting method, a sandblasting method, a lift-off method, or the like may be used to process thin films. Alternatively, island-shaped thin films may be directly formed by a film formation method using a shielding mask such as a metal mask.

As a lithography method, for example, a photolithography method can be used. There are two typical examples of photolithography methods. In one of the methods, a resist mask is formed over a thin film that is to be processed, the thin film is processed by etching, for example, and then the resist mask is removed. In the other method, a photosensitive thin film is formed and then processed into a desired shape by light exposure and development.

As light used for exposure in the photolithography method, for example, light with an i-line (wavelength: 365 nm), light with a g-line (wavelength: 436 nm), light with an h-line (wavelength: 405 nm), or light in which the i-line, the g-line, and the h-line are mixed can be used. Alternatively, ultraviolet rays, KrF laser light, ArF laser light, or the like can be used. Exposure may be performed by liquid immersion exposure technique. As the light for exposure, extreme ultraviolet (EUV) light or X-rays may also be used. Furthermore, instead of the light used for exposure, an electron beam can be used. It is preferable to use EUV light, X-rays, or an electron beam to perform extremely minute processing. Note that when exposure is performed by scanning of a beam such as an electron beam, a photomask is not needed.

For etching of thin films, a dry etching method, a wet etching method, a sandblasting method, or the like can be used.

First, as illustrated in FIG. 4A, the insulating layer 171 is formed over a substrate (not illustrated). Next, the conductive layer 172 and a conductive layer 179 are formed over the insulating layer 171, and the insulating layer 173 is formed over the insulating layer 171 so as to cover the conductive layer 172 and the conductive layer 179. Then, the insulating layer 174 is formed over the insulating layer 173, and the insulating layer 175 is formed over the insulating layer 174.

As the substrate, a substrate having heat resistance high enough to withstand at least heat treatment performed later can be used. When an insulating substrate is used, it is possible to use a glass substrate, a quartz substrate, a sapphire substrate, a ceramic substrate, an organic resin substrate, or the like. Alternatively, it is possible to use a semiconductor substrate such as a single crystal semiconductor substrate or a polycrystalline semiconductor substrate of silicon, silicon carbide, or the like, a compound semiconductor substrate of silicon germanium or the like, or an SOI substrate.

Next, as illustrated in FIG. 4A, openings reaching the conductive layer 172 are formed in the insulating layers 175, 174, and 173. Then, the plugs 176 are formed to fill the openings.

Next, as illustrated in FIG. 4A, a conductive film 151f to be the conductive layers 151R, 151G, 151B, and 151C is formed over the plugs 176 and the insulating layer 175. The conductive film 151f can be formed by a sputtering method or a vacuum evaporation method, for example. A metal material can be used for the conductive film 151f, for example.

Next, as illustrated in FIG. 4A, a conductive film 152f to be the conductive layers 152R, 152G, 152B, and 152C is formed over the conductive film 151f. The conductive film 152f can be formed by a sputtering method or a vacuum evaporation method, for example. A conductive oxide can be used for the conductive film 152f, for example. The conductive film 152f can be a stack of a film formed using a metal material and a film formed thereover using a conductive oxide. For example, the conductive film 152f can be a stack of a film formed using titanium, silver, or an alloy containing silver and a film formed thereover using a conductive oxide.

The conductive film 152f can be formed by an ALD method. In this case, for the conductive film 152f, an oxide containing one or more selected from indium, tin, zinc, gallium, titanium, aluminum, and silicon can be used. In this case, the conductive film 152f can be formed by repeating a cycle of introduction of a precursor (generally referred to as a metal precursor or the like in some cases), purge of the precursor, introduction of an oxidizer (generally referred to as a reactant, a non-metal precursor, or the like in some cases), and purge of the oxidizer. Here, in the case where an oxide film including a plurality of kinds of metals (e.g., an indium tin oxide film) is formed as the conductive film 152f, the composition of the metals can be controlled by varying the number of cycles for different kinds of precursors.

For example, in the case where an indium tin oxide film is formed as the conductive film 152f, after a precursor containing indium is introduced, the precursor is purged, and an oxidizer is introduced to form an In—O film, and then a precursor containing tin is introduced, the precursor is purged, and an oxidizer is introduced to form a Sn—O film. Here, when the number of cycles of forming an In—O film is larger than the number of cycles of forming a Sn—O film, the number of In atoms included in the conductive film 152f can be larger than the number of Sn atoms included therein.

For example, to form a zinc oxide film as the conductive film 152f, a Zn—O film is formed in the above procedure. For another example, to form an aluminum zinc oxide film as the conductive film 152f, a Zn—O film and an Al—O film are formed in the above procedure. For another example, to form a titanium oxide film as the conductive film 152f, a Ti—O film is formed in the above procedure. For another example, to form an indium tin oxide film including silicon as the conductive film 152f, an In—O film, a Sn—O film, and a Si—O film are formed in the above procedure. For another example, to form a zinc oxide film including gallium, a Ga—O film and a Zn—O film are formed in the above procedure.

As a precursor containing indium, it is possible to use, for example, triethylindium, trimethylindium, or [1,1,1-trimethyl-N-(trimethylsilyl)amide]-indium. As a precursor containing tin, it is possible to use, for example, tin chloride or tetrakis(dimethylamido)tin. As a precursor containing zinc, it is possible to use, for example, diethylzinc or dimethylzinc. As a precursor containing gallium, it is possible to use, for example, triethylgallium. As a precursor containing titanium, it is possible to use, for example, titanium chloride, tetrakis(dimethylamido)titanium, or tetraisopropyl titanate. As a precursor containing aluminum, it is possible to use, for example, aluminum chloride or trimethylaluminum. As a precursor containing silicon, it is possible to use, for example, trisilylamine, bis(diethylamino)silane, tris(dimethylamino)silane, bis(tert-butylamino)silane, or bis(ethylmethylamino)silane. As the oxidizer, water vapor, oxygen plasma, or an ozone gas can be used.

Subsequently, a resist mask 191 is formed over the conductive films 151f and 152f as illustrated in FIG. 4A. The resist mask 191 can be formed by application of a photosensitive material (photoresist), light exposure, and development.

Subsequently, as illustrated in FIG. 4B, the conductive films 151f and 152f in a region not overlapping with the resist mask 191, for example, are removed by an etching method, specifically, a dry etching method, for instance, so that the pixel electrodes each including the conductive layers 151 and 152 are formed. Note that in the case where the conductive film 151f includes a layer formed using a conductive oxide such as indium tin oxide, for example, the layer may be removed by a wet etching method. Thus, the conductive layers 151 and 152 are formed. In the case where part of the conductive film 151f is removed by a dry etching method, for example, a recessed portion may be formed in a region of the insulating layer 175 not overlapping with the conductive layer 151.

Note that the following process may be employed: the conductive film 152f is processed by a lithography method to form the conductive layers 152R, 152G, 152B, and 152C, and then, the conductive film 151f is processed using the conductive layers 152R, 152G, 152B, and 152C as masks. Specifically, after a resist mask is formed, part of the conductive film 152f is removed by an etching method, for example. The conductive film 152f can be removed by a wet etching method, for example. The conductive film 152f may be removed by a dry etching method. After that, the conductive film 151f is preferably removed by a wet etching method.

Here, hydrophobization treatment is preferably performed on the conductive layer 152. The hydrophobization treatment can change the hydrophilic properties of the subject surface to hydrophobic properties or increase the hydrophobic properties of the subject surface. The hydrophobization treatment for the conductive layer 152 can increase the adhesion between the conductive layer 152 and the organic compound layer 103 formed in a later step and inhibit film peeling. Note that the hydrophobization treatment is not necessarily performed.

Next, the resist mask 191 is removed as illustrated in FIG. 4C. The resist mask 191 can be removed by ashing using oxygen plasma, for example. Alternatively, an oxygen gas and any of CF₄, C₄F₈, SF₆, CHF₃, Cl₂, H₂O, BCl₃, and a Group 18 element such as He may be used. Alternatively, the resist mask 191 may be removed by wet etching.

Then, as illustrated in FIG. 4D, an insulating film 156f to be an insulating layer 156R, an insulating layer 156G, an insulating layer 156B, and an insulating layer 156C is formed over the conductive layers 151R and 152R, the conductive layers 151G and 152G, the conductive layers 151B and 152B, the conductive layers 151C and 152C, and the insulating layer 175. The insulating film 156f can be formed by a CVD method, an ALD method, a sputtering method, or a vacuum evaporation method, for example.

For the insulating film 156f, an inorganic material can be used. As the insulating film 156f, an inorganic insulating film such as an oxide insulating film, a nitride insulating film, an oxynitride insulating film, or a nitride oxide insulating film can be used, for example. For example, an oxide insulating film including silicon, a nitride insulating film including silicon, an oxynitride insulating film including silicon, a nitride oxide insulating film including silicon, or the like can be used as the insulating film 156f. For the insulating film 156f, silicon oxynitride can be used, for example.

Subsequently, as illustrated in FIG. 4E, the insulating film 156f is processed to form the insulating layers 156R, 156G, 156B, and 156C. The insulating layer 156 can be formed by performing etching substantially uniformly on the top surface of the insulating film 156f, for example. Such uniform etching for planarization is also referred to as etch back treatment. Note that the insulating layer 156 may be formed by a lithography method.

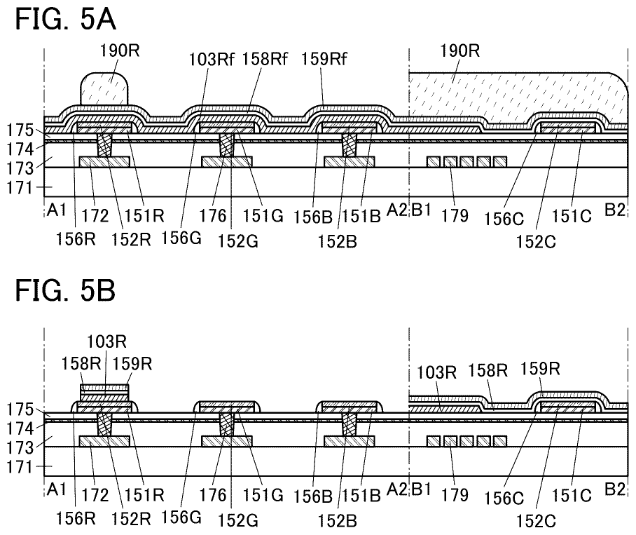

Next, as illustrated in FIG. 5A, an organic compound film 103Rf to be the organic compound layer 103R is formed over the conductive layers 152R, 152G, and 152B, the insulating layers 156R, 156G, and 156B, and the insulating layer 175.

As illustrated in FIG. 5A, the organic compound film 103Rf is not formed over the conductive layer 152C. For example, a mask for defining a film formation area (also referred to as an area mask, a rough metal mask, or the like to be distinguished from a fine metal mask) is used, so that the organic compound film 103Rf can be formed only in a desired region. Employing a film formation step using an area mask and a processing step using a resist mask enables a light-emitting device to be fabricated by a relatively easy process.

The organic compound film 103Rf can be formed by an evaporation method, specifically a vacuum evaporation method, for example. The organic compound film 103Rf may be formed by a transfer method, a printing method, an ink-jet method, a coating method, or the like.

Next, as illustrated in FIG. 5A, a sacrificial film 158Rf to be a sacrificial layer 158R and a mask film 159Rf to be a mask layer 159R are sequentially formed over the organic compound film 103Rf, the conductive layer 152C, and the insulating layer 175.

Although this embodiment describes an example where a mask film having a two-layer structure of the sacrificial film 158Rf and the mask film 159Rf is formed, a mask film may have a single-layer structure or a stacked-layer structure of three or more layers.

Providing the sacrificial layer over the organic compound film 103Rf can reduce damage to the organic compound film 103Rf in the manufacturing process of the display device, resulting in an increase in reliability of the light-emitting device.

As the sacrificial film 158Rf, a film that is highly resistant to the process conditions for the organic compound film 103Rf, specifically, a film having high etching selectivity with respect to the organic compound film 103Rf is used. As the mask film 159Rf, a film having high etching selectivity with respect to the sacrificial film 158Rf is used.

The sacrificial film 158Rf and the mask film 159Rf are formed at a temperature lower than the upper temperature limit of the organic compound film 103Rf. The typical substrate temperatures in formation of the sacrificial film 158Rf and the mask film 159Rf are each lower than or equal to 200° C., preferably lower than or equal to 150° C., further preferably lower than or equal to 120° C., still further preferably lower than or equal to 100° C., yet still further preferably lower than or equal to 80° C.

The sacrificial film 158Rf and the mask film 159Rf are preferably films that can be removed by a wet etching method. The use of a wet etching method can reduce damage to the organic compound film 103Rf in processing of the sacrificial film 158Rf and the mask film 159Rf, as compared to the case of using a dry etching method.

The sacrificial film 158Rf and the mask film 159Rf can be formed by a sputtering method, an ALD method (including a thermal ALD method and a PEALD method), a CVD method, or a vacuum evaporation method, for example. Alternatively, the sacrificial film 158Rf and the mask film 159Rf may be formed by the above-described wet film-formation method.

Note that the sacrificial film 158Rf that is formed over and in contact with the organic compound film 103Rf is preferably formed by a formation method that is less likely to damage the organic compound film 103Rf than a formation method of the mask film 159Rf. For example, the sacrificial film 158Rf is preferably formed by an ALD method or a vacuum evaporation method rather than a sputtering method.

As each of the sacrificial film 158Rf and the mask film 159Rf, one or more of a metal film, an alloy film, a metal oxide film, a semiconductor film, an organic insulating film, and an inorganic insulating film, for example, can be used.

For each of the sacrificial film 158Rf and the mask film 159Rf, it is possible to use a metal material such as gold, silver, platinum, magnesium, nickel, tungsten, chromium, molybdenum, iron, cobalt, copper, palladium, titanium, aluminum, yttrium, zirconium, or tantalum or an alloy material containing any of the metal materials, for example. It is particularly preferable to use a low-melting-point material such as aluminum or silver. A metal material that can block ultraviolet rays is preferably used for one or both of the sacrificial film 158Rf and the mask film 159Rf, in which case the organic compound film 103Rf can be inhibited from being irradiated with ultraviolet rays and deteriorating.

The sacrificial film 158Rf and the mask film 159Rf can each be formed using a metal oxide such as In—Ga—Zn oxide, indium oxide, In—Zn oxide, In—Sn oxide, indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or indium tin oxide containing silicon.

In place of gallium described above, an element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used.

As each of the sacrificial film and the mask film, a film including a material having a light-blocking property, particularly with respect to ultraviolet rays, is preferably used. Although a variety of materials such as a metal, an insulator, a semiconductor, and a metalloid that have a property of blocking ultraviolet rays can be used as a light-blocking material, each of the sacrificial film and the mask film is preferably a film capable of being processed by etching and is particularly preferably a film having good processability because part or the whole of each of the sacrificial film and the mask film is removed in a later step.

The sacrificial film and the mask film are preferably formed using a semiconductor material such as silicon or germanium, for example, for excellent compatibility with a semiconductor manufacturing process. Alternatively, an oxide or a nitride of the semiconductor material can be used. A non-metallic material such as carbon or a compound thereof can be used. A metal such as titanium, tantalum, tungsten, chromium, or aluminum or an alloy containing at least one of these metals can be used. Alternatively, an oxide containing the above-described metal, such as titanium oxide or chromium oxide, or a nitride such as titanium nitride, chromium nitride, or tantalum nitride can be used.

When a film including a material having a property of blocking ultraviolet rays is used as each of the sacrificial film and the mask film, the organic compound layer can be inhibited from being irradiated with ultraviolet rays in a light exposure step, for example. The organic compound layer is inhibited from being damaged by ultraviolet rays, so that the reliability of the light-emitting device can be improved.

Note that the same effect is obtained when a film including a material having a property of blocking ultraviolet rays is used for an after-mentioned inorganic insulating film 125f.

As each of the sacrificial film 158Rf and the mask film 159Rf, any of a variety of inorganic insulating films can be used. In particular, an oxide insulating film is preferable because its adhesion to the organic compound film 103Rf is higher than that of a nitride insulating film. For example, an inorganic insulating material such as aluminum oxide, hafnium oxide, or silicon oxide can be used for the sacrificial film 158Rf and the mask film 159Rf. As the sacrificial film 158Rf and the mask film 159Rf, aluminum oxide films can be formed by an ALD method, for example. An ALD method is preferably used, in which case damage to a base (in particular, the organic compound layer) can be reduced.

For example, an inorganic insulating film (e.g., an aluminum oxide film) formed by an ALD method can be used as the sacrificial film 158Rf, and an inorganic film (e.g., an In—Ga—Zn oxide film, an aluminum film, or a tungsten film) formed by a sputtering method can be used as the mask film 159Rf.

Note that the same inorganic insulating film can be used for both the sacrificial film 158Rf and the inorganic insulating layer 125 that is to be formed later. For example, an aluminum oxide film formed by an ALD method can be used for both the sacrificial film 158Rf and the inorganic insulating layer 125. For the sacrificial film 158Rf and the inorganic insulating layer 125, the same film formation conditions may be used or different film formation conditions may be used. For example, when the sacrificial film 158Rf is formed under conditions similar to those of the inorganic insulating layer 125, the sacrificial film 158Rf can be an insulating layer having a high barrier property against at least one of water and oxygen. Meanwhile, since the sacrificial film 158Rf is a layer a large part or the whole of which is to be removed in a later step, it is preferable that the processing of the sacrificial film 158Rf be easy. Therefore, the sacrificial film 158Rf is preferably formed with a substrate temperature lower than that for formation of the inorganic insulating layer 125.

One or both of the sacrificial film 158Rf and the mask film 159Rf may be formed using an organic material. For example, as the organic material, a material that can be dissolved in a solvent chemically stable with respect to at least the uppermost film of the organic compound film 103Rf may be used. Specifically, a material that will be dissolved in water or an alcohol can be suitably used. In forming a film of such a material, it is preferable to apply the material dissolved in a solvent such as water or an alcohol by a wet film-formation method and then perform heat treatment for evaporating the solvent. At this time, the heat treatment is preferably performed in a reduced-pressure atmosphere, in which case the solvent can be removed at a low temperature in a short time and thermal damage to the organic compound film 103Rf can be reduced accordingly.

The sacrificial film 158Rf and the mask film 159Rf may be formed using an organic resin such as polyvinyl alcohol (PVA), polyvinyl butyral, polyvinylpyrrolidone, polyethylene glycol, polyglycerin, pullulan, water-soluble cellulose, an alcohol-soluble polyamide resin, or a fluorine resin like perfluoropolymer.

For example, an organic film (e.g., a PVA film) formed by an evaporation method or any of the above wet film-formation methods can be used as the sacrificial film 158Rf, and an inorganic film (e.g., a silicon nitride film) formed by a sputtering method can be used as the mask film 159Rf.

Subsequently, a resist mask 190R is formed over the mask film 159Rf as illustrated in FIG. 5A. The resist mask 190R can be formed by application of a photosensitive material (photoresist), light exposure, and development.

The resist mask 190R may be formed using either a positive resist material or a negative resist material.

The resist mask 190R is provided at a position overlapping with the conductive layer 152R. The resist mask 190R is preferably provided also at a position overlapping with the conductive layer 152C. This can inhibit the conductive layer 152C from being damaged during the process of manufacturing the display device. Note that the resist mask 190R is not necessarily provided over the conductive layer 152C. The resist mask 190R is preferably provided to cover the area from an end portion of the organic compound film 103Rf to an end portion of the conductive layer 152C (the end portion closer to the organic compound film 103Rf), as illustrated in the cross-sectional view along the line B1-B2 in FIG. 5A.

Next, as illustrated in FIG. 5B, part of the mask film 159Rf is removed using the resist mask 190R, so that the mask layer 159R is formed. The mask layer 159R remains over the conductive layers 152R and 152C. After that, the resist mask 190R is removed. Then, part of the sacrificial film 158Rf is removed using the mask layer 159R as a mask (also referred to as a hard mask), so that the sacrificial layer 158R is formed.

Each of the sacrificial film 158Rf and the mask film 159Rf can be processed by a wet etching method or a dry etching method. The sacrificial film 158Rf and the mask film 159Rf are preferably processed by isotropic etching.

The use of a wet etching method can reduce damage to the organic compound film 103Rf in processing of the sacrificial film 158Rf and the mask film 159Rf, as compared to the case of using a dry etching method. In the case of using a wet etching method, it is preferable to use a developer, an aqueous solution of tetramethylammonium hydroxide (TMAH), dilute hydrofluoric acid, oxalic acid, phosphoric acid, acetic acid, nitric acid, or a chemical solution containing a mixed solution of any of these acids, for example.

Since the organic compound film 103Rf is not exposed in the processing of the mask film 159Rf, the range of choice for a processing method for the mask film 159Rf is wider than that for the sacrificial film 158Rf Specifically, even in the case where a gas containing oxygen is used as the etching gas in the processing of the mask film 159Rf, deterioration of the organic compound film 103Rf can be inhibited.

In the case of using a dry etching method to process the sacrificial film 158Rf, deterioration of the organic compound film 103Rf can be inhibited by not using a gas containing oxygen as the etching gas. In the case of using a dry etching method, it is preferable to use a gas containing CF₄, C₄F₈, SF₆, CHF₃, Cl₂, H₂O, BCl₃, or a Group 18 element such as He, for example, as the etching gas.

For example, in the case where an aluminum oxide film formed by an ALD method is used as the sacrificial film 158Rf, part of the sacrificial film 158Rf can be removed by a dry etching method using CHF₃ and He or a combination of CHF₃, He, and CH₄. In the case where an In—Ga—Zn oxide film formed by a sputtering method is used as the mask film 159Rf, part of the mask film 159Rf can be removed by a wet etching method using diluted phosphoric acid. Alternatively, part of the mask film 159Rf may be removed by a dry etching method using CH₄ and Ar. Alternatively, part of the mask film 159Rf can be removed by a wet etching method using diluted phosphoric acid. In the case where a tungsten film formed by a sputtering method is used as the mask film 159Rf, part of the mask film 159Rf can be removed by a dry etching method using a combination of SF₆, CF₄, and O₂ or a combination of CF₄, Cl₂, and O₂.

The resist mask 190R can be removed by a method similar to that for the resist mask 191. For example, the resist mask 190R can be removed by ashing using oxygen plasma. Alternatively, an oxygen gas and any of CF₄, C₄F₈, SF₆, CHF₃, Cl₂, H₂O, BCl₃, and a Group 18 element such as He may be used. Alternatively, the resist mask 190R may be removed by wet etching. At this time, the sacrificial film 158Rf is located on the outermost surface, and the organic compound film 103Rf is not exposed; thus, the organic compound film 103Rf can be inhibited from being damaged in the step of removing the resist mask 190R. In addition, the range of choice for the method for removing the resist mask 190R can be widened.

Next, as illustrated in FIG. 5B, the organic compound film 103Rf is processed, so that the organic compound layer 103R is formed. For example, part of the organic compound film 103Rf is removed using the mask layer 159R and the sacrificial layer 158R as a hard mask to form the organic compound layer 103R.

Accordingly, as illustrated in FIG. 5B, the stacked-layer structure of the organic compound layer 103R, the sacrificial layer 158R, and the mask layer 159R remains over the conductive layer 152R. The conductive layers 152G and 152B are exposed.

In the example illustrated in FIG. 5B, an end portion of the organic compound layer 103R is located inward from an end portion of the conductive layer 152R. This structure allows miniaturization of pixels, enabling fabricating a high-resolution display. Although not illustrated in FIG. 5B, by the above etching treatment, a recessed portion may be formed in the insulating layer 175 in a region not overlapping with the organic compound layer 103R.

As described above, the resist mask 190R is preferably provided to cover the area from the end portion of the organic compound layer 103R to the end portion of the conductive layer 152C (the end portion closer to the organic compound layer 103R) in the cross section along the dashed-dotted line B1-B2. In that case, as illustrated in FIG. 5B, the sacrificial layer 158R and the mask layer 159R are provided to cover the area from the end portion of the organic compound layer 103R to the end portion of the conductive layer 152C (the end portion closer to the organic compound layer 103R) in the cross section along the dashed-dotted line B1-B2. Hence, the insulating layer 175 can be inhibited from being exposed in the cross section along the dashed-dotted line B1-B2, for example. This can prevent the insulating layers 175, 174, and 173 from being partly removed by etching and thus prevent the conductive layer 179 from being exposed. Accordingly, the conductive layer 179 can be inhibited from being unintentionally electrically connected to another conductive layer. For example, a short circuit between the conductive layer 179 and a common electrode 155 formed in a later step can be inhibited.

The organic compound film 103Rf is preferably processed by anisotropic etching. Anisotropic dry etching is particularly preferable. Alternatively, wet etching may be used.

In the case of using a dry etching method, deterioration of the organic compound film 103Rf can be inhibited by not using a gas containing oxygen as the etching gas.

A gas containing oxygen may be used as the etching gas. When the etching gas contains oxygen, the etching rate can be increased. Therefore, the etching can be performed under a low-power condition while an adequately high etching rate is maintained. Accordingly, damage to the organic compound film 103Rf can be reduced. Furthermore, a defect such as attachment of a reaction product generated in the etching can be inhibited.

In the case of using a dry etching method, it is preferable to use a gas containing at least one of H₂, CF₄, C₄F₈, SF₆, CHF₃, Cl₂, H₂O, BCl₃, and a Group 18 element such as He or Ar as the etching gas, for example. Alternatively, a gas containing oxygen and at least one of the above is preferably used as the etching gas. Alternatively, an oxygen gas may be used as the etching gas. Specifically, for example, a gas containing H₂ and Ar or a gas containing CF₄ and He can be used as the etching gas. For another example, a gas containing CF₄, He, and oxygen can be used as the etching gas. For another example, a gas containing H₂ and Ar and a gas containing oxygen can be used as the etching gas.

As described above, in one embodiment of the present invention, the mask layer 159R is formed in the following manner: the resist mask 190R is formed over the mask film 159Rf and part of the mask film 159Rf is removed using the resist mask 190R. After that, part of the organic compound film 103Rf is removed using the mask layer 159R as a hard mask, so that the organic compound layer 103R is formed. In other words, the organic compound layer 103R is formed by processing the organic compound film 103Rf by a lithography method. Note that part of the organic compound film 103Rf may be removed using the resist mask 190R. Then, the resist mask 190R may be removed.

Next, hydrophobization treatment for the conductive layer 152G, for example, is preferably performed. At the time of processing the organic compound film 103Rf, the properties of a surface of the conductive layer 152G change to hydrophilic properties in some cases, for example. The hydrophobization treatment for the conductive layer 152G, for example, can increase the adhesion between the conductive layer 152G and a layer to be formed in a later step (which is the organic compound layer 103G here) and inhibit film peeling. Note that the hydrophobization treatment is not necessarily performed.

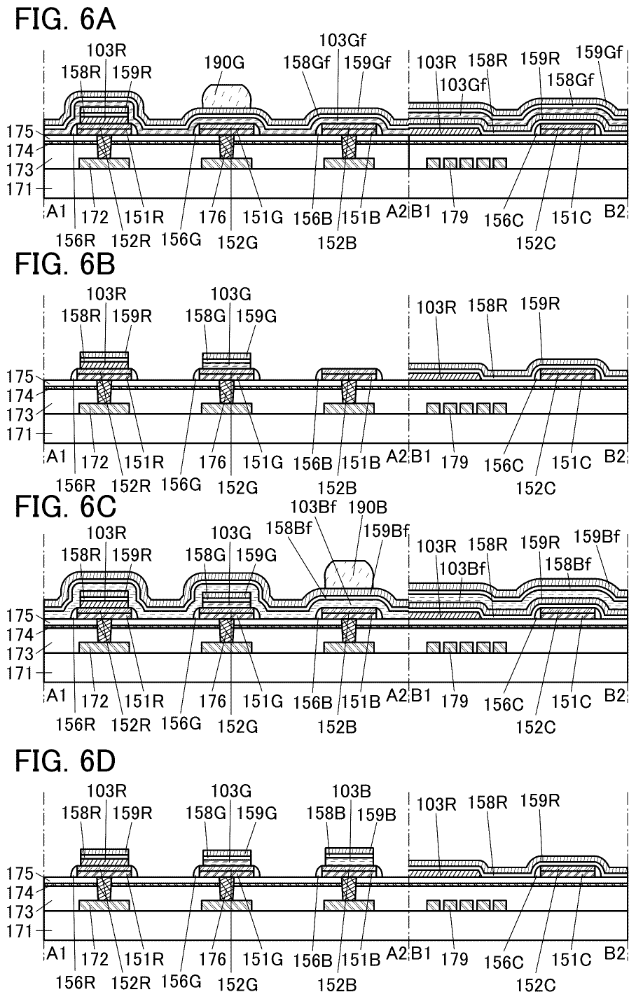

Next, as illustrated in FIG. 6A, an organic compound film 103Gf to be the organic compound layer 103G is formed over the conductive layers 152G and 152B, the insulating layers 156R, 156G, and 156B, the mask layer 159R, and the insulating layer 175.

The organic compound film 103Gf can be formed by a method similar to that for forming the organic compound film 103Rf. The organic compound film 103Gf can have a structure similar to that of the organic compound film 103Rf.

Then, as illustrated in FIG. 6A, a sacrificial film 158Gf to be a sacrificial layer 158G and a mask film 159Gf to be a mask layer 159G are sequentially formed over the organic compound film 103Gf and the mask layer 159R. After that, a resist mask 190G is formed. The materials and the formation methods of the sacrificial film 158Gf and the mask film 159Gf are similar to those of the sacrificial film 158Rf and the mask film 159Rf. The material and the formation method of the resist mask 190G are similar to those of the resist mask 190R.

The resist mask 190G is provided at a position overlapping with the conductive layer 152G.

Subsequently, as illustrated in FIG. 6B, part of the mask film 159Gf is removed using the resist mask 190G, so that the mask layer 159G is formed. The mask layer 159G remains over the conductive layer 152G. After that, the resist mask 190G is removed. Then, part of the sacrificial film 158Gf is removed using the mask layer 159G as a mask, so that the sacrificial layer 158G is formed. Next, the organic compound film 103Gf is processed, so that the organic compound layer 103G is formed. For example, part of the organic compound film 103Gf is removed using the mask layer 159G and the sacrificial layer 158G as a hard mask to form the organic compound layer 103G.

Accordingly, as illustrated in FIG. 6B, the stacked-layer structure of the organic compound layer 103G, the sacrificial layer 158G, and the mask layer 159G remains over the conductive layer 152G. The mask layer 159R and the conductive layer 152B are exposed.

Next, hydrophobization treatment for the conductive layer 152B, for example, is preferably performed. At the time of processing the organic compound film 103Gf, the properties of a surface of the conductive layer 152B change to hydrophilic properties in some cases, for example. The hydrophobization treatment for the conductive layer 152B, for example, can increase the adhesion between the conductive layer 152B and a layer to be formed in a later step (which is the organic compound layer 103B here) and inhibit film peeling. Note that the hydrophobization treatment is not necessarily performed.

Next, as illustrated in FIG. 6C, an organic compound film 103Bf to be the organic compound layer 103B is formed over the conductive layer 152B, the mask layer 159R, the insulating layers 156R, 156G, and 156B, the mask layer 159G, and the insulating layer 175.

The organic compound film 103Bf can be formed by a method similar to that for forming the organic compound film 103Rf. The organic compound film 103Bf can have a structure similar to that of the organic compound film 103Rf.

Then, as illustrated in FIG. 6C, a sacrificial film 158Bf to be a sacrificial layer 158B and a mask film 159Bf to be a mask layer 159B are sequentially formed over the organic compound film 103Bf and the mask layer 159R. After that, a resist mask 190B is formed. The materials and the formation methods of the sacrificial film 158Bf and the mask film 159Bf are similar to those of the sacrificial film 158Rf and the mask film 159Rf. The material and the formation method of the resist mask 190B are similar to those of the resist mask 190R.

The resist mask 190B is provided at a position overlapping with the conductive layer 152B.

Subsequently, as illustrated in FIG. 6D, part of the mask film 159Bf is removed using the resist mask 190B, so that the mask layer 159B is formed. The mask layer 159B remains over the conductive layer 152B. After that, the resist mask 190B is removed. Then, part of the sacrificial film 158Bf is removed using the mask layer 159B as a mask, so that the sacrificial layer 158B is formed. Next, the organic compound film 103Bf is processed, so that the organic compound layer 103B is formed. For example, part of the organic compound film 103Bf is removed using the mask layer 159B and the sacrificial layer 158B as a hard mask to form the organic compound layer 103B.

Accordingly, as illustrated in FIG. 6D, the stacked-layer structure of the organic compound layer 103B, the sacrificial layer 158B, and the mask layer 159B remains over the conductive layer 152B. The mask layers 159R and 159G are exposed.

Note that the side surfaces of the organic compound layers 103R, 103G, and 103B are preferably perpendicular or substantially perpendicular to their formation surfaces. For example, the angle between the formation surfaces and these side surfaces is preferably greater than or equal to 60° and less than or equal to 90°.

The distance between two adjacent layers among the organic compound layers 103R, 103G, and 103B, which are formed by a lithography method as described above, can be shortened to less than or equal to 8 μm, less than or equal to 5 μm, less than or equal to 3 μm, less than or equal to 2 μm, or less than or equal to 1 μm. Here, the distance can be specified, for example, by the distance between opposite end portions of two adjacent layers among the organic compound layers 103R, 103G, and 103B. Shortening the distance between the island-shaped organic compound layers can provide a display device having high resolution and a high aperture ratio. In addition, the distance between the first electrodes of adjacent light-emitting devices can also be shortened to be, for example, less than or equal to 10 μm, less than or equal to 8 μm, less than or equal to 5 μm, less than or equal to 3 μm, or less than or equal to 2 μm. Note that the distance between the first electrodes of adjacent light-emitting devices is preferably greater than or equal to 2 μm and less than or equal to 5 μm.

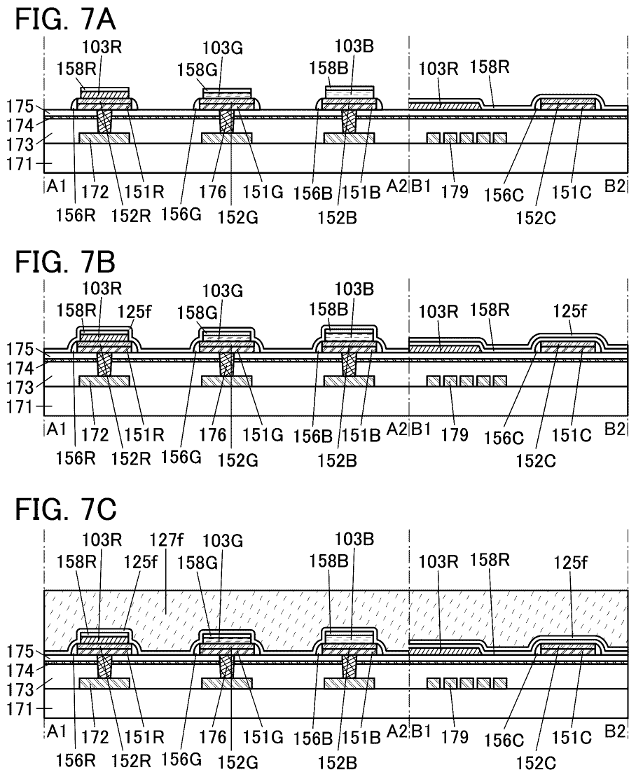

Next, as illustrated in FIG. 7A, the mask layers 159R, 159G, and 159B are preferably removed. The sacrificial layers 158R, 158G, and 158B and the mask layers 159R, 159G, and 159B remain in the display device in some cases depending on the subsequent steps. Removing the mask layers 159R, 159G, and 159B at this stage can inhibit the mask layers 159R, 159G, and 159B from being left in the display device. For example, in the case where a conductive material is used for the mask layers 159R, 159G, and 159B, removing the mask layers 159R, 159G, and 159B in advance can inhibit generation of a leakage current, formation of a capacitor, and the like due to the remaining mask layers 159R, 159G, and 159B.

This embodiment describes an example where the mask layers 159R, 159G, and 159B are removed; however, the mask layers 159R, 159G, and 159B are not necessarily removed. For example, in the case where the mask layers 159R, 159G, and 159B include the above-described material having a property of blocking ultraviolet rays, the procedure preferably proceeds to the next step without removing the mask layers 159R, 159G, and 159B, in which case the organic compound layers can be protected from ultraviolet rays.

The step of removing the mask layers can be performed by a method similar to that for the step of processing the mask films. Specifically, by using a wet etching method, damage applied to the organic compound layers 103R, 103G, and 103B at the time of removing the mask layers can be reduced as compared to the case of using a dry etching method.

The mask layers may be removed by being dissolved in a solvent such as water or an alcohol. Examples of an alcohol include ethyl alcohol, methyl alcohol, isopropyl alcohol (IPA), and glycerin.

After the mask layers are removed, drying treatment may be performed in order to remove water included in the organic compound layers 103R, 103G, and 103B and water adsorbed onto the surfaces of the organic compound layers 103R, 103G, and 103B. For example, heat treatment in an inert gas atmosphere or a reduced-pressure atmosphere can be performed. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 120° C. The heat treatment is preferably performed in a reduced-pressure atmosphere, in which case drying at a lower temperature is possible.

Next, as illustrated in FIG. 7B, the inorganic insulating film 125f to be the inorganic insulating layer 125 is formed to cover the organic compound layers 103R, 103G, and 103B and the sacrificial layers 158R, 158G, and 158B.

As described later, an insulating film 127f to be the insulating layer 127 is formed in contact with the top surface of the inorganic insulating film 125f. Therefore, the top surface of the inorganic insulating film 125f preferably has a high affinity for the material used for the insulating film (e.g., a photosensitive resin composition containing an acrylic resin). To improve the affinity, surface treatment is preferably performed so that the top surface of the inorganic insulating film 125f is made hydrophobic or its hydrophobic properties are improved. For example, it is preferable to perform the treatment using a silylation agent such as hexamethyldisilazane (HMDS). By making the top surface of the inorganic insulating film 125f hydrophobic in such a manner, the insulating film 127f can be formed with favorable adhesion. Note that the above-described hydrophobization treatment may be performed as the surface treatment.

Then, as illustrated in FIG. 7C, the insulating film 127f to be the insulating layer 127 is formed over the inorganic insulating film 125f.

The inorganic insulating film 125f and the insulating film 127f are preferably formed by a formation method by which the organic compound layers 103R, 103G, and 103B are less damaged. The inorganic insulating film 125f, which is formed in contact with the side surfaces of the organic compound layers 103R, 103G, and 103B, is particularly preferably formed by a formation method that causes less damage to the organic compound layers 103R, 103G, and 103B than the formation method of the insulating film 127f.

Each of the inorganic insulating film 125f and the insulating film 127f is formed at a temperature lower than the upper temperature limits of the organic compound layers 103R, 103G, and 103B. When the inorganic insulating film 125f is formed at a high substrate temperature, the formed inorganic insulating film 125f, even with a small thickness, can have a low impurity concentration and a high barrier property against at least one of water and oxygen.

The substrate temperature at the time of forming the inorganic insulating film 125f and the insulating film 127f is preferably higher than or equal to 60° C., higher than or equal to 80° C., higher than or equal to 100° C., or higher than or equal to 120° C. and lower than or equal to 200° C., lower than or equal to 180° C., lower than or equal to 160° C., lower than or equal to 150° C., or lower than or equal to 140° C.

As the inorganic insulating film 125f, an insulating film having a thickness greater than or equal to 3 nm, greater than or equal to 5 nm, or greater than or equal to 10 nm and less than or equal to 200 nm, less than or equal to 150 nm, less than or equal to 100 nm, or less than or equal to 50 nm is preferably formed in the above-described range of the substrate temperature.

The inorganic insulating film 125f is preferably formed by an ALD method, for example. An ALD method is preferably used, in which case damage due to film formation can be reduced and a film with good coverage can be formed. As the inorganic insulating film 125f, an aluminum oxide film is preferably formed by an ALD method, for example.

Alternatively, the inorganic insulating film 125f may be formed by a sputtering method, a CVD method, or a PECVD method, each of which has a higher film formation rate than an ALD method. In that case, a highly reliable display device can be manufactured with high productivity.

The insulating film 127f is preferably formed by the aforementioned wet film-formation method. The insulating film 127f is preferably formed by spin coating using a photosensitive material, for example, and specifically preferably formed using a photosensitive resin composition containing an acrylic resin.

The insulating film 127f is preferably formed using a resin composition containing a polymer, an acid-generating agent, and a solvent, for example. The polymer is formed using one or more kinds of monomers and has a structure where one or more kinds of structural units (also referred to as building blocks) are repeated regularly or irregularly. As the acid-generating agent, one or both of a compound that generates an acid by light irradiation and a compound that generates an acid by heating can be used. The resin composition may also include one or more of a photosensitizing agent, a sensitizer, a catalyst, an adhesive aid, a surface-active agent, and an antioxidant.

Heat treatment (also referred to as prebaking) is preferably performed after the insulating film 127f is formed. The heat treatment is performed at a temperature lower than the upper temperature limits of the organic compound layers 103R, 103G, and 103B. The substrate temperature in the heat treatment is preferably higher than or equal to 50° C. and lower than or equal to 200° C., further preferably higher than or equal to 60° C. and lower than or equal to 150° C., still further preferably higher than or equal to 70° C. and lower than or equal to 120° C. Accordingly, the solvent included in the insulating film 127f can be removed.

Then, part of the insulating film 127f is exposed to visible light or ultraviolet rays. Here, when a positive photosensitive resin composition containing an acrylic resin is used for the insulating film 127f, a region where the insulating layer 127 is not formed in a later step is irradiated with visible light or ultraviolet rays. The insulating layer 127 is formed in regions that are sandwiched between any two of the conductive layers 152R, 152G, and 152B and around the conductive layer 152C. Thus, the top surfaces of the conductive layers 152R, 152G, 152B, and 152C are irradiated with visible light or ultraviolet rays. Note that when a negative photosensitive material is used for the insulating film 127f, the region where the insulating layer 127 is to be formed is irradiated with visible light or ultraviolet rays.

The width of the insulating layer 127 that is to be formed later can be controlled with the exposed region of the insulating film 127f. In this embodiment, processing is performed such that the insulating layer 127 includes a portion overlapping with the top surface of the conductive layer 151.

The light used for the exposure preferably includes the i-line (wavelength: 365 nm). Furthermore, the light used for the exposure may include at least one of the g-line (wavelength: 436 nm) and the h-line (wavelength: 405 nm).

Here, when a barrier insulating layer against oxygen (e.g., an aluminum oxide film) is provided as one or both of the sacrificial layer 158 (the sacrificial layers 158R, 158G, and 158B) and the inorganic insulating film 125f, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layer 158 and the inorganic insulating film 125f over the island-shaped organic compound layer, bonding of oxygen in the atmosphere to the organic compound included in the organic compound layer can be inhibited.

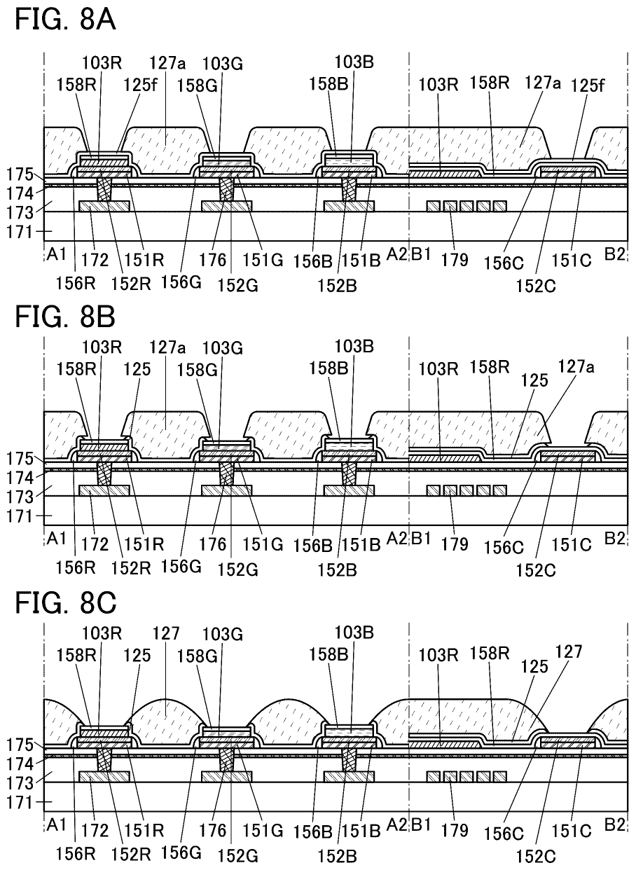

Next, as illustrated in FIG. 8A, development is performed to remove the exposed region of the insulating film 127f, so that an insulating layer 127a is formed. The insulating layer 127a is formed in regions that are sandwiched between any two of the conductive layers 152R, 152G, and 152B and a region surrounding the conductive layer 152C. Here, when an acrylic resin is used for the insulating film 127f, an alkaline solution, such as TMAH, can be used as a developer.

Then, a residue (scum) due to the development may be removed. For example, the residue can be removed by ashing using oxygen plasma.

Etching may be performed to adjust the surface level of the insulating layer 127a. The insulating layer 127a may be processed by ashing using oxygen plasma, for example. In the case where a non-photosensitive material is used for the insulating film 127f, the surface level of the insulating film 127f can be adjusted by the ashing, for example.

Next, as illustrated in FIG. 8B, etching treatment is performed using the insulating layer 127a as a mask to remove part of the inorganic insulating film 125f. This etching treatment may reduce the thickness of part of the sacrificial layers 158R, 158G, and 158B. Thus, the inorganic insulating layer 125 is formed under the insulating layer 127a. Moreover, the surfaces of the sacrificial layers 158R, 158G, and 158B are partly exposed. Note that the etching treatment using the insulating layer 127a as a mask may be hereinafter referred to as first etching treatment.

The first etching treatment can be performed by dry etching or wet etching. Note that the inorganic insulating film 125f is preferably formed using a material similar to that of the sacrificial layers 158R, 158G, and 158B, in which case the first etching treatment can be performed collectively.

By etching using the insulating layer 127a with a tapered side surface as a mask, the side surface of the inorganic insulating layer 125 and upper end portions of the side surfaces of the sacrificial layers 158R, 158G, and 158B can be made to have a tapered shape relatively easily.

In the case of performing dry etching, a chlorine-based gas is preferably used. As the chlorine-based gas, one of Cl₂, BCl₃, SiCl₄, CCl₄, and the like or a mixture of two or more of them can be used. Moreover, one of an oxygen gas, a hydrogen gas, a helium gas, an argon gas, and the like or a mixture of two or more of them can be added as appropriate to the chlorine-based gas. By the dry etching, the exposed regions of the sacrificial layers 158R, 158G, and 158B can be formed with favorable in-plane uniformity.

As a dry etching apparatus, a dry etching apparatus including a high-density plasma source can be used. As the dry etching apparatus including a high-density plasma source, an inductively coupled plasma (ICP) etching apparatus can be used, for example. Alternatively, a capacitively coupled plasma (CCP) etching apparatus including parallel plate electrodes can be used. The capacitively coupled plasma etching apparatus including parallel plate electrodes may have a structure where a high-frequency voltage is applied to one of the parallel plate electrodes. Alternatively, the capacitively coupled plasma etching apparatus may have a structure where different high-frequency voltages are applied to one of the parallel-plate electrodes. Alternatively, the capacitively coupled plasma etching apparatus may have a structure where high-frequency voltages with the same frequency are applied to the parallel-plate electrodes. Alternatively, the capacitively coupled plasma etching apparatus may have a structure where high-frequency voltages with different frequencies are applied to the parallel-plate electrodes.

In the case of performing dry etching, a by-product or the like generated by the dry etching might be deposited on the top surface and the side surface of the insulating layer 127a, for example. Accordingly, a component of the etching gas, a component of the inorganic insulating film 125f, a component of the sacrificial layers 158R, 158G, and 158B, and the like might be included in the insulating layer 127 in the completed display device.

The first etching treatment is preferably performed by wet etching. The use of a wet etching method can reduce damage to the organic compound layers 103R, 103G, and 103B, as compared to the case of using a dry etching method. For example, the wet etching can be performed using an alkaline solution. For instance, TMAH, which is an alkaline solution, can be used for the wet etching of an aluminum oxide film. In this case, puddle wet etching can be performed. Note that the inorganic insulating film 125f is preferably formed using a material similar to that of the sacrificial layers 158R, 158G, and 158B, in which case the above etching treatment can be performed collectively.

Note that in the case where the first etching treatment reduces the thicknesses of the sacrificial layers 158R, 158G, and 158B, the sacrificial layers 158R, 158G, and 158B remain over the corresponding organic compound layers 103R, 103G, and 103B. This can shorten the treatment time of a later step and can prevent the organic compound layers 103R, 103G, and 103B from being damaged.

Next, the insulating layer 127a is preferably irradiated with visible light or ultraviolet rays by performing light exposure on the entire substrate. The energy density for the light exposure is preferably greater than 0 mJ/cm² and less than or equal to 800 mJ/cm², further preferably greater than 0 mJ/cm² and less than or equal to 500 mJ/cm². Performing such light exposure after the development can sometimes increase the degree of transparency of the insulating layer 127a. In addition, it is sometimes possible to lower the substrate temperature required for subsequent heat treatment for changing the shape of the insulating layer 127a to a tapered shape.

Here, when a barrier insulating layer against oxygen (e.g., an aluminum oxide film) is present as each of the sacrificial layers 158R, 158G, and 158B, diffusion of oxygen into the organic compound layers 103R, 103G, and 103B can be inhibited. When the organic compound layer is irradiated with light (visible light or ultraviolet rays), the organic compound included in the organic compound layer is brought into an excited state and a reaction between the organic compound and oxygen in the atmosphere is promoted in some cases. Specifically, when the organic compound layer is irradiated with light (visible light or ultraviolet rays) in an atmosphere containing oxygen, oxygen might be bonded to the organic compound included in the organic compound layer. By providing the sacrificial layers 158R, 158G, and 158B over the island-shaped organic compound layers, bonding of oxygen in the atmosphere to the organic compounds included in the organic compound layers can be inhibited.

Then, heat treatment (also referred to as post-baking) is performed. The heat treatment can change the insulating layer 127a into the insulating layer 127 having a tapered side surface (FIG. 8C). The heat treatment is performed at a temperature lower than the upper temperature limits of the organic compound layers. The heat treatment can be performed at a substrate temperature higher than or equal to 50° C. and lower than or equal to 200° C., preferably higher than or equal to 60° C. and lower than or equal to 150° C., further preferably higher than or equal to 70° C. and lower than or equal to 130° C. The heating atmosphere may be an air atmosphere or an inert gas atmosphere. Moreover, the heating atmosphere may be an atmospheric-pressure atmosphere or a reduced-pressure atmosphere. The substrate temperature in the heat treatment of this step is preferably higher than that in the heat treatment (prebaking) after the formation of the insulating film 127f. In that case, adhesion between the insulating layer 127 and the inorganic insulating layer 125 can be improved, and corrosion resistance of the insulating layer 127 can be increased.

Note that the side surface of the insulating layer 127 may have a concave shape depending on the material of the insulating layer 127 and the temperature, time, and atmosphere of the post-baking. For example, when the temperature of the post-baking is higher or the duration of the post-baking is longer, the insulating layer 127 is more likely to change in shape and thus the concave shape may be more likely to be formed.

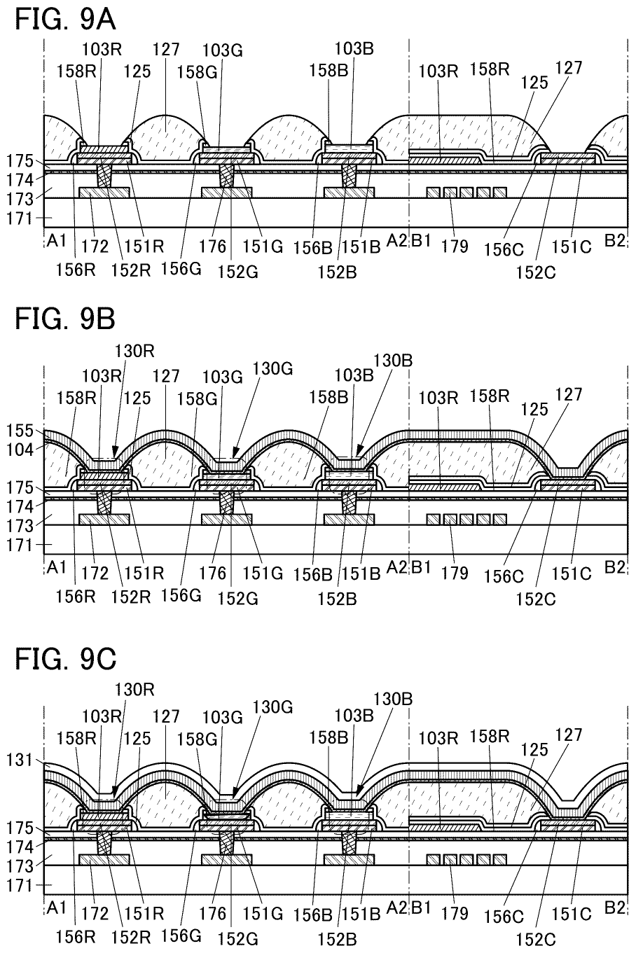

Next, as illustrated in FIG. 9A, etching treatment is performed using the insulating layer 127 as a mask to partly remove the sacrificial layers 158R, 158G, and 158B. Note that part of the inorganic insulating layer 125 is also removed in some cases. Thus, openings are formed in the sacrificial layers 158R, 158G, and 158B, and the top surfaces of the organic compound layers 103R, 103G, and 103B and the conductive layer 152C are exposed. Note that the etching treatment using the insulating layer 127 as a mask may be hereinafter referred to as second etching treatment.

An end portion of the inorganic insulating layer 125 is covered with the insulating layer 127. FIG. 9A illustrates an example where part of an end portion of the sacrificial layer 158G (specifically a tapered portion formed by the first etching treatment) is covered with the insulating layer 127 and a tapered portion formed by the second etching treatment is exposed.

If the first etching treatment is not performed and the inorganic insulating layer 125 and the sacrificial layer are collectively etched after the post-baking, the inorganic insulating layer 125 and the sacrificial layer under an end portion of the insulating layer 127 may disappear because of side etching and a void may be formed. The void causes unevenness on the formation surface of the common electrode 155, so that step disconnection is more likely to be caused in the common electrode 155. Even when a void is formed owing to side etching of the inorganic insulating layer 125 and the sacrificial layer by the first etching treatment, the post-baking performed subsequently can make the insulating layer 127 fill the void. After that, the sacrificial layer is etched by the second etching treatment; thus, the amount of side etching decreases, a void is less likely to be formed, and even if a void is formed, it can be extremely small. Consequently, the formation surface of the common electrode 155 can be made flatter.

Note that the insulating layer 127 may cover the entire end portion of the sacrificial layer 158G. For example, the end portion of the insulating layer 127 may droop to cover the end portion of the sacrificial layer 158G. For another example, the end portion of the insulating layer 127 may be in contact with the top surface of at least one of the organic compound layers 103R, 103G, and 103B. As described above, when light exposure is not performed on the insulating layer 127a after the development, the shape of the insulating layer 127 may be likely to change.

The second etching treatment is performed by wet etching. The use of a wet etching method can reduce damage to the organic compound layers 103R, 103G, and 103B, as compared to the case of using a dry etching method. The wet etching can be performed using an alkaline solution such as TMAH, for example.

Meanwhile, in the case where the second etching treatment is performed by a wet etching method and gaps due to, for example, poor adhesion between the organic compound layer 103 and another layer exist at the interface between the organic compound layer 103 and the sacrificial layer 158, the interface between the organic compound layer 103 and the inorganic insulating layer 125, and the interface between the organic compound layer 103 and the insulating layer 175, the chemical solution used in the second etching treatment sometimes enters the gaps to come into contact with the pixel electrode. Here, when the chemical solution comes into contact with both the conductive layer 151 and the conductive layer 152, one of the conductive layers 151 and 152 that has a lower spontaneous potential than the other suffers from galvanic corrosion in some cases. For example, when the conductive layer 151 is formed using aluminum and the conductive layer 152 is formed using indium tin oxide, the conductive layer 152 sometimes corrodes. As a result, the yield of the display device decreases in some cases. Moreover, the reliability of the display device decreases in some cases.

As described above, when the insulating layer 156 is formed to cover the side surfaces of the conductive layers 151 and 152, the step disconnection of the inorganic insulating layer 125 can be prevented, whereby the chemical solution can be prevented from coming into contact with a lower component such as the conductive layer 151 in the second etching treatment, for example. Thus, corrosion of the pixel electrode can be prevented.

As described above, by providing the insulating layer 127, the inorganic insulating layer 125, and the sacrificial layers 158R, 158G, and 158B, poor connection due to a disconnected portion and an increase in electrical resistance due to a locally thinned portion can be inhibited from occurring in the common electrode 155 between the light-emitting devices. Thus, the display device of one embodiment of the present invention can have improved display quality.

Heat treatment is performed after the organic compound layers 103R, 103G, and 103B are partly exposed. By the heat treatment, water included in the organic compound layers and water adsorbed onto the surfaces of the organic compound layers, for example, can be removed. The shape of the insulating layer 127 may be changed by the heat treatment. Specifically, the insulating layer 127 may be widened to cover at least one of the end portion of the inorganic insulating layer 125, the end portions of the sacrificial layers 158R, 158G, and 158B, and the top surfaces of the organic compound layers 103R, 103G, and 103B.

If the temperature of the heat treatment is too low, water included in the organic compound layers and water adsorbed onto the surfaces of the organic compound layers, for example, cannot be sufficiently removed. If the temperature of the heat treatment is too high, the organic compound layer 103 might deteriorate and the insulating layer 127 might change in shape excessively. Therefore, the temperature of the heat treatment is preferably higher than the temperature at which water is released from the organic compound layer 103 and lower than the glass transition temperature of the organic compound included in the organic compound layer 103, further preferably lower than the glass transition temperature of the organic compound included in the upper surface of the organic compound layer 103. Specifically, the substrate temperature is preferably higher than or equal to 80° C. and lower than or equal to 130° C., further preferably higher than or equal to 90° C. and lower than or equal to 120° C., still further preferably higher than or equal to 100° C. and lower than or equal to 120° C., yet still further preferably higher than or equal to 100° C. and lower than or equal to 110° C. The heating atmosphere may be an air atmosphere or an inert gas atmosphere. Although the heating atmosphere may be an atmospheric-pressure atmosphere or a reduced-pressure atmosphere, a reduced-pressure atmosphere is preferably employed to prevent re-adsorption of water released from the organic compound layer 103.

By the heat treatment, water included in the organic compound layers and water adsorbed onto the surfaces of the organic compound layers, for example, can be sufficiently removed without deterioration of the organic compound layers 103R, 103G, and 103B and an excessive change in the shape of the insulating layer 127. Thus, degradation of the characteristics of the light-emitting devices can be prevented.

Next, as illustrated in FIG. 9B, the common layer 104 and the common electrode 155 are formed over the organic compound layers 103R, 103G, and 103B, the conductive layer 152C, and the insulating layer 127. The common layer 104 and the common electrode 155 can be formed by a sputtering method, a vacuum evaporation method, or the like. The common layer 104 may be formed by an evaporation method while the common electrode 155 may be formed by a sputtering method.

Next, as illustrated in FIG. 9C, the protective layer 131 is formed over the common electrode 155. The protective layer 131 can be formed by a vacuum evaporation method, a sputtering method, a CVD method, an ALD method, or the like.

Then, the substrate 120 is attached to the protective layer 131 using the resin layer 122, so that the display device can be manufactured. In the method for manufacturing the display device of one embodiment of the present invention, the insulating layer 156 is provided on the side surfaces of the conductive layer 151 and the conductive layer 152 as described above. This can increase the yield of the display device and inhibit generation of defects.

As described above, in the method for manufacturing the display device of one embodiment of the present invention, the island-shaped organic compound layers 103R, 103G, and 103B are each formed not by using a fine metal mask but by processing a film formed on the entire surface; thus, the island-shaped layers can be formed to have a uniform thickness. Consequently, a high-resolution display device or a display device with a high aperture ratio can be obtained. Furthermore, even when the resolution or the aperture ratio is high and the distance between the subpixels is extremely short, the organic compound layers 103R, 103G, and 103B can be inhibited from being in contact with each other in the adjacent subpixels. As a result, generation of a leakage current between the subpixels can be inhibited. This can prevent crosstalk, so that a display device with extremely high contrast can be obtained. Moreover, even a display device that includes tandem light-emitting devices formed by a lithography method can have favorable characteristics.

The structure described in this embodiment can be used in combination with any of the structures described in other embodiments as appropriate.

### Embodiment 4

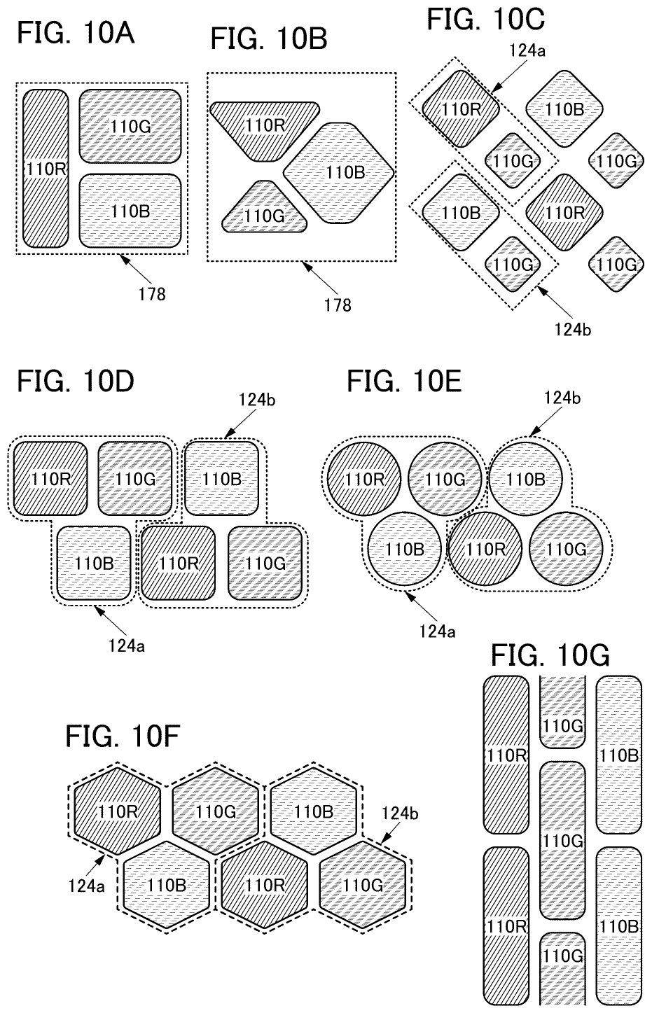

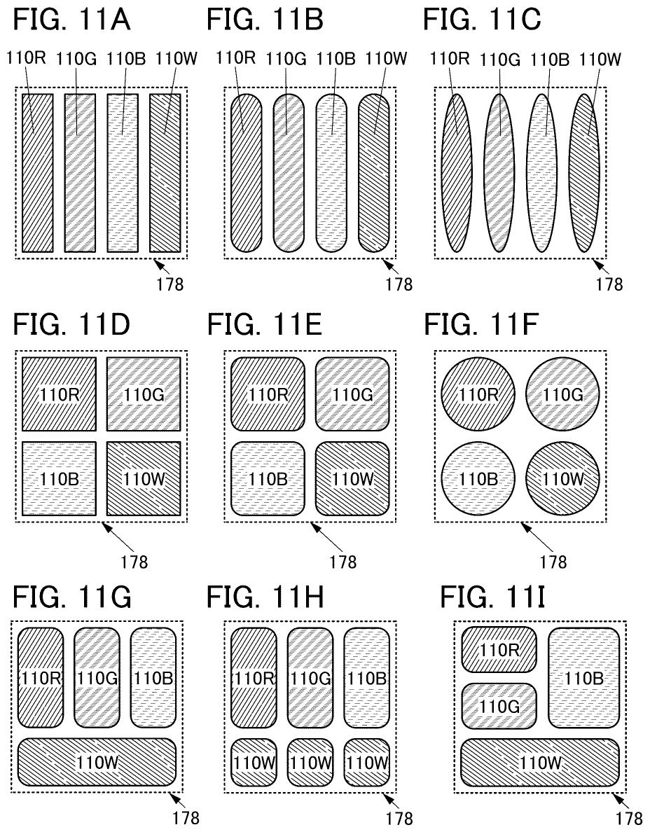

In this embodiment, the light-emitting apparatus of one embodiment of the present invention will be described with reference to FIGS. 10A to 10G and FIGS. 11A to 11I.

### Pixel Layout

In this embodiment, pixel layouts different from that in FIG. 3A will be mainly described. There is no particular limitation on the arrangement of subpixels, and a variety of methods can be employed. Examples of the arrangement of subpixels include stripe arrangement, S-stripe arrangement, matrix arrangement, delta arrangement, Bayer arrangement, and PenTile arrangement.

In this embodiment, the top-view shapes of the subpixels shown in the diagrams correspond to the top-view shapes of light-emitting regions.

Examples of the top-view shape of the subpixel include polygons such as a triangle, a tetragon (including a rectangle and a square), and a pentagon; these polygons with rounded corners; an ellipse; and a circle.

The circuit constituting the subpixel is not necessarily placed within the dimensions of the subpixel illustrated in the diagrams and may be placed outside the subpixel.

The pixel 178 illustrated in FIG. 10A employs S-stripe arrangement. The pixel 178 illustrated in FIG. 10A includes three subpixels, the subpixel 110R, the subpixel 110G, and the subpixel 110B.

The pixel 178 illustrated in FIG. 10B includes the subpixel 110R whose top-view shape is a rough trapezoid or a rough triangle with rounded corners, the subpixel 110G whose top-view shape is a rough trapezoid or a rough triangle with rounded corners, and the subpixel 110B whose top-view shape is a rough tetragon or a rough hexagon with rounded corners. The subpixel 110R has a larger light-emitting area than the subpixel 110G. In this manner, the shapes and sizes of the subpixels can be determined independently. For example, the size of a subpixel including a light-emitting device with higher reliability can be smaller.

Pixels 124a and 124b illustrated in FIG. 10C employ PenTile arrangement. FIG. 10C illustrates an example in which the pixels 124a including the subpixels 110R and 110G and the pixels 124b including the subpixels 110G and 110B are alternately arranged.

The pixels 124a and 124b illustrated in FIGS. 10D to 10F employ delta arrangement. The pixel 124a includes two subpixels (the subpixels 110R and 110G) in the upper row (first row) and one subpixel (the subpixel 110B) in the lower row (second row). The pixel 124b includes one subpixel (the subpixel 110B) in the upper row (first row) and two subpixels (the subpixels 110R and 110G) in the lower row (second row).

FIG. 10D illustrates an example where the top-view shape of each subpixel is a rough tetragon with rounded corners, FIG. 10E illustrates an example where the top-view shape of each subpixel is a circle, and FIG. 10F illustrates an example where the top-view shape of each subpixel is a rough hexagon with rounded corners.

In FIG. 10F, subpixels are placed in respective hexagonal regions that are arranged densely. Focusing on one of the subpixels, the subpixel is placed so as to be surrounded by six subpixels. The subpixels are arranged such that subpixels that emit light of the same color are not adjacent to each other. For example, focusing on the subpixel 110R, the subpixel 110R is surrounded by three of the subpixels 110G and three of the subpixels 110B that are alternately arranged.

FIG. 10G illustrates an example where subpixels of different colors are arranged in a zigzag manner. Specifically, the positions of the top sides of two subpixels arranged in the row direction (e.g., the subpixels 110R and 110G or the subpixels 110G and 110B) are not aligned in the top view.

In the pixels illustrated in FIGS. 10A to 10G, for example, it is preferable that the subpixel 110R be a subpixel R that emits red light, the subpixel 110G be a subpixel G that emits green light, and the subpixel 110B be a subpixel B that emits blue light. Note that the structures of the subpixels are not limited thereto, and the colors and the order of the subpixels can be determined as appropriate. For example, the subpixel 110G may be the subpixel R that emits red light, and the subpixel 110R may be the subpixel G that emits green light.

In a photolithography method, as a pattern to be formed by processing becomes finer, the influence of light diffraction becomes more difficult to ignore; thus, the fidelity in transferring a photomask pattern by light exposure is degraded, and it becomes difficult to process a resist mask into a desired shape. Thus, a pattern with rounded corners is likely to be formed even with a rectangular photomask pattern. Consequently, the top-view shape of a subpixel is a polygon with rounded corners, an ellipse, a circle, or the like in some cases.

Furthermore, in the method for fabricating the light-emitting apparatus of one embodiment of the present invention, the organic compound layer is processed into an island shape with the use of a resist mask. A resist film formed over the organic compound layer needs to be cured at a temperature lower than the upper temperature limit of the organic compound layer. Thus, the resist film is insufficiently cured in some cases depending on the upper temperature limit of the material of the organic compound layer and the curing temperature of the resist material. An insufficiently cured resist film may have a shape different from a desired shape by processing. As a result, the top-view shape of the organic compound layer may be a polygon with rounded corners, an ellipse, a circle, or the like. For example, when a resist mask whose top-view shape is a square is intended to be formed, a resist mask whose top-view shape is a circle may be formed, and the top-view shape of the organic compound layer may be a circle.

To obtain a desired top-view shape of the organic compound layer, a technique of correcting a mask pattern in advance so that a transferred pattern agrees with a design pattern (an optical proximity correction (OPC) technique) may be used. Specifically, with the OPC technique, a pattern for correction is added to a corner portion of a figure on a mask pattern, for example.

As illustrated in FIGS. 11A to 11I, the pixel can include four types of subpixels.

The pixels 178 illustrated in FIGS. 11A to 11C employ stripe arrangement.

FIG. 11A illustrates an example where each subpixel has a rectangular top-view shape. FIG. 11B illustrates an example where each subpixel has a top-view shape formed by combining two half circles and a rectangle. FIG. 11C illustrates an example where each subpixel has an elliptical top-view shape.

The pixels 178 illustrated in FIGS. 11D to 11F employ matrix arrangement.

FIG. 11D illustrates an example where each subpixel has a square top-view shape. FIG. 11E illustrates an example where each subpixel has a substantially square top-view shape with rounded corners. FIG. 11F illustrates an example where each subpixel has a circular top-view shape.

FIGS. 11G and 11H each illustrate an example where one pixel 178 is composed of two rows and three columns.

The pixel 178 illustrated in FIG. 11G includes three subpixels (the subpixels 110R, 110G, and 110B) in the upper row (first row) and one subpixel (a subpixel 110W) in the lower row (second row). In other words, the pixel 178 includes the subpixel 110R in the left column (first column), the subpixel 110G in the middle column (second column), the subpixel 110B in the right column (third column), and the subpixel 110W across these three columns.

The pixel 178 illustrated in FIG. 11H includes three subpixels (the subpixels 110R, 110G, and 110B) in the upper row (first row) and three of the subpixels 110W in the lower row (second row). In other words, the pixel 178 includes the subpixels 110R and 110W in the left column (first column), the subpixels 110G and 110W in the middle column (second column), and the subpixels 110B and 110W in the right column (third column). Matching the positions of the subpixels in the upper row and the lower row as illustrated in FIG. 11H enables dust that would be produced in the fabrication process, for example, to be removed efficiently. Thus, a light-emitting apparatus having high display quality can be provided.

In the pixel 178 illustrated in FIGS. 11G and 11H, the subpixels 110R, 110G, and 110B are arranged in a stripe pattern, whereby the display quality can be improved.

FIG. 11I illustrates an example where one pixel 178 is composed of three rows and two columns.

The pixel 178 illustrated in FIG. 11I includes the subpixel 110R in the upper row (first row), the subpixel 110G in the middle row (second row), the subpixel 110B across the first row and the second row, and one subpixel (the subpixel 110W) in the lower row (third row). In other words, the pixel 178 includes the subpixels 110R and 110G in the left column (first column), the subpixel 110B in the right column (second column), and the subpixel 110W across these two columns.

In the pixel 178 illustrated in FIG. 11I, the subpixels 110R, 110G, and 110B are arranged in what is called an S-stripe pattern, whereby the display quality can be improved.

The pixel 178 illustrated in each of FIGS. 11A to 11I is composed of four subpixels, which are the subpixels 110R, 110G, 110B, and 110W. For example, the subpixel 110R can be a subpixel that emits red light, the subpixel 110G can be a subpixel that emits green light, the subpixel 110B can be a subpixel that emits blue light, and the subpixel 110W can be a subpixel that emits white light. Note that at least one of the subpixels 110R, 110G, 110B, and 110W may be a subpixel that emits cyan light, magenta light, yellow light, or near-infrared light.

As described above, the pixel composed of the subpixels each including the light-emitting device can employ any of a variety of layouts in the light-emitting apparatus of one embodiment of the present invention.

This embodiment can be combined as appropriate with the other embodiments or the examples. In this specification, in the case where a plurality of structure examples are described in one embodiment, the structure examples can be combined as appropriate.

### Embodiment 5

In this embodiment, a display device of one embodiment of the present invention will be described.

The display device in this embodiment can be a high-resolution display device. Thus, the display device in this embodiment can be used for display portions of information terminals (wearable devices) such as watch-type and bracelet-type information terminals and display portions of wearable devices capable of being worn on a head, such as a VR device like a head mounted display (HMD) and a glasses-type AR device.

The display device in this embodiment can be a high-definition display device or a large-sized display device. Accordingly, the display device in this embodiment can be used for display portions of a digital camera, a digital video camera, a digital photo frame, a mobile phone, a portable game console, a portable information terminal, and an audio reproducing device, in addition to display portions of electronic appliances with a relatively large screen, such as a television device, desktop and notebook personal computers, a monitor of a computer and the like, digital signage, and a large game machine such as a pachinko machine.

### Display Module

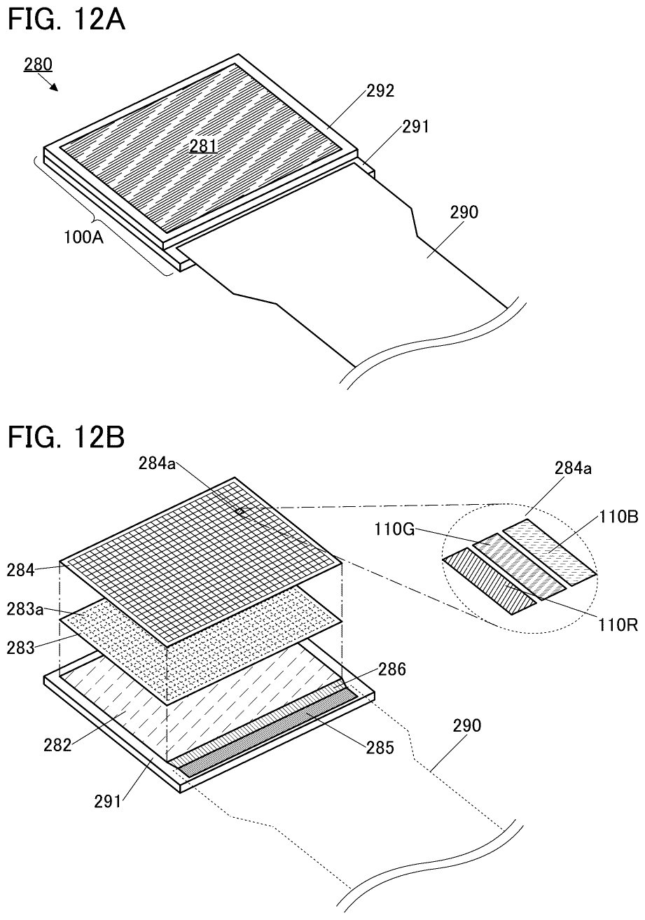

FIG. 12A is a perspective view of a display module 280. The display module 280 includes a display device 100A and an FPC 290. Note that the display device included in the display module 280 is not limited to the display device 100A and may be any of a display device 100B, a display device 100C, a display device 100D, a display device 100D2, a display device 100E, and a display device 100E2 described later.

The display module 280 includes a substrate 291 and a substrate 292. The display module 280 includes a display portion 281. The display portion 281 is a region of the display module 280 where an image is displayed, and is a region where light emitted from pixels provided in a pixel portion 284 described later can be seen.

FIG. 12B is a perspective view schematically illustrating the structure on the substrate 291 side. Over the substrate 291, a circuit portion 282, a pixel circuit portion 283 over the circuit portion 282, and the pixel portion 284 over the pixel circuit portion 283 are stacked. In addition, a terminal portion 285 for connection to the FPC 290 is included in a portion not overlapping with the pixel portion 284 over the substrate 291. The terminal portion 285 and the circuit portion 282 are electrically connected to each other through a wiring portion 286 formed of a plurality of wirings.

The pixel portion 284 includes a plurality of pixels 284a arranged periodically. An enlarged view of one pixel 284a is illustrated on the right side in FIG. 12B. The pixels 284a can employ any of the structures described in the above embodiments.

The pixel circuit portion 283 includes a plurality of pixel circuits 283a arranged periodically.

One pixel circuit 283a is a circuit that controls driving of a plurality of devices included in one pixel 284a.

The circuit portion 282 includes a circuit for driving the pixel circuits 283a in the pixel circuit portion 283. For example, the circuit portion 282 preferably includes one or both of a gate line driver circuit and a source line driver circuit. The circuit portion 282 may also include at least one of an arithmetic circuit, a memory circuit, a power supply circuit, and the like.

The FPC 290 functions as a wiring for supplying a video signal, a power supply potential, or the like to the circuit portion 282 from the outside. An IC may be mounted on the FPC 290.

The display module 280 can have a structure in which one or both of the pixel circuit portion 283 and the circuit portion 282 are stacked below the pixel portion 284; hence, the aperture ratio (effective display area ratio) of the display portion 281 can be significantly high.

Such a display module 280 has extremely high resolution, and thus can be suitably used for a VR device such as an HMD or a glasses-type AR device. For example, even in the case of a structure in which the display portion of the display module 280 is seen through a lens, pixels of the extremely-high-resolution display portion 281 included in the display module 280 are prevented from being recognized when the display portion is enlarged by the lens, so that display providing a high sense of immersion can be performed. Without being limited thereto, the display module 280 can be suitably used for electronic appliances including a relatively small display portion.

### Display Device 100A

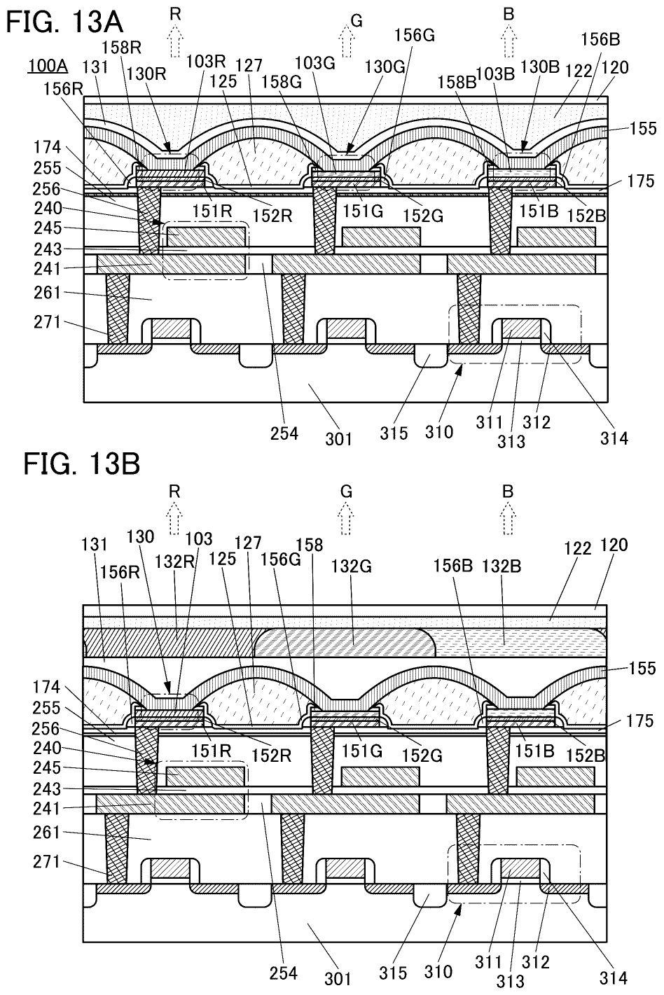

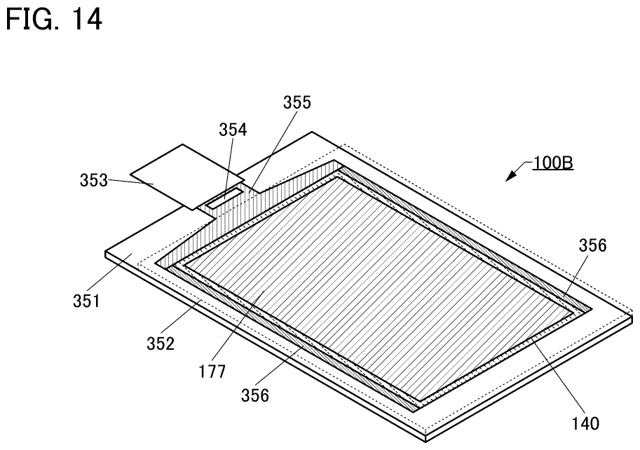

The display device 100A illustrated in FIG. 13A includes a substrate 301, the light-emitting devices 130R, 130G, and 130B, a capacitor 240, and a transistor 310.

The substrate 301 corresponds to the substrate 291 in FIGS. 12A and 12B. The transistor 310 includes a channel formation region in the substrate 301. As the substrate 301, a semiconductor substrate such as a single crystal silicon substrate can be used, for example. The transistor 310 includes part of the substrate 301, a conductive layer 311, a low-resistance region 312, an insulating layer 313, and an insulating layer 314. The conductive layer 311 functions as a gate electrode. The insulating layer 313 is positioned between the substrate 301 and the conductive layer 311 and functions as a gate insulating layer. The low-resistance region 312 is a region where the substrate 301 is doped with an impurity, and functions as a source or a drain. The insulating layer 314 is provided to cover the side surface of the conductive layer 311.

An element isolation layer 315 is provided between two adjacent transistors 310 to be embedded in the substrate 301.

An insulating layer 261 is provided to cover the transistor 310, and the capacitor 240 is provided over the insulating layer 261.

The capacitor 240 includes a conductive layer 241, a conductive layer 245, and an insulating layer 243 between the conductive layers 241 and 245. The conductive layer 241 functions as one electrode of the capacitor 240, the conductive layer 245 functions as the other electrode of the capacitor 240, and the insulating layer 243 functions as a dielectric of the capacitor 240.

The conductive layer 241 is provided over the insulating layer 261 and is embedded in an insulating layer 254. The conductive layer 241 is electrically connected to one of the source and the drain of the transistor 310 through a plug 271 embedded in the insulating layer 261. The insulating layer 243 is provided to cover the conductive layer 241. The conductive layer 245 is provided in a region overlapping with the conductive layer 241 with the insulating layer 243 therebetween.

An insulating layer 255 is provided to cover the capacitor 240. The insulating layer 174 is provided over the insulating layer 255. The insulating layer 175 is provided over the insulating layer 174. The light-emitting devices 130R, 130G, and 130B are provided over the insulating layer 175. An insulator is provided in regions between adjacent light-emitting devices.

The insulating layer 156R is provided to cover the side surface of the conductive layer 151R. The insulating layer 156G is provided to cover the side surface of the conductive layer 151G. The insulating layer 156B is provided to cover the side surface of the conductive layer 151B. The sacrificial layer 158R is positioned over the organic compound layer 103R. The sacrificial layer 158G is positioned over the organic compound layer 103G. The sacrificial layer 158B is positioned over the organic compound layer 103B.

Each of the conductive layers 151R, 151G, and 151B is electrically connected to one of the source and the drain of the corresponding transistor 310 through a plug 256 embedded in the insulating layers 243, 255, 174, and 175, the conductive layer 241 embedded in the insulating layer 254, and the plug 271 embedded in the insulating layer 261. Any of a variety of conductive materials can be used for the plugs.

The protective layer 131 is provided over the light-emitting devices 130R, 130G, and 130B. The substrate 120 is attached to the protective layer 131 with the resin layer 122. Embodiment 4 can be referred to for the details of the light-emitting device 130 and the components thereover up to the substrate 120. The substrate 120 corresponds to the substrate 292 in FIG. 12A.

FIG. 13B illustrates a variation example of the display device 100A illustrated in FIG. 13A. The display device illustrated in FIG. 13B includes a coloring layer 132R, a coloring layer 132G, and a coloring layer 132B, and each of the light-emitting devices 130 includes a region overlapping with one of the coloring layers 132R, 132G, and 132B. In the display device illustrated in FIG. 13B, the light-emitting device 130 can emit white light, for example. The coloring layer 132R, the coloring layer 132G, and the coloring layer 132B can transmit red light, green light, and blue light, respectively, for example.

### Display Device 100B

FIG. 14 is a perspective view of the display device 100n.

In the display device 100B, a substrate 352 and a substrate 351 are bonded to each other. In FIG. 14, the substrate 352 is denoted by a dashed line.

The display device 100B includes the pixel portion 177, the connection portion 140, a circuit 356, a wiring 355, and the like. FIG. 14 illustrates an example in which an IC 354 and an FPC 353 are mounted on the display device 100B. Thus, the structure illustrated in FIG. 14 can be regarded as a display module including the display device 100B, the integrated circuit (IC), and the FPC. Here, a display device in which a substrate is equipped with a connector such as an FPC or mounted with an IC is referred to as a display module.

The connection portion 140 is provided outside the pixel portion 177. The number of connection portions 140 may be one or more. In the connection portion 140, a common electrode of a light-emitting device is electrically connected to a conductive layer, so that a potential can be supplied to the common electrode.

As the circuit 356, a scan line driver circuit can be used, for example.

The wiring 355 has a function of supplying a signal and power to the pixel portion 177 and the circuit 356. The signal and power are input to the wiring 355 from the outside through the FPC 353 or from the IC 354.

FIG. 14 illustrates an example in which the IC 354 is provided over the substrate 351 by a chip on glass (COG) method, a chip on film (COF) method, or the like. An IC including a scan line driver circuit, a signal line driver circuit, or the like can be used as the IC 354, for example. Note that the display device 100B and the display module are not necessarily provided with an IC. Alternatively, the IC may be mounted on the FPC by a COF method, for example.

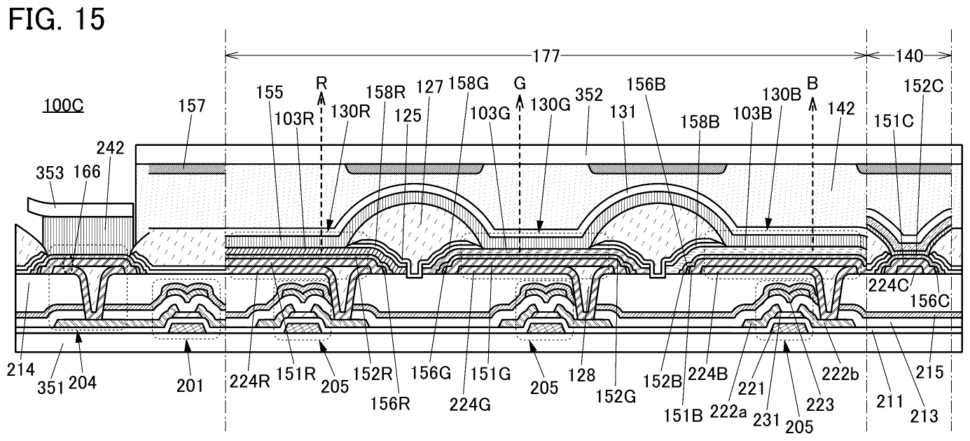

FIG. 15 illustrates, as the display device 100C, an example of cross sections of part of a region including the FPC 353, part of the circuit 356, part of the pixel portion 177, part of the connection portion 140, and part of a region including an end portion of the display device 100B in FIG. 14.

### Display Device 100C

The display device 100C illustrated in FIG. 15 includes a transistor 201, a transistor 205, the light-emitting device 130R that emits red light, the light-emitting device 130G that emits green light, the light-emitting device 130B that emits blue light, and the like between the substrate 351 and the substrate 352.

Embodiment 4 can be referred to for the details of the light-emitting devices 130R, 130G, and 130B.

The light-emitting device 130R includes a conductive layer 224R, the conductive layer 151R over the conductive layer 224R, and the conductive layer 152R over the conductive layer 151R. The light-emitting device 130G includes a conductive layer 224G, the conductive layer 151G over the conductive layer 224G, and the conductive layer 152G over the conductive layer 151G. The light-emitting device 130B includes a conductive layer 224B, the conductive layer 151B over the conductive layer 224B, and the conductive layer 152B over the conductive layer 151B.

The conductive layer 224R is connected to a conductive layer 222b included in the transistor 205 through the opening provided in an insulating layer 214. An end portion of the conductive layer 151R is positioned outward from an end portion of the conductive layer 224R. The insulating layer 156R is provided to include a region that is in contact with the side surface of the conductive layer 151R, and the conductive layer 152R is provided to cover the conductive layer 151R and the insulating layer 156R.

The conductive layers 224G, 151G, and 152G and the insulating layer 156G in the light-emitting device 130G are not described in detail because they are respectively similar to the conductive layers 224R, 151R, and 152R and the insulating layer 156R in the light-emitting device 130R; the same applies to the conductive layers 224B, 151B, and 152B and the insulating layer 156B in the light-emitting device 130B.

The conductive layers 224R, 224G, and 224B each have a depression portion covering the opening provided in the insulating layer 214. A layer 128 is embedded in the depression portion.

The layer 128 has a function of filling the depression portions of the conductive layers 224R, 224G, and 224B to enable planarity. Over the conductive layers 224R, 224G, and 224B and the layer 128, the conductive layers 151R, 151G, and 151B that are respectively electrically connected to the conductive layers 224R, 224G, and 224B are provided. Thus, the regions overlapping with the depression portions of the conductive layers 224R, 224G, and 224B can also be used as light-emitting regions, whereby the aperture ratio of the pixel can be increased.

The layer 128 may be an insulating layer or a conductive layer. Any of a variety of inorganic insulating materials, organic insulating materials, and conductive materials can be used for the layer 128 as appropriate. Specifically, the layer 128 is preferably formed using an insulating material and is particularly preferably formed using an organic insulating material. The layer 128 can be formed using an organic insulating material usable for the insulating layer 127, for example.

The protective layer 131 is provided over the light-emitting devices 130R, 130G, and 130B. The protective layer 131 and the substrate 352 are bonded to each other with an adhesive layer 142. The substrate 352 is provided with a light-blocking layer 157. A solid sealing structure, a hollow sealing structure, or the like can be employed to seal the light-emitting device 130. In FIG. 15, a solid sealing structure is employed, in which a space between the substrate 352 and the substrate 351 is filled with the adhesive layer 142. Alternatively, the space may be filled with an inert gas (e.g., nitrogen or argon), i.e., a hollow sealing structure may be employed. In that case, the adhesive layer 142 may be provided not to overlap with the light-emitting device. Furthermore, the space may be filled with a resin other than the frame-shaped adhesive layer 142.

FIG. 15 illustrates an example in which the connection portion 140 includes a conductive layer 224C obtained by processing the same conductive film as the conductive layers 224R, 224G, and 224B; the conductive layer 151C obtained by processing the same conductive film as the conductive layers 151R, 151G, and 151B; and the conductive layer 152C obtained by processing the same conductive film as the conductive layers 152R, 152G, and 152B. In the example illustrated in FIG. 15, the insulating layer 156C is provided to include a region overlapping with the side surface of the conductive layer 151C.

The display device 100C has a top-emission structure. Light from the light-emitting device is emitted toward the substrate 352. For the substrate 352, a material having a high visible-light-transmitting property is preferably used. The pixel electrode includes a material that reflects visible light, and a counter electrode (the common electrode 155) includes a material that transmits visible light.

An insulating layer 211, an insulating layer 213, an insulating layer 215, and the insulating layer 214 are provided in this order over the substrate 351. Part of the insulating layer 211 functions as a gate insulating layer of each transistor. Part of the insulating layer 213 functions as a gate insulating layer of each transistor. The insulating layer 215 is provided to cover the transistors. The insulating layer 214 is provided to cover the transistors and has a function of a planarization layer. Note that the number of gate insulating layers and the number of insulating layers covering the transistors are not limited and may each be one or more.

An inorganic insulating film is preferably used for each of the insulating layers 211, 213, and 215.

An organic insulating layer is suitable as the insulating layer 214 functioning as a planarization layer.

Each of the transistors 201 and 205 includes a conductive layer 221 functioning as a gate, the insulating layer 211 functioning as the gate insulating layer, a conductive layer 222a and the conductive layer 222b functioning as a source and a drain, a semiconductor layer 231, the insulating layer 213 functioning as the gate insulating layer, and a conductive layer 223 functioning as a gate.

A connection portion 204 is provided in a region of the substrate 351 not overlapping with the substrate 352. In the connection portion 204, a source electrode or a drain electrode of the transistor 201 is electrically connected to the FPC 353 through a conductive layer 166 and a connection layer 242. An example is described in which the conductive layer 166 has a stacked-layer structure of a conductive film obtained by processing the same conductive film as the conductive layers 224R, 224G, and 224B; a conductive film obtained by processing the same conductive film as the conductive layers 151R, 151G, and 151B; and a conductive film obtained by processing the same conductive film as the conductive layers 152R, 152G, and 152B. On the top surface of the connection portion 204, the conductive layer 166 is exposed. Thus, the connection portion 204 and the FPC 353 can be electrically connected to each other through the connection layer 242.

The light-blocking layer 157 is preferably provided on the surface of the substrate 352 on the substrate 351 side. The light-blocking layer 157 can be provided over a region between adjacent light-emitting devices, in the connection portion 140, in the circuit 356, and the like. A variety of optical members can be arranged on the outer surface of the substrate 352.

A material that can be used for the substrate 120 can be used for each of the substrates 351 and 352.

A material that can be used for the resin layer 122 can be used for the adhesive layer 142.

As the connection layer 242, an anisotropic conductive film (ACF), an anisotropic conductive paste (ACP), or the like can be used.

### Display Device 100D

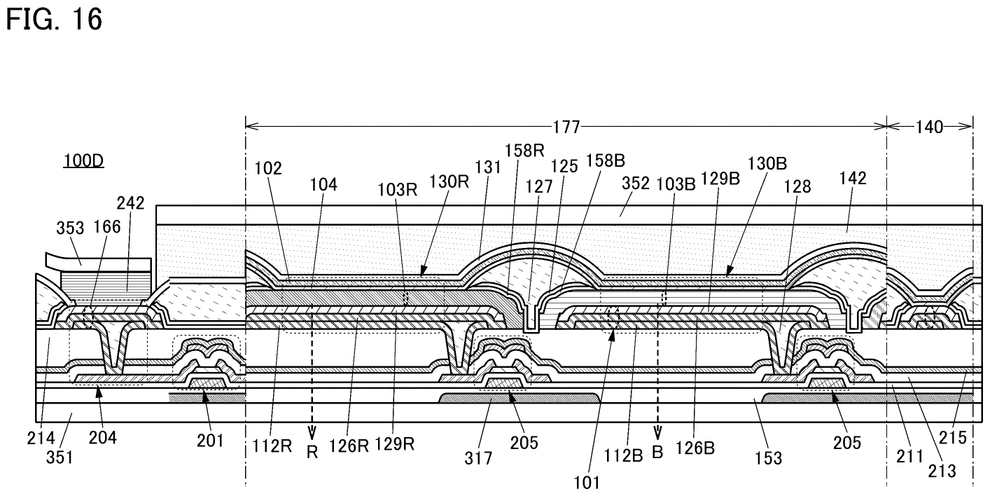

The display device 100D illustrated in FIG. 16 differs from the display device 100C illustrated in FIG. 15 mainly in having a bottom-emission structure.

Light from the light-emitting device is emitted toward the substrate 351. For the substrate 351, a material having a high visible-light-transmitting property is preferably used. By contrast, there is no limitation on the light-transmitting property of a material used for the substrate 352.

A light-blocking layer 317 is preferably formed between the substrate 351 and the transistor 201 and between the substrate 351 and the transistor 205. FIG. 16 illustrates an example in which the light-blocking layer 317 is provided over the substrate 351, an insulating layer 153 is provided over the light-blocking layer 317, and the transistors 201 and 205 and the like are provided over the insulating layer 153.

The light-emitting device 130R includes a conductive layer 112R, a conductive layer 126R over the conductive layer 112R, and a conductive layer 129R over the conductive layer 126R.

The light-emitting device 130B includes a conductive layer 112B, a conductive layer 126B over the conductive layer 112B, and a conductive layer 129B over the conductive layer 126B.

A material having a high visible-light-transmitting property is used for each of the conductive layers 112R, 112B, 126R, 126B, 129R, and 129B. A material that reflects visible light is preferably used for the second electrode 102.

Although not illustrated in FIG. 16, the light-emitting device 130G is also provided.

Although FIG. 16 and the like illustrate an example in which the top surface of the layer 128 includes a flat portion, the shape of the layer 128 is not particularly limited.

### Display Device 100D2

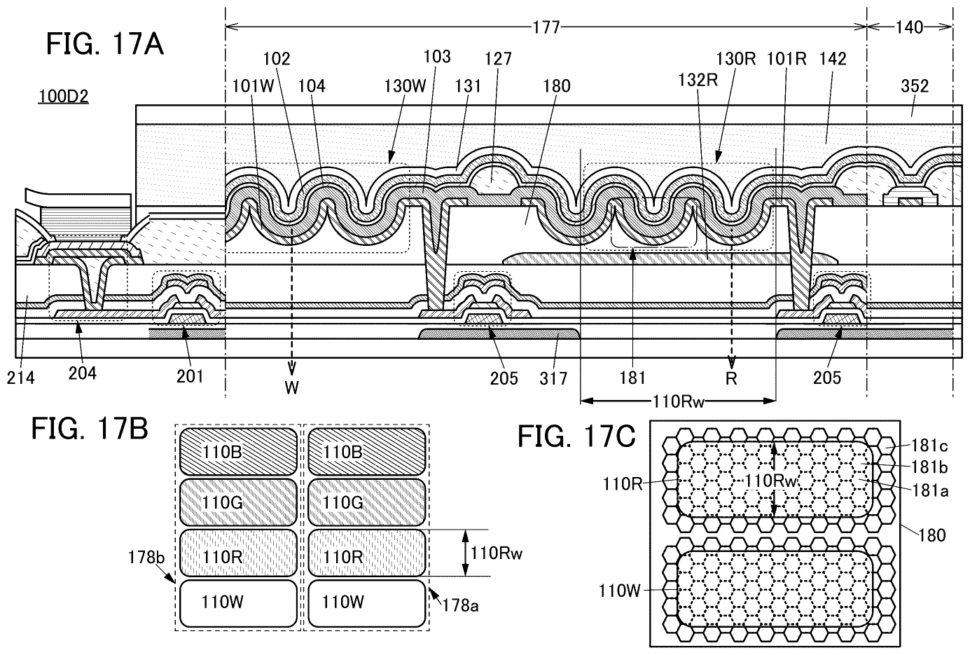

The display device 100D2 illustrated in FIG. 17A is an example of a bottom-emission display device different from the display device 100D illustrated in FIG. 16. The display device 100D2 is different from the display device 100D in including an organic resin layer 180. Note that in the drawing, reference numerals of some of the components that are shown in FIG. 16 are omitted; for the details of the components, the description made with reference to FIG. 16 can be referred to.

FIG. 17B shows a top-view layout of the pixels 178 (a pixel 178a and a pixel 178b) each including the subpixels 110 (the subpixels 110R, 110G, 110B, and 110W), and FIG. 17C shows a top view of the organic resin layer 180 in a region where the subpixels 110R and 110W of the pixel 178 are formed. Note that the width between the light-blocking layer 317 and another light-blocking layer 317 corresponds to a width 110Rw in the light-emitting region of the subpixel 110R.

As illustrated in FIG. 17A, the organic resin layer 180 is provided over the insulating layer 214. As illustrated in FIG. 17C and the region surrounded by the dashed-dotted line in FIG. 17A, the organic resin layer 180 includes depressed portions 181 (depressed portions 181a and depressed portions 181b) each having a curved surface, at least in a region where the subpixels are formed. Note that the depressed portion 181 outside the light-emitting region, like a depressed portion 181c, may also be provided. When the depressed portion 181c is provided, light that has been emitted in the region overlapping with the light-blocking layer 317 or light that has progressed to the region overlapping with the light-blocking layer 317 can be refracted and extracted from the light-emitting region, increasing the emission efficiency.

A plurality of the depressed portions 181 may be formed in a matrix. The depressed portion 181a and the depressed portion 181b may be provided in contact with each other or may have a flat surface therebetween.

Although the top-view shape and the cross-sectional shape of the depressed portion are hexagonal (FIG. 17C) and semicircular (FIG. 17A), respectively, other shapes may be employed as needed. Examples of the top-view shape of the depressed portion include polygons such as a triangle, a tetragon (including a rectangle and a square), and a pentagon; these polygons with rounded corners; an ellipse; and a circle.

As the organic resin layer 180, an insulating layer including an organic material can be used. For the organic resin layer 180, an acrylic resin, a polyimide resin, an epoxy resin, an imide resin, a polyamide resin, a polyimide-amide resin, a silicone resin, a siloxane resin, a benzocyclobutene-based resin, a phenol resin, or a precursor of any of these resins can be used, for example. Alternatively, an organic material such as polyvinyl alcohol (PVA), polyvinyl butyral, polyvinylpyrrolidone, polyethylene glycol, polyglycerin, pullulan, water-soluble cellulose, or an alcohol-soluble polyamide resin may be used for the organic resin layer 180.

Further alternatively, a photosensitive resin can be used for the organic resin layer 180. A photoresist may be used as the photosensitive resin. As the photosensitive resin, a positive photosensitive material or a negative photosensitive material can be used.

The organic resin layer 180 may include a material absorbing visible light. For example, the organic resin layer 180 itself may be made of a material absorbing visible light, or the organic resin layer 180 may include a pigment absorbing visible light. For the organic resin layer 180, for example, a resin that can be used as a color filter transmitting red, blue, or green light and absorbing light of the other colors or a resin that includes carbon black as a pigment and functions as a black matrix can be used.

The first electrodes 101 (a first electrode 101R and a first electrode 101W) are provided over the organic resin layer 180, and the organic compound layer 103 is provided over the first electrodes 101. End portions of the first electrode 101 and the organic compound layer 103 may be covered with the insulating layer 127.

The first electrode 101 formed over the organic resin layer 180 has a depressed portion along the depressed portion of the organic resin layer 180. Furthermore, the organic compound layer 103 formed over the first electrode 101 has a depressed portion along the depressed portion of the first electrode 101. Furthermore, the common layer 104 formed over the organic compound layer 103 has a depressed portion along the depressed portion of the organic compound layer 103. Furthermore, the second electrode 102 formed over the common layer 104 has a depressed portion along the depressed portion of the common layer 104. That is, the depressed portions of the organic resin layer 180, the first electrode 101, the organic compound layer 103, the common layer 104, and the second electrode 102 overlap with each other.

The common layer 104 is provided over the organic compound layer 103 and the insulating layer 127, and the second electrode 102 is provided over the common layer 104. The protective layer 131 is provided over the second electrode 102, and the substrate 352 is bonded with the use of the adhesive layer 142.

Although not shown in FIG. 17A, the light-emitting device 130G and the light-emitting device 130B are also provided.

### [Display Device 100E]

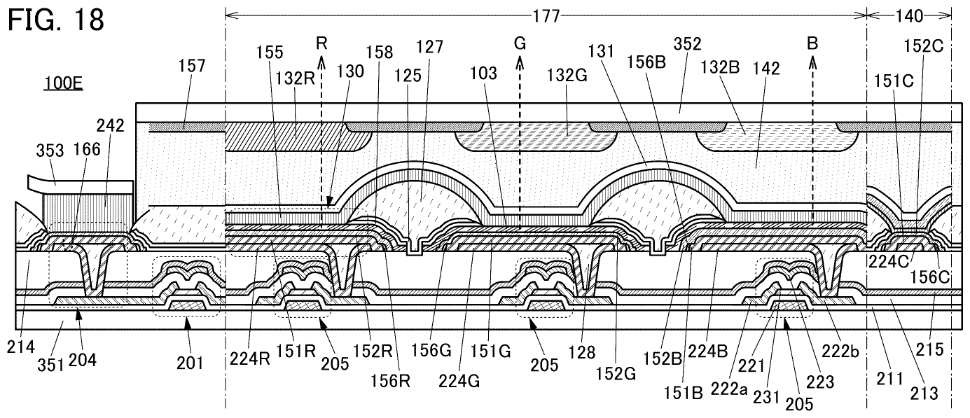

The display device 100E illustrated in FIG. 18 is a variation example of the display device 100C illustrated in FIG. 15 and differs from the display device 100C mainly in including the coloring layers 132R, 132G, and 132B.

In the display device 100E, the light-emitting device 130 includes a region overlapping with one of the coloring layers 132R, 132G, and 132B. The coloring layers 132R, 132G, and 132B can be provided on the surface of the substrate 352 on the substrate 351 side. End portions of the coloring layers 132R, 132G, and 132B can overlap with the light-blocking layer 157.

In the display device 100E, the light-emitting device 130 can emit white light, for example. The coloring layer 132R, the coloring layer 132G, and the coloring layer 132B can transmit red light, green light, and blue light, respectively, for example. Note that in the display device 100E, the coloring layers 132R, 132G, and 132B may be provided between the protective layer 131 and the adhesive layer 142.

### Display Device 100E2

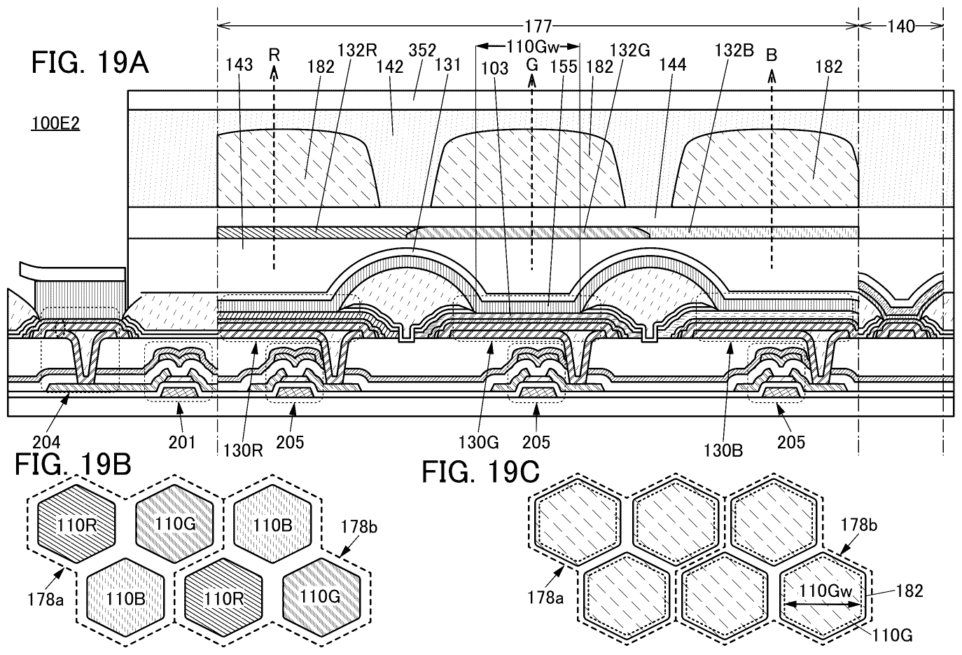

The display device 100E2 illustrated in FIG. 19A is a variation example of the display device 100E illustrated in FIG. 18 and includes microlenses 182 over the coloring layers 132R, 132G, and 132B. Note that in the drawing, reference numerals of some of the components that are shown in FIG. 18 are omitted; for the details of the components, the description made with reference to FIG. 18 can be referred to.

FIG. 19B shows a top-view layout of the pixels 178 (the pixels 178a and 178b) each including the subpixels 110 (the subpixels 110R, 110G, and 110B), and FIG. 19C shows a top view of the microlenses 182 in a region where the subpixels 110R, 110G, and 110B of the pixels 178 are formed. Note that the width of the region where the common electrode 155 and the organic compound layer 103 are in contact with each other corresponds to a width 110Gw in the light-emitting region of the subpixel 110G.

In the display device 100E2 illustrated in FIG. 19A, a planarization film 143 is provided over the protective layer 131, and the coloring layers 132R, 132G, and 132B are provided over the planarization film 143. A planarization film 144 is provided to cover the coloring layers 132R, 132G, and 132B. The microlenses 182 are provided over the planarization film 144.

Note that as illustrated in FIG. 19C, the microlenses 182 are preferably provided on a subpixel basis in the region where the subpixels are formed.

Although the top-view shape of the microlens 182 is illustrated as a hexagon in FIG. 19C, other shapes may be employed as needed. Examples of the top-view shape of the microlens 182 include polygons such as a triangle, a tetragon (including a rectangle and a square), and a pentagon; these polygons with rounded corners; an ellipse; and a circle.

The microlens 182 can be formed using a material similar to that for the organic resin layer 180.

This embodiment can be combined as appropriate with the other embodiments or the examples. In this specification, in the case where a plurality of structure examples are described in one embodiment, the structure examples can be combined as appropriate.

### Embodiment 6

In this embodiment, electronic appliances of embodiments of the present invention will be described.

Electronic appliances of this embodiment include the light-emitting apparatus of one embodiment of the present invention in their display portions. The light-emitting apparatus of one embodiment of the present invention is highly reliable and can be easily increased in resolution and definition. Thus, the light-emitting apparatus of one embodiment of the present invention can be used for display portions of a variety of electronic appliances.

Examples of the electronic appliances include a digital camera, a digital video camera, a digital photo frame, a mobile phone, a portable game console, a portable information terminal, and an audio reproducing device, in addition to electronic appliances with a relatively large screen, such as a television device, desktop and notebook personal computers, a monitor of a computer and the like, digital signage, and a large game machine such as a pachinko machine.

In particular, the light-emitting apparatus of one embodiment of the present invention can have high resolution, and thus can be favorably used for an electronic appliance having a relatively small display portion. Examples of such an electronic appliance include watch-type and bracelet-type information terminal devices (wearable devices) and wearable devices capable of being worn on a head, such as a VR device like a head-mounted display, a glasses-type AR device, and a mixed reality (MR) device.

The definition of the light-emitting apparatus of one embodiment of the present invention is preferably as high as HD (number of pixels: 1280×720), FHD (number of pixels: 1920×1080), WQHD (number of pixels: 2560×1440), WQXGA (number of pixels: 2560×1600), 4 K (number of pixels: 3840×2160), or 8 K (number of pixels: 7680×4320). In particular, definition of 4 K, 8 K, or higher is preferable. The pixel density (resolution) of the light-emitting apparatus of one embodiment of the present invention is preferably higher than or equal to 100 ppi, further preferably higher than or equal to 300 ppi, still further preferably higher than or equal to 500 ppi, yet still further preferably higher than or equal to 1000 ppi, yet still further preferably higher than or equal to 2000 ppi, yet still further preferably higher than or equal to 3000 ppi, yet still further preferably higher than or equal to 5000 ppi, yet still further preferably higher than or equal to 7000 ppi. The use of the light-emitting apparatus having one or both of such high definition and high resolution can further increase realistic sensation, sense of depth, and the like. There is no particular limitation on the screen ratio (aspect ratio) of the light-emitting apparatus of one embodiment of the present invention. For example, the light-emitting apparatus is compatible with a variety of screen ratios such as 1:1 (a square), 4:3, 16:9, and 16:10.

The electronic appliance in this embodiment may include a sensor (a sensor having a function of measuring force, displacement, position, speed, acceleration, angular velocity, rotational frequency, distance, light, liquid, magnetism, temperature, a chemical substance, sound, time, hardness, electric field, current, voltage, electric power, radiation, flow rate, humidity, gradient, oscillation, odor, or infrared rays).

The electronic appliance in this embodiment can have a variety of functions. For example, the electronic appliance in this embodiment can have a function of displaying a variety of information (e.g., a still image, a moving image, and a text image) on the display portion, a touch panel function, a function of displaying a calendar, date, time, and the like, a function of executing a variety of software (programs), a wireless communication function, and a function of reading out a program or data stored in a recording medium.

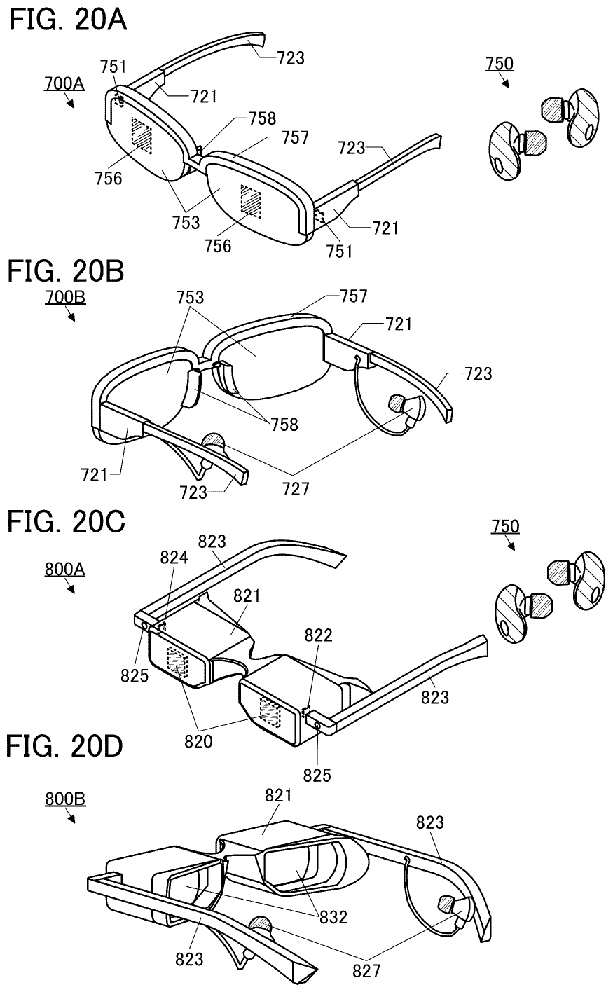

Examples of wearable devices capable of being worn on a head are described with reference to FIGS. 20A to 20D. These wearable devices have at least one of a function of displaying AR contents, a function of displaying VR contents, a function of displaying substitutional reality (SR) contents, and a function of displaying MR contents. The electronic appliance having a function of displaying contents of at least one of AR, VR, SR, MR, and the like enables the user to feel a higher level of immersion.

An electronic appliance 700A illustrated in FIG. 20A and an electronic appliance 700B illustrated in FIG. 20B each include a pair of display panels 751, a pair of housings 721, a communication portion (not illustrated), a pair of wearing portions 723, a control portion (not illustrated), an image capturing portion (not illustrated), a pair of optical members 753, a frame 757, and a pair of nose pads 758.

The light-emitting apparatus of one embodiment of the present invention can be used for the display panels 751. Thus, a highly reliable electronic appliance is obtained.

The electronic appliances 700A and 700B can each project images displayed on the display panels 751 onto display regions 756 of the optical members 753. Since the optical members 753 have a light-transmitting property, the user can see images displayed on the display regions, which are superimposed on transmission images seen through the optical members 753. Accordingly, the electronic appliances 700A and 700B are electronic appliances capable of performing AR display.

In the electronic appliances 700A and 700B, a camera capable of capturing images of the front side may be provided as the image capturing portion. Furthermore, when the electronic appliances 700A and 700B are provided with an acceleration sensor such as a gyroscope sensor, the orientation of the user's head can be sensed and an image corresponding to the orientation can be displayed on the display regions 756.

The communication portion includes a wireless communication device, and a video signal, for example, can be supplied by the wireless communication device. Instead of or in addition to the wireless communication device, a connector that can be connected to a cable for supplying a video signal and a power supply potential may be provided.

The electronic appliances 700A and 700B are provided with a battery, so that they can be charged wirelessly and/or by wire.

A touch sensor module may be provided in the housing 721. The touch sensor module has a function of detecting a touch on the outer surface of the housing 721. Detecting a tap operation, a slide operation, or the like by the user with the touch sensor module enables various types of processing. For example, a moving image can be paused or restarted by a tap operation, and can be fast-forwarded or fast-reversed by a slide operation. When the touch sensor module is provided in each of the two housings 721, the range of the operation can be increased.

Various touch sensors can be applied to the touch sensor module. For example, any of touch sensors of the following types can be used: a capacitive type, a resistive type, an infrared type, an electromagnetic induction type, a surface acoustic wave type, and an optical type. In particular, a capacitive sensor or an optical sensor is preferably used for the touch sensor module.

In the case of using an optical touch sensor, a photoelectric conversion device (also referred to as a photoelectric conversion element) can be used as a light-receiving element. One or both of an inorganic semiconductor and an organic semiconductor can be used for an active layer of the photoelectric conversion device.

An electronic appliance 800A illustrated in FIG. 20C and an electronic appliance 800B illustrated in FIG. 20D each include a pair of display portions 820, a housing 821, a communication portion 822, a pair of wearing portions 823, a control portion 824, a pair of image capturing portions 825, and a pair of lenses 832.

The light-emitting apparatus of one embodiment of the present invention can be used in the display portions 820. Thus, a highly reliable electronic appliance is obtained.

The display portions 820 are positioned inside the housing 821 so as to be seen through the lenses 832. When the pair of display portions 820 display different images, three-dimensional display using parallax can be performed.

The electronic appliances 800A and 800B can be regarded as electronic appliances for VR. The user who wears the electronic appliance 800A or 800B can see images displayed on the display portions 820 through the lenses 832.

The electronic appliances 800A and 800B preferably include a mechanism for adjusting the lateral positions of the lenses 832 and the display portions 820 so that the lenses 832 and the display portions 820 are positioned optimally in accordance with the positions of the user's eyes. Moreover, the electronic appliances 800A and 800B preferably include a mechanism for adjusting focus by changing the distance between the lenses 832 and the display portions 820.

The electronic appliance 800A or 800B can be worn on the user's head with the wearing portions 823. FIG. 20C, for instance, shows an example where the wearing portion 823 has a shape like a temple of glasses; however, one embodiment of the present invention is not limited thereto. The wearing portion 823 can have any shape with which the user can wear the electronic appliance, for example, a shape of a helmet or a band.

The image capturing portion 825 has a function of obtaining information on the external environment. Data obtained by the image capturing portion 825 can be output to the display portion 820. An image sensor can be used for the image capturing portion 825. Moreover, a plurality of cameras may be provided so as to cover a plurality of fields of view, such as a telescope field of view and a wide field of view.

Although an example where the image capturing portions 825 are provided is described here, a range sensor (hereinafter also referred to as a sensing portion) capable of measuring the distance between the user and an object just needs to be provided. In other words, the image capturing portion 825 is one embodiment of the sensing portion. As the sensing portion, an image sensor or a range image sensor such as a light detection and ranging (LiDAR) sensor can be used, for example. By using images obtained by the camera and images obtained by the range image sensor, more information can be obtained and a gesture operation with higher accuracy is possible.

The electronic appliance 800A may include a vibration mechanism that functions as bone-conduction earphones. For example, at least one of the display portion 820, the housing 821, and the wearing portion 823 can include the vibration mechanism. Thus, without additionally requiring an audio device such as headphones, earphones, or a speaker, the user can enjoy video and sound only by wearing the electronic appliance 800A.

The electronic appliances 800A and 800B may each include an input terminal. To the input terminal, a cable for supplying a video signal from a video output device or the like, power for charging a battery provided in the electronic appliance, and the like can be connected.

The electronic appliance of one embodiment of the present invention may have a function of performing wireless communication with earphones 750. The earphones 750 include a communication portion (not illustrated) and have a wireless communication function. The earphones 750 can receive information (e.g., audio data) from the electronic appliance with the wireless communication function. For example, the electronic appliance 700A in FIG. 20A has a function of transmitting information to the earphones 750 with the wireless communication function. For another example, the electronic appliance 800A in FIG. 20C has a function of transmitting information to the earphones 750 with the wireless communication function.

The electronic appliance may include an earphone portion. The electronic appliance 700B in FIG. 20B includes earphone portions 727. For example, the earphone portion 727 can be connected to the control portion by wire. Part of a wiring that connects the earphone portion 727 and the control portion may be positioned inside the housing 721 or the wearing portion 723.

Similarly, the electronic appliance 800B in FIG. 20D includes earphone portions 827. For example, the earphone portion 827 can be connected to the control portion 824 by wire. Part of a wiring that connects the earphone portion 827 and the control portion 824 may be positioned inside the housing 821 or the wearing portion 823. Alternatively, the earphone portions 827 and the wearing portions 823 may include magnets. This is preferable because the earphone portions 827 can be fixed to the wearing portions 823 with magnetic force and thus can be easily housed.

The electronic appliance may include an audio output terminal to which earphones, headphones, or the like can be connected. The electronic appliance may include one or both of an audio input terminal and an audio input mechanism. As the audio input mechanism, a sound collecting device such as a microphone can be used, for example. The electronic appliance may have a function of a headset by including the audio input mechanism.

As described above, both the glasses-type device (e.g., the electronic appliances 700A and 700B) and the goggles-type device (e.g., the electronic appliances 800A and 800B) are preferable as the electronic appliance of one embodiment of the present invention.

The electronic appliance of one embodiment of the present invention can transmit information to earphones by wire or wirelessly.

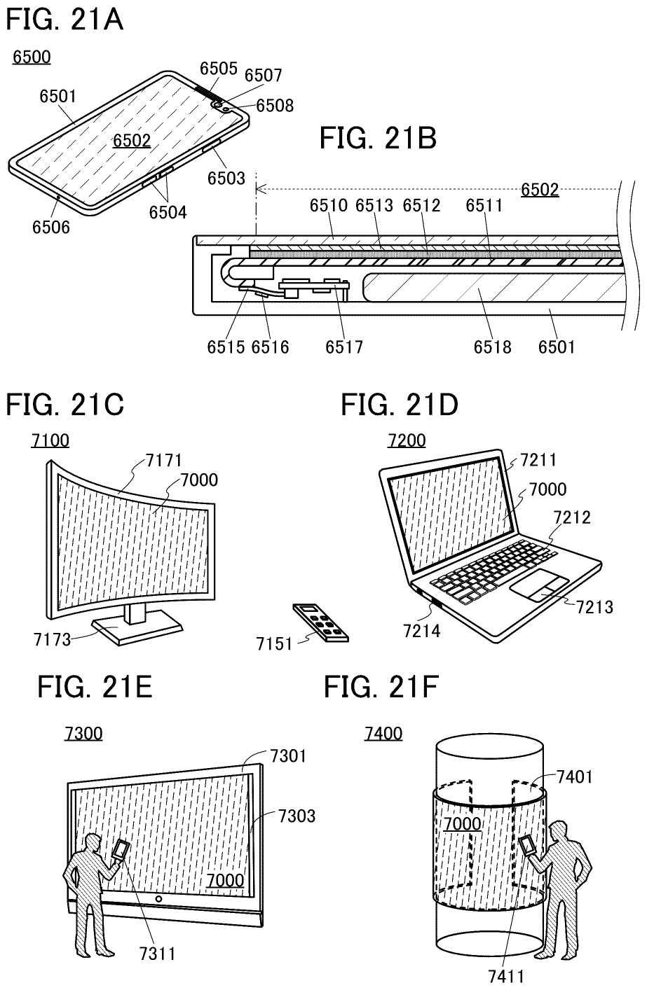

An electronic appliance 6500 illustrated in FIG. 21A is a portable information terminal that can be used as a smartphone.

The electronic appliance 6500 includes a housing 6501, a display portion 6502, a power button 6503, buttons 6504, a speaker 6505, a microphone 6506, a camera 6507, a light source 6508, and the like. The display portion 6502 has a touch panel function.

The light-emitting apparatus of one embodiment of the present invention can be used in the display portion 6502. Thus, a highly reliable electronic appliance is obtained.

FIG. 21B is a schematic cross-sectional view including an edge portion of the housing 6501 on the microphone 6506 side.

A protection member 6510 having a light-transmitting property is provided on the display surface side of the housing 6501. A display panel 6511, an optical member 6512, a touch sensor panel 6513, a printed circuit board 6517, a battery 6518, and the like are provided in a space surrounded by the housing 6501 and the protection member 6510.

The display panel 6511, the optical member 6512, and the touch sensor panel 6513 are fixed to the protection member 6510 with an adhesive layer (not illustrated).

Part of the display panel 6511 is folded back in a region outside the display portion 6502, and an FPC 6515 is connected to the part that is folded back. An IC 6516 is mounted on the FPC 6515. The FPC 6515 is connected to a terminal provided on the printed circuit board 6517.

The light-emitting apparatus of one embodiment of the present invention can be used in the display panel 6511. Thus, an extremely lightweight electronic appliance can be obtained. Since the display panel 6511 is extremely thin, the battery 6518 with high capacity can be mounted without an increase in the thickness of the electronic appliance. An electronic appliance with a narrow bezel can be obtained when part of the display panel 6511 is folded back so that the portion connected to the FPC 6515 is provided on the back side of a pixel portion.

FIG. 21C illustrates an example of a television device. In a television device 7100, a display portion 7000 is incorporated in a housing 7171. Here, the housing 7171 is supported by a stand 7173.

The light-emitting apparatus of one embodiment of the present invention can be used in the display portion 7000. Thus, a highly reliable electronic appliance is obtained.

Operation of the television device 7100 illustrated in FIG. 21C can be performed with an operation switch provided in the housing 7171 and a separate remote control 7151. Alternatively, the display portion 7000 may include a touch sensor, and the television device 7100 may be operated by touch on the display portion 7000 with a finger or the like. The remote control 7151 may be provided with a display portion for displaying information output from the remote control 7151. With operation keys or a touch panel of the remote control 7151, channels and volume can be controlled and video displayed on the display portion 7000 can be controlled.

Note that the television device 7100 includes a receiver, a modem, and the like. A general television broadcast can be received with the receiver. When the television device is connected to a communication network with or without wires via the modem, one-way (from a transmitter to a receiver) or two-way (e.g., between a transmitter and a receiver or between receivers) information communication can be performed.

FIG. 21D illustrates an example of a notebook personal computer. A notebook personal computer 7200 includes a housing 7211, a keyboard 7212, a pointing device 7213, an external connection port 7214, and the like. The display portion 7000 is incorporated in the housing 7211.

The light-emitting apparatus of one embodiment of the present invention can be used in the display portion 7000. Thus, a highly reliable electronic appliance is obtained.

FIGS. 21E and 21F illustrate examples of digital signage.

Digital signage 7300 illustrated in FIG. 21E includes a housing 7301, the display portion 7000, a speaker 7303, and the like. The digital signage 7300 can also include an LED lamp, operation keys (including a power switch or an operation switch), a connection terminal, a variety of sensors, a microphone, and the like.

FIG. 21F shows digital signage 7400 attached to a cylindrical pillar 7401. The digital signage 7400 includes the display portion 7000 provided along a curved surface of the pillar 7401.

In FIGS. 21E and 21F, the light-emitting apparatus of one embodiment of the present invention can be used in the display portion 7000. Thus, a highly reliable electronic appliance is obtained.

A larger area of the display portion 7000 can increase the amount of information that can be provided at a time. The display portion 7000 having a larger area attracts more attention, so that the effectiveness of the advertisement can be increased, for example.

A touch panel is preferably used in the display portion 7000, in which case in addition to display of still or moving images on the display portion 7000, intuitive operation by a user is possible. Moreover, in the case of an application for providing information such as route information or traffic information, usability can be enhanced by intuitive operation.

As illustrated in FIGS. 21E and 21F, it is preferable that the digital signage 7300 or the digital signage 7400 can work with an information terminal 7311 or an information terminal 7411, such as a smartphone that a user has, through wireless communication. For example, information of an advertisement displayed on the display portion 7000 can be displayed on a screen of the information terminal 7311 or the information terminal 7411. By operation of the information terminal 7311 or the information terminal 7411, a displayed image on the display portion 7000 can be switched.

It is possible to make the digital signage 7300 or the digital signage 7400 execute a game with the use of the screen of the information terminal 7311 or the information terminal 7411 as an operation means (controller). Thus, an unspecified number of users can join in and enjoy the game concurrently.

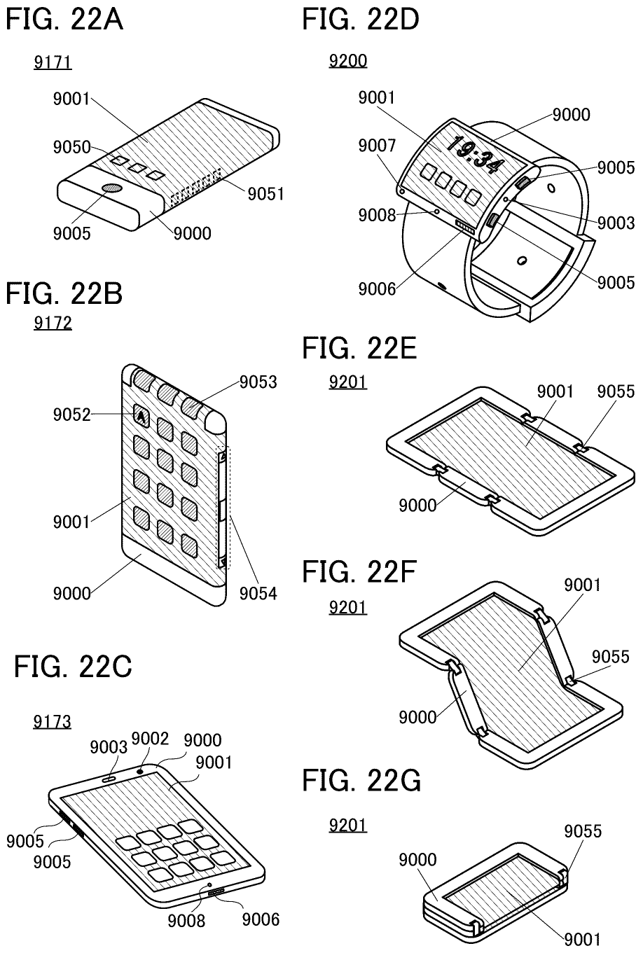

Electronic appliances illustrated in FIGS. 22A to 22G include a housing 9000, a display portion 9001, a speaker 9003, an operation key 9005 (including a power switch or an operation switch), a connection terminal 9006, a sensor 9007 (a sensor having a function of measuring force, displacement, position, speed, acceleration, angular velocity, rotational frequency, distance, light, liquid, magnetism, temperature, chemical substance, sound, time, hardness, electric field, current, voltage, electric power, radiation, flow rate, humidity, gradient, oscillation, odor, or infrared rays), a microphone 9008, and the like.

The electronic appliances illustrated in FIGS. 22A to 22G have a variety of functions. For example, the electronic appliances can have a function of displaying a variety of information (e.g., a still image, a moving image, and a text image) on the display portion, a touch panel function, a function of displaying a calendar, date, time, and the like, a function of controlling processing with the use of a variety of software (programs), a wireless communication function, and a function of reading out and processing a program or data stored in a recording medium. Note that the functions of the electronic appliances are not limited thereto, and the electronic appliances can have a variety of functions. The electronic appliances may include a plurality of display portions. The electronic appliances may be provided with a camera or the like and have a function of taking a still image or a moving image, a function of storing the taken image in a storage medium (an external storage medium or a storage medium incorporated in the camera), a function of displaying the taken image on the display portion, and the like.

The electronic appliances in FIGS. 22A to 22G are described in detail below.

FIG. 22A is a perspective view of a portable information terminal 9171. The portable information terminal 9171 can be used as a smartphone, for example. The portable information terminal 9171 may include the speaker 9003, the connection terminal 9006, the sensor 9007, or the like. The portable information terminal 9171 can display text and image information on its plurality of surfaces. FIG. 22A illustrates an example in which three icons 9050 are displayed. Furthermore, information 9051 indicated by dashed rectangles can be displayed on another surface of the display portion 9001. Examples of the information 9051 include notification of reception of an e-mail, an SNS message, an incoming call, or the like, the title and sender of an e-mail, an SNS message, or the like, the date, the time, remaining battery, and the radio field intensity. Alternatively, the icon 9050 or the like may be displayed at the position where the information 9051 is displayed.

FIG. 22B is a perspective view of a portable information terminal 9172. The portable information terminal 9172 has a function of displaying information on three or more surfaces of the display portion 9001. Here, an example in which information 9052, information 9053, and information 9054 are displayed on different surfaces is described. For example, the user of the portable information terminal 9172 can check the information 9053 displayed such that it can be seen from above the portable information terminal 9172, with the portable information terminal 9172 put in a breast pocket of his/her clothes. Thus, the user can see the display without taking out the portable information terminal 9172 from the pocket and decide whether to answer the call, for example.

FIG. 22C is a perspective view of a tablet terminal 9173. The tablet terminal 9173 is capable of executing a variety of applications such as mobile phone calls, e-mailing, viewing and editing texts, music reproduction, Internet communication, and a computer game, for example. The tablet terminal 9173 includes the display portion 9001, the camera 9002, the microphone 9008, and the speaker 9003 on the front surface of the housing 9000; the operation keys 9005 as buttons for operation on the left side surface of the housing 9000; and the connection terminal 9006 on the bottom surface of the housing 9000.

FIG. 22D is a perspective view of a watch-type portable information terminal 9200. The portable information terminal 9200 can be used as a Smartwatch (registered trademark), for example. The display surface of the display portion 9001 is curved, and an image can be displayed on the curved display surface. Furthermore, for example, mutual communication between the portable information terminal 9200 and a headset capable of wireless communication can be performed, and thus hands-free calling is possible. With the connection terminal 9006, the portable information terminal 9200 can perform mutual data transmission with another information terminal and charging. Note that the charging operation may be performed by wireless power feeding.

FIGS. 22E to 22G are perspective views of a foldable portable information terminal 9201. FIG. 22E is a perspective view showing the portable information terminal 9201 that is opened. FIG. 22G is a perspective view showing the portable information terminal 9201 that is folded. FIG. 22F is a perspective view showing the portable information terminal 9201 that is shifted from one of the states in FIGS. 22E and 22G to the other. The portable information terminal 9201 is highly portable when folded. When the portable information terminal 9201 is opened, a seamless large display region is highly browsable. The display portion 9001 of the portable information terminal 9201 is supported by three of the housings 9000 joined together by hinges 9055. The display portion 9001 can be folded with a radius of curvature greater than or equal to 0.1 mm and less than or equal to 150 mm, for example.

This embodiment can be combined as appropriate with the other embodiments or the examples. In this specification, in the case where a plurality of structure examples are described in one embodiment, the structure examples can be combined as appropriate.

### Example 1

### Synthesis Example 1

In this example, physical properties of an organic compound of one embodiment of the present invention and a method for synthesizing the organic compound are described. Specifically, a method for synthesizing N,N′-bis-2-[4-(1-pyrrolidinyl)]pyridinyl-N,N′-diphenyl-[4-(1-pyrrolidinyl)]pyridine-2,6-diamine (abbreviation: Prd3-PyA2Py), which is represented by Structural Formula (100) in Embodiment 1, is described. The structure of Prd3-PyA2Py is shown below.

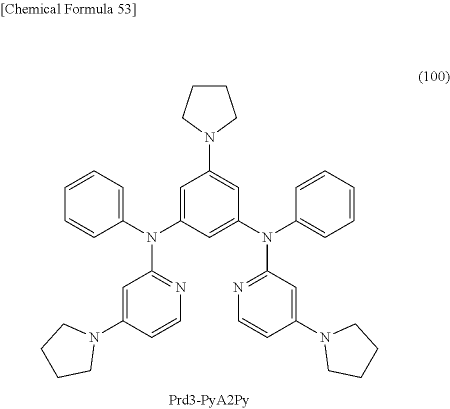 [SMILES: c1ccc(N(c2cc(N3CCCC3)cc(N(c3ccccc3)c3cc(N4CCCC4)ccn3)c2)c2cc(N3CCCC3)ccn2)cc1]

### Step 1: Synthesis of 4-(1-pyrrolidinyl)-N-phenyl-2-pyridinamine

Into a 200-mL three-neck flask were put 1.5 g (6.5 mmol) of 2-bromo-4-(1-pyrrolidinyl)pyridine, 0.66 g (7.1 mmol) of aniline, and 1.4 g (15 mmol) of sodium tert-butoxide, and the air in the flask was replaced with nitrogen. To this mixture was added 30 mL of toluene, and the mixture was degassed under reduced pressure; then, 0.11 g (0.19 mmol) of 4,5-bis(diphenylphosphino)-9,9-dimethylxanthene (abbreviation: Xantphos) and 60 mg (66 μmol) of tris(dibenzylideneacetone)dipalladium(0) (abbreviation: Pd₂(dba)₃) were added to the mixture, and the resulting mixture was stirred at 100° C. under a nitrogen stream for six hours.

After the stirring, water was added to this mixture, and the resulting mixture was separated by suction filtration to give a filtrate. The obtained filtrate was subjected to extraction with toluene. The obtained solution of the extract was concentrated to give a brown oily substance.

Toluene, ethyl acetate, and hexane were added to the obtained oily substance, and irradiation with ultrasonic waves was performed, so that 0.96 g of a white solid of the target substance was obtained in a yield of 62%. The synthesis scheme of Step 1 is shown in (a-1) below.

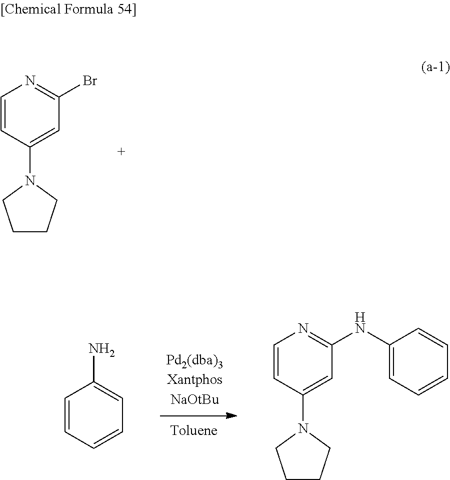 [SMILES: Brc1cc(N2CCCC2)ccn1 ; Nc1ccccc1 ; c1ccc(Nc2cc(N3CCCC3)ccn2)cc1]

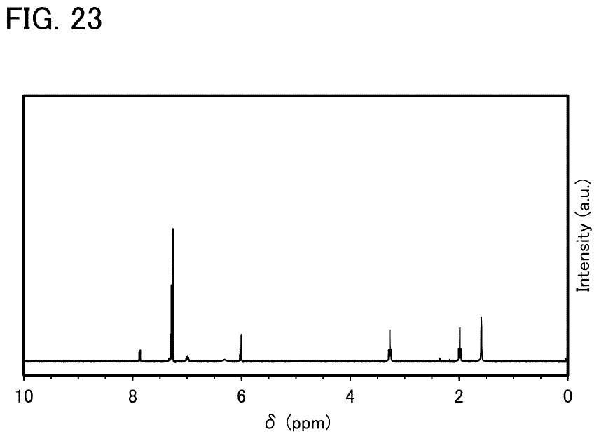

FIG. 23 shows the ¹H NMR spectrum of a deuterochloroform (abbreviation: CDCl₃) solution of the obtained white solid. The results of ¹H NMR measurement of the white solid are shown below. The results show that 4-(1-pyrrolidinyl)-N-phenyl-2-pyridinamine was obtained.

¹H NMR (CDCl₃, 300 MHz): δ=7.88 (m, 1H), 7.31 (m, 4H), 7.02 (m, 1H), 6.32 (bs, 1H), 6.03 (m, 2H), 3.30 (m, 4H), 2.03 (m, 4H).

### Step 2: Synthesis of Prd3-PyA2Py

Into a 200-mL recovery flask were put 0.76 g (2.5 mmol) of 2,6-dibromo-4-(1-pyrrolidinyl)pyridine, 1.4 g (5.7 mmol) of 4-(1-pyrrolidinyl)-N-phenyl-2-pyridinamine, and 1.1 g (11 mmol) of sodium tert-butoxide, and the air in the flask was replaced with nitrogen. To this mixture was added 30 mL of toluene, and the mixture was degassed under reduced pressure; then, 0.20 g (0.35 mmol) of 4,5-bis(diphenylphosphino)-9,9-dimethylxanthene (abbreviation: Xantphos) and 0.1 g (0.11 mol) of tris(dibenzylideneacetone)dipalladium(0) (abbreviation: Pd₂(dba)₃) were added to the mixture, and the resulting mixture was stirred at 100° C. under a nitrogen stream for 14 hours.

After the stirring, a solid was obtained; 300 mL of toluene was added to the solid, and then, suction filtration was performed through Celite (Catalog No. 537-02305 produced by FUJIFILM Wako Pure Chemical Corporation) to give a filtrate. Water was added to the obtained filtrate, and an aqueous layer was subjected to extraction with toluene. The obtained solution of the extract was concentrated to give a solid.

Toluene and hexane were added to the obtained solid, and irradiation with ultrasonic waves was performed, so that 1.1 g of a white solid of the target substance was obtained in a yield of 72%. The synthesis scheme of Step 2 is shown in (a-2) below.

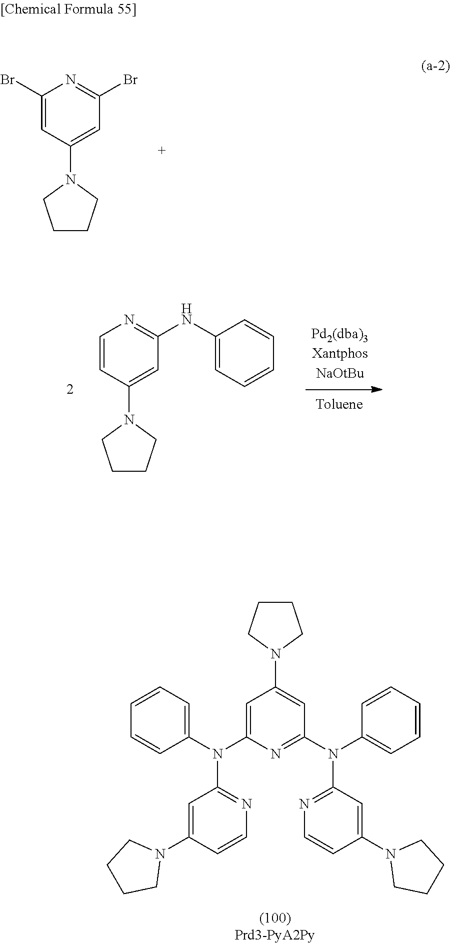 [SMILES: Brc1cc(N2CCCC2)cc(Br)n1 ; c1ccc(Nc2cc(N3CCCC3)ccn2)cc1 ; c1ccc(N(c2cc(N3CCCC3)ccn2)c2cc(N3CCCC3)cc(N(c3ccccc3)c3cc(N4CCCC4)ccn3)n2)cc1]

By a train sublimation method, 2.6 g of the obtained white solid was purified. In the purification by sublimation, the white solid was heated at 290° C. under a pressure of 3.2 Pa for 15 hours. After the purification by sublimation, 2.0 g of a white solid of the target substance was obtained at a collection rate of 77%.

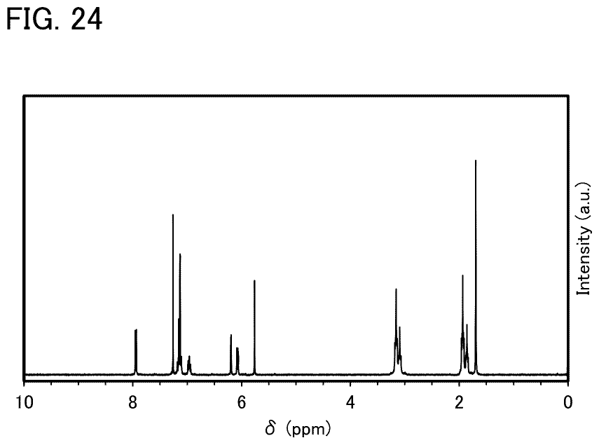

FIG. 24 shows the ¹H NMR spectrum of a deuterochloroform (abbreviation: CDCl₃) solution of the obtained white solid. The results of ¹H NMR measurement of the white solid are shown below. The results show that Prd3-PyA2Py was obtained.

¹H NMR (CDCl₃, 300 MHz): δ=7.95 (d, J=5.7 Hz, 2H), 7.18 (m, 8H), 6.98 (m, 2H), 6.20 (d, J=2.1 Hz, 2H), 6.09 (dd, J=2.1 Hz, 5.7 Hz, 2H), 5.76 (s, 2H), 3.18 (m, 12H), 1.96 (m, 12H).

### Example 2

In this example, a light-emitting device 2A including N,N′-bis-2-[4-(1-pyrrolidinyl)]pyridinyl-N,N′-diphenyl-[4-(1-pyrrolidinyl)]pyridine-2,6-diamine (abbreviation: Prd3-PyA2Py), an organic compound of one embodiment of the present invention represented by Structural Formula (100), was fabricated through a continuous vacuum process, and the results of evaluating the characteristics are described.

The structural formulae of organic compounds used in the light-emitting device 2A are shown below.

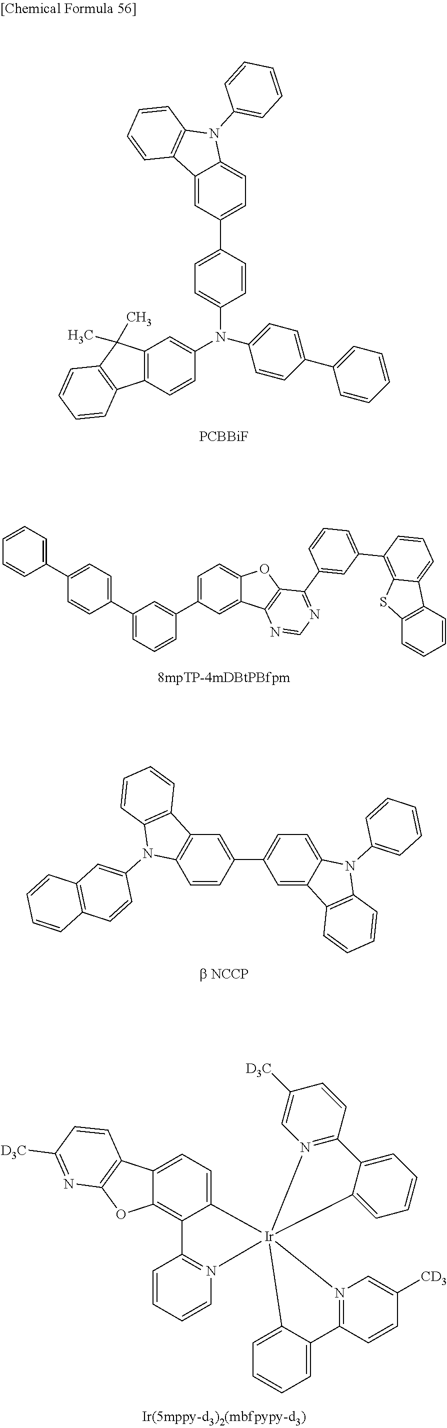

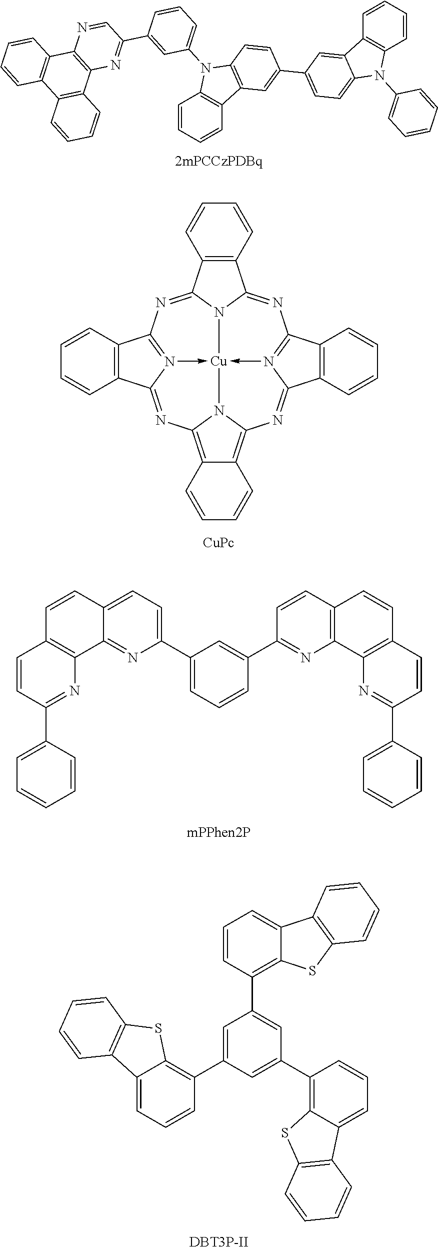

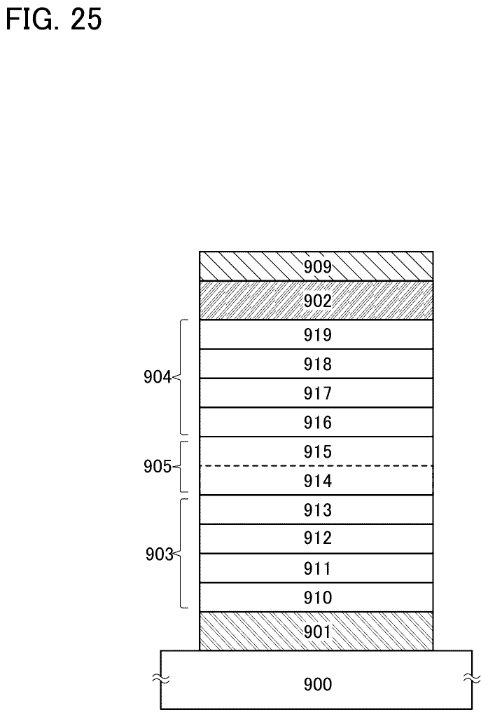

As illustrated in FIG. 25, the light-emitting device 2A has a tandem structure in which a first EL layer 903, an intermediate layer 905, a second EL layer 904, and a second electrode 902 are stacked over a first electrode 901 formed over a substrate 900 that is a glass substrate. A cap layer 909 is formed over the second electrode 902.

The first EL layer 903 has a structure in which a hole-injection layer 910, a first hole-transport layer 911, a first light-emitting layer 912, and a first electron-transport layer 913 are stacked in this order. The intermediate layer 905 includes an electron-injection buffer region 914 and a layer 915 including an electron-relay region and a charge-generation region. The second EL layer 904 has a structure in which a second hole-transport layer 916, a second light-emitting layer 917, a second electron-transport layer 918, and an electron-injection layer 919 are stacked in this order.

### Method for Fabricating Light-Emitting Device 2A

First, as a reflective electrode, silver (Ag) was deposited over the substrate 900 that is a glass substrate to a thickness of 100 nm by a sputtering method, and then, as a transparent electrode, indium tin oxide containing silicon oxide (ITSO) was deposited to a thickness of 85 nm by a sputtering method, so that the first electrode 901 was formed. The electrode area was set to 4 mm² (2 mm×2 mm). Note that the first electrode 901 is a transparent electrode, and the transparent electrode and the reflective electrode can be collectively regarded as the first electrode 901.

Next, the first EL layer 903 was provided. First, in pretreatment for forming the light-emitting device 2A over the substrate, a surface of the substrate was washed with water and baking was performed at 200° C. for one hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for approximately 30 minutes.

Then, the substrate provided with the first electrode 901 was fixed to a substrate holder provided in the vacuum evaporation apparatus such that the surface on which the first electrode 901 was formed faced downward. Over the first electrode 901, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation using resistance heating to a thickness of 10 nm such that the weight ratio of PCBBiF to OCHD-003 was 1:0.03, whereby the hole-injection layer 910 was formed.

Next, PCBBiF was deposited by evaporation to a thickness of 90 nm over the hole-injection layer 910, so that the first hole-transport layer 911 was formed.

Next, the first light-emitting layer 912 was formed over the first hole-transport layer 911. The first light-emitting layer 912 was formed by depositing 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-3,3′-bi-9H-carbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) by co-evaporation using resistance heating to a thickness of 40 nm such that the weight ratio of 8mpTP-4mDBtPBfpm to βNCCP to Ir(5mppy-d₃)₂(mbfpypy-d₃) was 0.5:0.5:0.1.

Then, over the first light-emitting layer 912, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm, so that the first electron-transport layer 913 was formed.

Next, the intermediate layer 905 was provided. First, over the first electron-transport layer 913, Prd3-PyA2Py and lithium oxide (Li₂O) were deposited by co-evaporation using resistance heating to a thickness of 5 nm such that the volume ratio of Prd3-PyA2Py to Li₂O was 1.0:0.02, whereby a layer serving as the electron-injection buffer region 914 was formed.

Then, as the electron-relay region, a film of copper phthalocyanine (abbreviation: CuPc) was formed to have a thickness of 2 nm. Next, as the charge-generation region, PCBBiF and a fluorine-containing electron acceptor material with a molecular weight of 672 (OCHD-003) were deposited by co-evaporation using resistance heating to a thickness of 10 nm such that the weight ratio of PCBBiF to OCHD-003 was 1:0.15, whereby the layer 915 including the electron-relay region and the charge-generation region was formed.

Next, the second EL layer 904 was provided. First, PCBBiF was deposited by evaporation to a thickness of 50 nm, so that the second hole-transport layer 916 was formed.

Next, 8mpTP-4mDBtPBfpm, βNCCP, and Ir(5mppy-d₃)₂(mbfpypy-d₃) were deposited by co-evaporation using resistance heating to a thickness of 40 nm such that the weight ratio of 8mpTP-4mDBtPBfpm to βNCCP to Ir(5mppy-d₃)₂(mbfpypy-d₃) was 0.5:0.5:0.1, whereby the second light-emitting layer 917 was formed.

Next, over the second light-emitting layer 917, 2mPCCzPDBq was deposited by evaporation to a thickness of 20 nm, and then 2,2′-(1,3-phenylene)bis(9-phenyl-1,10-phenanthroline) (abbreviation: mPPhen2P) was deposited by evaporation to a thickness of 20 nm, so that the second electron-transport layer 918 was formed.

Next, over the second electron-transport layer 918, lithium fluoride (LiF) and ytterbium (Yb) were deposited by co-evaporation to a thickness of 1.5 nm such that the volume ratio of LiF to Yb was 2:1, whereby the electron-injection layer 919 was formed.

Next, over the electron-injection layer 919, Ag and Mg were deposited by co-evaporation to a thickness of 15 nm such that the volume ratio of Ag to Mg was 1:0.1, whereby the second electrode 902 was formed. Note that the second electrode 902 is a semi-transmissive and semi-reflective electrode having functions of transmitting light and reflecting light.

Then, as the cap layer 909, 4,4′,4″-(benzene-1,3,5-triyl)tri(dibenzothiophene) (abbreviation: DBT3P-II) was deposited by evaporation to a thickness of 70 nm.

Through the above process, the light-emitting device 2A was fabricated. The structure of the light-emitting device 2A is shown in the table below.

TABLE 1

| | Thick- | |
|---|---|---|
| | ness | |
| | [nm] | Light-emitting device 2A |
| Cap layer | 70 | DBT3P-II |
| Second electrode | 15 | Ag:Mg (1:0.1) |
| Electron-injection | 1.5 | LiF:Yb (2:1) |
| layer | | |
| Second electron- | 20 | mPPhen2P |
| transport layer | 20 | 2mPCCzPDBq |
| Second light- | 40 | 8mpTP-4mDBtPBfpm:βNCCP:Ir(5mppy- |
| emitting layer | | d₃)₂(mbfpypy-d₃) (0.5:0.5:0.1) |
| Second hole- | 50 | PCBBiF |
| transport layer | | |
| Charge-generation | 10 | PCBBiF:OCHD-003 (1:0.15) |
| region | | |
| Electron-relay | 2 | CuPc |
| region | | |
| Electron-injection | 5 | Prd3-PyA2Py:Li₂O |
| buffer region | | (1.0:0.02) |
| First electron- | 10 | 2mPCCzPDBq |
| transport layer | | |
| First light-emitting | 40 | 8mpTP-4mDBtPBfpm:βNCCP:Ir(5mppy- |
| layer | | d₃)₂(mbfpypy-d₃) (0.5:0.5:0.1) |
| First hole- | 90 | PCBBiF |
| transport layer | | |
| Hole-injection | 10 | PCBBiF:OCHD-003 (1:0.03) |
| layer | | |
| First electrode | 85 | ITSO |
| | 100 | Ag |

### Device Characteristics

The light-emitting device 2A was sealed using a glass substrate in a glove box containing a nitrogen atmosphere so as not to be exposed to the air (a sealing material was applied to surround the device and UV treatment and heat treatment at 80° C. for one hour were performed at the time of sealing). Then, the emission characteristics of the light-emitting device 2A were measured.

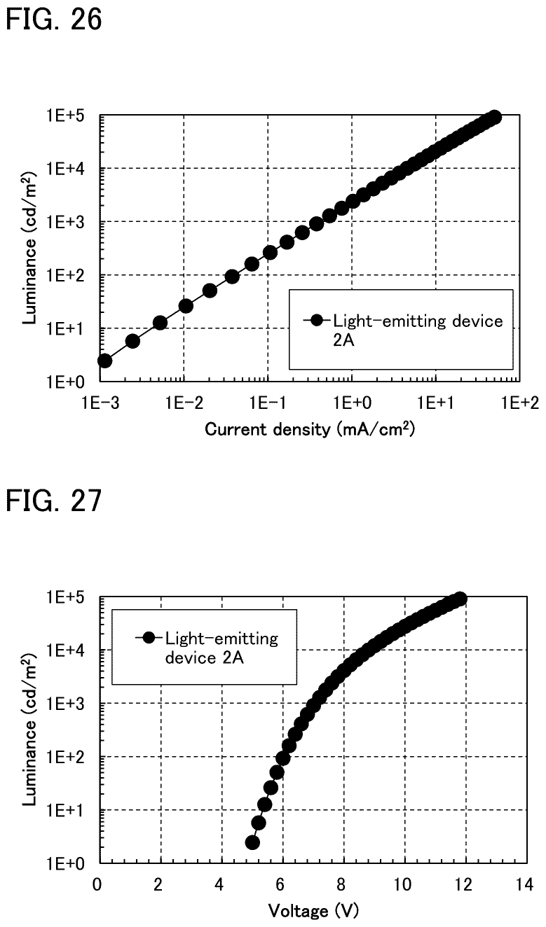

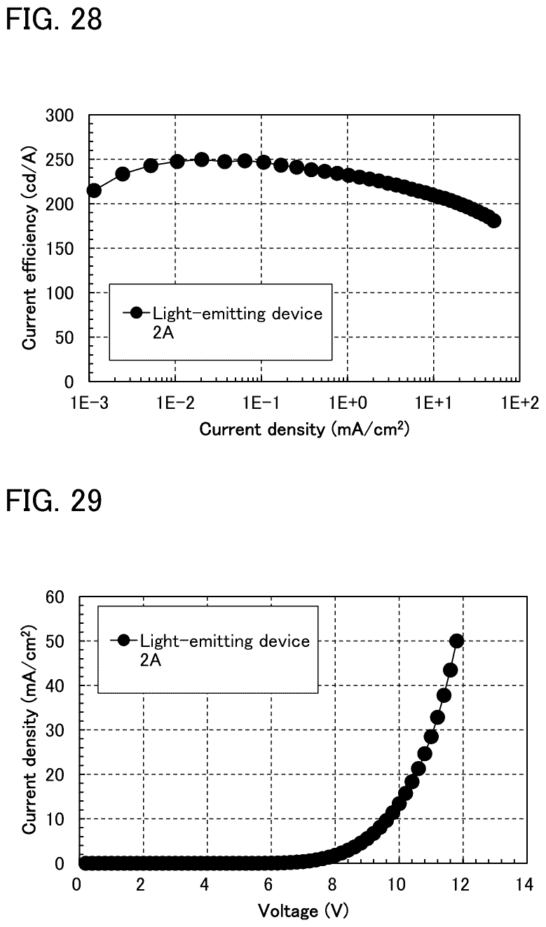

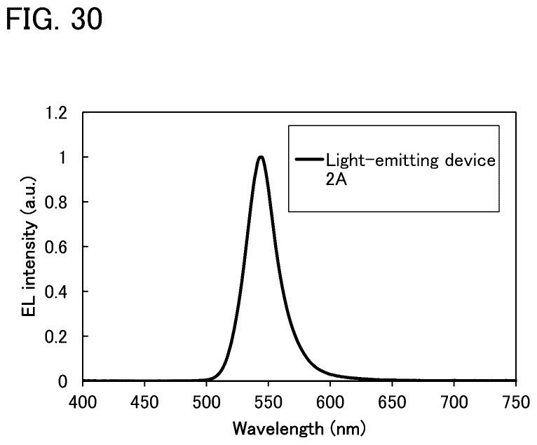

FIG. 26 shows the luminance-current density characteristics of the light-emitting device 2A. FIG. 27 shows the luminance-voltage characteristics thereof. FIG. 28 shows the current efficiency-current density characteristics thereof. FIG. 29 shows the current density-voltage characteristics thereof. FIG. 30 shows the electroluminescence spectrum thereof. The main characteristics of the light-emitting device 2A at a luminance of approximately 1000 cd/m² are shown in the table below. The luminance, CIE chromaticity, and electroluminescence spectrum were measured with a spectroradiometer (SR-UL1R produced by TOPCON TECHNOHOUSE CORPORATION).

TABLE 2

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Light-emitting | 7.0 | 0.0152 | 0.380 | 0.30 | 0.68 | 906 | 238 |
| device 2A | | | | | | | |

As shown in FIG. 30, the light-emitting device 2A emitted green light with a peak wavelength of 544 nm in its electroluminescence spectrum. FIG. 26 to FIG. 29 and the above table show that the light-emitting device 2A is a tandem light-emitting device with high current efficiency.

### Reliability Test Result

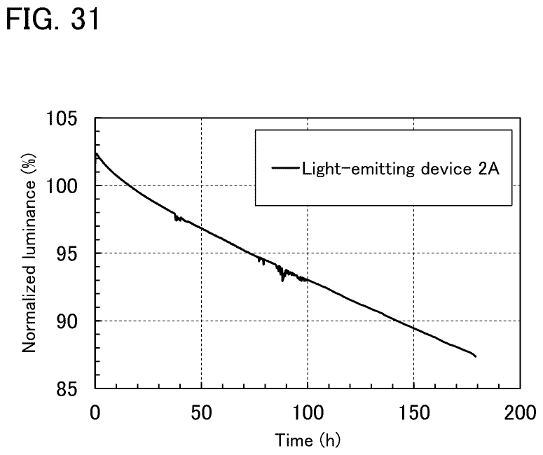

Moreover, a reliability test was conducted on the light-emitting device 2A. FIG. 31 shows a time-dependent change in normalized luminance during driving at a current density of 50 mA/cm². In FIG. 31, the vertical axis represents the luminance (%) normalized with the luminance at the time of the start of light emission being regarded as 100%, and the horizontal axis represents time (h).

In FIG. 31, LT95 (h), which is the time required for the measured luminance to decrease to 95% of the initial luminance, of the light-emitting device 2A is approximately 73 hours, showing that the light-emitting device 2A has high reliability.

It was found from the above that a tandem light-emitting device with a low driving voltage and high emission efficiency can be provided by using one embodiment of the present invention.

### Example 3

In this example, a light-emitting device 3A including N,N′-bis-2-[4-(1-pyrrolidinyl)]pyridinyl-N,N′-diphenyl-[4-(1-pyrrolidinyl)]pyridine-2,6-diamine (abbreviation: Prd3-PyA2Py), an organic compound of one embodiment of the present invention represented by Structural Formula (100), was fabricated through an MML process, which involves processing, and the results of evaluating the characteristics are described.

The structural formulae of organic compounds used in the light-emitting device 3A are shown below.

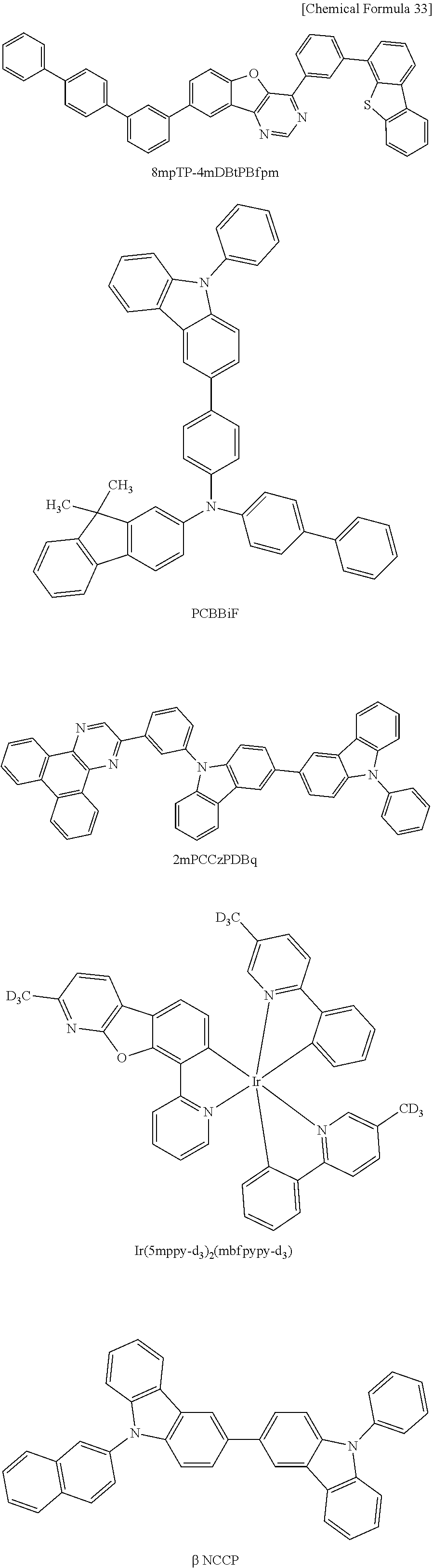

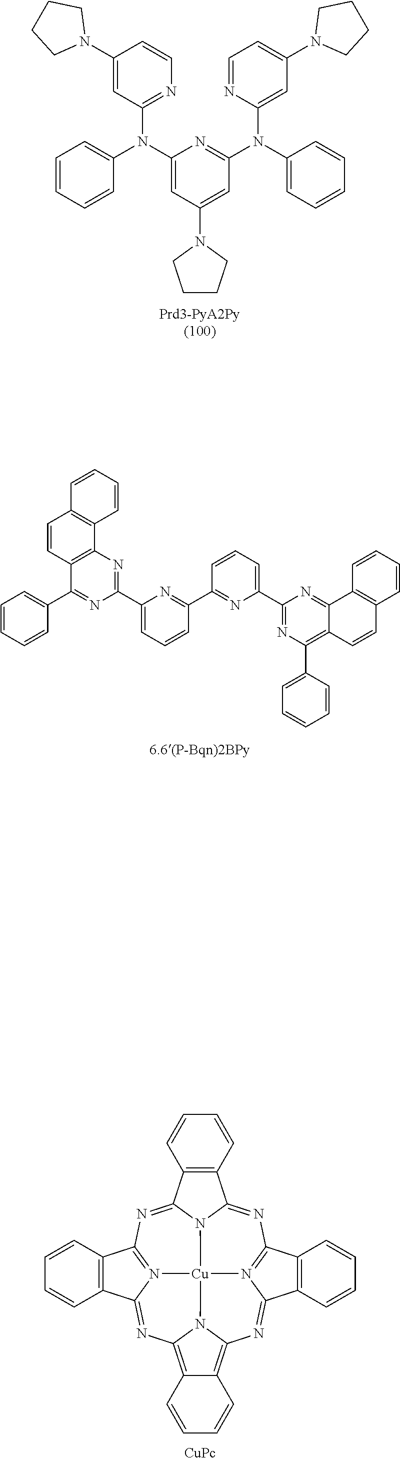

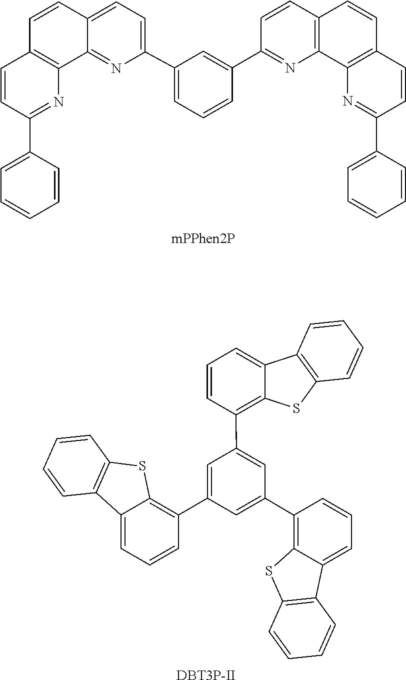 [SMILES: c1ccc(-c2ccc3ccc4ccc(-c5cccc(-c6ccc7ccc8ccc(-c9ccccc9)nc8c7n6)c5)nc4c3n2)cc1 ; c1ccc2c(c1)sc1c(-c3cc(-c4cccc5c4sc4ccccc45)cc(-c4cccc5c4sc4ccccc45)c3)cccc12 ; "DBT3P-II"]

As illustrated in FIG. 25, the light-emitting device 3A has a tandem structure in which the first EL layer 903, the intermediate layer 905, the second EL layer 904, and the second electrode 902 are stacked over the first electrode 901 formed over the substrate 900 that is a glass substrate. The cap layer 909 is formed over the second electrode 902.

The first EL layer 903 has a structure in which the hole-injection layer 910, the first hole-transport layer 911, the first light-emitting layer 912, and the first electron-transport layer 913 are stacked in this order. The intermediate layer 905 includes the electron-injection buffer region 914 and the layer 915 including an electron-relay region and a charge-generation region. The second EL layer 904 has a structure in which the second hole-transport layer 916, the second light-emitting layer 917, the second electron-transport layer 918, and the electron-injection layer 919 are stacked in this order.

### Method for Fabricating Light-Emitting Device 3A

First, as a reflective electrode, an alloy of silver, palladium, and copper (APC: Ag—Pd—Cu) was deposited over the substrate 900 that is a glass substrate to a thickness of 100 nm by a sputtering method, and then, as a transparent electrode, indium tin oxide containing silicon oxide (ITSO) was deposited to a thickness of 50 nm by a sputtering method, so that the first electrode 901 was formed. The electrode area was set to 4 mm² (2 mm×2 mm). Note that the first electrode 901 is a transparent electrode, and the transparent electrode and the reflective electrode can be collectively regarded as the first electrode 901.

Next, the first EL layer 903 was provided. First, in pretreatment for forming the light-emitting device 3A over the substrate, a surface of the substrate was washed with water and baking was performed at 200° C. for one hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for approximately 30 minutes.

Then, the substrate provided with the first electrode 901 was fixed to a substrate holder provided in the vacuum evaporation apparatus such that the surface on which the first electrode 901 was formed faced downward. Over the first electrode 901, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation using resistance heating to a thickness of 10 nm such that the weight ratio of PCBBiF to OCHD-003 was 1:0.03, whereby the hole-injection layer 910 was formed.

Next, PCBBiF was deposited by evaporation to a thickness of 125 nm over the hole-injection layer 910, so that the first hole-transport layer 911 was formed.

Next, the first light-emitting layer 912 was formed over the first hole-transport layer 911. The first light-emitting layer 912 was formed by depositing 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-3,3′-bi-9H-carbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) by co-evaporation using resistance heating to a thickness of 40 nm such that the weight ratio of 8mpTP-4mDBtPBfpm to βNCCP to Ir(5mppy-d₃)₂(mbfpypy-d₃) was 0.5:0.5:0.1.

Then, over the first light-emitting layer 912, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 10 nm, so that the first electron-transport layer 913 was formed.

Next, the intermediate layer 905 was provided. First, over the first electron-transport layer 913, 2,2′-([2,2′-bipyridine]-6,6′-diyl)bis(4-phenylbenzo[h]quinazoline) (abbreviation: 6,6′(P-Bqn)₂BPy), Prd3-PyA2Py, and lithium oxide (Li₂O) were deposited by co-evaporation using resistance heating to a thickness of 5 nm such that the volume ratio of 6,6′(P-Bqn)₂BPy to Prd3-PyA2Py to Li₂O was 0.5:0.5:0.02, whereby a layer serving as the electron-injection buffer region 914 was formed.

Then, as the electron-relay region, a film of copper phthalocyanine (abbreviation: CuPc) was formed to have a thickness of 2 nm. Next, as the charge-generation region, PCBBiF and a fluorine-containing electron acceptor material with a molecular weight of 672 (OCHD-003) were deposited by co-evaporation using resistance heating to a thickness of 10 nm such that the weight ratio of PCBBiF to OCHD-003 was 1:0.15, whereby the layer 915 including the electron-relay region and the charge-generation region was formed.

Next, the second EL layer 904 was provided. First, PCBBiF was deposited by evaporation to a thickness of 50 nm, so that the second hole-transport layer 916 was formed.

Next, 8mpTP-4mDBtPBfpm, βNCCP, and Ir(5mppy-d₃)₂(mbfpypy-d₃) were deposited by co-evaporation using resistance heating to a thickness of 40 nm such that the weight ratio of 8mpTP-4mDBtPBfpm to βNCCP to Ir(5mppy-d₃)₂(mbfpypy-d₃) was 0.5:0.5:0.1, whereby the second light-emitting layer 917 was formed.

Next, over the second light-emitting layer 917, 2mPCCzPDBq was deposited by evaporation to a thickness of 20 nm, and then 2,2′-(1,3-phenylene)bis(9-phenyl-1,10-phenanthroline) (abbreviation: mPPhen2P) was deposited by evaporation to a thickness of 20 nm, so that the second electron-transport layer 918 was formed.

Here, the substrate 900 was exposed to the air. Then, an aluminum oxide (abbreviation: AlOₓ) film was formed to a thickness of 30 nm by an ALD method, and molybdenum (Mo) was deposited to a thickness of 50 nm by a sputtering method. After that, a resist was formed using a photoresist, and the molybdenum was processed into a predetermined shape by a lithography method. Specifically, a 3-μm-wide slit was formed to be 3.5 μm apart from an end portion of the first electrode 901.

Next, using the molybdenum as a mask, the stacked-layer structure formed of the aluminum oxide film, the first EL layer 903, the intermediate layer 905, the second hole-transport layer 916, the second light-emitting layer 917, and the second electron-transport layer 918 was processed into a predetermined shape. After that, the molybdenum was removed by a dry etching method, and then the aluminum oxide film was removed. The aluminum oxide film was removed by wet etching using an acidic chemical solution.

Then, heat treatment was performed at 100° C. for one hour in a vacuum where the internal pressure was reduced to approximately 1×10⁻⁴ Pa. The heat treatment can remove moisture or the like attached by the above-described processing, the exposure to the air, or the like.

Next, over the second electron-transport layer 918, lithium fluoride (LiF) and ytterbium (Yb) were deposited by co-evaporation to a thickness of 1.5 nm such that the volume ratio of LiF to Yb was 2:1, whereby the electron-injection layer 919 was formed.

Next, over the electron-injection layer 919, Ag and Mg were deposited by co-evaporation to a thickness of 15 nm such that the volume ratio of Ag to Mg was 1:0.1, whereby the second electrode 902 was formed. Note that the second electrode 902 is a semi-transmissive and semi-reflective electrode having functions of transmitting light and reflecting light.

Then, as the cap layer 909, 4,4′,4″-(benzene-1,3,5-triyl)tri(dibenzothiophene) (abbreviation: DBT3P-II) was deposited by evaporation to a thickness of 70 nm.

Through the above process, the light-emitting device 3A was fabricated. The structure of the light-emitting device 3A is shown in the table below.

TABLE 3

| | Thick- | |
|---|---|---|
| | ness | |
| | [nm] | Light-emitting device 3A |
| Cap layer | 70 | DBT3P-II |
| Second electrode | 15 | Ag:Mg (1:0.1) |
| Electron-injection | 1.5 | LiF:Yb (2:1) |
| layer | | |

Processing

| | | |
|---|---|---|
| Second electron- | 20 | mPPhen2P |
| transport layer | 20 | 2mPCCzPDBq |
| Second light- | 40 | 8mpTP-4mDBtPBfpm:βNCCP:Ir(5mppy- |
| emitting layer | | d₃)₂(mbfpypy-d₃) (0.5:0.5:0.1) |
| Second hole- | 50 | PCBBiF |
| transport layer | | |
| Charge-generation | 10 | PCBBiF:OCHD-003 (1:0.15) |
| region | | |
| Electron-relay | 2 | CuPc |
| region | | |
| Electron-injection | 5 | 6,6′(P-Bqn)₂BPy:Prd3-PyA2Py:Li₂O |
| buffer region | | (0.5:0.5:0.02) |
| First electron- | 10 | 2mPCCzPDBq |
| transport layer | | |
| First light-emitting | 40 | 8mpTP-4mDBtPBfpm:βNCCP:Ir(5mppy- |
| layer | | d₃)₂(mbfpypy-d₃) (0.5:0.5:0.1) |
| First hole- | 125 | PCBBiF |
| transport layer | | |
| Hole-injection | 10 | PCBBiF:OCHD-003 (1:0.03) |
| layer | | |
| First electrode | 50 | ITSO |
| | 100 | APC |

Here, the HOMO and LUMO levels of 6,6′(P-Bqn)₂BPy and Prd3-PyA2Py were calculated by cyclic voltammetry (CV) measurement. An electrochemical analyzer (ALS model 600A or 600C, manufactured by BAS Inc.) was used for the measurement. The solvent of the solution used in the measurement was dehydrated dimethylformamide (DMF). In the measurement, the potential of a working electrode with respect to a reference electrode was changed within an appropriate range, so that the oxidation peak potential and the reduction peak potential were obtained. A platinum electrode (PTE platinum electrode, manufactured by BAS Inc.) was used as the working electrode, another platinum electrode (Pt counter electrode for VC-3 (5 cm), manufactured by BAS Inc.) was used as an auxiliary electrode, and an Ag/Ag⁺ electrode (RE7 reference electrode for nonaqueous solvent, manufactured by BAS Inc.) was used as the reference electrode. The HOMO and LUMO levels of the compound were calculated from the estimated redox potential of the reference electrode of −4.94 eV and the obtained peak potentials. As a result, the reduction potential of Prd3-PyA2Py was not observed, revealing that the LUMO level thereof is higher than or equal to −2.0 eV and the HOMO level thereof is −5.4 eV. The LUMO level of 6,6′(P-Bqn)₂BPy is −2.92 eV, and the oxidation potential thereof was not observed, revealing that the HOMO level thereof is lower than or equal to −6.0 eV. It was thus found that 6,6′(P-Bqn)₂BPy has a lower LUMO level than Prd3-PyA2Py.

### Device Characteristics

The light-emitting device 3A was sealed using a glass substrate in a glove box containing a nitrogen atmosphere so as not to be exposed to the air (a sealing material was applied to surround the device and UV treatment and heat treatment at 80° C. for one hour were performed at the time of sealing). Then, the emission characteristics of the light-emitting device 3A were measured.

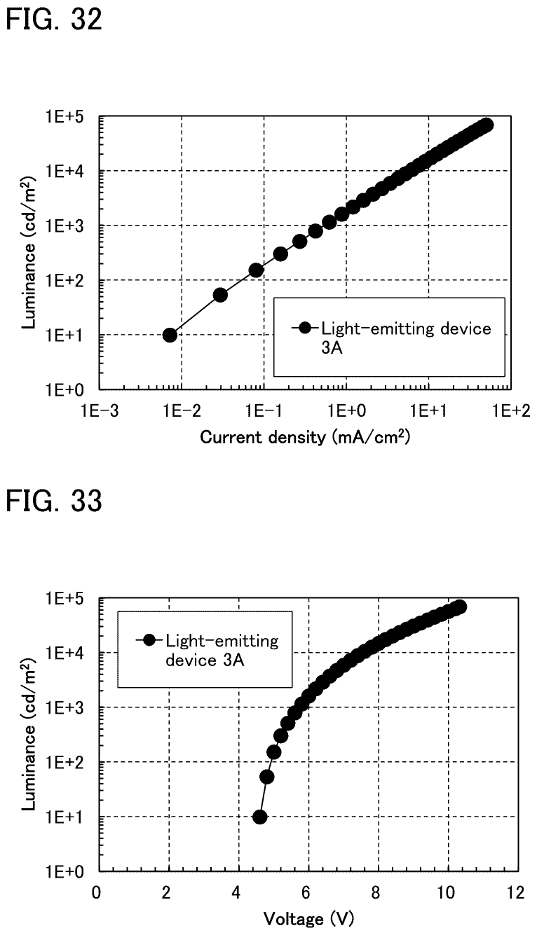

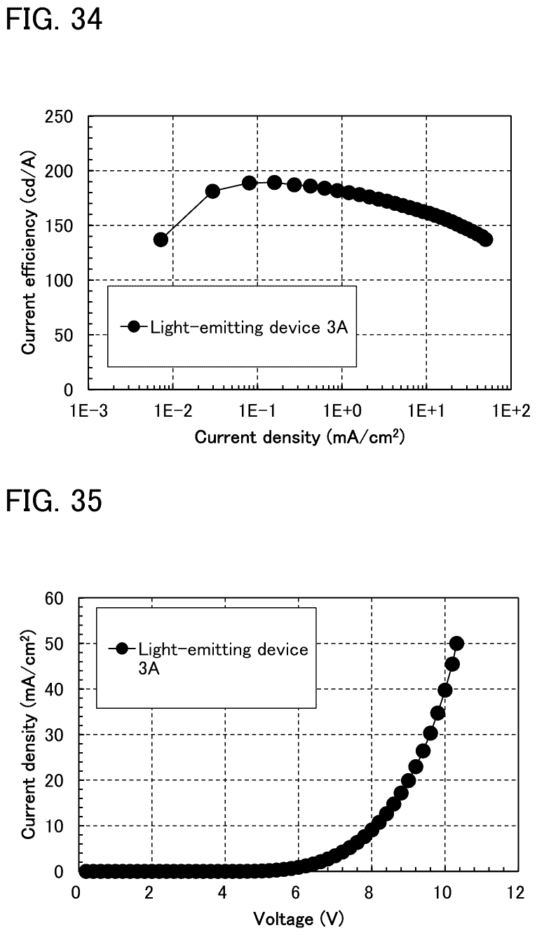

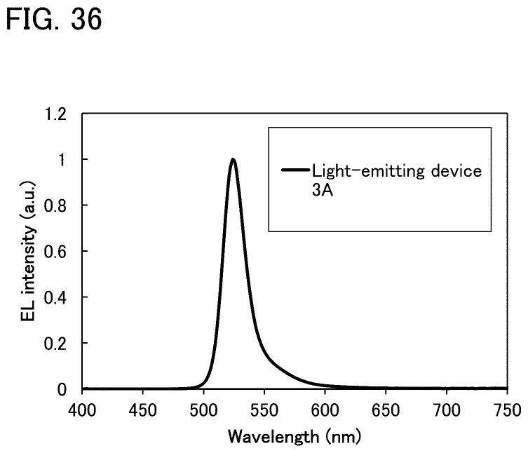

FIG. 32 shows the luminance-current density characteristics of the light-emitting device 3A. FIG. 33 shows the luminance-voltage characteristics thereof. FIG. 34 shows the current efficiency-current density characteristics thereof. FIG. 35 shows the current density-voltage characteristics thereof. FIG. 36 shows the electroluminescence spectrum thereof. The main characteristics of the light-emitting device 3A at a luminance of approximately 1000 cd/m² are shown in the table below. The luminance, CIE chromaticity, and electroluminescence spectrum were measured with a spectroradiometer (SR-UL1R produced by TOPCON TECHNOHOUSE CORPORATION).

TABLE 4

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Light-emitting | 5.8 | 0.0249 | 0.623 | 0.20 | 0.75 | 1147 | 184 |
| device 3A | | | | | | | |

As shown in FIG. 36, the light-emitting device 3A emitted green light with a peak wavelength of 524 nm in its electroluminescence spectrum. FIG. 32 to FIG. 35 and the above table show that the light-emitting device 3A has high current efficiency and is driven as a tandem light-emitting device even after being subjected to the process involving exposure to oxygen, water, and the chemical solution.

### Reliability Test Result

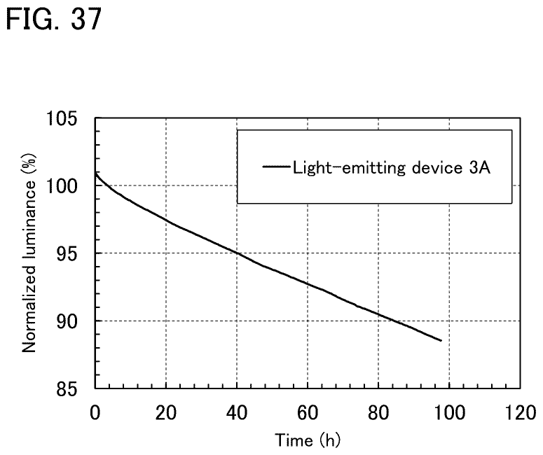

Moreover, a reliability test was conducted on the light-emitting device 3A. FIG. 37 shows a time-dependent change in normalized luminance during driving at a current density of 50 mA/cm². In FIG. 37, the vertical axis represents the luminance (%) normalized with the luminance at the time of the start of light emission being regarded as 100%, and the horizontal axis represents time (h).

In FIG. 37, LT95 (h), which is the time required for the measured luminance to decrease to 95% of the initial luminance, of the light-emitting device 3A is approximately 40 hours, showing that the light-emitting device 3A has high reliability.

It was found from the above that a tandem light-emitting device with a low driving voltage and high emission efficiency can be provided by using one embodiment of the present invention.

### Example 4

### Synthesis Example 2

In this example, physical properties of an organic compound of one embodiment of the present invention and a method for synthesizing the organic compound are described. Specifically, a method for synthesizing 2,2′-(pyridine-2,6-diyl)bis{4,6-bis[4-(1-pyrrolidinyl)phenyl]pyrimidine}(abbreviation: (PrdP2Pm)2Py), which is represented by Structural Formula (101) in Embodiment 1, is described. The structure of (PrdP2Pm)2Py is shown below.

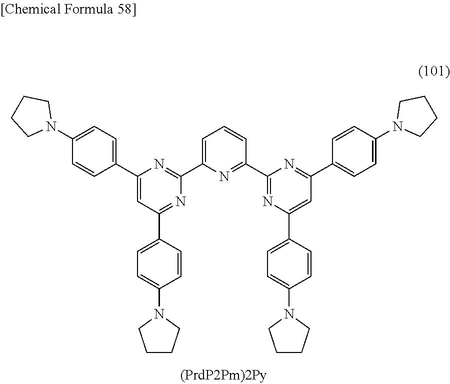 [SMILES: c1cc(-c2nc(-c3ccc(N4CCCC4)cc3)cc(-c3ccc(N4CCCC4)cc3)n2)nc(-c2nc(-c3ccc(N4CCCC4)cc3)cc(-c3ccc(N4CCCC4)cc3)n2)c1]

### Step 1: Synthesis of 2,2′-(pyridine-2,6-diyl)bis[4,6-bis(4-chlorophenyl)pyrimidine]

Into a 200-mL three-neck flask were put 4.7 g (20 mmol) of 2,6-pyridinedicarboxamidine dihydrochloride and 12 g (43 mmol) of 4,4′-dichlorochalcone, and the air in the flask was replaced with nitrogen. To this mixture was added 100 mL of ethanol. To this mixture was added 8.0 g (200 mmol) of sodium hydroxide. The resulting mixture was stirred at 80° C. under a nitrogen stream for 12 hours.

After the stirring, dichloromethane and water were added to this mixture; the resulting mixture was separated by suction filtration to give 6.3 g of a yellowish white solid of the target substance in a yield of 47%. The synthesis scheme of Step 1 is shown in (b-1) below.

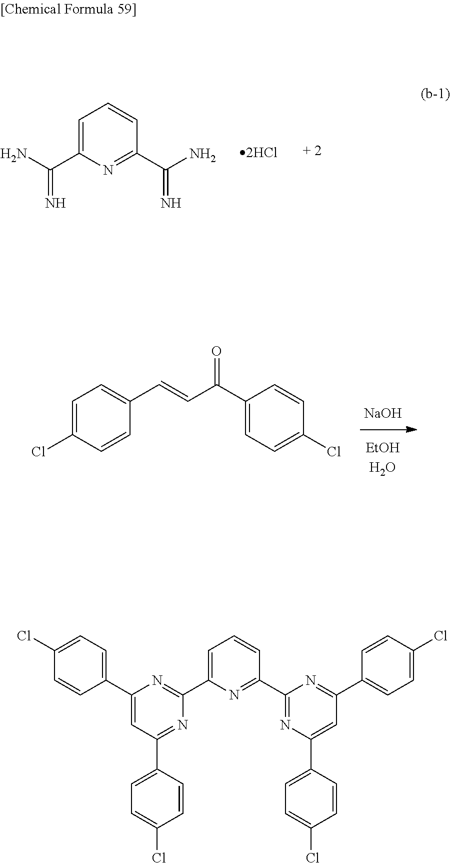 [SMILES: N=C(N)c1cccc(C(=N)N)n1 ; O=C(/C=C/c1ccc(Cl)cc1)c1ccc(Cl)cc1 ; "NaOH" ; Clc1ccc(-c2cc(-c3ccc(Cl)cc3)nc(-c3cccc(-c4nc(-c5ccc(Cl)cc5)cc(-c5ccc(Cl)cc5)n4)n3)n2)cc1]

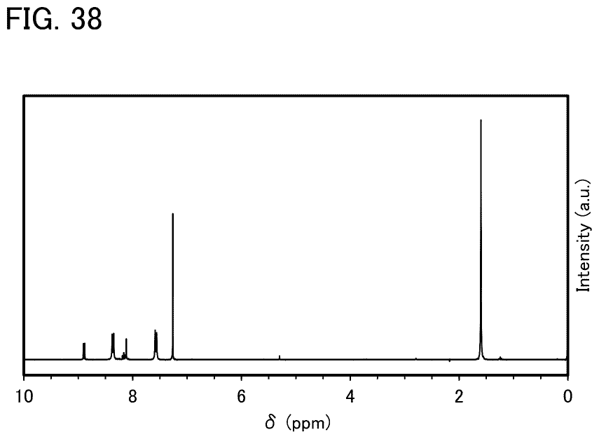

FIG. 38 shows the ¹H NMR spectrum of a deuterochloroform (abbreviation: CDCl₃) solution of the obtained yellowish white solid. The results of ¹H NMR measurement of the yellowish white solid are shown below. The results show that 2,2′-(pyridine-2,6-diyl)bis[4,6-bis(4-chlorophenyl)pyrimidine]was obtained.

¹H NMR (CDCl₃, 300 MHz): δ=8.91 (d, J=7.5 Hz, 2H), 8.38 (m, 8H), 8.19 (m, 3H), 7.59 (m, 8H).

### Step 2: Synthesis of (PrdP2Pm)2Py

Into a 200-mL three-neck flask were put 3.0 g (4.4 mmol) of 2,2′-(pyridine-2,6-diyl)bis[4,6-bis(4-chlorophenyl)pyrimidine] and 30 mL of 1,8-diazabicyclo[5.4.0]-7-undecene (abbreviation: DBU), and the air in the flask was replaced with nitrogen. To this mixture was added 3.2 mL (39 mmol) of pyrrolidine. The resulting mixture was stirred at 190° C. under a nitrogen stream for 24 hours.

After the stirring, toluene was added to this mixture; the resulting mixture was separated by suction filtration to give 1.7 g of a yellowish white solid of the target substance in a yield of 47%. The synthesis scheme of Step 2 is shown in (b-2) below.

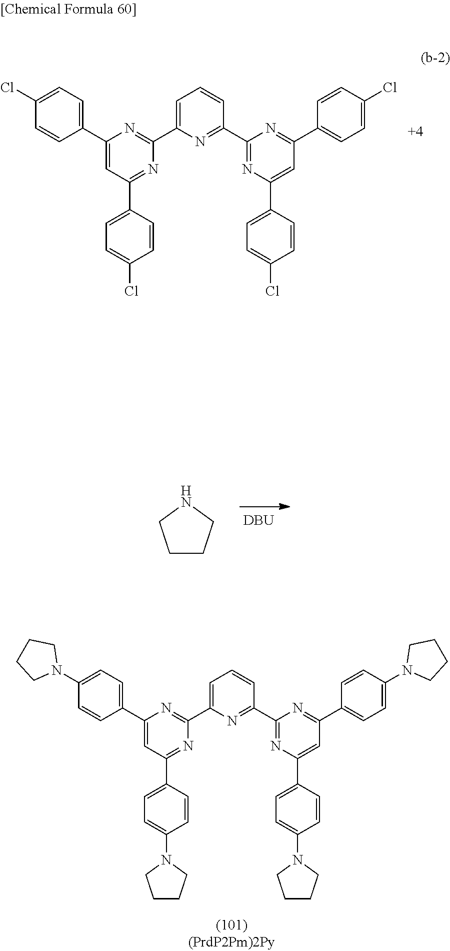

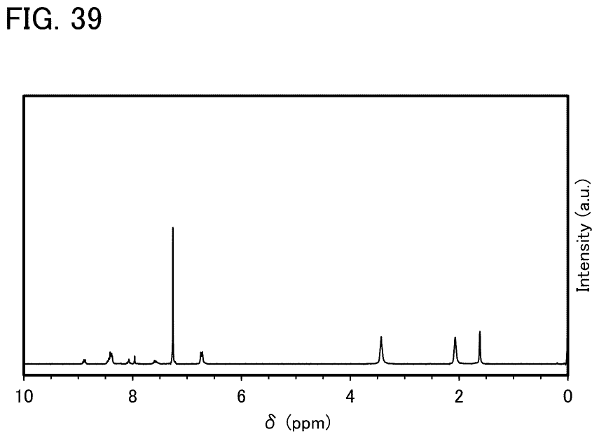

FIG. 39 shows the ¹H NMR spectrum of a deuterochloroform (abbreviation: CDCl₃) solution of the obtained yellowish white solid. The results of ¹H NMR measurement of the yellowish white solid are shown below. The results show that (PrdP2Pm)2Py was obtained.

¹H NMR (CDCl₃, 300 MHz): δ=8.91 (m, 2H), 8.44 (m, 8H), 8.07 (m, 3H), 6.75 (m, 8H), 3.43 (m, 16H), 2.07 (m, 16H).

### Example 5

### Synthesis Example 3

In this example, physical properties of an organic compound of one embodiment of the present invention and a method for synthesizing the organic compound are described. Specifically, a method for synthesizing 2,2′-[4-(1-pyrrolidinyl)pyridine-2,6-diyl]bis{4,6-bis[4-(1-pyrrolidinyl)phenyl]pyrimidine}(abbreviation: (PrdP2Pm)2PrdPy), which is represented by Structural Formula (102) in Embodiment 1, is described. The structure of (PrdP2Pm)2PrdPy is shown below.

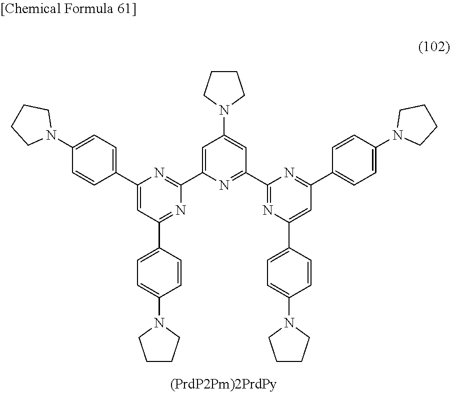 [SMILES: c1cc(N2CCCC2)ccc1-c1cc(-c2ccc(N3CCCC3)cc2)nc(-c2cc(N3CCCC3)cc(-c3nc(-c4ccc(N5CCCC5)cc4)cc(-c4ccc(N5CCCC5)cc4)n3)n2)n1]

### Step 1: Synthesis of 2,2′-(4-chloropyridine-2,6-diyl)bis[4,6-bis(4-chlorophenyl)pyrimidine]

Into a 500-mL three-neck flask were put 10 g (38 mmol) of 4-chloro-2,6-pyridinedicarboxamidine dihydrochloride and 25 g (91 mmol) of 4,4′-dichlorochalcone, and the air in the flask was replaced with nitrogen. To this mixture were added 190 mL of ethanol and 60 mL of water. To this mixture was added 15 g (380 mmol) of sodium hydroxide. The resulting mixture was stirred at 80° C. under a nitrogen stream for 11 hours.

After the stirring, dichloromethane and water were added to this mixture; the resulting mixture was separated by suction filtration to give 12 g of a white solid of the target substance in a yield of 44%. The synthesis scheme of Step 1 is shown in (c-1) below.

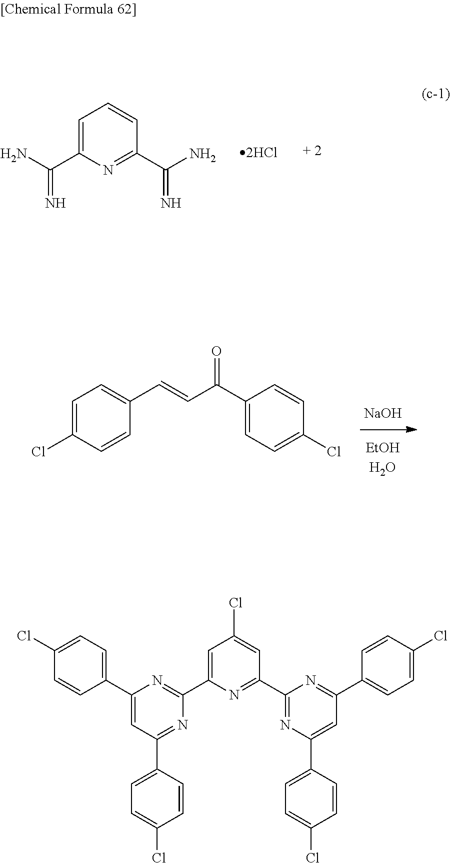 [SMILES: N=C(N)c1cccc(C(=N)N)n1 ; O=C(/C=C/c1ccc(Cl)cc1)c1ccc(Cl)cc1 ; "NaOH" ; Clc1ccc(-c2cc(-c3ccc(Cl)cc3)nc(-c3cc(Cl)cc(-c4nc(-c5ccc(Cl)cc5)cc(-c5ccc(Cl)cc5)n4)n3)n2)cc1]

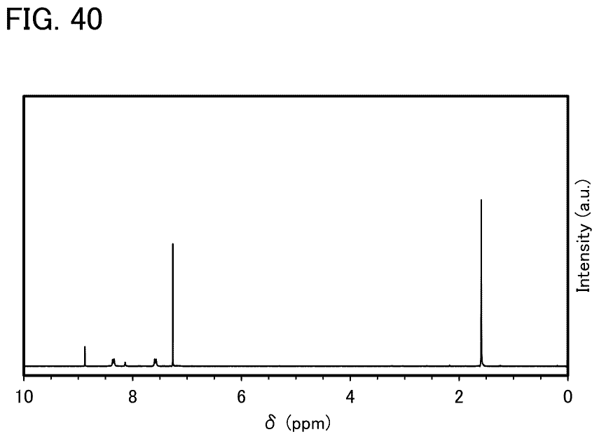

FIG. 40 shows the ¹H NMR spectrum of a deuterochloroform (abbreviation: CDCl₃) solution of the obtained white solid. The results of ¹H NMR measurement of the white solid are shown below. The results show that 2,2′-(4-chloropyridine-2,6-diyl)bis[4,6-bis(4-chlorophenyl)pyrimidine]was obtained.

¹H NMR (CDCl₃, 300 MHz): δ=8.88 (s, 2H), 8.37 (m, 8H), 8.14 (s, 2H), 7.60 (m, 8H).

### Step 2: Synthesis of (PrdP2Pm)2PrdPy

Into a 200-mL three-neck flask were put 3.0 g (4.2 mmol) of 2,2′-(4-chloropyridine-2,6-diyl)bis[4,6-bis(4-chlorophenyl)pyrimidine] and 15 mL of 1,8-diazabicyclo[5.4.0]-7-undecene (abbreviation: DBU), and the air in the flask was replaced with nitrogen. To this mixture was added 6.0 mL (73 mmol) of pyrrolidine. The resulting mixture was stirred at 180° C. under a nitrogen stream for 12 hours.

After the stirring, toluene was added to this mixture; the resulting mixture was separated by suction filtration to give 1.0 g of a yellowish white solid of the target substance in a yield of 28%. The synthesis scheme of Step 2 is shown in (c-2) below.

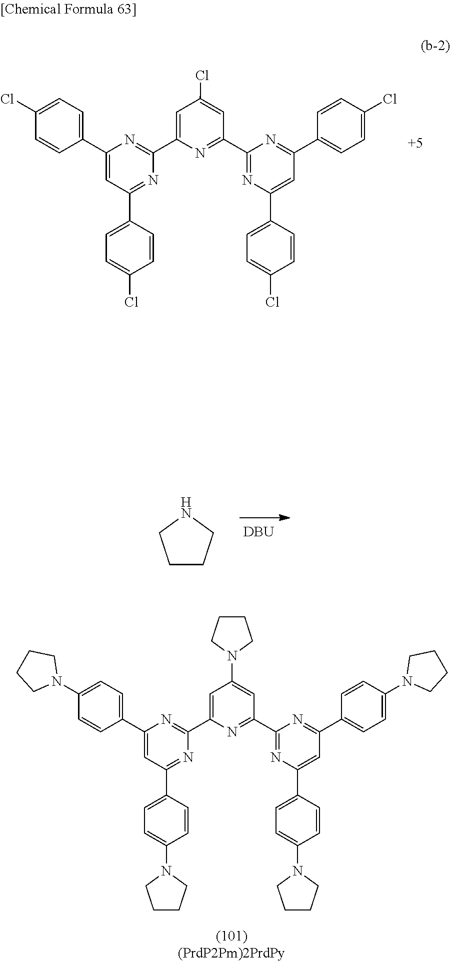

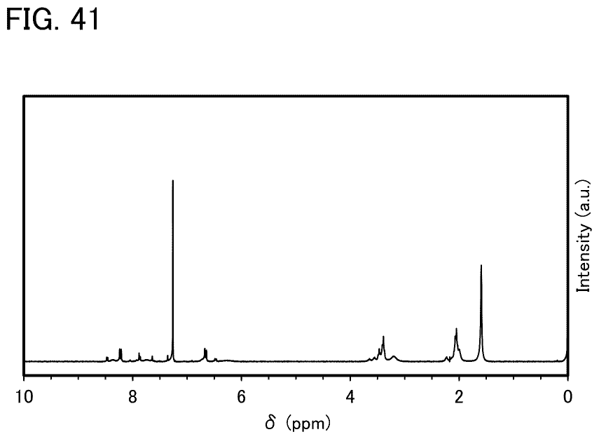

FIG. 41 shows the ¹H NMR spectrum of a deuterochloroform (abbreviation: CDCl₃) solution of the obtained yellowish white solid. The results of ¹H NMR measurement of the yellowish white solid are shown below. The results show that (PrdP2Pm)2PrdPy was obtained.

¹H NMR (CDCl₃, 300 MHz): δ=8.48 (m, 2H), 8.24 (m, 8H), 7.88 (m, 2H), 6.67 (m, 8H), 3.47 (m, 20H), 2.07 (m, 20H).

### Example 6

In this example, a light-emitting device 6A whose electron-transport layer includes N,N-bis-2-[4-(1-pyrrolidinyl)]pyridinyl-N,N′-diphenyl-[4-(1-pyrrolidinyl)]pyridine-2,6-diamine (abbreviation: Prd3-PyA2Py), an organic compound of one embodiment of the present invention represented by Structural Formula (100), was fabricated.

The structural formulae of organic compounds used in the light-emitting device 6A are shown below.

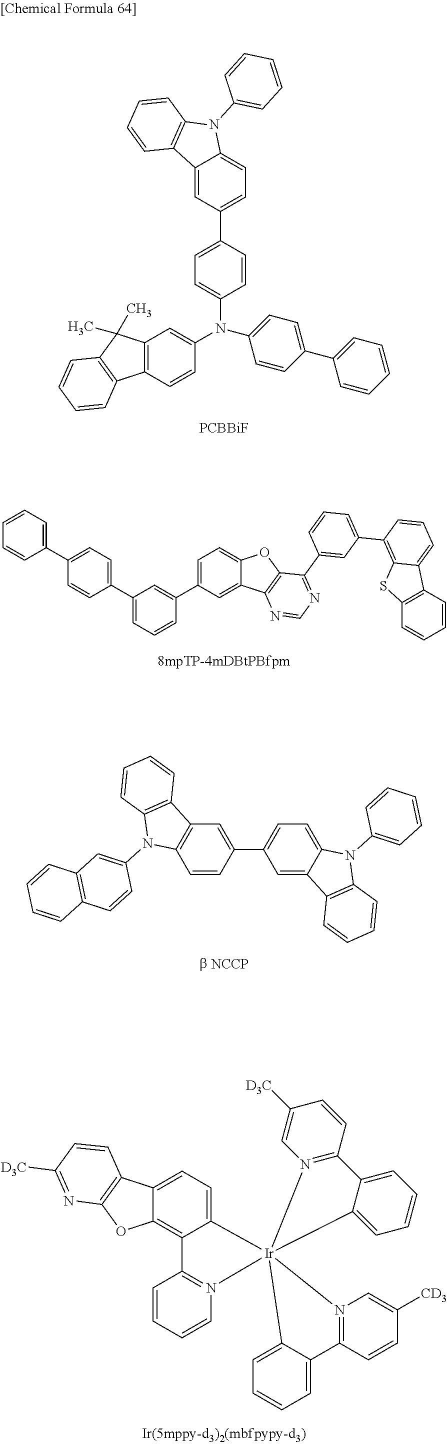

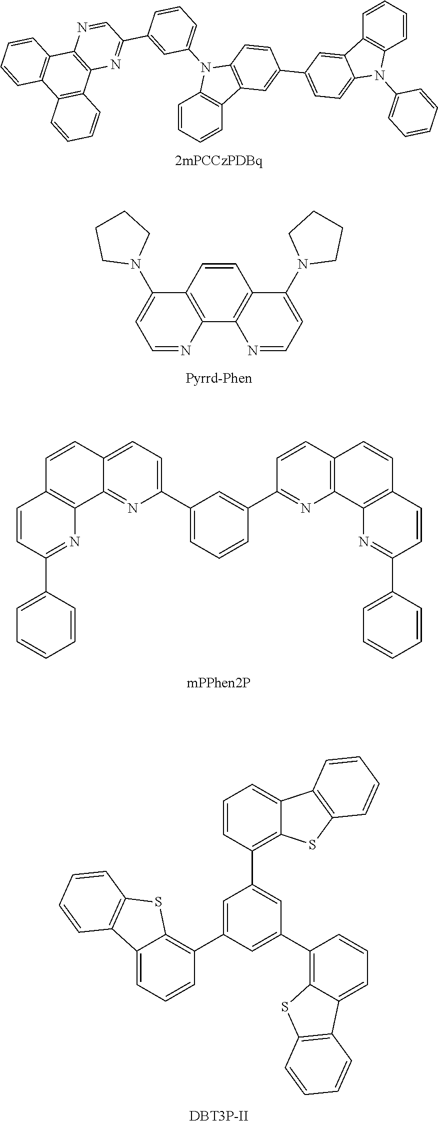 [SMILES: c1ccc(-n2c3ccccc3c3cc(-c4ccc5c(c4)c4ccccc4n5-c4cccc(-c5cnc6c7ccccc7c7ccccc7c6n5)c4)ccc32)cc1 ; c1cc(N2CCCC2)c2ccc3c(N4CCCC4)ccnc3c2n1 ; c1ccc(-c2ccc3ccc4ccc(-c5cccc(-c6ccc7ccc8ccc(-c9ccccc9)nc8c7n6)c5)nc4c3n2)cc1 ; c1ccc2c(c1)sc1c(-c3cc(-c4cccc5c4sc4ccccc45)cc(-c4cccc5c4sc4ccccc45)c3)cccc12 ; "DBT3P-II"]

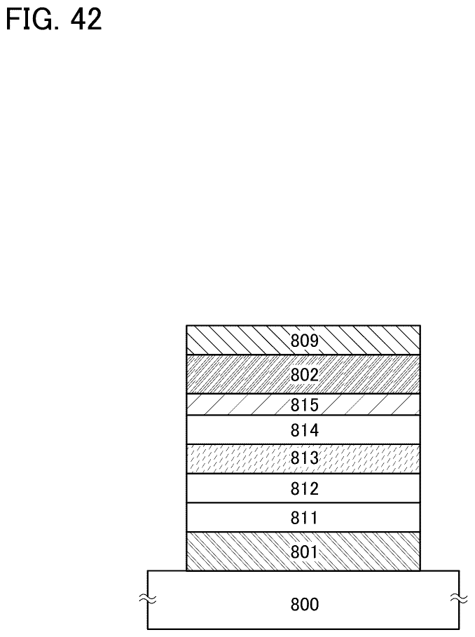

In the device, as illustrated in FIG. 42, a hole-injection layer 811, a hole-transport layer 812, a light-emitting layer 813, an electron-transport layer 814, and an electron-injection layer 815 are stacked in this order over a first electrode 801 formed over a glass substrate 800, and a second electrode 802 is stacked over the electron-injection layer 815.

### Method for Fabricating Light-Emitting Device 6A

As a reflective electrode, silver (Ag) was deposited over the glass substrate 800 to a thickness of 100 nm by a sputtering method, and then, as a transparent electrode, indium tin oxide containing silicon oxide (ITSO) was deposited to a thickness of 85 nm by a sputtering method, so that the first electrode 801 was formed. The electrode area was set to 4 mm² (2 mm×2 mm).

Next, in pretreatment for forming the light-emitting device 6A over the substrate, a surface of the substrate was washed with water and baking was performed at 200° C. for one hour. Then, the substrate was transferred into a vacuum evaporation apparatus where the pressure was reduced to approximately 1×10⁻⁴ Pa, and vacuum baking was performed at 170° C. for 30 minutes in a heating chamber of the vacuum evaporation apparatus. After that, natural cooling was performed for 30 minutes.

Then, the substrate provided with the first electrode 801 was fixed to a substrate holder provided in the vacuum evaporation apparatus such that the surface on which the first electrode 801 was formed faced downward. Over the first electrode 801, N-(biphenyl-4-yl)-N-[4-(9-phenyl-9H-carbazol-3-yl)phenyl]-9,9-dimethyl-9H-fluoren-2-amine (abbreviation: PCBBiF) and a fluorine-containing electron acceptor material with a molecular weight of 672 (OCHD-003) were deposited by co-evaporation to a thickness of 10 nm such that the weight ratio of PCBBiF to OCHD-003 was 1:0.03, whereby the hole-injection layer 811 was formed.

Next, over the hole-injection layer 811, PCBBiF was deposited by evaporation using resistance heating to a thickness of 60 nm, whereby the hole-transport layer 812 was formed.

Next, the light-emitting layer 813 was formed over the hole-transport layer 812 by depositing 8-(p-terphenyl-3-yl)-4-[3-(dibenzothiophen-4-yl)phenyl]-[1]benzofuro[3,2-d]pyrimidine (abbreviation: 8mpTP-4mDBtPBfpm), 9-(2-naphthyl)-9′-phenyl-3,3′-bi-9H-carbazole (abbreviation: βNCCP), and [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)) by co-evaporation using resistance heating to a thickness of 40 nm such that the weight ratio of 8mpTP-4mDBtPBfpm to βNCCP to Ir(5mppy-d₃)₂(mbfpypy-d₃) was 0.5:0.5:0.1.

Then, over the light-emitting layer 813, 2-{3-[3-(N-phenyl-9H-carbazol-3-yl)-9H-carbazol-9-yl]phenyl}dibenzo[f,h]quinoxaline (abbreviation: 2mPCCzPDBq) was deposited by evaporation to a thickness of 35 nm, and then, Prd3-PyA2Py was deposited by evaporation to a thickness of 5 nm, so that the electron-transport layer 814 was formed.

Next, over the electron-transport layer 814, lithium fluoride (LiF) and ytterbium (Yb) were deposited by co-evaporation to a thickness of 1.5 nm such that the volume ratio of LiF to Yb was 2:1, whereby the electron-injection layer 815 was formed.

Next, over the electron-injection layer 815, Ag and Mg were deposited by co-evaporation to a thickness of 15 nm such that the volume ratio of Ag to Mg was 1:0.1, whereby the second electrode 802 was formed. Note that the second electrode 802 is a semi-transmissive and semi-reflective electrode having functions of transmitting light and reflecting light.

Then, as a cap layer 809, 4,4,4″-(benzene-1,3,5-triyl)tri(dibenzothiophene) (abbreviation: DBT3P-JJ) was deposited by evaporation to a thickness of 70 nm over the second electrode 802.

The structure of the light-emitting device 6A is shown in the table below.

TABLE 5

| | Thick- | |
|---|---|---|
| | ness | |
| | [nm] | Light-emitting device 6A |
| Cap layer | 70 | DBT3P-II |
| Second electrode | 15 | Ag:Mg (1:0.1) |
| Electron-injection | 1.5 | LiF:Yb (1:0.5) |
| layer | | |
| Electron-transport | 5 | Prd3-PyA2Py |
| layer | 35 | 2mPCCzPDBq |
| Light-emitting | 40 | 8mpTP-4mDBtPBfpm:βNCCP:Ir(5mppy- |
| layer | | d₃)₂(mbfpypy-d₃) (0.5:0.5:0.1) |
| Hole-transport | 60 | PCBBiF |
| layer | | |
| Hole-injection | 10 | PCBBiF:OCHD-003 (1:0.03) |
| layer | | |
| First electrode | 85 | ITSO |
| | 100 | Ag |

### Device Characteristics

The light-emitting device 6A was sealed using a glass substrate in a glove box containing a nitrogen atmosphere so as not to be exposed to the air (a sealing material was applied to surround the device and UV treatment and heat treatment at 80° C. for one hour were performed at the time of sealing). Then, the emission characteristics of the light-emitting device 6A were measured.

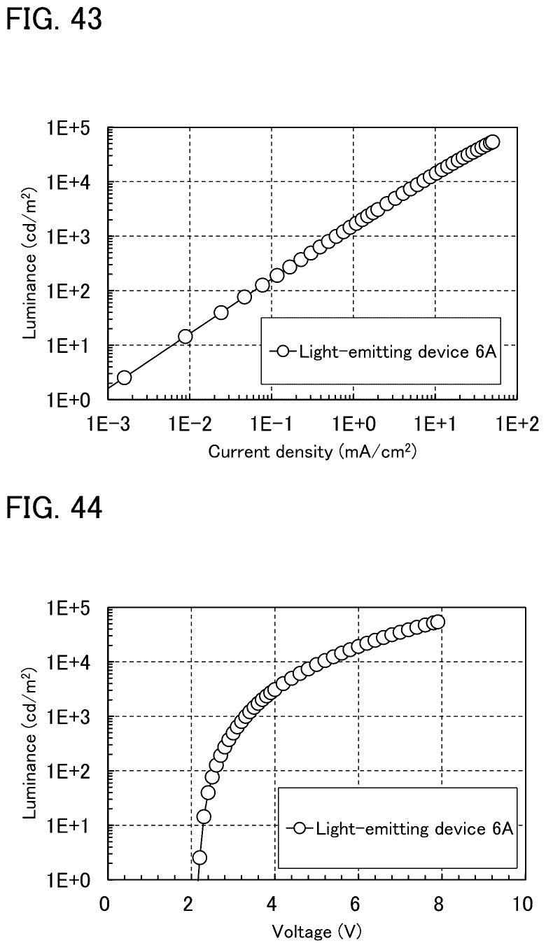

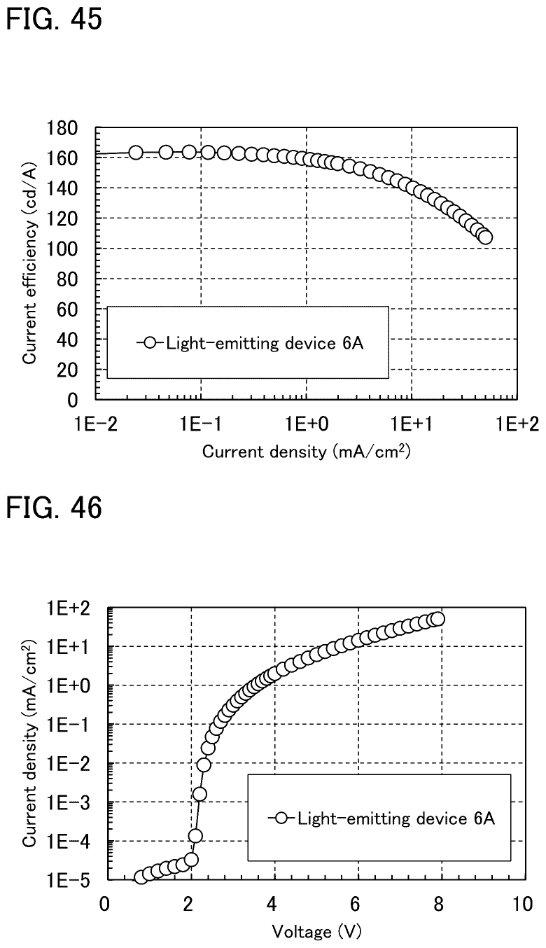

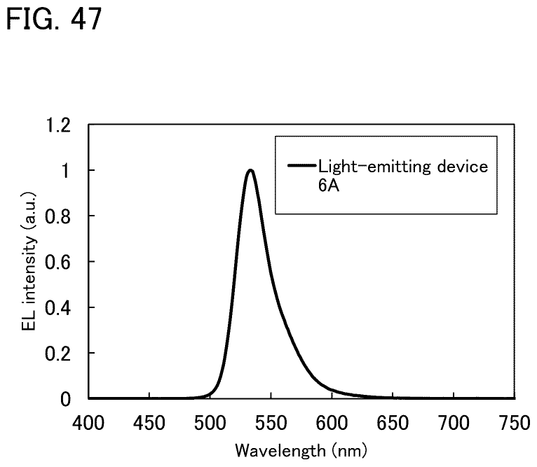

FIG. 43 shows the luminance-current density characteristics of the light-emitting device 6A. FIG. 44 shows the luminance-voltage characteristics thereof. FIG. 45 shows the current efficiency-current density characteristics thereof. FIG. 46 shows the current density-voltage characteristics thereof. FIG. 47 shows the electroluminescence spectrum thereof.

The main characteristics of the light-emitting device 6A at a luminance of approximately 1000 cd/m² are shown in the table below. The luminance, CIE chromaticity, and electroluminescence spectrum were measured with a spectroradiometer (SR-UL1R produced by TOPCON TECHNOHOUSE CORPORATION).

TABLE 6

| | | | Current | | | | Current |
|---|---|---|---|---|---|---|---|
| | Voltage | Current | density | Chromaticity | Chromaticity | Luminance | efficiency |
| | (V) | (mA) | (mA/cm²) | x | y | (cd/m²) | (cd/A) |
| Light-emitting | 3.30 | 0.0247 | 0.617 | 0.273 | 0.699 | 993 | 161 |
| device 6A | | | | | | | |

The above table and FIG. 43 to FIG. 47 show that the light-emitting device 6A is driven at a low voltage and have high efficiency to emit green light with high color purity.

From the above, the organic compound of one embodiment of the present invention was found to be suitable for an electron-transport layer of a light-emitting device.

This application is based on Japanese Patent Application Serial No. 2024-148422 filed with Japan Patent Office on Aug. 30, 2024, the entire contents of which are hereby incorporated by reference.

What is claimed is:

1. An organic compound represented by General Formula (G1-1): 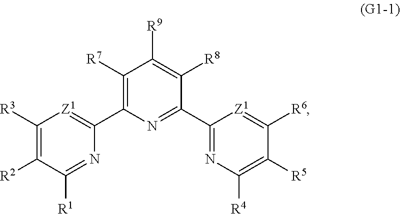
wherein Z¹ represents carbon or nitrogen,
wherein each of R¹ to R⁹ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1),
wherein at least one of R¹ to R³ and at least one of R⁴ to R⁶ each represent a group represented by General Formula (g2), 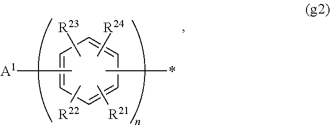
wherein A¹ represents the aliphatic amine group represented by General Formula (g1),
wherein each of R²¹ to R²⁴ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1),
wherein n represents an integer of 0 to 2, 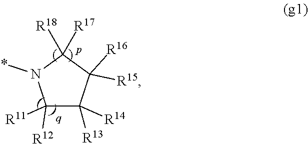
wherein each of R¹¹ to R¹⁸ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein each of p and q independently represents an integer of 0 to 3,
wherein any two of R¹¹ to R¹⁸ bonded to different carbon atoms are bonded to each other to form a ring or not bonded to each other, and
wherein an asterisk (*) represents a bond in the organic compound represented by General Formula (G1-1).

2. An organic compound represented by General Formula (G1-2): 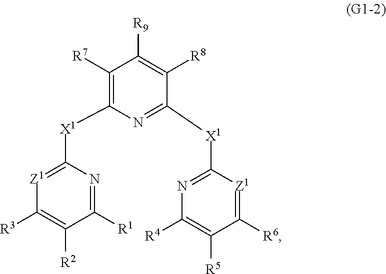
wherein X¹ represents oxygen, sulfur, or nitrogen,
wherein when X¹ represents nitrogen, a substituted or unsubstituted aryl group having 6 to 30 carbon atoms or a substituted or unsubstituted heteroaryl group having 1 to 30 carbon atoms is bonded to the nitrogen,
wherein Z¹ represents carbon or nitrogen,
wherein each of R¹ to R⁹ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1),
wherein at least one of R¹ to R³ and at least one of R⁴ to R⁶ each represent a group represented by General Formula (g2), 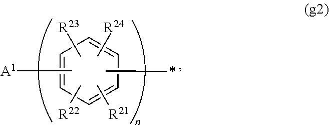
wherein A¹ represents the aliphatic amine group represented by General Formula (g1),
wherein each of R²¹ to R²⁴ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1),
wherein n represents an integer of 0 to 2, 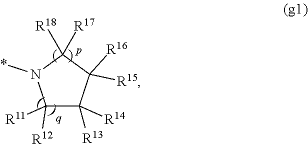
wherein each of R¹¹ to R¹⁸ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein each of p and q independently represents an integer of 0 to 3,
wherein any two of R¹¹ to R¹⁸ bonded to different carbon atoms are bonded to each other to form a ring or not bonded to each other, and
wherein an asterisk (*) represents a bond in the organic compound represented by General Formula (G1-2).

3. The organic compound according to claim 2, wherein the organic compound is represented by General Formula (G1-3): 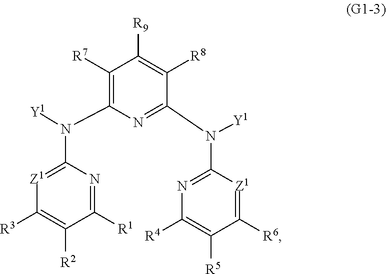
wherein Y¹ represents a substituted or unsubstituted aryl group having 6 to 30 carbon atoms or a substituted or unsubstituted heteroaryl group having 1 to 30 carbon atoms.

4. The organic compound according to claim 1, wherein the organic compound is represented by General Formula (G2-1):

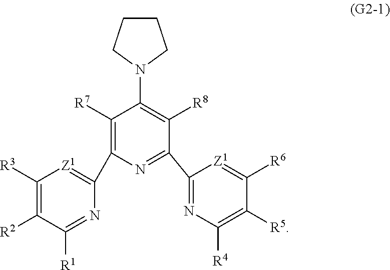

5. The organic compound according to claim 2, wherein the organic compound is represented by General Formula (G2-2):

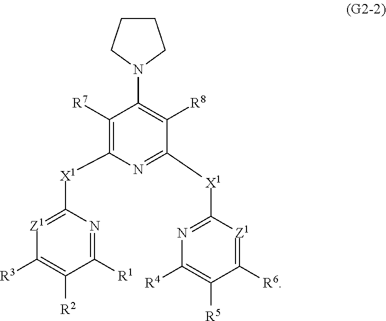

6. The organic compound according to claim 3, wherein the organic compound is represented by General Formula (G2-3):

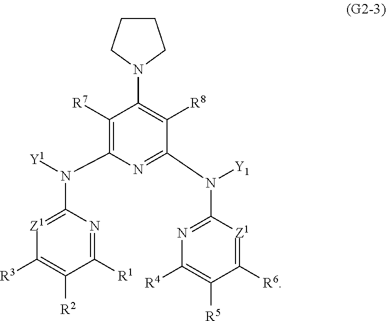

7. An organic compound represented by General Formula (G3): 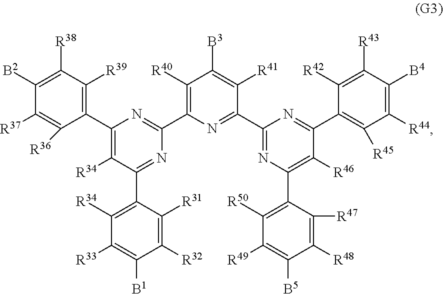
wherein each of B¹ to B⁵ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1),
wherein at least one of B¹ to B⁵ represents the aliphatic amine group represented by General Formula (g1),
wherein each of R³¹ to R⁵⁰ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1), 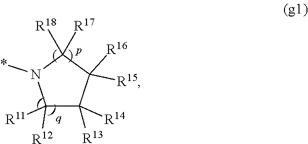
wherein each of R¹¹ to R¹⁸ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein each of p and q independently represents an integer of 0 to 3,
wherein any two of R¹¹ to R¹⁸ bonded to different carbon atoms are bonded to each other to form a ring or not bonded to each other, and
wherein an asterisk (*) represents a bond in the organic compound represented by General Formula (G3).

8. The organic compound according to claim 3, wherein the organic compound is represented by General Formula (G4): 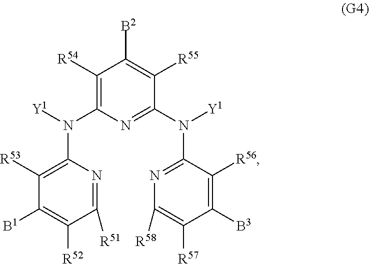
wherein Y¹ represents a substituted or unsubstituted aryl group having 6 to 30 carbon atoms or a substituted or unsubstituted heteroaryl group having 1 to 30 carbon atoms,
wherein each of B¹ to B³ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and an aliphatic amine group represented by General Formula (g1),
wherein at least one of B¹ to B³ represents the aliphatic amine group represented by General Formula (g1),
wherein each of R⁵¹ to R⁵⁸ independently represents any one of hydrogen, an alkyl group having 1 to 10 carbon atoms, an alkoxy group having 1 to 10 carbon atoms, a silyl group having 1 to 20 carbon atoms, a substituted or unsubstituted aryl group having 6 to 30 carbon atoms, a substituted or unsubstituted heteroaryl group having 1 to 30 carbon atoms, a cyano group, and the aliphatic amine group represented by General Formula (g1), 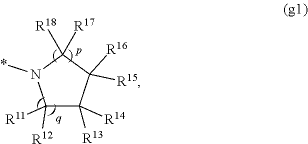
wherein each of R¹¹ to R¹⁸ independently represents any one of hydrogen, an alkyl group having 1 to 10 carbon atoms, a cycloalkyl group having 3 to 10 carbon atoms, an alkoxy group having 1 to 10 carbon atoms, a substituted or unsubstituted secondary amino group having 2 to 10 carbon atoms, a substituted or unsubstituted monovalent aromatic hydrocarbon group having 6 to 30 carbon atoms, a substituted or unsubstituted heteroaryl group having 1 to 30 carbon atoms, a cyano group, a halogen, a hydroxy group, an amide group, and a carbonyl group,
wherein each of p and q independently represents an integer of 0 to 3,
wherein any two of R¹¹ to R¹⁸ bonded to different carbon atoms are bonded to each other to form a ring or not bonded to each other, and
wherein an asterisk (*) represents a bond in the organic compound represented by General Formula (G4).

9. The organic compound according to claim 8, wherein the organic compound is represented by General Formula (G5):

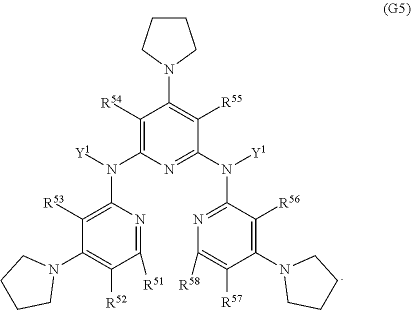

10. The organic compound according to claim 6, wherein the organic compound is represented by Structural Formula (100):

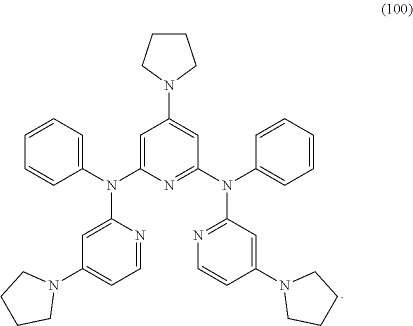 [SMILES: c1ccc(N(c2cc(N3CCCC3)ccn2)c2cc(N3CCCC3)cc(N(c3ccccc3)c3cc(N4CCCC4)ccn3)n2)cc1]

11. The organic compound according to claim 7, wherein the organic compound is represented by Structural Formula (101) or (102):

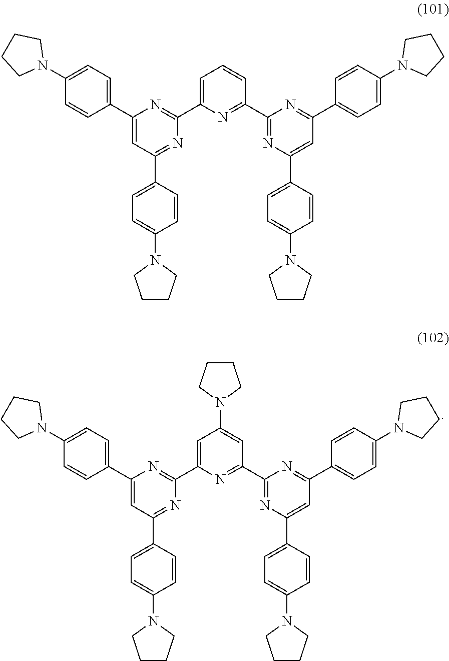 [SMILES: c1cc(-c2nc(-c3ccc(N4CCCC4)cc3)cc(-c3ccc(N4CCCC4)cc3)n2)nc(-c2nc(-c3ccc(N4CCCC4)cc3)cc(-c3ccc(N4CCCC4)cc3)n2)c1 ; c1cc(N2CCCC2)ccc1-c1cc(-c2ccc(N3CCCC3)cc2)nc(-c2cc(N3CCCC3)cc(-c3nc(-c4ccc(N5CCCC5)cc4)cc(-c4ccc(N5CCCC5)cc4)n3)n2)n1]

12. A light-emitting device comprising the organic compound according to claim 1.

13. A light-emitting device comprising the organic compound according to claim 2.

14. A light-emitting device comprising the organic compound according to claim 7.
